(19) United States
(12) Patent Application Publication
(10) Pub. No.: US 2017/0248616 A1
(43) Pub. Date: Aug. 31, 2017

(54) IN VITRO COMPOSITIONS COMPRISING HUMAN SAMPLE AND AMYLOID TARGETING AGENT

(71) Applicant: Amydis Diagnostics, Inc., San Diego, CA (US)

(72) Inventor: Stella Sarraf, Beverley Hills, CA (US)

(21) Appl. No.: 15/508,659

(22) PCT Filed: Sep. 11, 2015
(86) PCT No.: PCT/US2015/049825

§ 371 Date: Mar. 3, 2017

Related U.S. Application Data
(60) Provisional application No. 62/049,948, filed on Sep. 12, 2014.

(51) Int. Cl. G01N 33/68; G01N 33/58

(52) U.S. Cl. CPC G01N 33/6896; G01N 33/582; G01N 2333/4709; G01N 2800/2821

(57) ABSTRACT

Provided herein are compositions and methods useful for detection of amyloid related disorders in samples, such as human tissue, cell or body fluid. Use of the compositions and methods herein allows for the rapid, in vitro detection of amyloid accumulation, often before amyloid disorder symptoms are manifest or without introduction of foreign fluorophore molecules into a subject.

### CONTINUITY INFORMATION

The present application claims priority to U.S. Provisional Application Ser. No. 62/049,948, filed Sep. 12, 2014, which is hereby incorporated by reference in its entirety.

### BACKGROUND OF THE INVENTION

Amyloid accumulation is the hallmark of a large and growing number of disorders. Amyloid formation has been implicated in a number of disorders including Alzheimer's disease (AD), Down's syndrome, Parkinson disease, Creutzfeldt-Jakob disease (CJD), Huntington's disease, Rheumatoid Arthritis, Familial amyloid polyneuropathy, Hereditary non-neuropathic systemic amyloidosis, Diabetes mellitus type 2, Systemic AL amyloidosis, and is also implicated in sexual transmission of viruses such as HIV, human conception success rates, and bacterial homeostasis.

A common issue with many amyloid disorders is that they are difficult to detect until symptoms of irreparable damage manifest themselves. Early detection, prior to the presentation of amyloid disorder symptoms, may enable the medical community to slow or prevent the progression of disease symptoms, which may substantially improve the quality of life of individuals identified thereby.

Approaches to clinically diagnose and monitor the progression of these diseases include targeting of amyloid deposits with small-molecule imaging agents. Accordingly, fluorescence-based small molecule imaging of amyloids is a low cost, accessible, and non-radioactive technique for to detection of the amyloid deposits. Although there are several classes of compounds that fluoresce upon binding to amyloids a majority of these compounds are not suitable in vivo due to their poor stability or biocompatibility, or due to the inaccessibility of the known sites of amyloid accumulation.

### SUMMARY OF THE INVENTION

The disclosure relates to compositions and methods involved in the detection of amyloid complexes in samples, such as human samples. In some cases the detection of amyloid complexes in samples, such as human samples, relates to the presence or the likelihood of future presence of an amyloid disorder or the manifestation of symptoms of an amyloid disorder in an individual from which a sample is provided.

In some aspects, the disclosure provides a sample, such as a human sample, in contact with a compound used in the detection of amyloid in the sample. In some cases the sample is drawn, collected, obtained, or originates from a human, such as a human at risk of an amyloid disorder, a human suspected of suffering from an amyloid disorder, a human with a familial history of an amyloid disorder, or a human undergoing a general medical health survey.

A broad range of samples are consistent with the compositions and methods disclosed herein. In some cases, the sample comprises at least one of a tissue, a cell such as a leukocyte, monocyte, or peripheral blood leukocyte (PBL), a bodily fluid such as urine, blood, serum, plasma, lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, gastric acid, or a tumor or cancerous tissue, tumor or cancerous cell, a fluid from a tumor or cancerous tissue or cell, or an extract derived from any of the aforementioned tissues, cells or fluids.

In some aspects the sample comprises an amyloid. In some aspects the sample comprises at least one of an amyloid beta, an alpha synuclein, a prion, huntingtin (HTT), serum amyloid A (SAA), transthyretin (ATTR), lysozyme (ALys), amylin (AIPP), immunoglobulin light chain (AL), semen derived enhancer of viral infection (SEVI), PAP₈₅₋₁₂₀, SEM1₄₉₋₁₀₇, protegrin-1, and Curli (CsgA-R5 or CsgA-R1).

In some aspects a sample such as a sample of the types disclosed herein is provided in a composition comprising a compound of Formula I, or a pharmaceutically acceptable salt thereof:

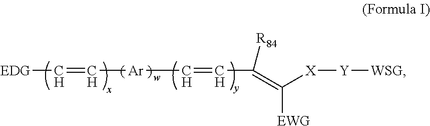

wherein:
EDG is an electron donating group;
each Ar is independently C₁-C₁₄ arylene or C₁-C₁₄ heteroarylene, each optionally substituted with one more R₁;
each R₁ is independently halogen, —OR₂, —NR₃R₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R₅;
R₂, R₃ and R₄ are independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₅;
each R₅ is independently halogen, —OR₆, —NR₇R₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
R₆, R₇, R₈ and R₈₄ are independently hydrogen or C₁-C₁₀ alkyl;
EWG is an electron withdrawing group;
WSG is a water soluble group;
X is C═O or SO₂;
Y is NH, or S;
each x is independently an integer from 0-10;
each w is independently an integer from 1-5;
each y is independently an integer from 0-10; and
z is an integer from 1-10.

In some embodiments of a compound of Formula I, the substituent R₈₄ is hydrogen or C₁-C₁₀ alkyl. In some embodiments of a compound of Formula I, R₈₄ is hydrogen. In some embodiments of a compound of Formula I, R₈₄ is methyl.

In some embodiments of a compound of Formula I, the substituent EDG is any electron donating group. In some embodiments of a compound of Formula I, EDG is OR₉, NR₁₀R₁₁, —SR₁₂, —PR₁₃R₁₄, —NR₁₅C(O)R₁₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₁₇; wherein each R₁₇ is independently halogen, —OR₁₈, —NR₁₉R₂₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; each of R₉, R₁₀, R₁₁, R₁₂, R₁₃, R₁₄, R₁₅, R₁₆, R₁₈, R₁₉ and R₂₀ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₂₁ and wherein R₁₀ and R₁₁ are optionally joined together to form a heterocycloalkyl or heteroaryl optionally substituted with R₂₁; each of R₂₁ is independently halogen, —OR₂₂, —NR₂₃R₂₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₂₅; each of R₂₂, R₂₃ and R₂₄ is independently hydrogen or C₁-C₁₀ alkyl; and each R₂₅ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula I, the EDG is selected from a group consisting of

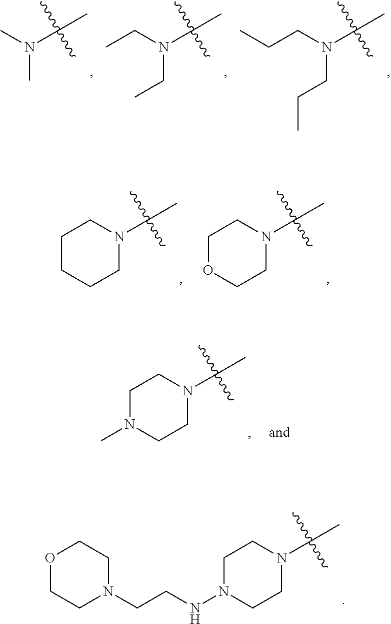

In some embodiments of a compound of Formula I, the EDG is

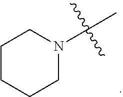 [SMILES: *N1CCCCC1]

In some embodiments of a compound of Formula I, the substituent EWG in Formula I is any electron withdrawing group. In some embodiments of a compound of Formula I, EWG is halogen, —CN, —NO₂, —SO₃H, —CR₂₆R₂₇R₂₈, COR₂₉, or COOR₃₀; wherein each R₂₆, R₂₇ and R₂₈ is independently hydrogen or halogen; R₂₉ is halogen, hydrogen, C₁-C₁₀ alkyl C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₁; R₃₀ is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₂; and each R₃₁ and R₃₂ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula I, the EWG is selected from a group consisting of F, Cl, Br, —C═O, NO₂, —CF₃, —CCl₃, —SO₃ and —CN. In some embodiments of a compound of Formula I, the EWG is —CN.

In some embodiment of a compound of Formula I the substituent WSG is a water soluble group. In some embodiments of a compound of Formula I, WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₃; wherein each R₃₃ is independently halogen, —OR₃₄, —NR₃₅R₃₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇; each R₃₄, R₃₅ and R₃₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇; each R₃₇ is independently halogen, —OR₃₈, —NR₃₉R₄₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and each of R₃₈, R₃₉ and R₄₀ is independently hydrogen or C₁-C₁₀ alkyl. In some embodiments of a compound of Formula I, WSG is

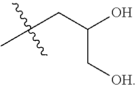 [SMILES: *CC(O)CO]

In some embodiments of a compound of Formula I, WSG comprise polyethylene glycol, polypropylene glycol, co-polymer of polyethylene glycol and polypropylene glycol, or alkoxy derivatives thereof In some embodiments of a compound of Formula I, WSG is

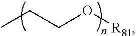

wherein n is an integer from 1-50 and R₈₁ is hydrogen, a C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula I, R₈₁ is methyl. In some embodiments of a compound of Formula I, R₈₁ is CH₂—C≡CH. In some embodiments of a compound of Formula I, WSG is

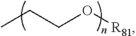

wherein n is an integer from 1-50 and R₈₁ is hydrogen or C₁-C₁₀ alkyl. In some embodiments of a compound of Formula I, R₈₁ is methyl. In some embodiments of a compound of Formula I, the variable n is an integer from 1-10. In some embodiments of a compound of Formula I, n is 3 or 6. In some embodiments of a compound of Formula I, the WSG is

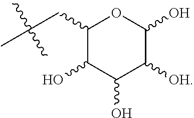 [SMILES: *CC1OC(O)C(O)C(O)C1O]

In some embodiments of a compound of Formula I, WSG is

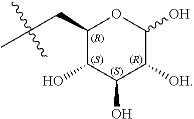

In some embodiments of a compound of Formula I, WSG is

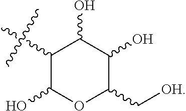

In some embodiments of a compound of Formula I, the WSG is

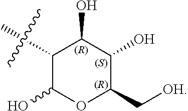

In some embodiments of a compound of Formula I, the compound of the disclosure is selected from a group consisting of

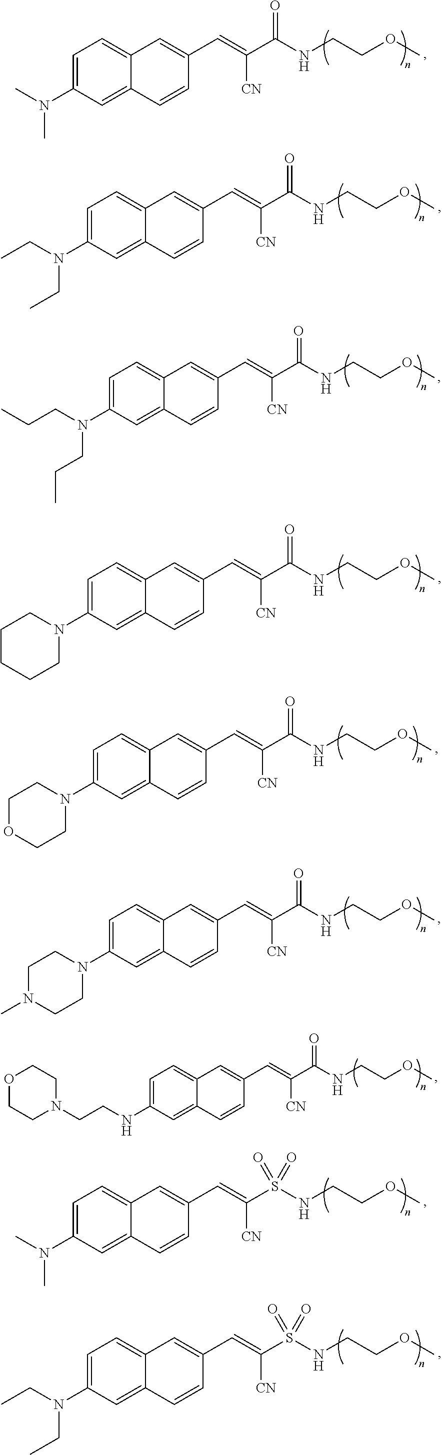

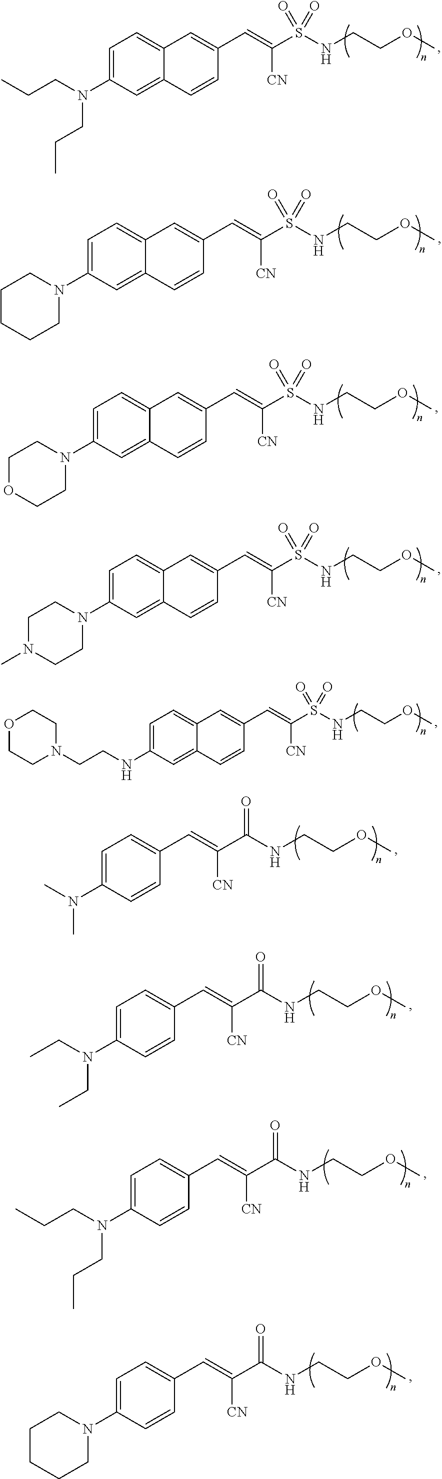

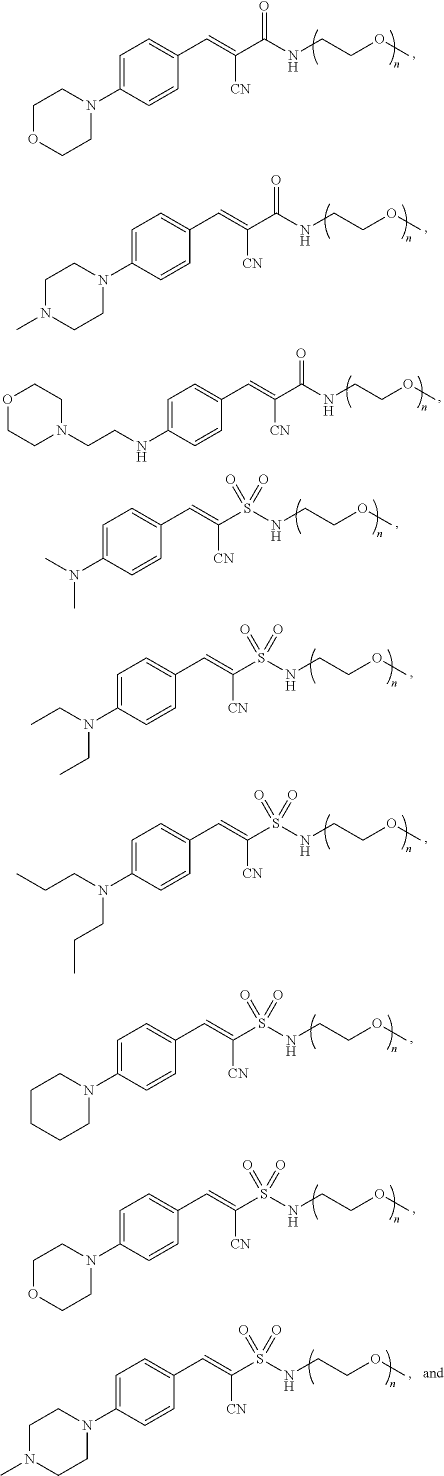

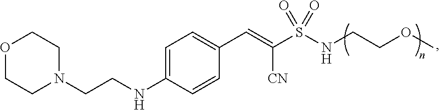

wherein n is an integer with value 1-10.

In some embodiments of a compound of Formula I, the compound of the disclosure is selected from a group consisting of

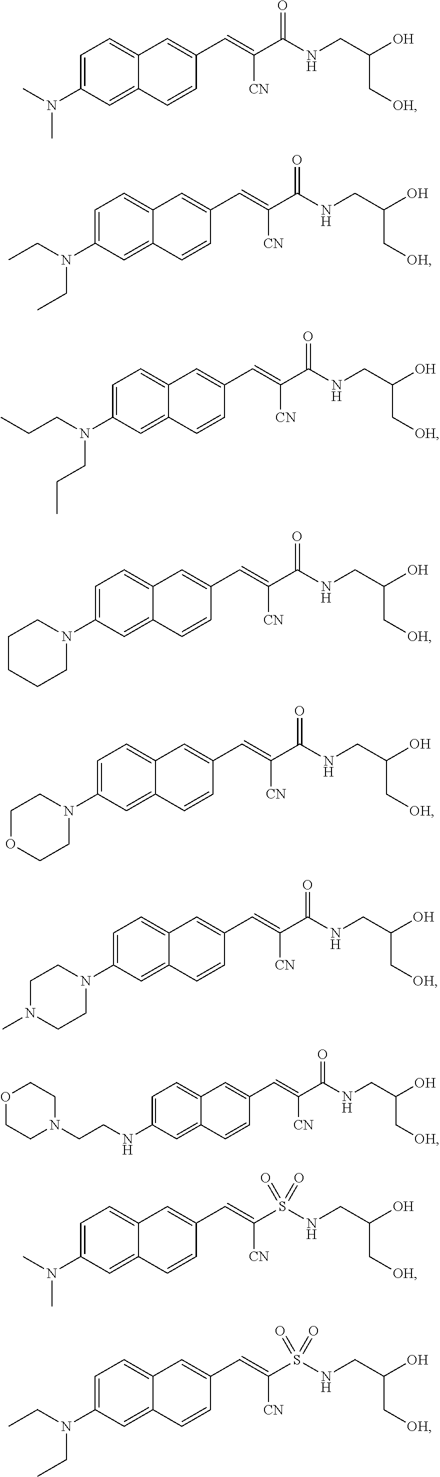

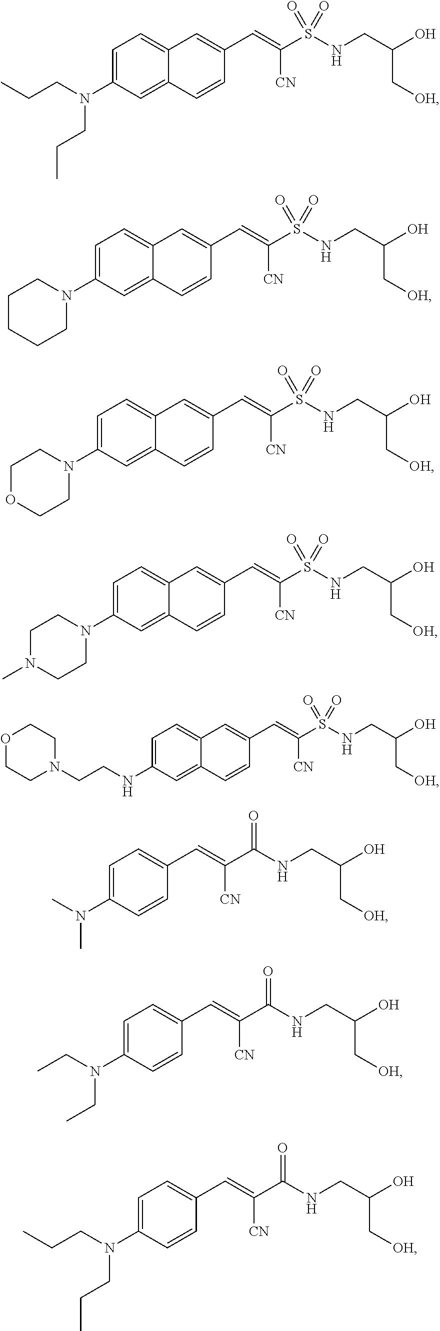

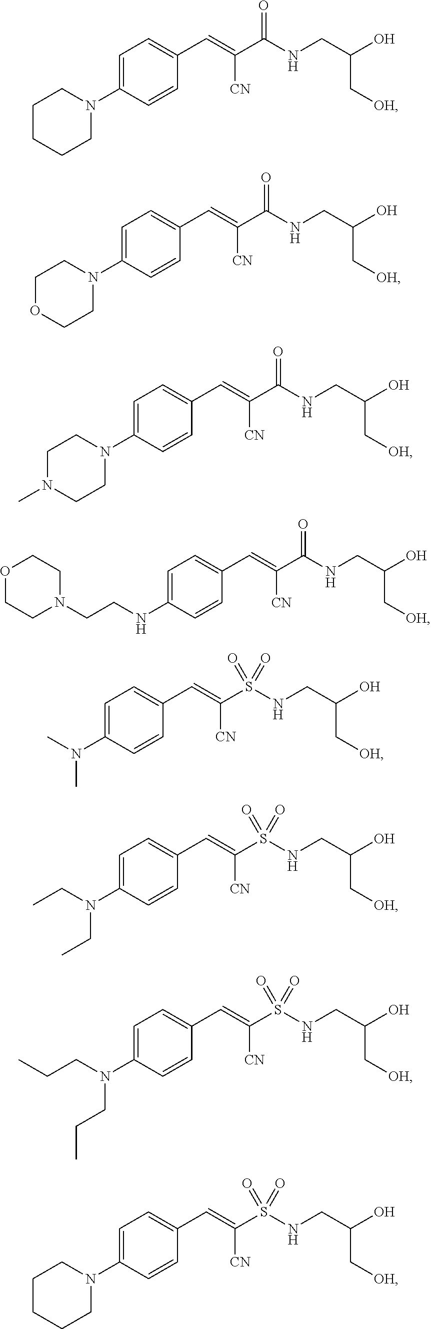

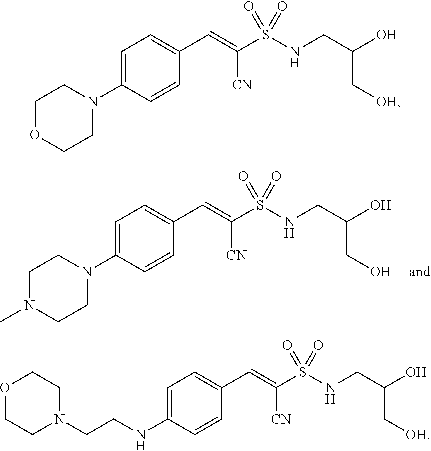 [SMILES: N#C/C(=C\c1ccc(N2CCOCC2)cc1)S(=O)(=O)NCC(O)CO ; CN1CCN(c2ccc(/C=C(\C#N)S(=O)(=O)NCC(O)CO)cc2)CC1 ; N#C/C(=C\c1ccc(NCCN2CCOCC2)cc1)S(=O)(=O)NCC(O)CO]

In some embodiments of a compound of Formula I, the compound is

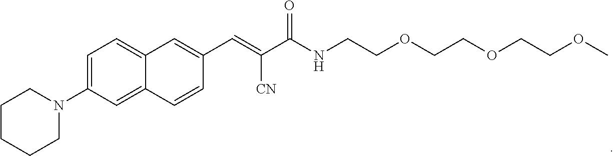 [SMILES: COCCOCCOCCNC(=O)/C(C#N)=C/c1ccc2cc(N3CCCCC3)ccc2c1]

In some embodiments of a compound of Formula I, the compound is

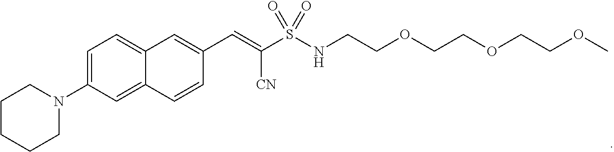 [SMILES: COCCOCCOCCNS(=O)(=O)/C(C#N)=C/c1ccc2cc(N3CCCCC3)ccc2c1]

In some embodiments of a compound of Formula I, the compound is

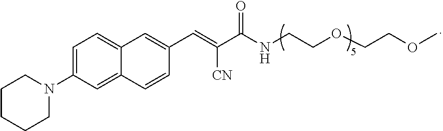

In some embodiments of a compound of Formula I, the compound is

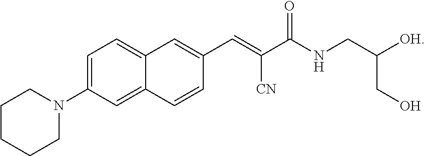 [SMILES: N#C/C(=C\c1ccc2cc(N3CCCCC3)ccc2c1)C(=O)NCC(O)CO]

In some embodiments of a compound of Formula I, the compound is

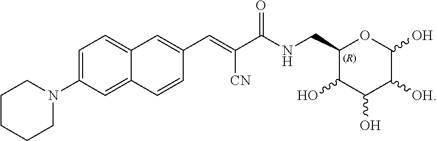

In some embodiments of a compound of Formula I, the compound is

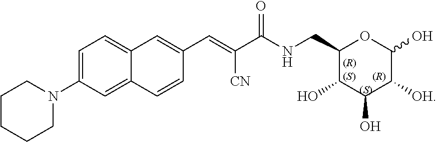

In some embodiments of a compound of Formula I, the compound is

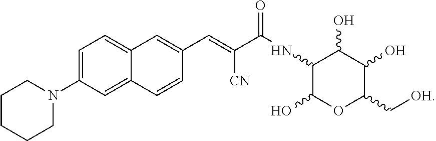 [SMILES: N#C/C(=C\c1ccc2cc(N3CCCCC3)ccc2c1)C(=O)NC1C(O)OC(CO)C(O)C1O]

In some embodiments of a compound of Formula I, the compound is

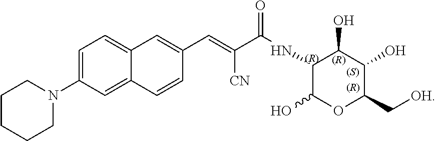

In some embodiments of a compound of Formula I, the compound is

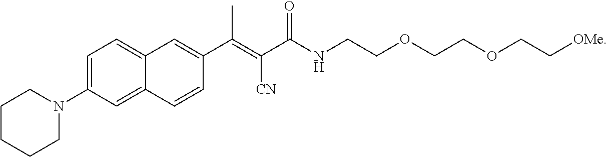 [SMILES: COCCOCCOCCNC(=O)/C(C#N)=C(\C)c1ccc2cc(N3CCCCC3)ccc2c1]

In some embodiments of a compound of Formula I, the compound is

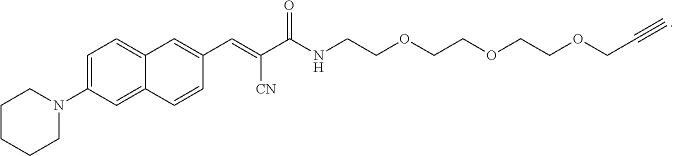 [SMILES: C#CCOCCOCCOCCNC(=O)/C(C#N)=C/c1ccc2cc(N3CCCCC3)ccc2c1]

In some embodiments of a compound of Formula I, the compound is

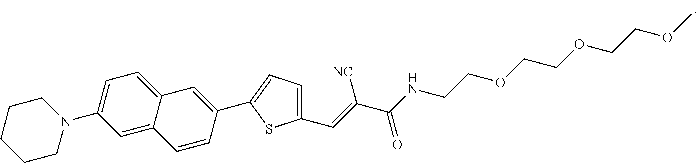 [SMILES: COCCOCCOCCNC(=O)/C(C#N)=C/c1ccc(-c2ccc3cc(N4CCCCC4)ccc3c2)s1]

In some embodiments of a compound of Formula I, the compound is

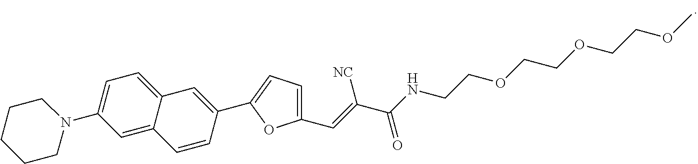 [SMILES: COCCOCCOCCNC(=O)/C(C#N)=C/c1ccc(-c2ccc3cc(N4CCCCC4)ccc3c2)o1]

In some embodiments of a compound of Formula I, the compound is

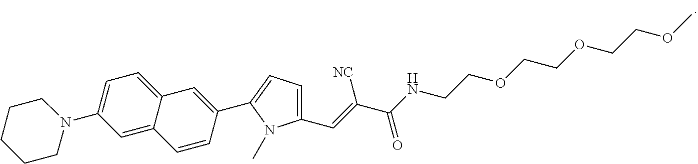 [SMILES: COCCOCCOCCNC(=O)/C(C#N)=C/c1ccc(-c2ccc3cc(N4CCCCC4)ccc3c2)n1C]

In some embodiments of a compound of Formula I, the compound is

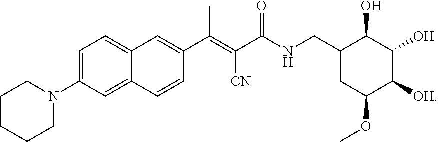 [SMILES: CO[C@H]1CC(CNC(=O)/C(C#N)=C(\C)c2ccc3cc(N4CCCCC4)ccc3c2)[C@@H](O)[C@H](O)[C@H]1O]

In some embodiments of a compound of Formula I, the compound is

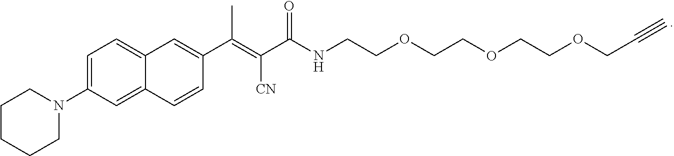 [SMILES: C#CCOCCOCCOCCNC(=O)/C(C#N)=C(\C)c1ccc2cc(N3CCCCC3)ccc2c1]

In some embodiments of a compound of Formula I, the compound is

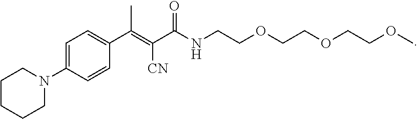 [SMILES: COCCOCCOCCNC(=O)/C(C#N)=C(\C)c1ccc(N2CCCCC2)cc1]

In some embodiments of a compound of Formula I, the compound is

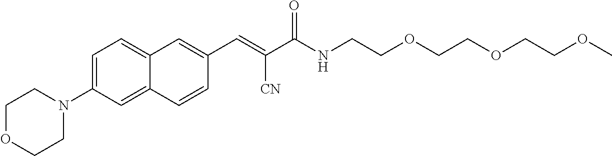 [SMILES: COCCOCCOCCNC(=O)/C(C#N)=C/c1ccc2cc(N3CCOCC3)ccc2c1]

In some aspects the compound of Formula I as described herein forms a complex with an amyloid in the sample. In some aspects the compound of Formula I as described herein forms a complex with an amyloid in the sample that is detectable through florescence.

In some aspects a sample such as a sample of the types disclosed herein is provided in a composition comprising a compound of Formula II, or a pharmaceutically acceptable salt thereof:

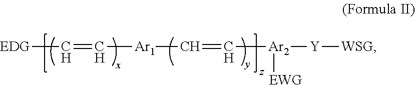

wherein EDG is an electron donating group;
Ar₂ and each Ar₁ is independently C₁-C₁₄ arylene or C₁-C₁₄ heteroarylene, each optionally substituted with one more R₄₁;
each R₄₁ is independently halogen,—CN, —OR₄₂, —NR₄₃R₄₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R₄₅;
R₄₂, R₄₃ and R₄₄ are independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₄₅;
each R₄₅ is independently halogen, —OR₄₆, —NR₄₇R₄₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
R₄₆, R₄₇ and R₄₈ are independently hydrogen or C₁-C₁₀ alkyl;
EWG is an electron withdrawing group;
Y is absent, O, NH, or S;
WSG is hydrogen or a water soluble group;
x is an integer from 0-10;
y is an integer from 0-10; and
z is an integer from 1-10.

In some aspects the substituent EDG in Formula II is an electron donating group. In some embodiments of a compound of Formula II, EDG is OR₄₉, NR₅₀R₅₁, —SR₅₂, —PR₅₃R₅₄, —NR₅₅C(O)R₅₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₅₇; wherein each R₅₇ is independently halogen, —OR₅₈, —NR₅₉R₆₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; each of R₄₉, R₅₀, R₅₁, R₅₂, R₅₃, R₅₄, R₅₅, R₅₆, R₅₈, R₅₉ and R₆₀ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₆₁ and wherein R₅₀ and R₅₁ are optionally joined together to form a heterocycloalkyl or heteroaryl optionally substituted with R₆₁; each of R₆₁ is independently halogen, —OR₆₂, —NR₆₃R₆₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₆₅; each of R₆₂, R₆₃ and R₆₄ is independently hydrogen or C₁-C₁₀ alkyl; and each R₆₅ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

In some embodiments of a compound of Formula II, EDG is selected from a group consisting of

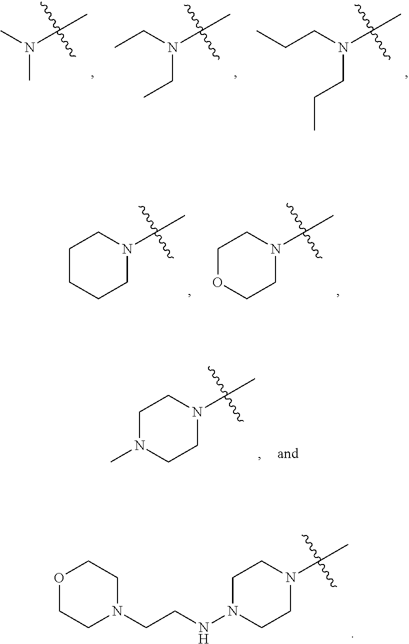

In some embodiments of a compound of Formula II, EDG is

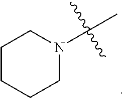 [SMILES: *N1CCCCC1]

In some embodiments of a compound of Formula II, EWG is an electron withdrawing group. In some embodiments of a compound of Formula II, EWG is halogen, —CN, —NO₂, —SO₃H, —CR₆₆R₆₇R₆₈, COR₆₉, or COOR₇₀; wherein each R₆₆, R₆₇ and R₆₈ is independently hydrogen or halogen; R₆₉ is halogen, hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₁; R₇₀ is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₂; each R₇₁ and R₇₂ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula II, EWG is selected from a group consisting of F, Cl, Br, —C═O, NO₂, —CF₃, —CCl₃, —SO₃ and —CN. In some embodiments of a compound of Formula II, EWG is —CN.

In some embodiments of a compound of Formula II, Y in Formula II is absent, O, NH, or S. In some embodiments of a compound of Formula II, Y is absent.

In some embodiments of a compound of Formula II, WSG in Formula II is a water soluble group. In some embodiments of a compound of Formula II, WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₃; wherein each R₇₃ is independently halogen, —OR₇₄, —NR₇₅R₇₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₇; each R₇₄, R₇₅ and R₇₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₇; each R₇₇ is independently halogen, —OR₇₈, —NR₇₉R₈₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and each of R₇₈, R₇₉ and R₈₀ is independently hydrogen or C₁-C₁₀ alkyl. In some embodiments of a compound of Formula II, WGS is hydrogen. In some embodiments of a compound of Formula II, WSG is

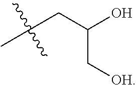 [SMILES: *CC(O)CO]

In some embodiments of a compound of Formula II, WSG is polyethylene glycol, polypropylene glycol, co-polymer of polyethylene glycol and polypropylene glycol, or alkoxy derivatives thereof In some embodiments of a compound of Formula II, WSG is

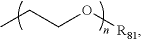

wherein n is an integer from 0-50 and R₈₁ is hydrogen, C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula II, R₈₁ is hydrogen. In some embodiments of a compound of Formula II, R₈₁ is methyl. In some embodiments of a compound of Formula II, R₈₁ is ethyl. In some embodiments of a compound of Formula II, R₈₁ is CH₂—C≡CH. In some embodiments of a compound of Formula II, the variable n is any integer of value 0-10. In some embodiments of a compound of Formula II, n is 0, 3 or 6. In some embodiments of a compound of Formula II, WSG is

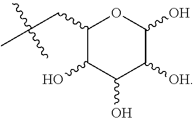 [SMILES: *CC1OC(O)C(O)C(O)C1O]

In some embodiments, WSG is

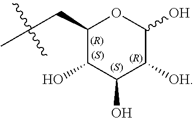

In some embodiments, WSG is

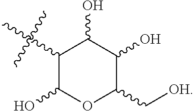

In some embodiments of a compound of Formula II, WSG is

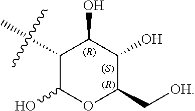

In some embodiments of a compound of Formula II, each of Ar₁ is independently a naphthylene or a phenylene. In some embodiments of a compound of Formula II, Ar₂ is a naphthylene or a phenylene.

In some cases, the compound of Formula II is selected from a group consisting of

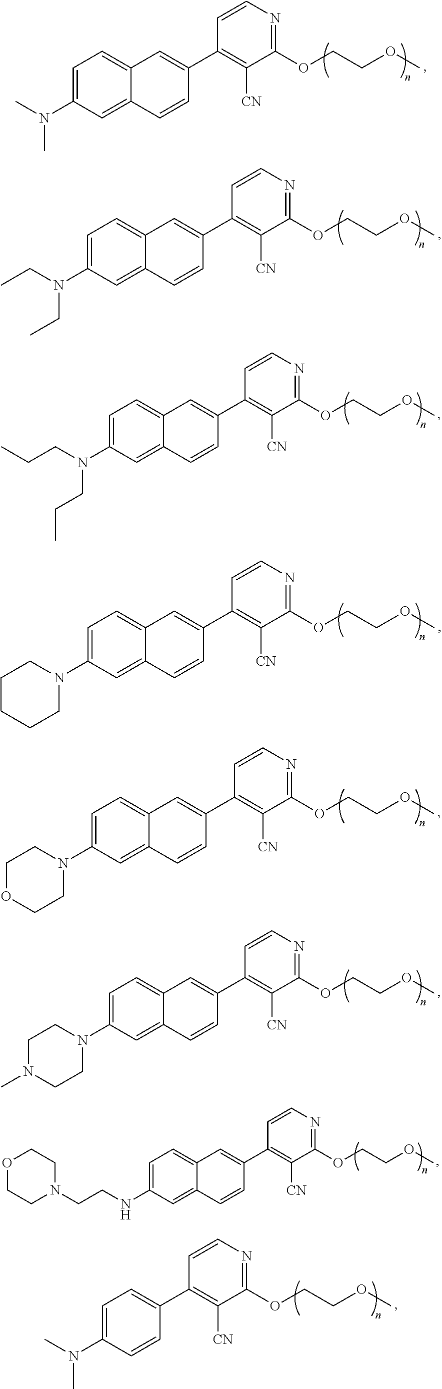

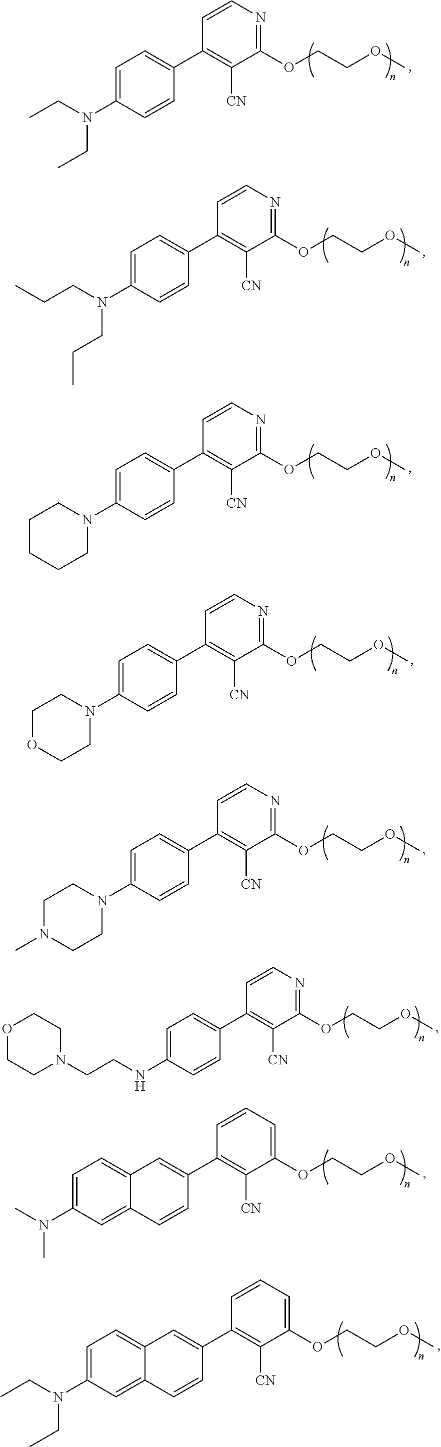

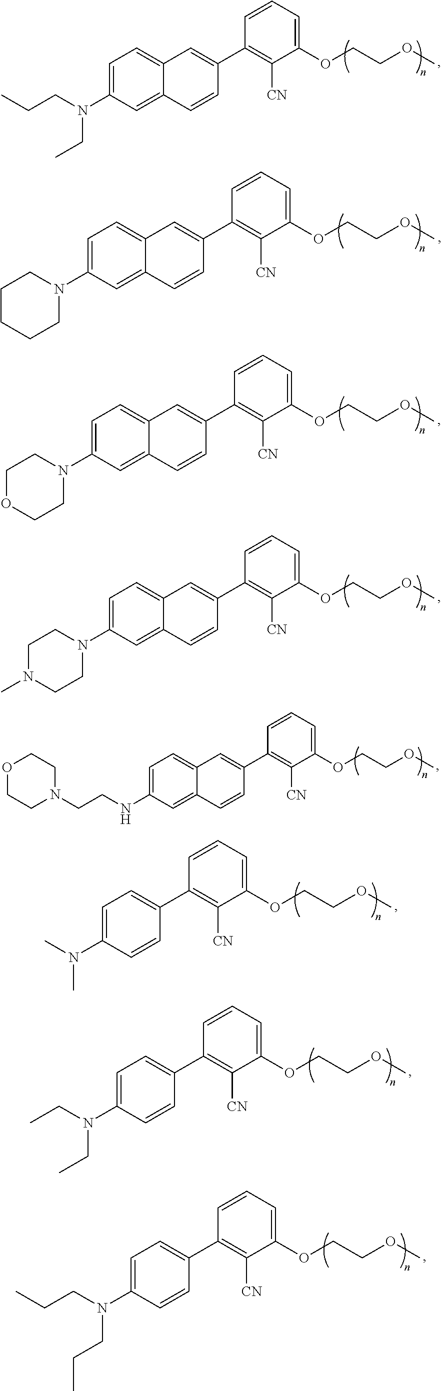

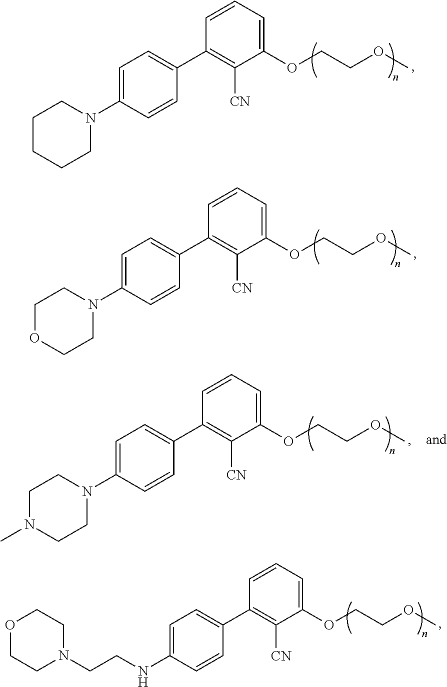

wherein n is an integer with value 0-10.

In some embodiments, the compound of Formula II is selected from a group consisting of

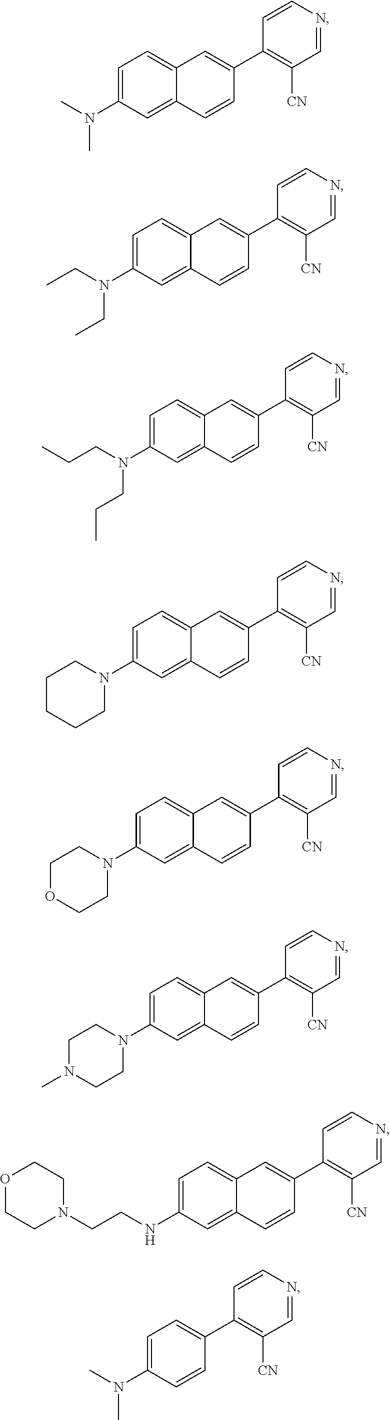

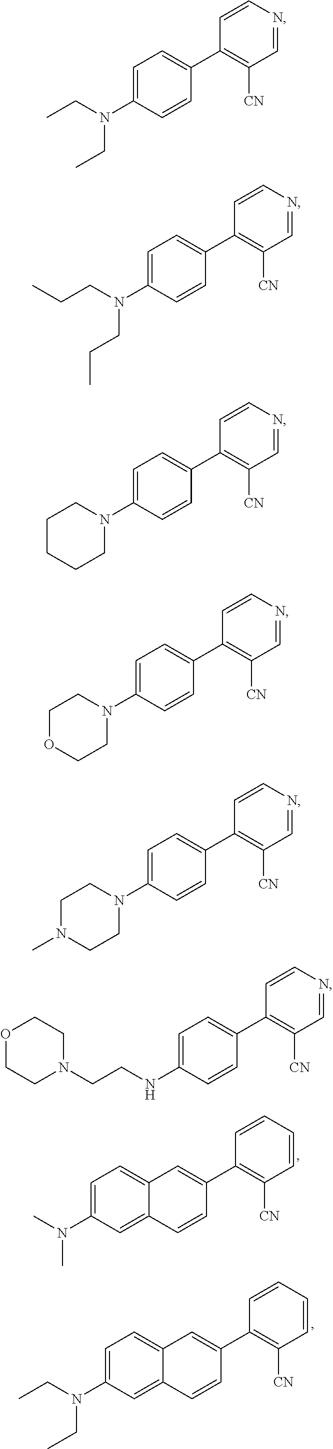

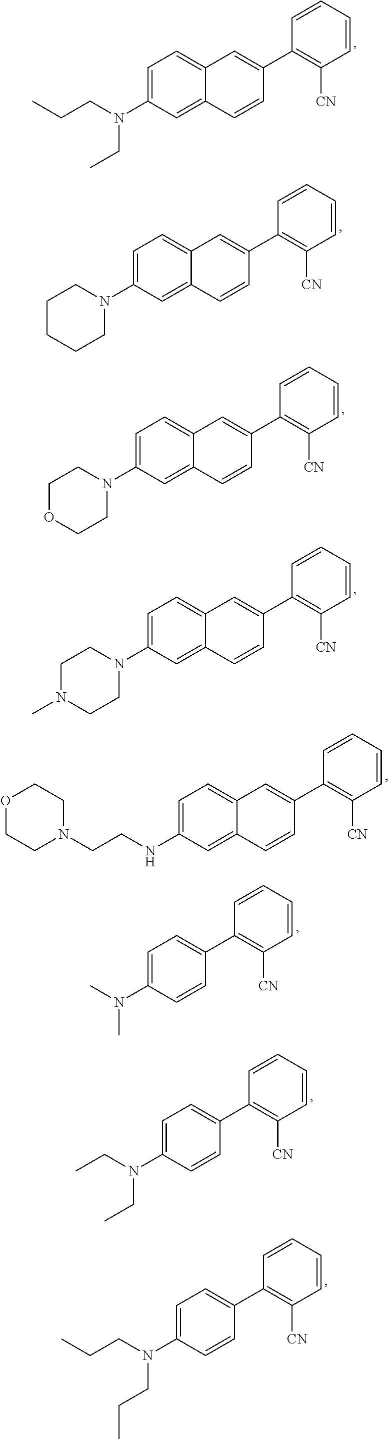

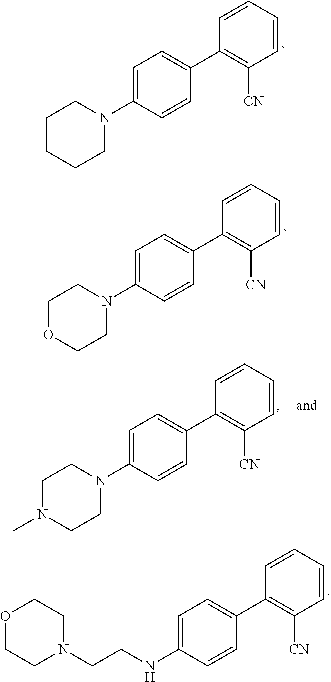 [SMILES: N#Cc1ccccc1-c1ccc(N2CCCCC2)cc1 ; N#Cc1ccccc1-c1ccc(N2CCOCC2)cc1 ; CN1CCN(c2ccc(-c3ccccc3C#N)cc2)CC1 ; N#Cc1ccccc1-c1ccc(NCCN2CCOCC2)cc1]

In some embodiments of a compound of Formula II, the compound of Formula II is

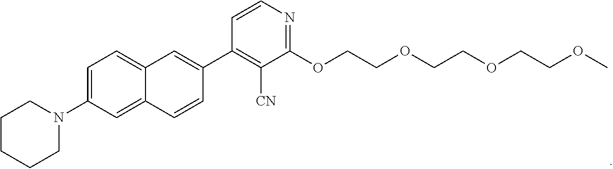 [SMILES: COCCOCCOCCOc1nccc(-c2ccc3cc(N4CCCCC4)ccc3c2)c1C#N]

In some embodiments of a compound of Formula II, the compound of Formula II is

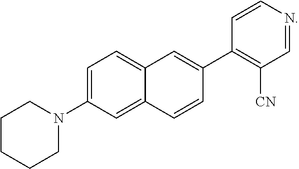 [SMILES: N#Cc1cnccc1-c1ccc2cc(N3CCCCC3)ccc2c1]

In some embodiments of a compound of Formula II, the compound of Formula II is

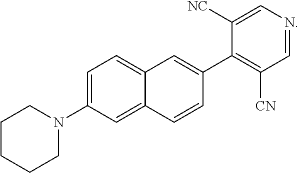 [SMILES: N#Cc1cncc(C#N)c1-c1ccc2cc(N3CCCCC3)ccc2c1]

In some embodiments of a compound of Formula II, the compound of Formula II is

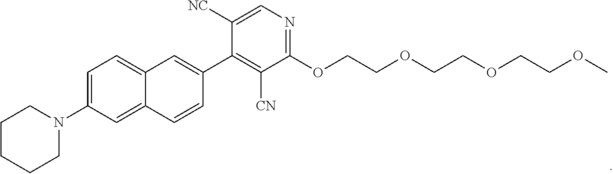 [SMILES: COCCOCCOCCOc1ncc(C#N)c(-c2ccc3cc(N4CCCCC4)ccc3c2)c1C#N]

In some aspects the compound of Formula II as described herein forms a complex with an amyloid in the sample. In some aspects the compound of Formula II as described herein forms a complex with an amyloid in the sample that is detectable through florescence. In another aspect, the disclosure provides methods of detecting an amyloid or amyloid like protein in a sample, such as a human sample, in contact with a compound used in the detection of amyloid in the sample. In some cases the sample is drawn, collected, obtained, or originates from a human, such as a human at risk of an amyloid disorder, a human suspected of suffering from an amyloid disorder, a human with a familial history of an amyloid disorder, or a human undergoing a general medical health survey. In some cases, the sample comprises at least one of a tissue, a cell such as a leukocyte, monocyte, or peripheral blood leukocyte (PBL), a bodily fluid such as urine, blood, serum, plasma, lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, gastric acid, or a tumor or cancerous tissue, tumor or cancerous cell, a fluid from a tumor or cancerous tissue or cell, or an extract derived from any of the aforementioned tissues, cells or fluids. In some aspects the sample comprises an amyloid. In some aspects the sample comprises at least one of an amyloid beta, an alpha synuclein, a prion, huntingtin (HTT), serum amyloid A (SAA), transthyretin (ATTR), lysozyme (ALys), amylin (AIPP), immunoglobulin light chain (AL), semen derived enhancer of viral infection (SEVI), PAP₈₅₋₁₂₀, SEM1₄₉₋₁₀₇, protegrin-1, and Curli (CsgA-R5 or CsgA-R1).

In some aspects the method comprise contacting a compound according to Formula I as described herein or according to Formula II as described herein with a sample potentially comprising the amyloid or amyloid like protein, wherein in the presence of an amyloid or amyloid like protein in the sample as described herein the compound forms a detectable complex, and detecting the formation of the detectable complex such that the presence or absence of the detectable complex correlates with the detectable presence or absence of the amyloid or amyloid like protein. The detection of the formation of the detectable complex is performed by measuring a signal generated by the detectable complex in some aspects.

The signal is an electromagnetic signal in some aspects, for example a fluorescence signal. The amyloid or amyloid like protein may be Aβ peptide, prion peptide, alpha-synuclein, or superoxide dismutase, for example the amyloid or amyloid like protein may be beta amyloid (1-42) (Aβ (1-42)). Detection may be performed within about 1 sec, about 5 sec, about 1 min, about 10 min, about 30 min or about 60 min of the contacting of the compound of Formula I or Formula II with the sample. In some embodiments, detection may be performed within about 1-5 minutes of the contacting of the compound of Formula I or Formula II.

In another aspect, the disclosure provides a method of determining the presence or absence of one or more disease or condition in a subject. In some embodiments, the disease or condition is a disease or condition characterized by protein aggregation or protein misfolding. The method comprises administering to the subject an effective amount of a compound according to Formula I or Formula II, or a pharmaceutical composition thereof, wherein in presence of the disease or condition the administered compound forms a detectable complex, and detecting the detectable complex such that presence or absence of detectable complex correlates with the presence or absence of the disease or condition. In some embodiments, the disease or condition is amyloid based disease or condition characterized by accumulation of amyloid in the subject. In some embodiments, the disease or condition is accompanied by protein that produces amyloid like morphology. In some embodiments, the disease or condition is Alzheimer's disease (AD), Parkinson's disease, Huntington's disease, amyotrophic lateral sclerosis (ALS), Lewy body dementia (LBD), or Down's syndrome. In some embodiments, the disease of condition is Alzheimer's disease. In some embodiments, the disease or condition is a prion disease or condition, for example Creutzfeldt-Jakob disease (CJD). In some embodiments, administration is systemic or topical. In some embodiments, the compound is administered to the eye of the subject. In some embodiments, detection is performed within about 1 sec, about 5 sec, about 1 min, about 10 min, about 30 min or about 60 min of the administration of the compound of Formula I or Formula II to the subject. For example, within about 1-5 min of the administration of the compound of Formula I or Formula II to the subject. In some embodiments, detection of the formation of the detectable complex is performed by measuring a signal generated by the detectable complex. In some embodiments, the signal is an electromagnetic signal, for example a fluorescence signal. In some embodiments, the effective amount of the compound correspond to about 50-500 mg of compound per adult subject.

In another aspect, the disclosure provides a screening method. In some embodiments, the method comprises administering to a subject an effective amount of a compound according to Formula I or Formula II or a pharmaceutical composition thereof, wherein the administration of the compound according to Formula I or Formula II results in formation of a detectable complex. In some embodiments, the screening method further comprises measuring a signal generated by the compound of Formula I or Formula II administered to the subject, and/or by the detectable complex formed by compound of Formula I or Formula II. In some embodiments, the method comprises making a clinical decision based on the measured signal. In some embodiments, the signal is an electromagnetic signal, for example a fluorescence signal. In some embodiments, the administering is systemic or topical administration. In some embodiments, the administering is done to the eye of the subject.

In some embodiments, the disclosure provides methods of detecting an amyloid disorder in a human subject, such as methods comprising the steps of: contacting a sample from the human subject to a molecule having a first spectal profile when unbound and a second spectal profile when bound to a protein aggregate; determining a spectal profile for the sample contated to the molecule; wherein the second spectal profile indicates presence of the amyloid disorder; and wherein the method has a sensitivity of at least 80% and a specificity of at least 80%. In some aspects, the molecule is a compound of Formula I as described herein or a compound of Formula II as described herein. In some embodiments of a method of detecting an amyloid disorder in a human subject, the sensitivity is at least 90%, at least 95%, or least 99%. In some embodiments of a method of detecting an amyloid disorder in a human subject, the specificity is at least 85%. In some embodiments of a method of detecting an amyloid disorder in a human subject, the first spectral profile indicates absence of the amyloid disorder. In some embodiments of a method of detecting an amyloid disorder in a human subject, the amyloid disorder is selected from the list consisting of Alzheimer's disease, Amyloid amyloidosis, Amyloid light chain amyloidosis, amyotrophic lateral sclerosis, apolipoprotein A1, myloidosis, bacterial homeostasis, breast tumors, Cerebral Amyloid Angiopathy, Creutzfeld-Jakob disease, Creutzfeldt-Jacob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, lymphoma, Lysozomal storage disorders, lysozyme amyloidosis, nephrogenic diabetes insipidus, p53-related cancers, Parkinson's disease, Pre-eclampsia, Rheumatoid arthritis, senile systemic amyloidosis, skin tumors, Spongiform encephalitis, systemic AL amyloidosis, tumoral amyloidosis, and Type II diabetes. In some embodiments, the amyloid disorder is pre-eclampsia. In some embodiments of a method of detecting an amyloid disorder in a human subject, the amyloid disorder comprises an aggregate of at least one of Aβ peptide, α-Synuclein, prion peptide, huntingtin, serum amyloid A, transthyretin, lysozyme, amylin, immunoglobulin light chain, semen derived enhancer of viral infection, PAB, SEMI, protegrin-1, CsgA-R5, and CsgA-R1, superoxide dismutase, insulin, and p53. In some embodiments of a method of detecting an amyloid disorder in a human subject, the sample comprises a cell selected from the list consisting of a leukocyte, a monocyte, a peripheral blood leukocyte (PBL), a white blood cell, a red blood cell, a skin cell, cheek cell, a hair follicle cell, and a nerve cell. In some embodiments of a method of detecting an amyloid disorder in a human subject, the sample comprises a fluid selected from the list of fluids consisting of as urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, and gastric acid. In some embodiments, the sample comprises urine. In some embodiments, the sample comprises a tumor sample. In some embodiments, the tumor sample is selected from the list consisting of a tumor tissue, a tumor cell, a tumor fluid, a partially homogenized tumor extract, and a fully homogenized tumor extract. In some embodiments of a method of detecting an amyloid disorder in a human subject, the amyloid disorder is pre-eclampsia, the sample comprises urine, the specificity is at least 99%, the sensitivity is at least 85%, and the molecule is compound 1 or a pharmaceutically acceptable salt thereof In some embodiments of a method of detecting an amyloid disorder in a human subject, the amyloid disorder is pre-eclampsia, the sample comprises urine, the specificity is at least 99%, the sensitivity is at least 85%, and the molecule is a molecule of Formula I or a pharmaceutically acceptable salt thereof In some embodiments, the amyloid disorder is pre-eclampsia, the sample comprises urine, the specificity is at least 99%, the sensitivity is at least 85%, and the molecule is a molecule of Formula II or a pharmaceutically acceptable salt thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a synthetic strategy towards the synthesis of compound 1.

FIG. 2 shows the fluorescence excitation and emission profiles of compound 1, measures using 4 μM compound 1 and 5 μM Aβ (1-42) in 5% DMSO in water.

FIG. 3 shows a synthetic strategy towards the synthesis of compound 2.

FIG. 4 shows the fluorescence excitation and emission profiles of compound 2, measures using 4 μM compound 2 and 5 μM Aβ (1-42) in 5% DMSO in water.

FIG. 5 shows a synthetic strategy towards the synthesis of compound 3.

FIG. 6 shows the fluorescence excitation and emission profiles of compound 3, measures using 4 μM compound 3 and 5 μM Aβ (1-42) in 5% DMSO in water.

FIG. 7 shows a synthetic strategy towards the synthesis of compound 5.

FIG. 8 shows the fluorescence excitation and emission profiles of compound 5, measures using 4 μM compound 5 and 5 μM Aβ (1-42) in 5% DMSO in water.

FIG. 9 shows a synthetic strategy towards the synthesis of compound 17.

FIG. 10 shows the fluorescence excitation and emission profiles of compound 17, measures using 4 μM compound 17 and 5 μM Aβ (1-42) in 5% DMSO in water.

FIG. 11 shows a synthetic strategy towards the synthesis of compound 18.

FIG. 12 shows the fluorescence excitation and emission profiles of compound 18, measured using 4 μM compound 18 and 5 μM or 10 μM Aβ (1-42) in 5% DMSO in water.

FIG. 13A Shows representative fluorescence micrographs showing the fluorescence labeling of amyloid deposits in hippocampal brain sections from a mouse model for Alzheimer's disease using compound 1. An excitation wavelength of 488 nm was used to excite the fluorophores.

FIG. 13. B Shows representative fluorescence micrographs showing the fluorescence labeling of amyloid deposits in hippocampal brain sections from a mouse model for Alzheimer's disease using compound 2. An excitation wavelength of 488 nm was used to excite the fluorophores.

FIG. 14A Shows normalized fluorescence intensity at 534 nm of urine incubated with compound 1 from urine specimens obtained from non-PE individuals (controls) or pregnant women that were diagnosed with pre-eclampsia (patients).

FIG. 14B Shows receiver operating characteristics (ROC) curve showing sensitivity and specificity of compound 1 for pre-eclampsia from urine specimens obtained from non-PE individuals (controls) or pregnant women that were diagnosed with pre-eclampsia (patients).

FIG. 15A Shows normalized fluorescence intensity at 534 nm of urine incubated with compound 2 from urine specimens obtained from non-PE individuals (controls) or pregnant women that were diagnosed with pre-eclampsia (patients).

FIG. 15B Shows receiver operating characteristics (ROC) curve showing sensitivity and specificity of compound 2 for pre-eclampsia from urine specimens obtained from non-PE individuals (controls) or pregnant women that were diagnosed with pre-eclampsia (patients).

FIG. 16A Shows normalized fluorescence intensity at 534 nm of urine incubated with compound 5 from urine specimens obtained from non-PE individuals (controls) or pregnant women that were diagnosed with pre-eclampsia (patients).

FIG. 16B Shows receiver operating characteristics (ROC) curve showing sensitivity and specificity of compound 5 for pre-eclampsia from urine specimens obtained from non-PE individuals (controls) or pregnant women that were diagnosed with pre-eclampsia (patients).

FIG. 17A Shows normalized fluorescence intensity at 534 nm of urine incubated with compound 21 from urine specimens obtained from non-PE individuals (controls) or pregnant women that were diagnosed with pre-eclampsia (patients).

FIG. 17B Shows receiver operating characteristics (ROC) curve showing sensitivity and specificity of compound 21 for pre-eclampsia from urine specimens obtained from non-PE individuals (controls) or pregnant women that were diagnosed with pre-eclampsia (patients)

FIG. 18A Shows normalized fluorescence intensity at 534 nm of urine incubated with compound 22 from urine specimens obtained from non-PE individuals (controls) or pregnant women that were diagnosed with pre-eclampsia (patients).

FIG. 18B Shows receiver operating characteristics (ROC) curve showing sensitivity and specificity of compound 22 for pre-eclampsia from urine specimens obtained from non-PE individuals (controls) or pregnant women that were diagnosed with pre-eclampsia (patients).

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The disclosure provides compositions and methods for the identification of amyloid or amyloid-like protein complexes in a sample, such as a sample from a human. In some cases, the presence or detection of such amyloid or amyloid like complexes indicates the presence of an amyloid-related disorder in the sample source. In some cases the presence or detection of such amyloid or amyloid-like protein complexes indicates an increased risk of developing symptoms of an amyloid or amyloid-related disorder.

Amyloids are insoluble fibrous protein aggregates. They arise from a number of inappropriately folded versions of proteins and polypeptides present naturally in the body. These misfolded structures interact with one another or other cell components forming insoluble fibrils. They have been associated with the pathology of more than 20 serious human diseases. Accumulation of amyloid fibrils in organs may lead to amyloidosis, and may play a role in a large and growing number of disorders.

Amyloids were originally understood as extracellular, proteinaceous deposit exhibiting beta sheet structure. They were generally identified by apple-green birefringence under polarized light when stained with the dye congo red. These deposits often recruit sugars and other components such as Serum Amyloid P, sometimes resulting in complex inhomogeneous structures. A more recent definition, adopted herein, includes as an amyloid any polypeptide that polymerizes to form a cross-beta structure, in vivo or in vitro, independent of classic histopathological characteristics such as the Congo-red birefringence.

A partial list of amyloid-forming proteins includes the following: a-1-antitrypsin, Alpha-synuclein, Amylin (IAAP), Apolipoprotein A1 and fragments, Atrial natriuretic factor, Beta₂ microglobulin, beta-amyloid, Calcitonin, Curli (CsgA-R1), Curli (CsgA-R5), Cystatin, Gelsolin, Huntingtin, Huntingtin, Immunoglobulin light chain AL, insulin, Keratoepithelin, Lysozyme, Medin, p53, PAP85-120, prion proteins or fragments (PrP^{Sc}), Prolactin, Protegrin-1, SEM1 49-107, Semen derived enhancer of viral infection, serum amyloid A, S-IBM, superoxide dismutase 1, Transthyretin, vasopressin receptor 2.

Amyloid-forming proteins often comprise peptide fragments that are able to form amyloids in combination with full length proteins or one another. Accordingly, some fragments of each of the above proteins are also included in a list of amyloid-forming proteins.

Research in the field is ongoing, and the discovery and characterization of amyloid-forming proteins is ongoing. Accordingly, the list of amyloid forming proteins and fragments presented herein is not comprehensive. In some cases, the methods and compositions disclosed herein are applicable to amyloid proteins and fragments listed herein, while in other cases the methods and compositions provided herein are applicable to all proteins and protein fragments characterized as amyloids. As discussed above, a common characteristic of many amyloid proteins is the capacity to form intermolecular beta-sheet bound aggregates. Accordingly, a number of proteins in some cases, the methods and compositions disclosed herein are applicable to proteins and protein fragments that form stable beta-sheet aggregates.

Amyloid formation has been implicated in a number of diseases and conditions. A partial list of diseases in which amyloids have been implicated includes the following: Alzheimer's disease, Amyloid amyloidosis, Amyloid light chain amyloidosis, amyotrophic lateral sclerosis, apolipoprotein A1, amyloidosis, bacterial homeostasis, breast tumors, Cerebral Amyloid Angiopathy (CAA), Creutzfeld-Jakob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, Lewy body dementia (LBD), lymphoma, Lysozomal storage disorders, lysozyme amyloidosis, nephrogenic diabetes insipidus, p53-related cancers, Parkinson's disease, Pre-eclampsia, protein degradation-related diseases, Rheumatoid arthritis, senile systemic amyloidosis, skin tumors, Spongiform encephalitis, systemic AL amyloidosis, tumoral amyloidosis, and Type II diabetes.

In the above lists of amyloid-forming proteins and amyloid-related diseases and disorders, a number of the listed members were identified as proteins related to disorders, or identified as disorders, well before any role of protein aggregation was identified. Insulin, diabetes mellitus type 2, pre-eclampsia, p53, and lymphoma, for example, were well studied long before a role of amyloid formation was identified. These examples demonstrate the breadth of disorders in which pre-eclampsia is indicated, and suggest that as the subject continues to be researched, additional disorders and proteins are likely to be implicated, and are likely to be compatible with the compositions and methods as disclosed herein.

For many amyloid disorders, diagnosis remains challenging. Early symptoms are often easily confused with those of other disorders, and even later symptoms are either not definitive in diagnosis or are indicative of disease progression to the point that irreparable damage has occurred. Compounding the challenge of diagnosis, many amyloid protein aggregates accumulate in inaccessible tissues, such as the brain or central nervous system, thus creating obstacles to tissue collection at the site of amyloid action. Alzheimer's disease, for example, is easily confused with a number of dementia-related disorders, and is often only definitively diagnosed post-mortem.

Some amyloids are known to accumulate in one or more human body fluids, such as urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, and gastric acid. Similarly, some amyloids accumulate in cell samples such as leukocytes, monocytes, peripheral blood leukocytes (PBL), white blood cells, red blood cells, skin cells, cheek cells, hair follicle cells, or nerve cells, many of which are readily obtainable.

Assays have been developed to detect the presence of amyloid complexes in these samples. However, many of these assays are time consuming and technically cumbersome. Seeding assays, for example, rely on the ability of an amyloid aggregate to catalyze the assembly of alternately folded proteins into amyloid structures. These assays represent a substantial advance over their predecessors but remain fairly slow, technically challenging and expensive.

Disclosed herein are methods and compositions for the rapid identification of amyloid aggregates in vitro in readily obtainable human samples. Amyloids in human samples such as urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, and gastric acid, or samples comprising at least one of a human cell such as a leukocyte, a monocyte, a peripheral blood leukocyte (PBL), a white blood cell, a red blood cell, a skin cell, cheek cell, a hair follicle cell, and a nerve cell, or comprising a tumor sample or tumor sample extract are obtained from a human, such as a human suspected of having an amyloid disorder or a human suspected of being at risk of developing an amyloid disorder or a human for which amyloid disorder screening is desired. The sample is contacted with a at least one compound as disclosed herein (a compound of Formula I or of Formula II or both Formula I and Formula II as characterized below), and optionally at least one additional reagent such as a buffer or sample stabilizer to form an amyloid detection composition as disclosed herein.

In some cases, within an amyloid detection composition as disclosed herein an amyloid and a compound as disclosed herein will form a complex. In some cases the complex formed thereby is a complex having a distinctive electromagnetic emission spectrum upon excitation with electromagnetic energy such as visible or UV light. Detection of such an emission spectrum may therefore be used to identify the formation of such a complex within an amyloid detection composition.

In some cases, the emission spectrum of an amyloid/compound complex in a composition as disclosed herein will vary among amyloid polypeptide constituents. In some of these cases, an emission spectrum of an amyloid compound complex will indicate the identity of a protein or polypeptide constituent of an amyloid/compound complex. Accordingly, through use of the amyloid detection compositions as disclosed herein, one may identify not only the presence of an amyloid complex in a readily obtained human sample assayed in vitro, but one may also identify at least one of the specific constituents of the amyloid complex.

In many cases, specific amyloid complexes are correlated with specific diseases. Thus, by identifying a protein or polypeptide constituent of a specific amyloid complex of an amyloid detection composition as disclosed herein, one may identify or suggest a corresponding amyloid disorder. Accordingly, through the use of an in vitro amyloid detection composition as disclosed herein, one may identify an amyloid disorder in a human or an increased risk of developing an amyloid disorder in a human as indicated by the detection of an amyloid complex in a sample from the human.

A number of disease specific amyloid proteins have been identified in human samples. For example, among neurological disorders, Alzheimer's disease is associated with beta-amyloid peptide, spongiform encephalitis is associated with prion protein or fragments thereof, Parkinson's disease is associated with alpha-synuclein, amyotrophic lateral sclerosis is associated with superoxide dismutase 1, Huntington's disease is associated with huntingtin fragments, familial amyloidotic polyneuropathy is associated with transthyretin mutant proteins; while among non-neuropathic disorders, systemic amyloidosis is associated with immunoglobulin light chain or its fragments, amyloid A amyloidosis is associated with serum amyloid A1 protein fragments, senile systemic amyloidosis is associated with transthyretin, haemodialysis-related amyloidosis is associated with beta₂-microglobulin, lysozyme amyloidosis is associated with lysozyme mutants, apolipoprotein A1 amyloidosis is associated with Apo A-1 fragments, type II diabetes is associated with amylin, and injection-localized amyloidosis is associated with insulin (Knowles et al., (2014) “The amyloid state and its association with protein misfolding diseases” Nature Reviews Molecular Cell Biology 15:384; PrP^{Sc} misfolded prion proteins have been identified in the urine of patients with variant Creutzfeldt-Jakob disease (Moda, et al., (2014) “Prions in the Urine of Patients with Variant Creutzfeldt-Jakob Disease” N. Engl. J. Med 371(6):530); misfolded p53 has been implicated in tumor malignancy (Silva et al., (2014) “Prion-like aggregation of mutant p53 in cancer” Trends in Biochemical Sciences 39(6):260); and amyloid precursor protein accumulation in urine has been associated with early diagnosis of pre-eclampsia (Buhimschi et al., (2014) “Protein misfolding, congophilia, oligomerization, and defective amyloid processing in pre-eclampsia” Sci Trans. Med. 6(245):245), to name but a few of the cases of correspondences between amyloid protein accumulation and a specific amyloid disorder.

Amyloid-compound complexes are identified through a number of distinct approaches in distinct embodiments of the disclosure herein. In some cases a distinct compound as disclosed herein is tagged, for example biotin tagged, such that compound/amyloid complexes can be isolated from compositions disclosed herein for further analysis. In some cases isolated amyloid/compound complexes are subjected to an immunoassay such as a western blot using an antibody specific to an amyloid of interest. In alternate cases, amyloid/compound complexes are subject to peptide digestion followed by mass-spectrometric analysis so as to identify polypeptide constituents.

In some cases spectrophotometric approaches are used to characterize amyloid/compound complexes. In exemplary embodiments, compositions as disclosed herein are assayed directly with no further purification. In some cases compositions as disclosed herein are diluted or subject to additional purification steps prior to amyloid/compound assaying. In some cases amyloid/compound complexes are characterized as disclosed in Cao, et al., (2012) “Aminonaphthaline 2-Cyanoacrylate (ANCA) Probes Fluorescently Discriminate between Amyloid-beta and Prion Plaques in Brain” Journal of the American Chemical Society 134:17338, the contents of which are hereby incorporated by reference in their entirety. In some embodiments, detection of the detectable complex in the methods of the disclosure comprises illuminating the composition with light of an appropriate wavelength and detecting light received from the sample. In some embodiments, the wavelength of the illuminating light is varied and selected according to the fluorescence excitation and emission spectrum of the detectable complex. In some embodiments, the detectable complex has a fluorescent excitation peak in the range of 350-500 nm, and the fluorescence emission spectrum of the detectable complex peaks at 500-550 nm and the illuminating light has a wavelength of 350-450 nm (example 400 nm).

The compounds of the disclosure are designed to form a detectable complex in presence of an amyloid or amyloid-like protein. In some embodiments, the compounds disclosed herein are classified as molecular rotor fluorophores. The compounds comprise an electron rich donor moiety covalently connected to a conjugated pi system (for example, to an aromatic pi network) and in electronic conjugation to an electron poor acceptor moiety covalently connected elsewhere on the pi system. The compounds also comprise one or more single bonds between the donor and acceptor that can rotate freely under standard thermal control of the environment in the temperature range of interest. The rotation of the single bond allows the donor and acceptor to remain substantially decoupled in the absence of binding to a protein and thus the compounds exhibit poor fluorescence signal. However, in presence of an amyloid or amyloid-like protein these compounds may bind to the amyloid or amyloid-like protein. Accordingly, the rotatable single bonds may become essentially frozen and the electronic coupling between the donor and acceptor may be substantially enhanced. This may lead to a strong fluorescence signal upon irradiation with an appropriate wavelength of light. The strong fluorescence enhancement of these compounds upon binding to amyloid or amyloid-like proteins compared to the free compound in solution results in excellent signal to noise ratio and make it possible to image amyloid or amyloid-like proteins with high sensitivity.

Also provided herein is a method for detecting an amyloid or amyloid-like protein. The method comprises contacting a compound of the disclosure with the sample potentially comprising the amyloid or amyloid-like protein, wherein in presence of an amyloid or amyloid-like protein the compound forms a detectable complex, and detecting the formation of the detectable complex such that the presence or absence of the detectable complex correlates with the presence or absence of the amyloid or amyloid like protein. In some embodiments, the detection of the detectable complex in the methods of the disclosure comprises illuminating the sample with light of an appropriate wavelength and detecting light received from the sample. In some embodiments, the wavelength of the illuminating light is varied and selected according to the fluorescence excitation and emission spectrum of the detectable complex. In some embodiments, the detectable complex has a fluorescent excitation peak in the range of 350-500 nm, and the fluorescence emission spectrum of the detectable complex peaks at 500-550 nm and the illuminating light has a wavelength of 350-450 nm (example 400 nm). In some embodiments, any amyloid or amyloid like protein or peptide are detected by the methods of the disclosure. In some embodiments, the method is used to detect the presence or absence of Aβ peptide, prion peptide, alpha-synuclein, or superoxide dismutase.

In some embodiments, the method includes comparing the amount of the detectable complex to a normal control value, wherein an increase in the amount of the detectable complex compared to a normal control value indicates that said patient is suffering from or is at risk of developing the disease or condition.

In some embodiments, the disease or condition is a disease or condition characterized by protein aggregation or misfolding. In some embodiments, the disease or condition is an amyloid based disease or condition. In some embodiments, the amyloid-based disease or condition is any disease or condition associated with the increased or decreased presence of amyloid or amyloid like proteins, such as the presence of amyloid plaques. In some embodiments, the disease is a neuronal disease for example a neurodegenerative diseases, in which amyloid-beta peptides, oligomers, fibrils, or plaques are implicated. For example, Alzheimer's disease, Parkinson's disease, Huntington's disease, amyotrophic lateral sclerosis (ALS), Lewy body dementia (LBD), or Down's syndrome. In some embodiments, the amyloid-based disease or condition includes ocular diseases associated with pathological abnormalities/changes in the tissues of the visual system, particularly associated with amyloid-beta-related pathological abnormalities/changes in the tissues of the visual system, such as, for example, neuronal degradation.

A more comprehensive list of disorders characterized by protein aggregation into amyloid or protein misfolding includes the following: Alzheimer's disease, Amyloid amyloidosis, Amyloid light chain amyloidosis, amyotrophic lateral sclerosis, apolipoprotein Al, myloidosis, bacterial homeostasis, breast tumors, Cerebral Amyloid Angiopathy, Creutzfeld-Jakob disease, Creutzfeldt-Jacob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, Lewy body dementia (LBD), lymphoma, Lysozomal storage disorders, lysozyme amyloidosis, nephrogenic diabetes insipidus, p53-related cancers, Parkinson's disease, Pre-eclampsia, protein degradation-related diseases, Rheumatoid arthritis, senile systemic amyloidosis, skin tumors, Spongiform encephalitis, systemic AL amyloidosis, tumoral amyloidosis, and Type II diabetes. In some cases this list remains partial.

In some embodiments, the compounds and the methods of the disclosure are used to monitor minimal residual disease in a patient following treatment with a compound or a mixture according to the disclosure. In some embodiments, a sample suspected to contain the amyloid antigen is contacted with a compound of the disclosure, and the compound is allowed to bind to the amyloid or amyloid like protein to form a detectable complex. In some embodiments, the formation of the detectable complex is detected and its presence or absence is correlated with the presence or absence of amyloid or amyloid like protein in the sample or specific body part or area. In some embodiments, the amount of said detectable complex is compared to a normal control value, wherein an increase in the amount of said detectable complex compared to a normal control value indicates that the patient is still be suffering from a minimal residual disease.

### Compounds

In one aspect the disclosure provides a compound of Formula I, or a pharmaceutically acceptable salt thereof:

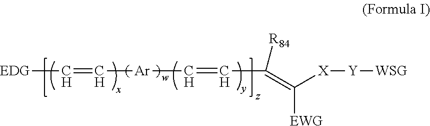

wherein
Each Ar is independently C₁-C₁₄ arylene or C₁-C₁₄ heteroarylene, each optionally substituted with one more R₁;
each R₁ is independently halogen, —CN, —OR₂, —NR₃R₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R₅;
R₂, R₃ and R₄ are independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₅;
each R₅ is independently halogen, —OR₆, —NR₇R₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
R₆, R₇ and R₈ are independently hydrogen or C₁-C₁₀ alkyl;
R₈₄ is hydrogen or C₁-C₁₀ alkyl;
EDG is an electron donating group;
EWG is an electron withdrawing group;
WSG is a water soluble group;
X is C═O or SO₂;
Y is NH, or S;
each x is independently an integer from 0-10;
each w is independently an integer from 1-5;
each y is independently an integer from 0-10; and
z is an integer from 1-10.

In some embodiments of a compound of Formula I, R₈₄ is hydrogen. In some embodiments of a compound of Formula I, R₈₄ is C₁-C₁₀ alkyl. In some embodiments of a compound of Formula I, R₈₄ is methyl, ethyl, propyl, butyl, pentyl, hexyl, heptyl, octyl, neptyl or decyl. In some embodiments of a compound of Formula I, R₈₄ is methyl.

In some embodiments of a compound of Formula I, EDG is an electron donor group, as known in the art. In some embodiments of a compound of Formula I, EDG is any atom or functional group that is capable of donating some of its electron density into a conjugated pi system, thus making the pi system more nucleophilic. In some embodiments of a compound of Formula I, the EDG is —OR₉, —NR₁₀R₁₁, —SR₁₂, —PR₁₃R₁₄, —NR₁₅C(O)R₁₆, C₁-C₁₀ alkyl, C₁-C₁₀ heteroalkyl, cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₁₇; wherein each R₁₇ is independently halogen, —OR₁₈, —NR₁₉R₂₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
each of R₉, R₁₀, R₁₁, R₁₂, R₁₃, R₁₄, R₁₅, R₁₆, R₁₈, R₁₉ and R₂₀ is independently hydrogen, C₁-C₁₀ alkyl, C₁-C₁₀ heteroalkyl, cycloalkyl, C₁-C₁₀ heterocycloalkyl, C₁-C₁₀ arylene, or C₁-C₁₀ heteroarylene, each of which except for hydrogen is optionally substituted with one or more R₂₁ and wherein R₁₀ and R₁₁ are optionally joined together to form a heterocycloalkyl or heteroaryl optionally substituted with R₂₁; each of R₂₁ is independently halogen, —OR₂₂, —NR₂₃R₂₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₂₅; each of R₂₂, R₂₃ and R₂₄ is independently hydrogen or C₁-C₁₀ alkyl; and each R₂₅ is independently C₁-C₁₀ alkyl, C₁-C₁₀ heteroalkyl, cycloalkyl, C₁-C₁₀ heterocycloalkyl, arylene, or C₁-C₁₀ heteroarylene.

In some embodiments of a compound of Formula I, the EDG is selected from a group consisting of

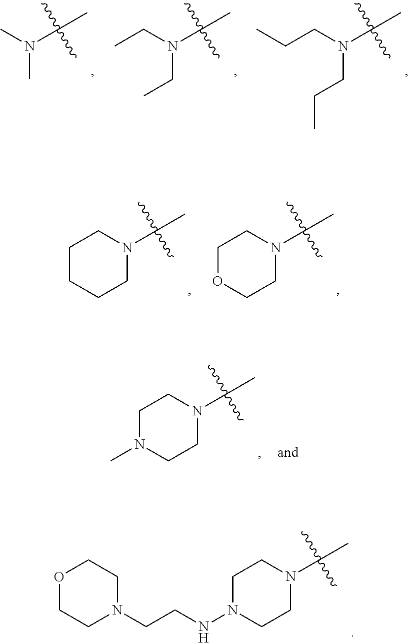

In some embodiments of a compound of Formula I, the EDG is

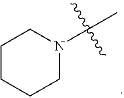 [SMILES: *N1CCCCC1]

In some embodiments of a compound of Formula I, EWG is an electron withdrawing group. In some embodiments of a compound of Formula I, the electron withdrawing group as used herein is any atom or group that is capable of drawing electron density from neighboring atoms towards itself, either by resonance or inductive effects. In some embodiments of a compound of Formula I, EWG is selected from a group consisting of halogen, —CN, —NO₂, —SO₃H, —CR₂₆R₂₇R₂₈, —COR₂₉, or —COOR₃₀; wherein each R₂₆, R₂₇ and R₂₈ is independently hydrogen or halogen; R₂₉ is halogen, hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₁; R₃₀ is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₂; and each R₃₁ and R₃₂ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

In some embodiments of a compound of Formula I, the EWG is selected from a group consisting of —F, —Cl, —Br, —C═O, NO₂, —CF₃, —CCl₃, —SO₃ and —CN. In some embodiments, the EWG is F, Cl, or Br. In some embodiments, the EWG is —CN.

In some embodiments of a compound of Formula I, WSG is a water soluble group. In some embodiments of a compound of Formula I, the WSG group serve to alter the solubility of the compounds of Formula I in an aqueous systems. In some embodiments of a compound of Formula I, WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₃; wherein each R₃₃ is independently halogen, —OR₃₄, —NR₃₅R₃₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇; each R₃₄, R₃₅ and R₃₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇; each R₃₇ is independently halogen, —OR₃₈, —NR₃₉R₄₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and each of R₃₈, R₃₉ and R₄₀ is independently hydrogen or C₁-C₁₀ alkyl.

In some embodiments of a compound of Formula I, the WSG is

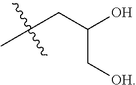 [SMILES: *CC(O)CO]

In some embodiments of a compound of Formula I, the WSG comprises polyethylene glycol, polypropylene glycol, co-polymer of polyethylene glycol and polypropylene glycol, or alkoxy derivatives thereof In some embodiments of a compound of Formula I, WSG is

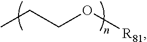

wherein n is an integer from 1-50 and R₈₁ is hydrogen, C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula I, R₈₁ is hydrogen. In some embodiments of a compound of Formula I, R₈₁ is methyl. In some embodiments of a compound of Formula I, R₈₁ is ethyl. In some embodiments of a compound of Formula I, R₈₁ is CH₂—C═CH. In some embodiments of a compound of Formula I, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments of a compound of Formula I, n is an integer of value 1-10, 1-20, 1-30, 1-40, 1-50, 10-20, 10-30, 10-40, 10-50, 20-30, 20-40, 20-50, 30-40, 30-50, or 40-50. In some embodiments of a compound of Formula I, n is 1, 2, 3, 4, 5, 6, 7, 8, 9, or 10. In some embodiments of a compound of Formula I, n is 3 or 6.

In some embodiments of a compound of Formula I, the WSG is

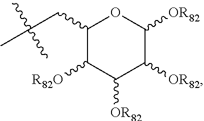

wherein each R₈₂ is hydrogen or C₁-C₁₀ alkyl. In some embodiments of a compound of Formula I, each R₈₂ is independently a hydrogen, methyl, ethyl, propyl, or butyl.

For embodiments of a compound of Formula I, the WSG is

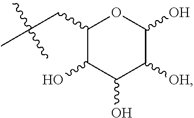 [SMILES: *CC1OC(O)C(O)C(O)C1O]

In some embodiments of a compound of Formula I, the WSG is

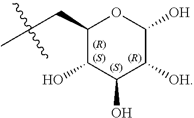

In some embodiments of a compound of Formula I, the WSG is

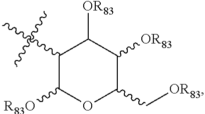

wherein each R₈₃ is hydrogen or C₁-C₁₀ alkyl. In some embodiments of a compound of Formula I, each R₈₃ is independently a hydrogen, methyl, ethyl, propyl, or butyl.

In some embodiments of a compound of Formula I,the WSG is

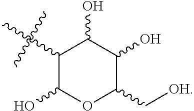

In some embodiments of a compound of Formula I, the WSG is

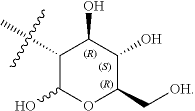

In some embodiments of a compound of Formula I, X is C═O or SO₂. In some embodiments of a compound of Formula I, X is C═O. In some embodiments of a compound of Formula I, X is SO₂.

In some embodiments of a compound of Formula I, Y is NH or S. In some embodiments of a compound of Formula I, Y is NH. In other embodiments Y is S.

In some embodiments of a compound of Formula I, the variable w is an integer from 1-5. In some embodiments, w is 1. In some embodiments, w is 2. In some embodiments, w is 3. In some embodiments, w is 4. In some embodiments, w is 5.

In some embodiments of a compound of Formula I, the variable x is an integer from 0-10. In some embodiments of a compound of Formula I, x is 0. In some embodiments of a compound of Formula I, x is 1. In some embodiments of a compound of Formula I, x is 2. In some embodiments of a compound of Formula I, x is 3. In some embodiments of a compound of Formula I, x is 4. In some embodiments of a compound of Formula I, xis 5. In some embodiments of a compound of Formula I, x is 6. In some embodiments of a compound of Formula I, x is 7. In some embodiments of a compound of Formula I, x is 8. In some embodiments of a compound of Formula I, x is 9. In some embodiments of a compound of Formula I, x is 10.

In some embodiments of a compound of Formula I, the variable y is an integer from 0-10. In some embodiments of a compound of Formula I, y is 0. In some embodiments of a compound of Formula I, y is 1. In some embodiments of a compound of Formula I, y is 2. In some embodiments of a compound of Formula I, y is 3. In some embodiments of a compound of Formula I, y is 4. In some embodiments of a compound of Formula I, y is 5. In some embodiments of a compound of Formula I, y is 6. In some embodiments of a compound of Formula I, y is 7. In some embodiments of a compound of Formula I, y is 8. In some embodiments of a compound of Formula I, y is 9. In some embodiments of a compound of Formula I, y is 10.

In some embodiments of a compound of Formula I, the variable z is an integer from 1-10. In some embodiments of a compound of Formula I, z is 1. In some embodiments of a compound of Formula I, z is 2. In some embodiments of a compound of Formula I, z is 3. In some embodiments of a compound of Formula I, z is 4. In some embodiments of a compound of Formula I, z is 5. In some embodiments of a compound of Formula I, z is 6. In some embodiments of a compound of Formula I, z is 7. In some embodiments of a compound of Formula I, z is 8. In some embodiments of a compound of Formula I, z is 9. In some embodiments of a compound of Formula I, z is 10.

In one aspect the disclosure provides a compound of Formula Ia:

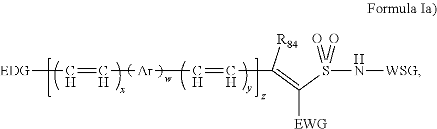

wherein EDG, Ar, R₈₄, x, w, y, z and WSG are as defined as above for Formula I.

In one aspect the disclosure provides a compound of Formula Ia:

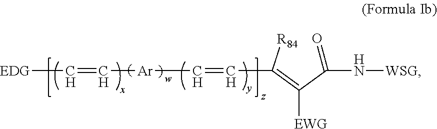

wherein EDG, Ar, R₈₄, x, w, y, z and WSG are as defined above for Formula I.

In some embodiments, the compound of Formula I is selected from a group consisting of

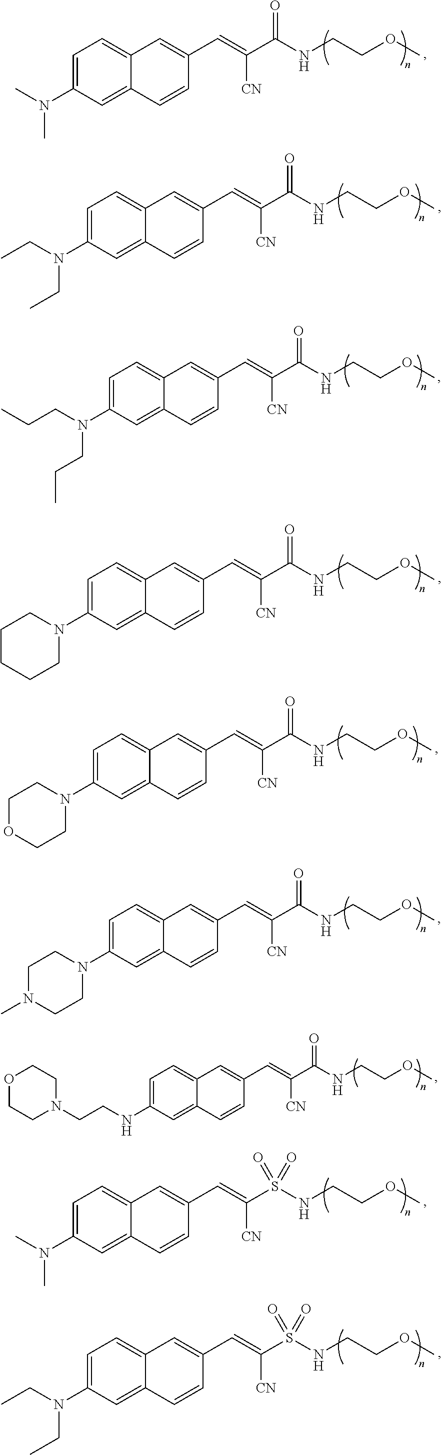

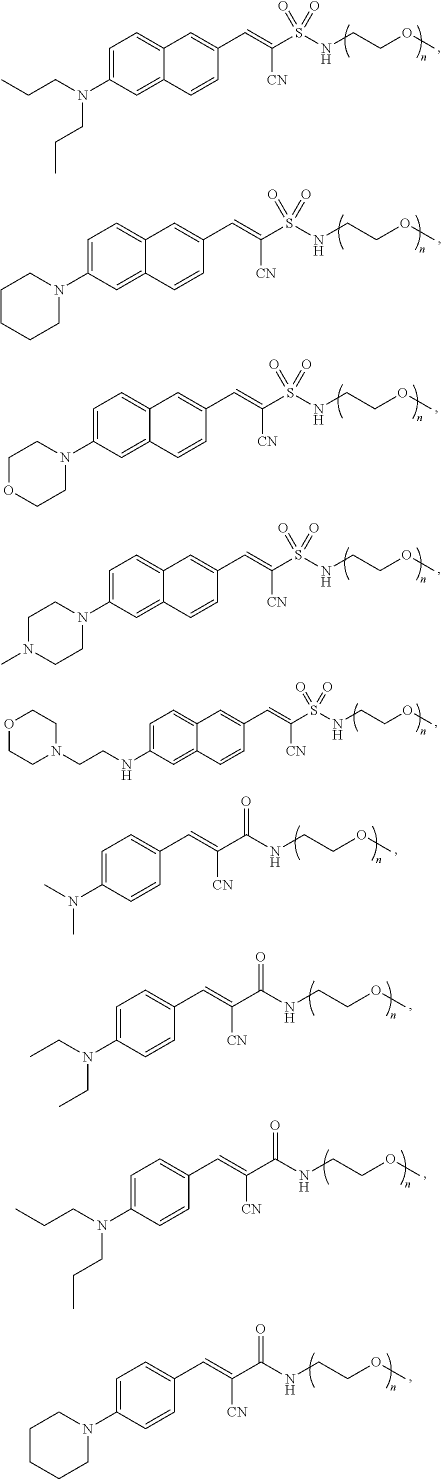

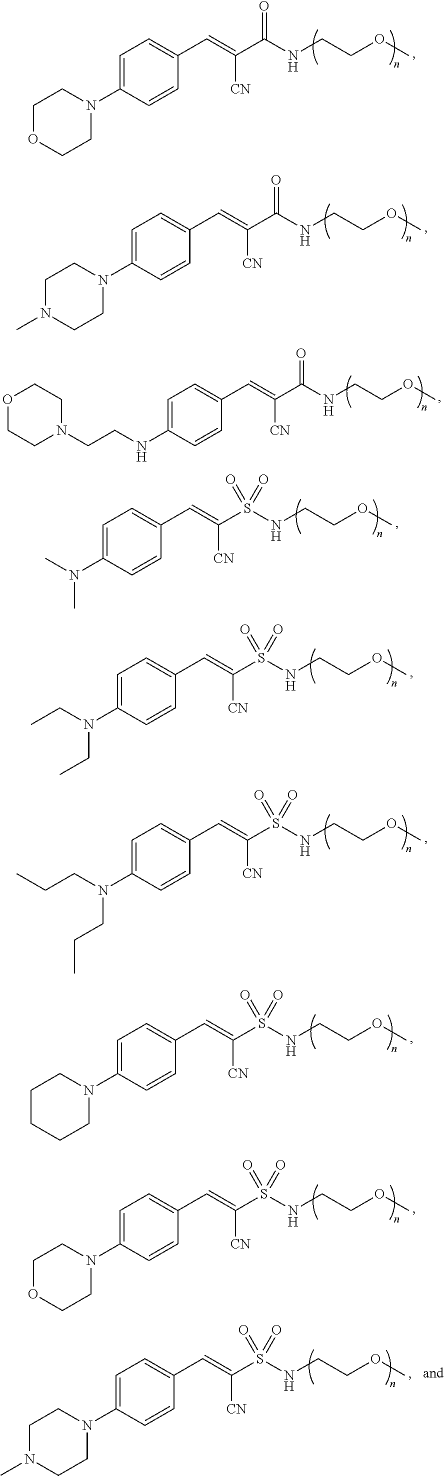

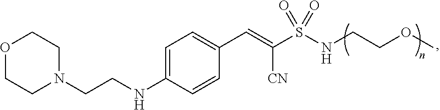

wherein n is an integer with value 1-50. In some embodiments of a compound of Formula I, n is a integer of value 1-10, e.g. n is 1, 2, 3, 4, 5, 6, 7, 8, 9, or 10.

In other embodiments the compound of Formula I is selected from a group consisting of

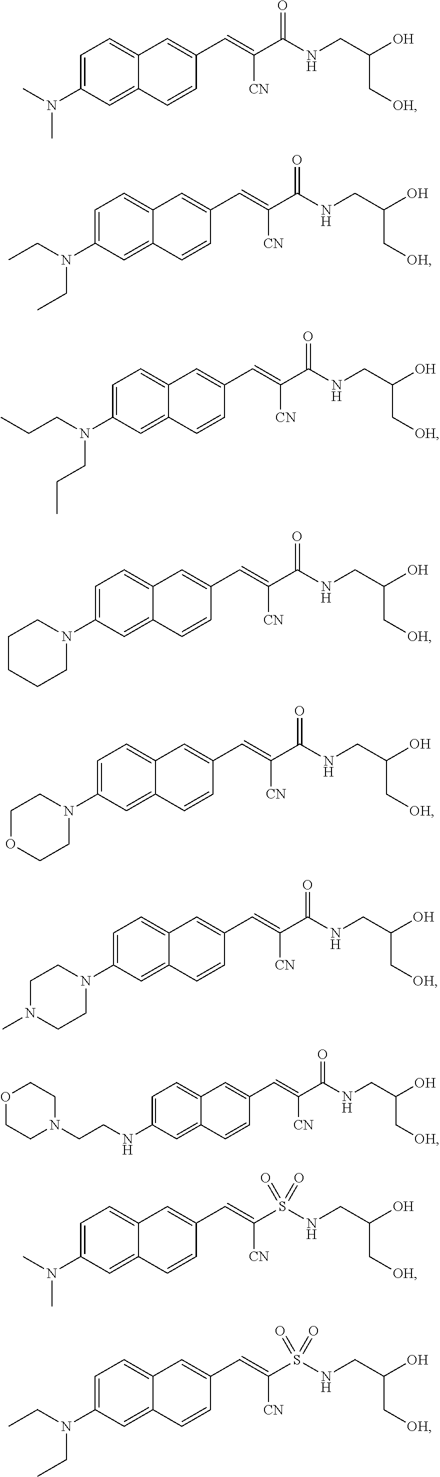

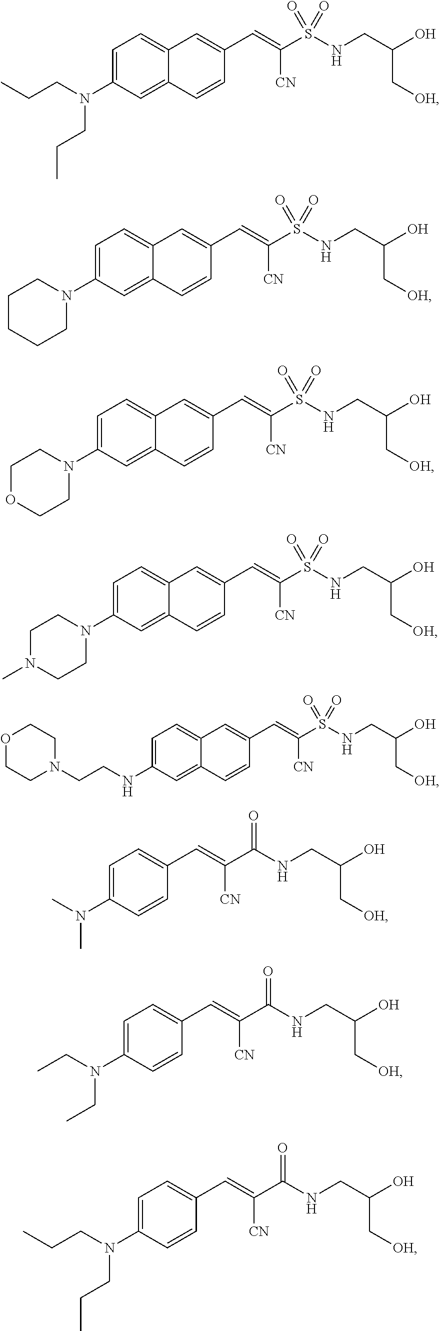

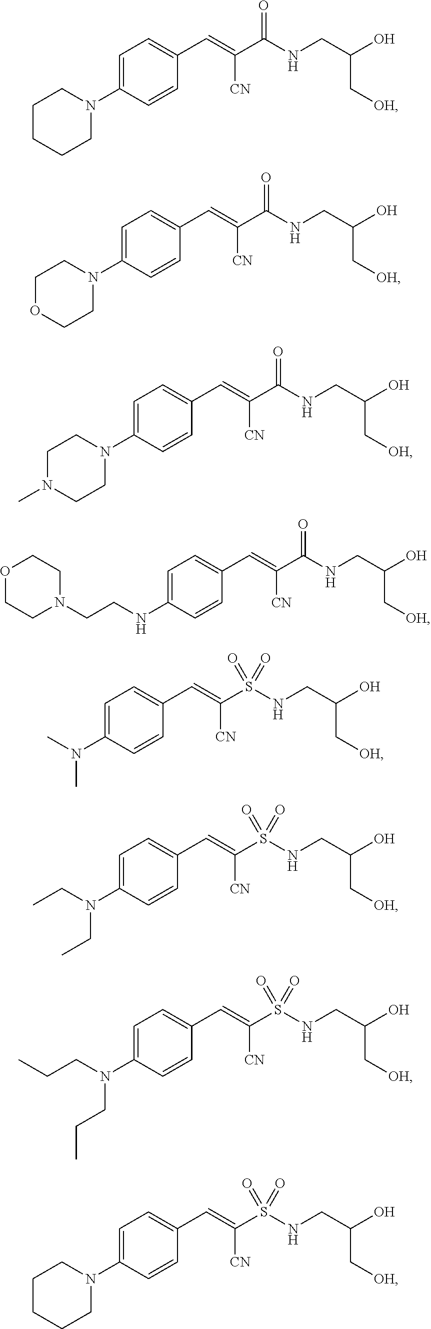

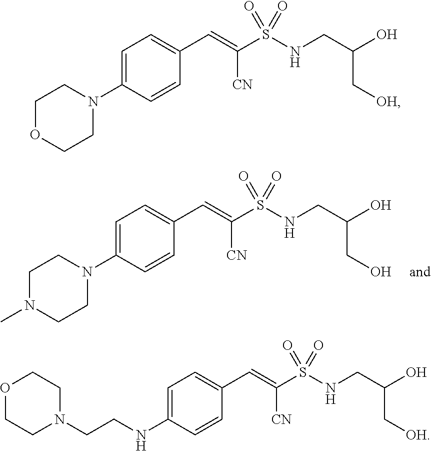 [SMILES: N#C/C(=C\c1ccc(N2CCOCC2)cc1)S(=O)(=O)NCC(O)CO ; CN1CCN(c2ccc(/C=C(\C#N)S(=O)(=O)NCC(O)CO)cc2)CC1 ; N#C/C(=C\c1ccc(NCCN2CCOCC2)cc1)S(=O)(=O)NCC(O)CO]

In some embodiments, the compound of Formula I is selected from a group consisting of

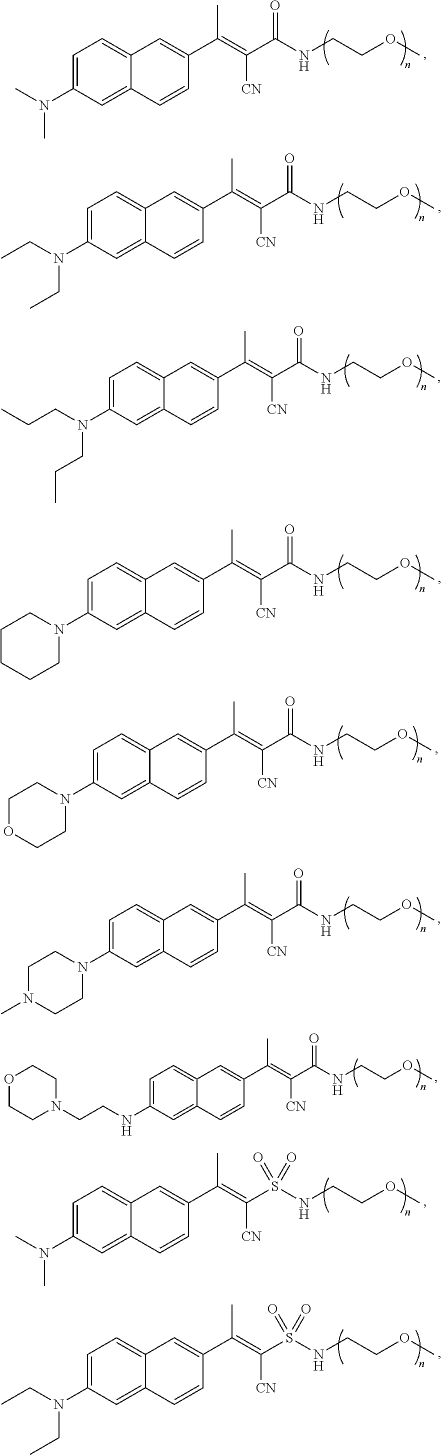

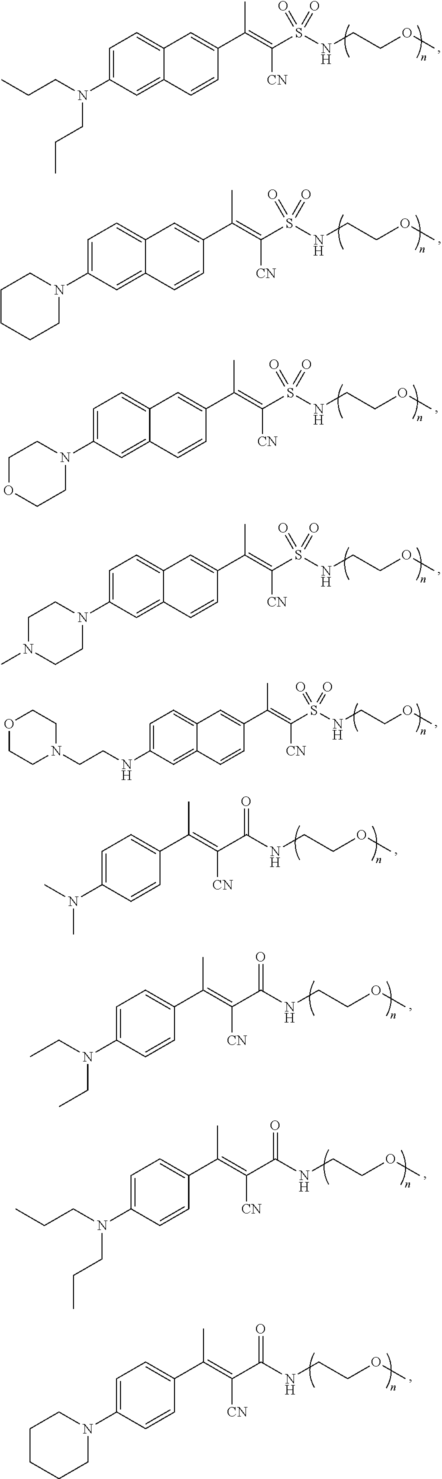

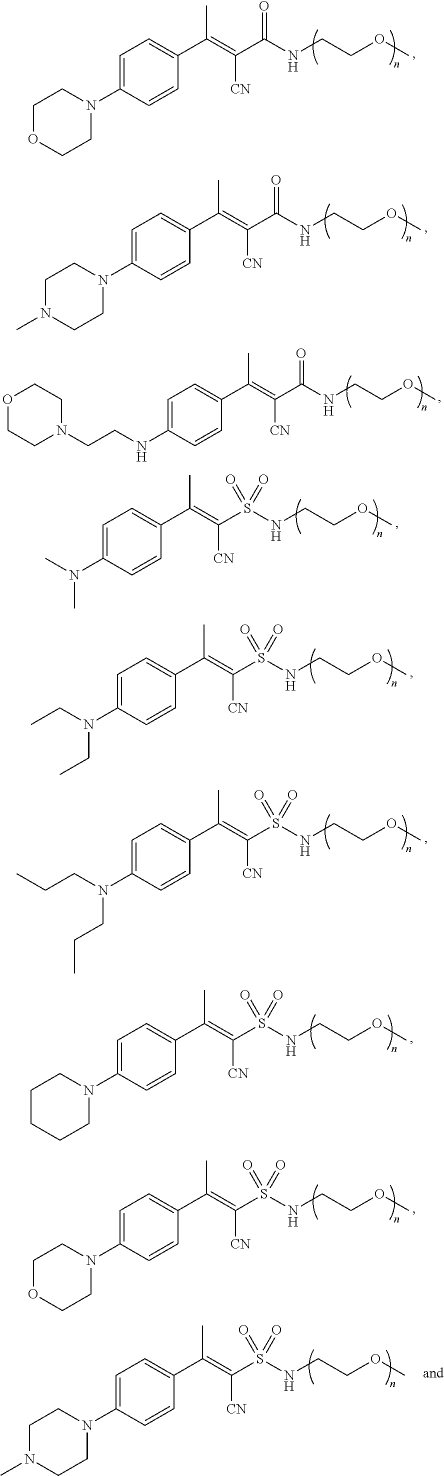

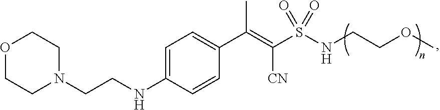

wherein n is an integer with value 1-50. In some embodiments of a compound of Formula I, n is a integer of value 1-10, e.g. n is 1, 2, 3, 4, 5, 6, 7, 8, 9, or 10.

In other embodiments the compound of Formula I is selected from a group consisting of

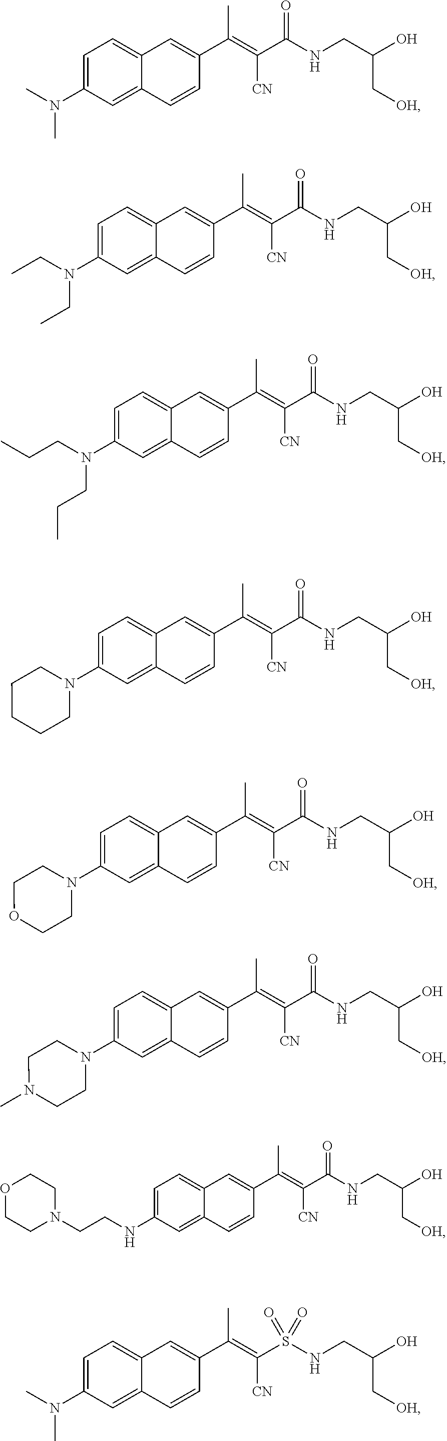

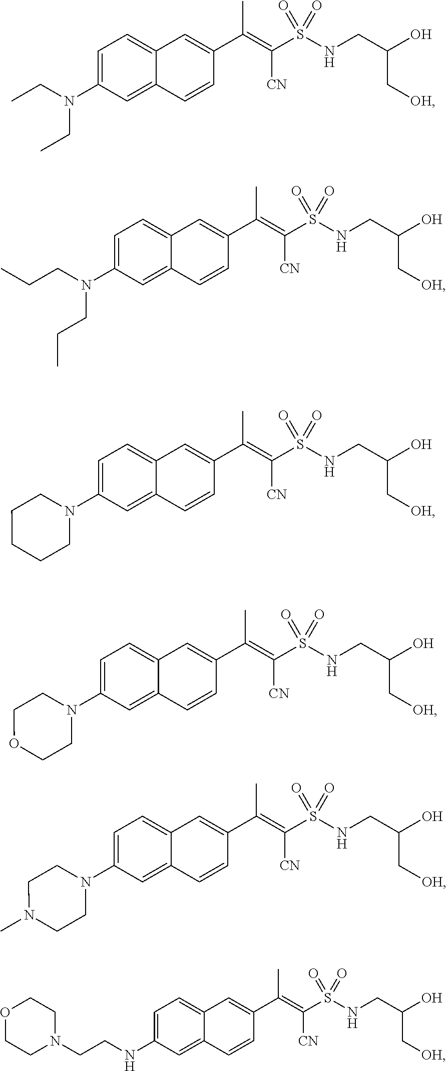 [SMILES: CCN(CC)c1ccc2cc(/C(C)=C(\C#N)S(=O)(=O)NCC(O)CO)ccc2c1 ; CCCN(CCC)c1ccc2cc(/C(C)=C(\C#N)S(=O)(=O)NCC(O)CO)ccc2c1 ; C/C(=C(/C#N)S(=O)(=O)NCC(O)CO)c1ccc2cc(N3CCCCC3)ccc2c1 ; C/C(=C(/C#N)S(=O)(=O)NCC(O)CO)c1ccc2cc(N3CCOCC3)ccc2c1 ; C/C(=C(/C#N)S(=O)(=O)NCC(O)CO)c1ccc2cc(N3CCN(C)CC3)ccc2c1 ; C/C(=C(/C#N)S(=O)(=O)NCC(O)CO)c1ccc2cc(NCCN3CCOCC3)ccc2c1]

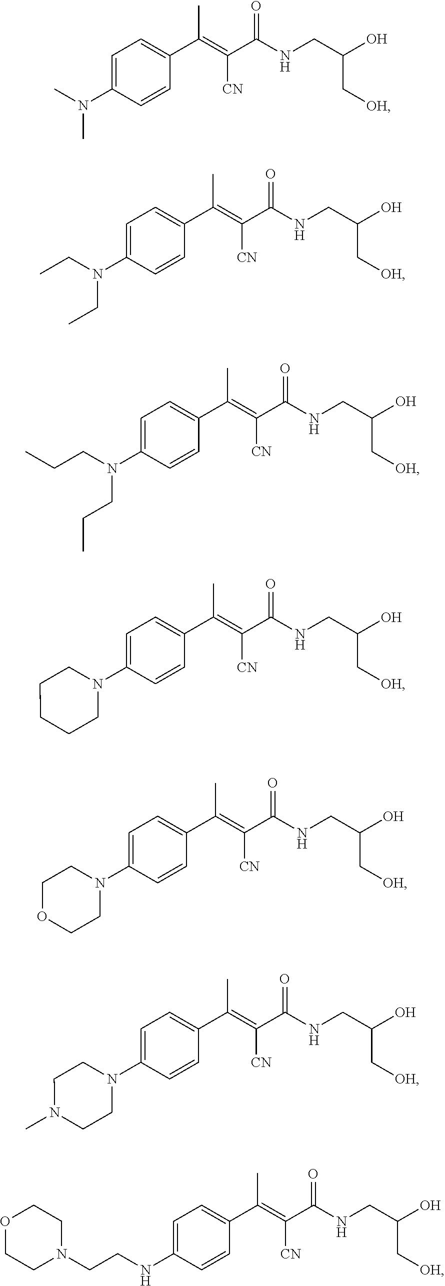

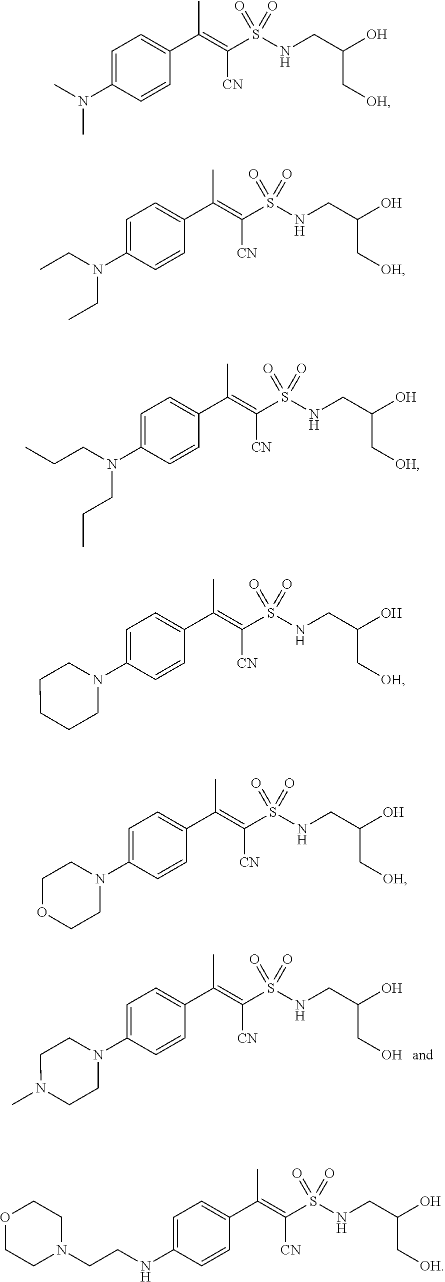

In some embodiments, the compound of Formula I is selected from a group consisting of

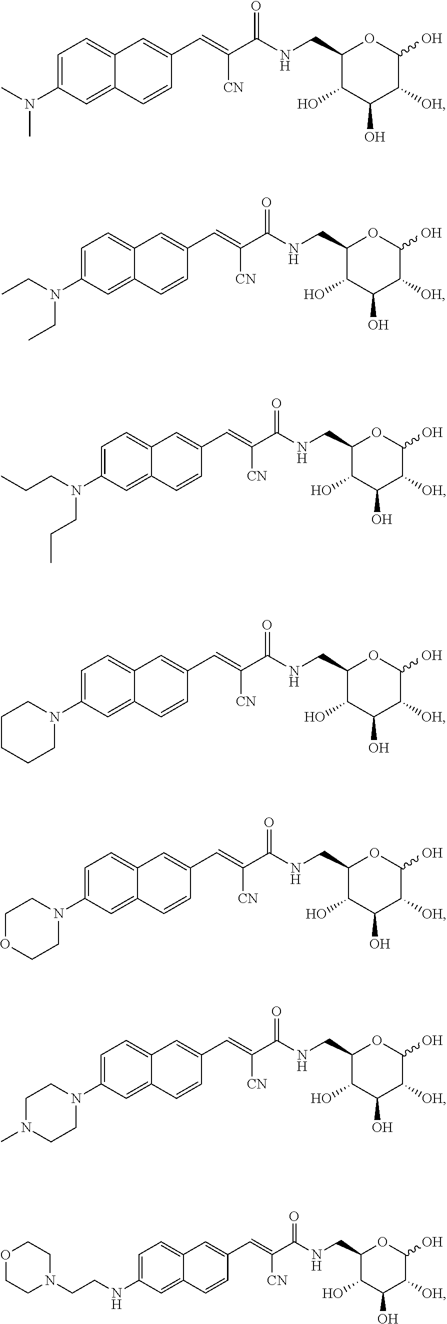

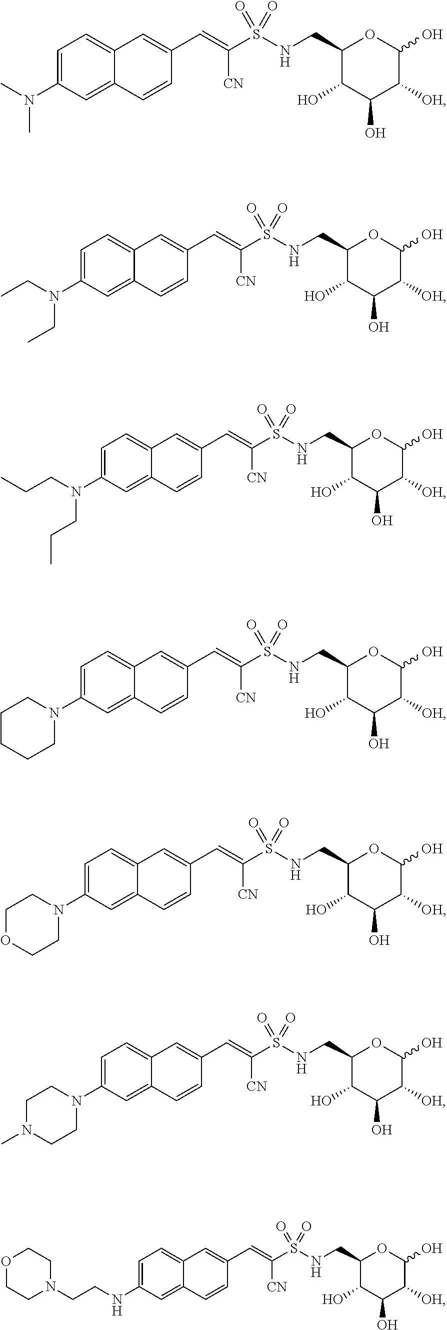

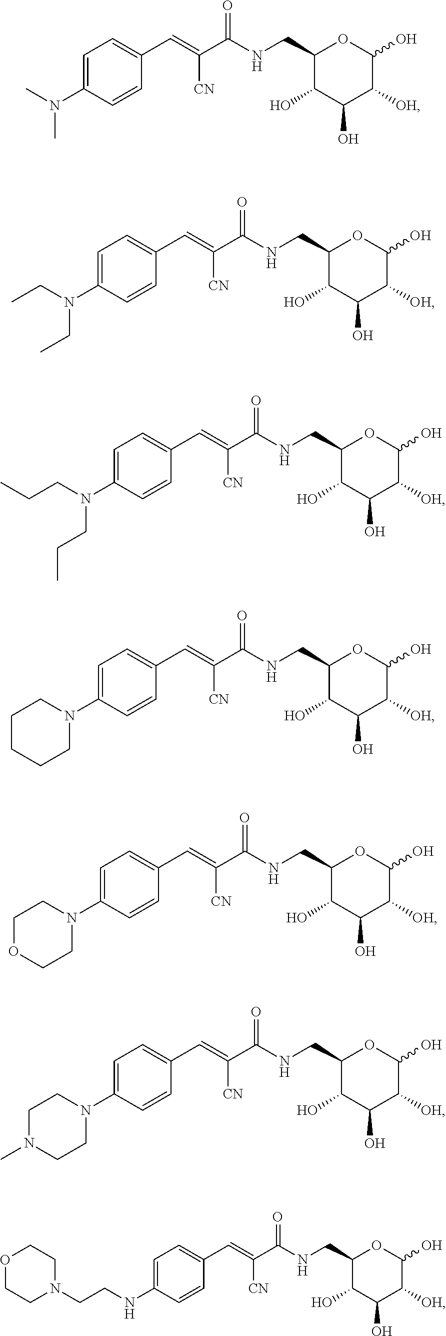

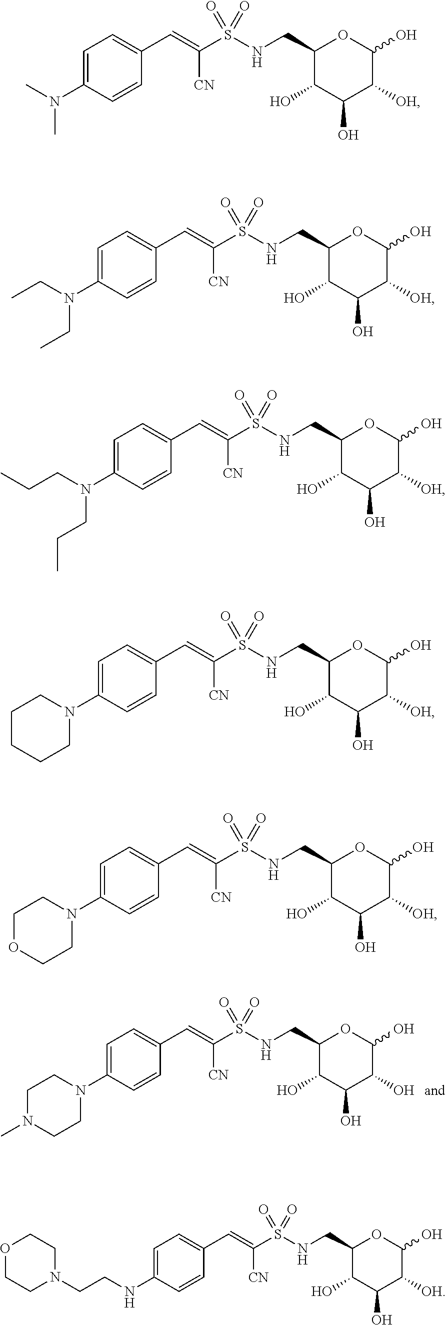

In other embodiments the compound of Formula I is selected from a group consisting of

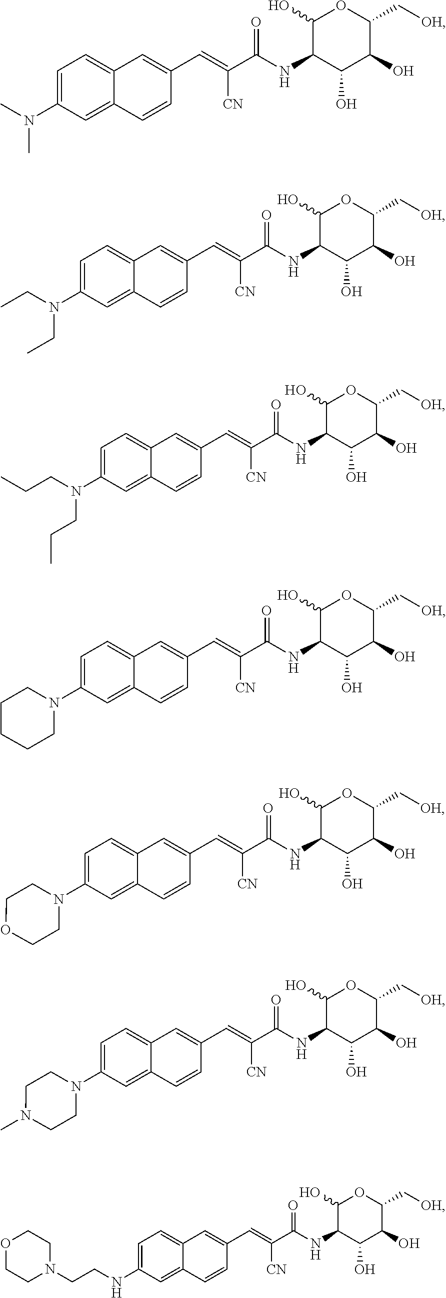

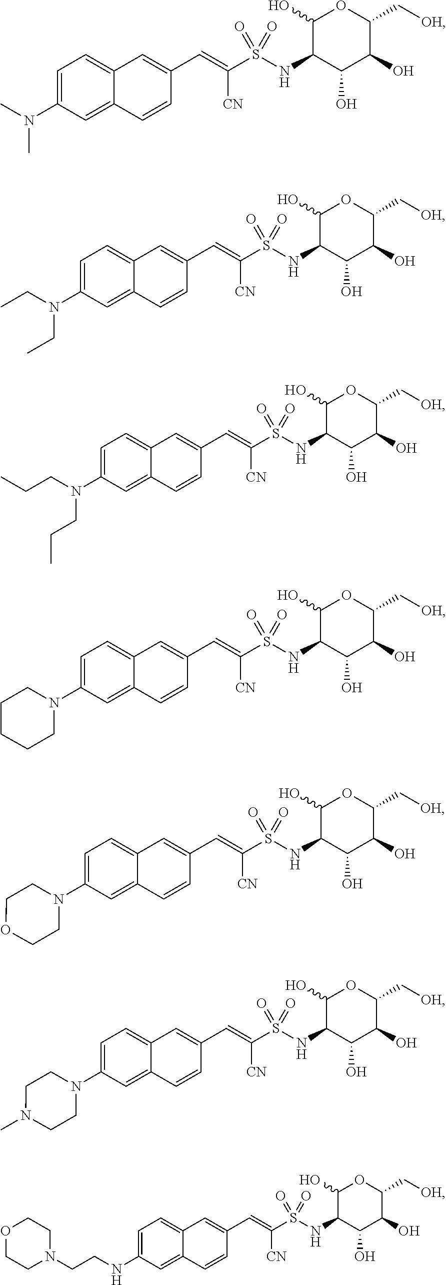

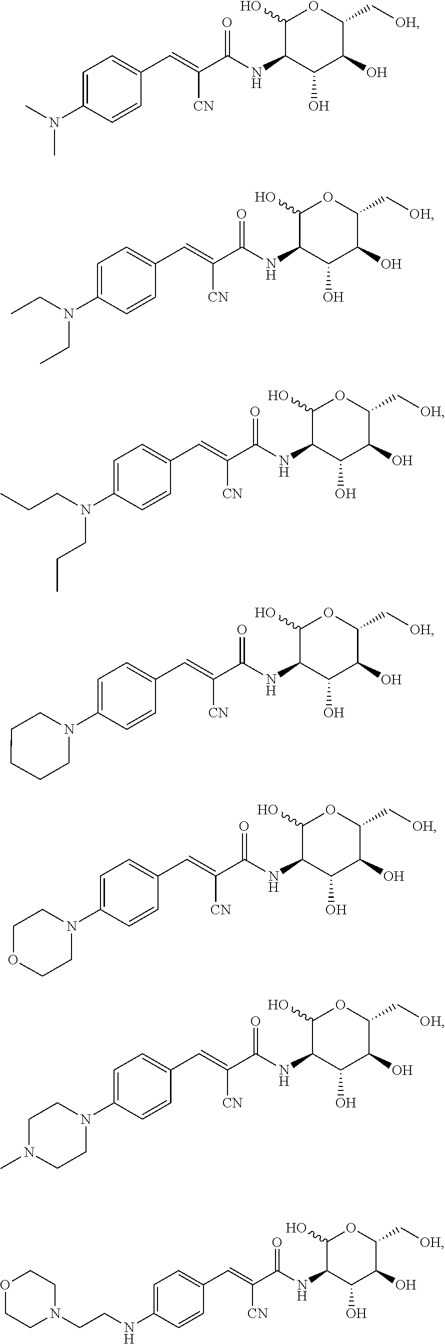

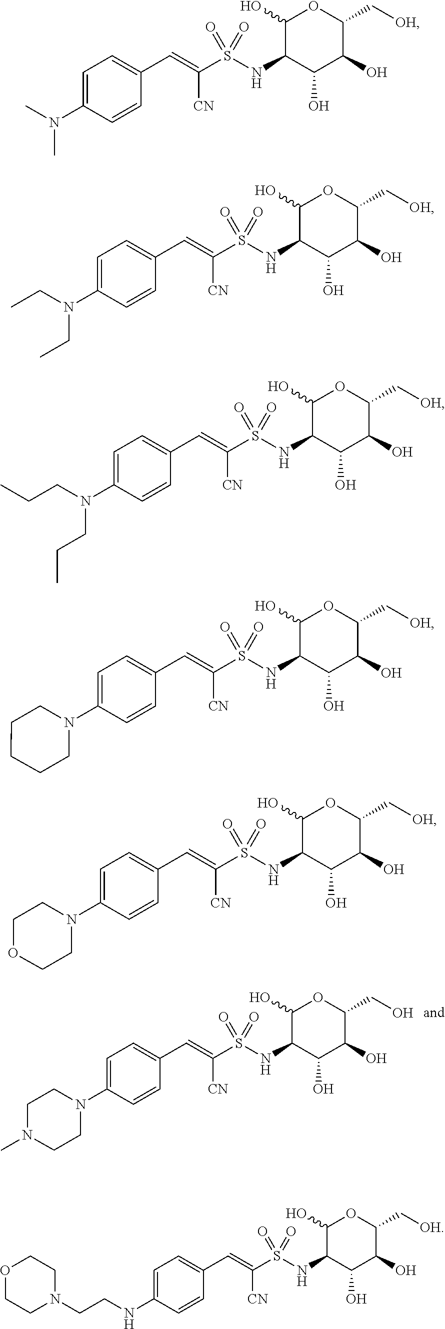

In other embodiments the compound of Formula I is selected from a group consisting of

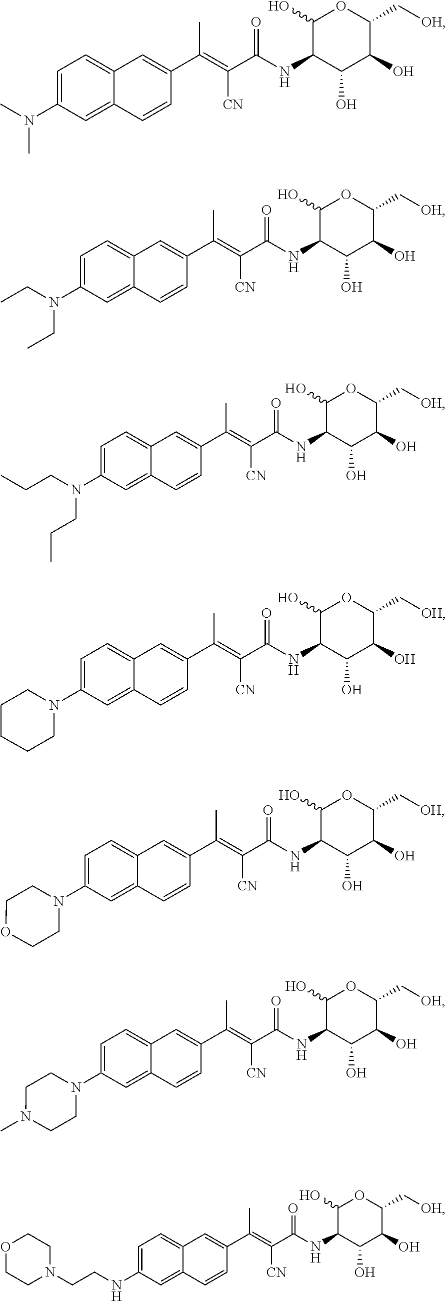

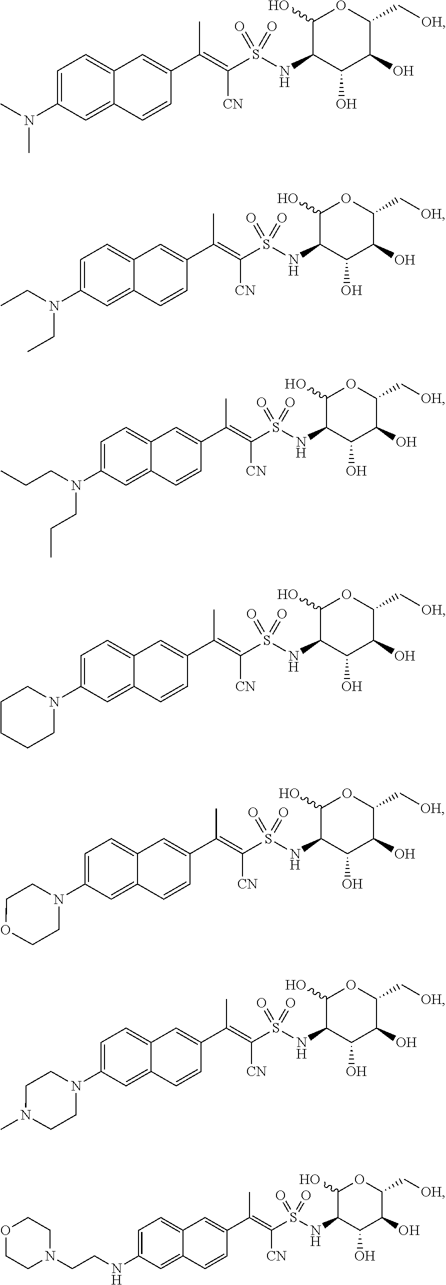

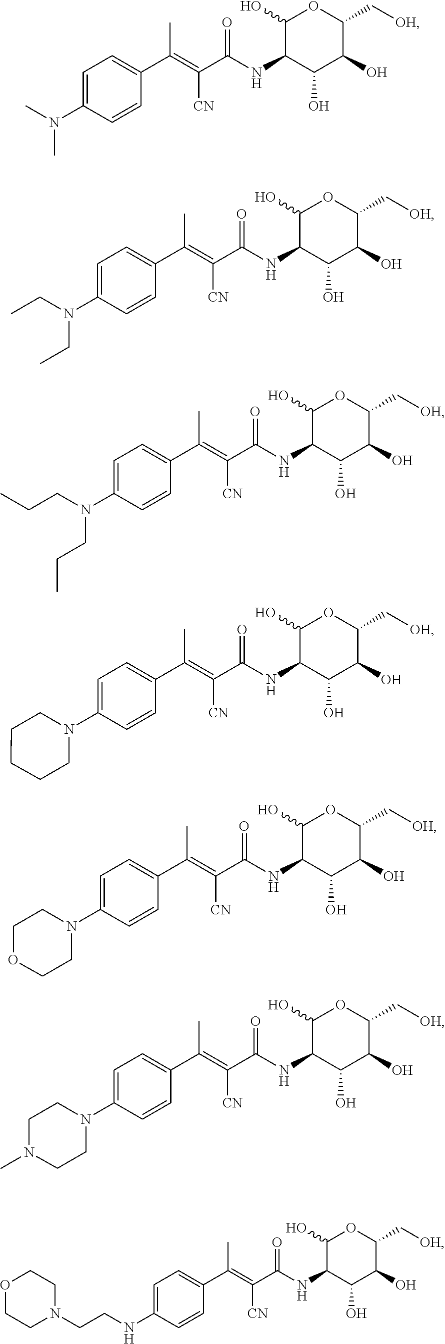

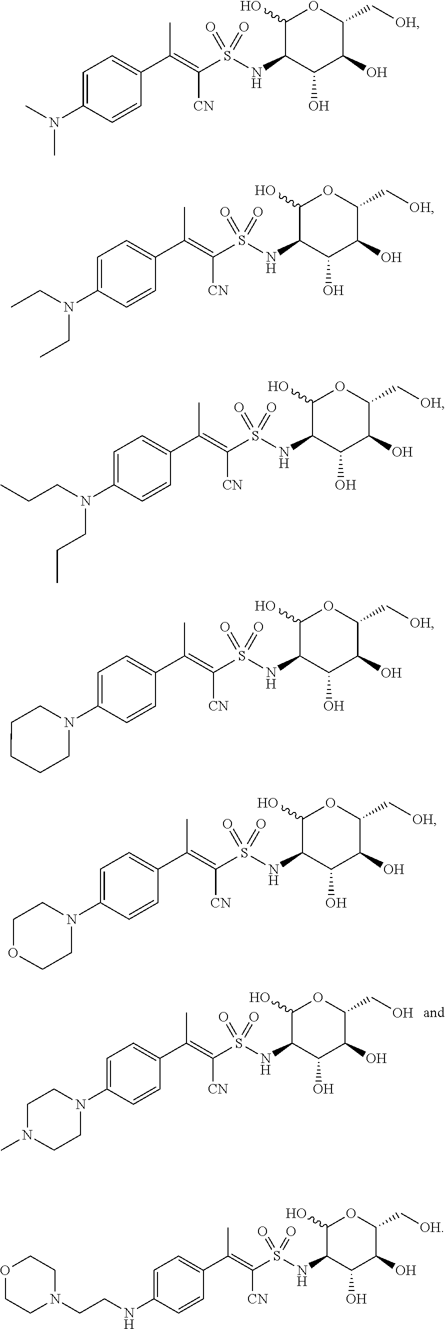

In some embodiments, the compound of Formula I is selected from a group consisting of

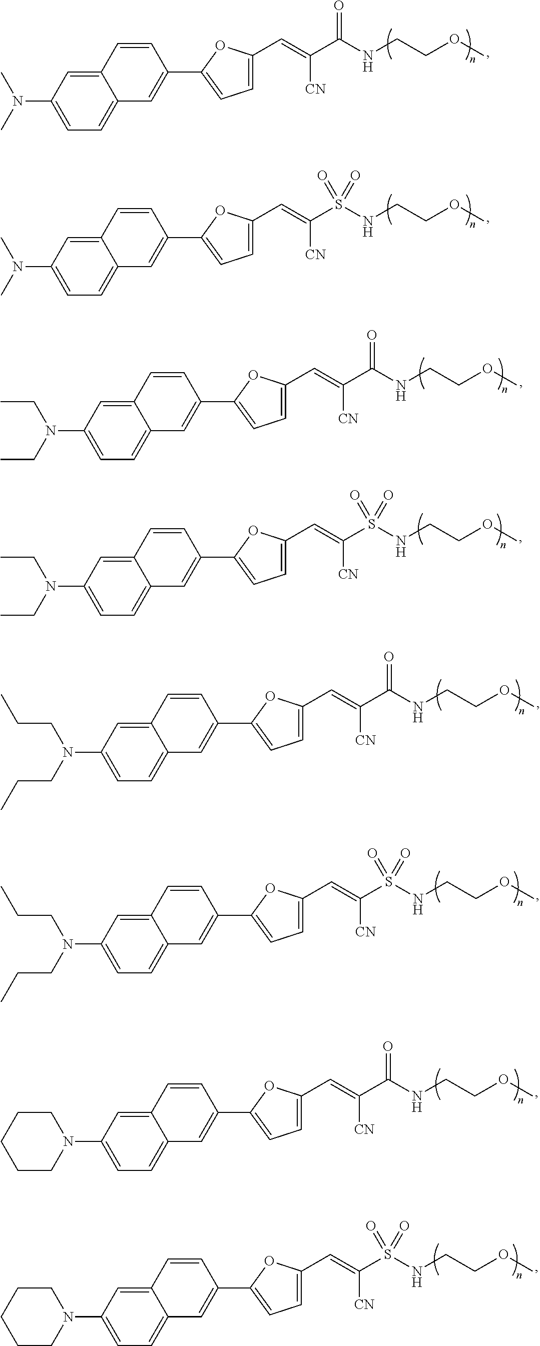

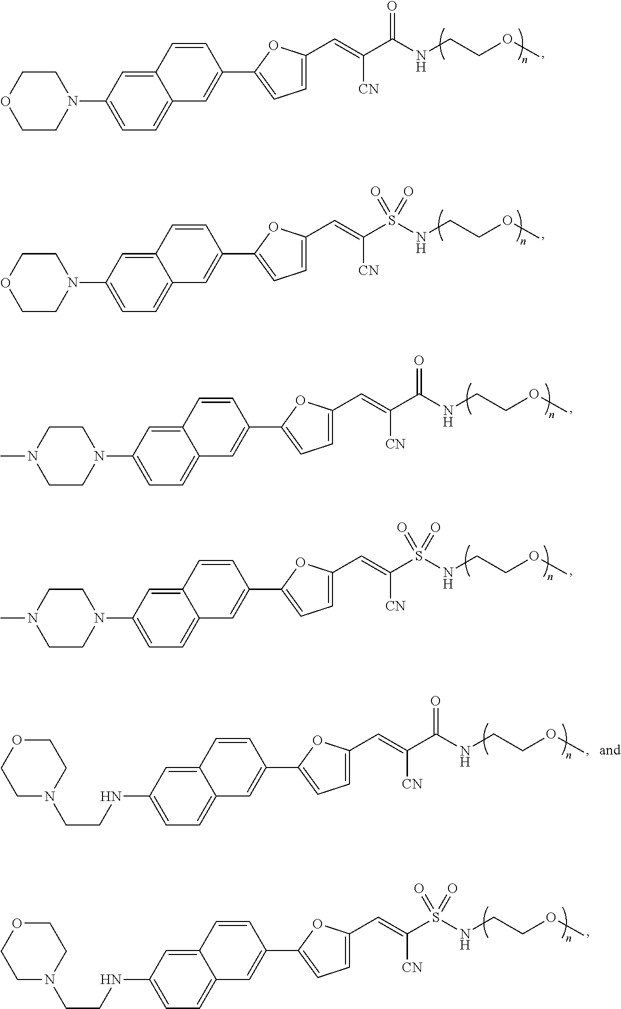

wherein n is an integer with value 1-50. In some embodiments of a compound of Formula I, n is a integer of value 1-10, e.g. n is 1, 2, 3, 4, 5, 6, 7, 8, 9, or 10.

In some embodiments, the compound of Formula I is selected from a group consisting of

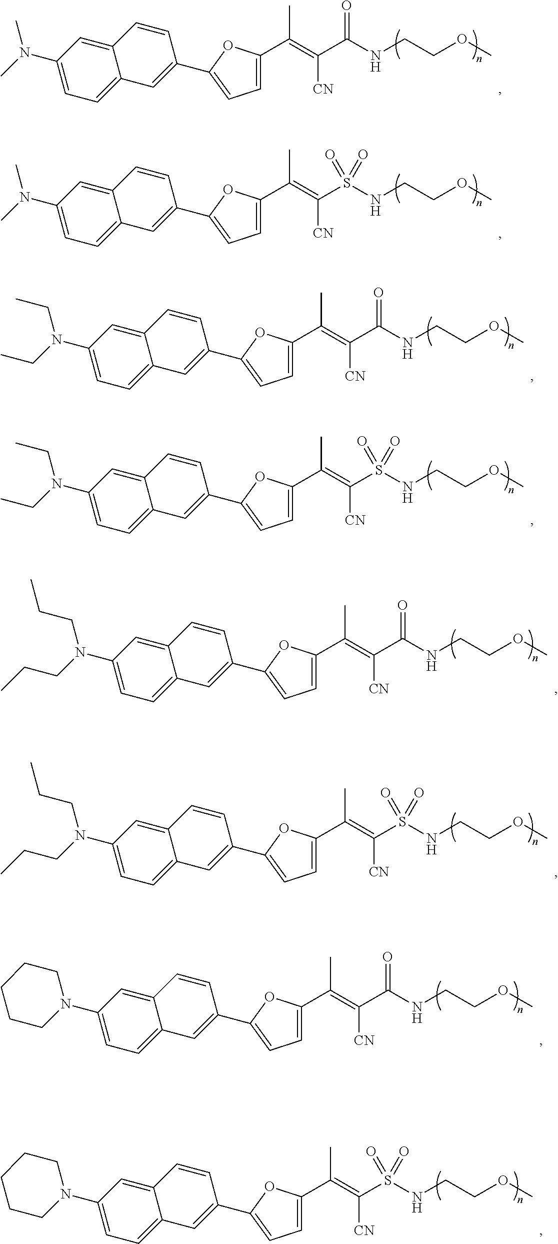

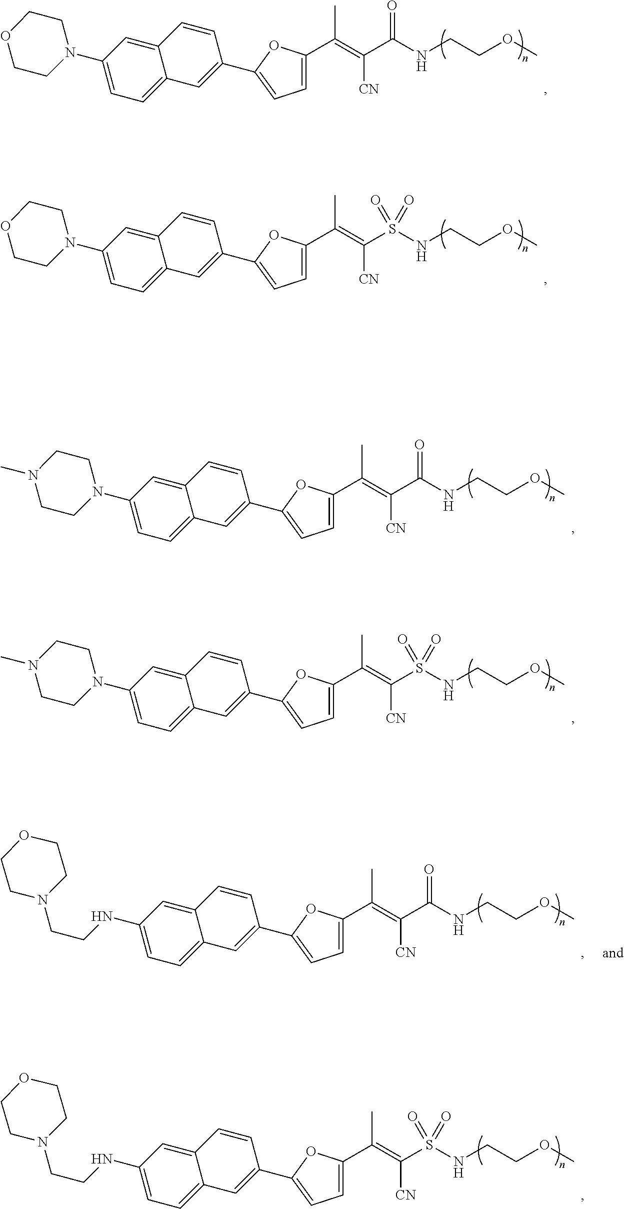

wherein n is an integer with value 1-50. In some embodiments of a compound of Formula I, n is a integer of value 1-10, e.g. n is 1, 2, 3, 4, 5, 6, 7, 8, 9, or 10.

In some embodiments, the compound of Formula I is selected from a group consisting of

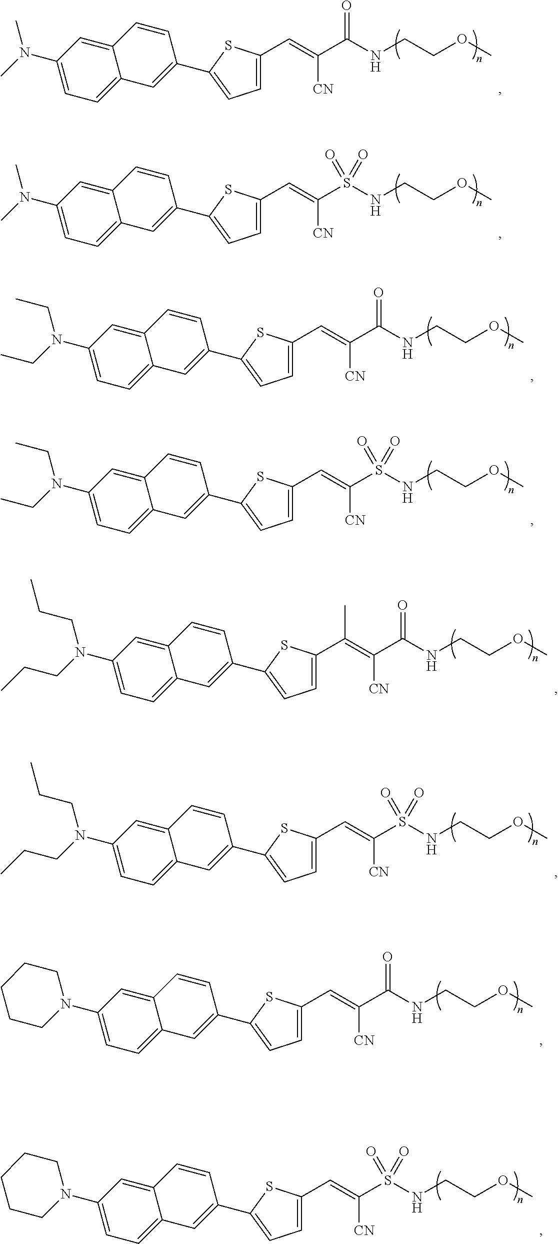

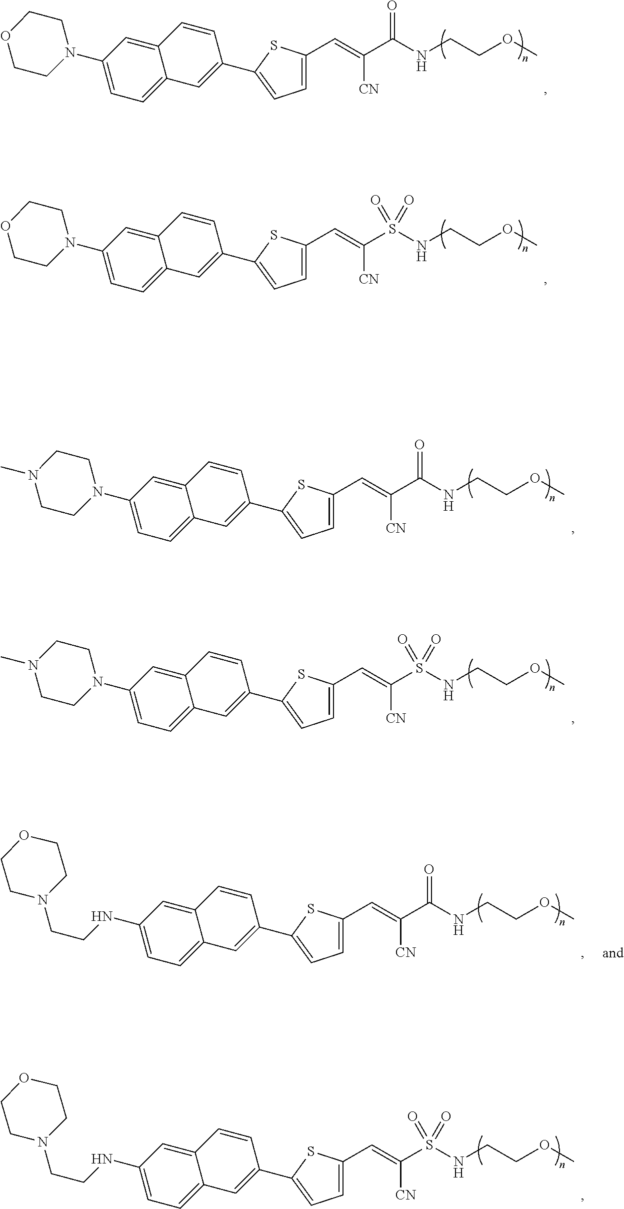

wherein n is an integer with value 1-5. In some embodiments of a compound of Formula I, n is an integer of value 1-10, e.g. n is 1, 2, 3, 4, 5, 6, 7, 8, 9, or 10.

In some embodiments, the compound of Formula I is selected from a group consisting of

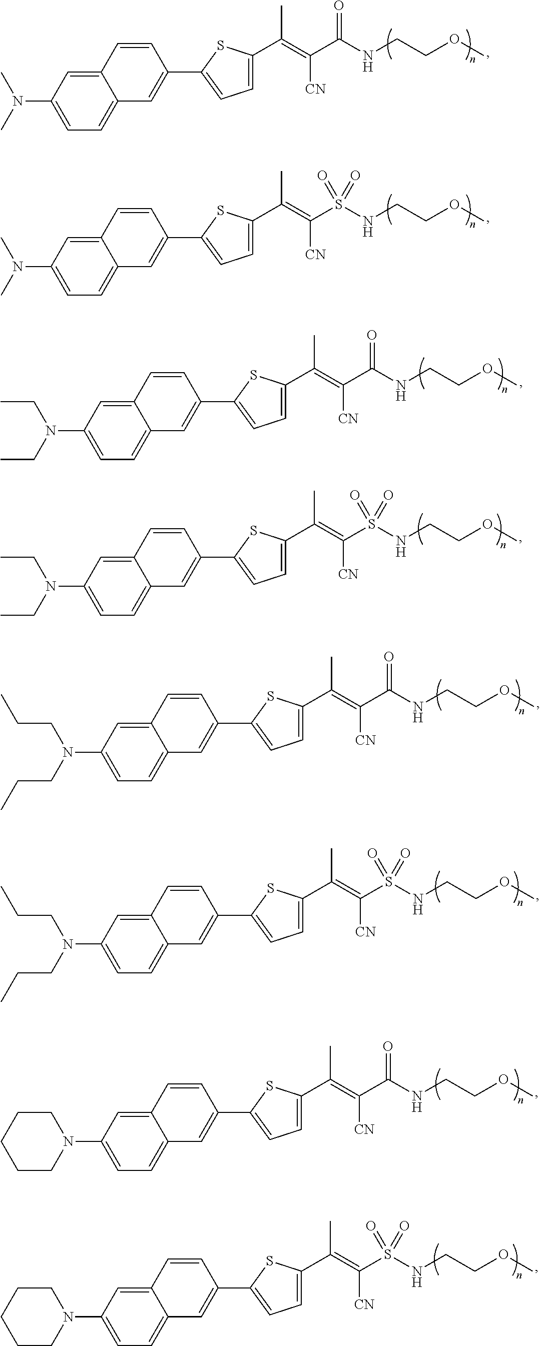

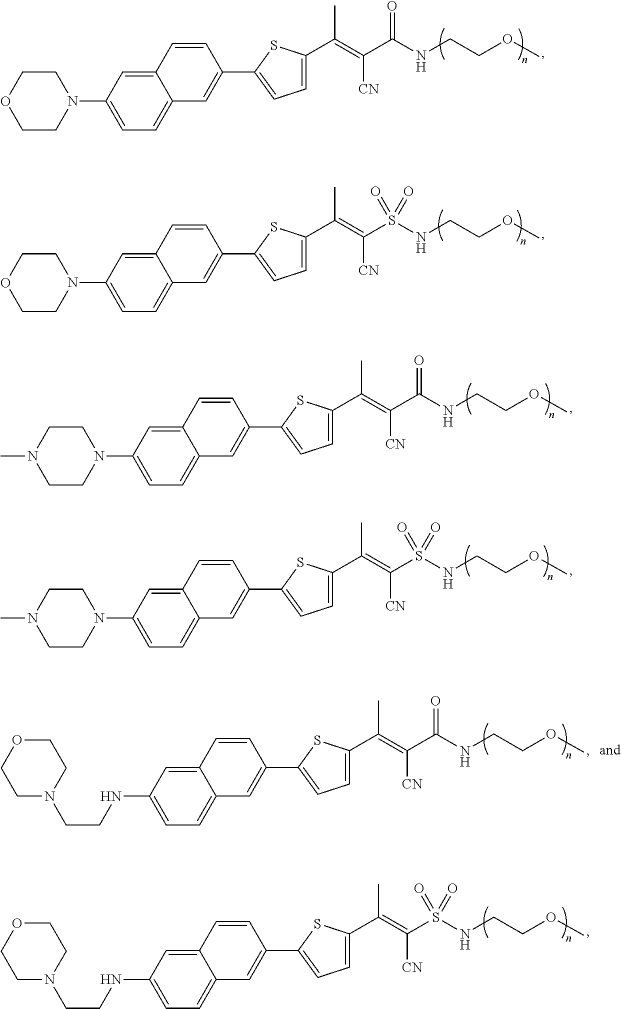

wherein n is an integer with value 1-50. In some embodiments of a compound of Formula I, n is an integer of value 1-10, e.g. n is 1, 2, 3, 4, 5, 6, 7, 8, 9, or 10.

In some embodiments, the compound of Formula I is selected from a group consisting of

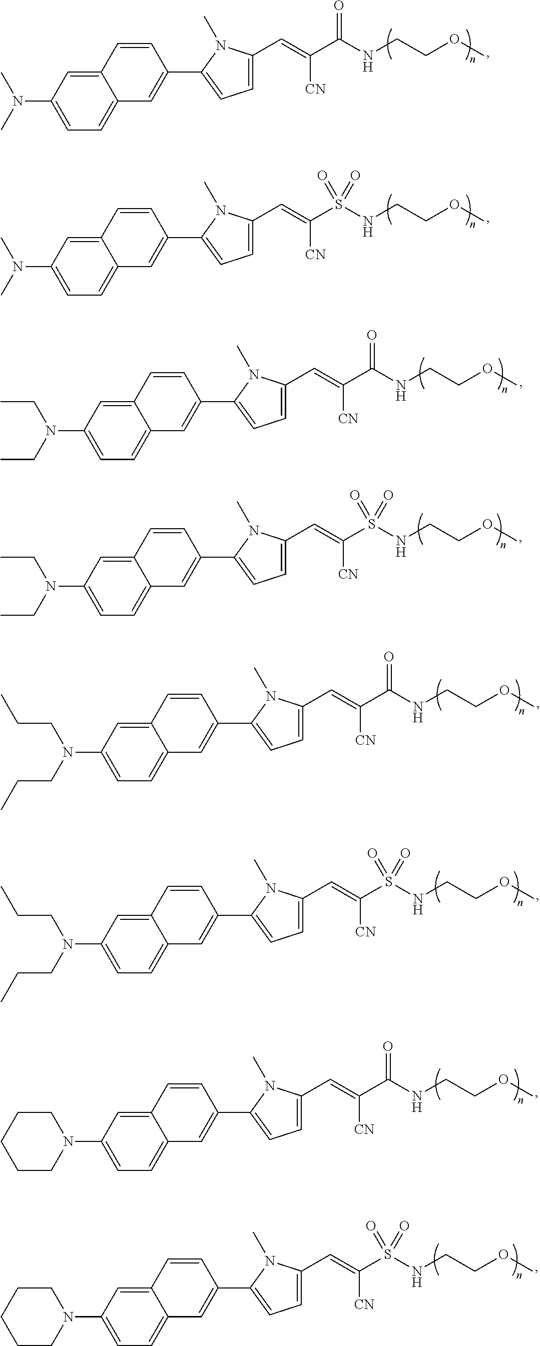

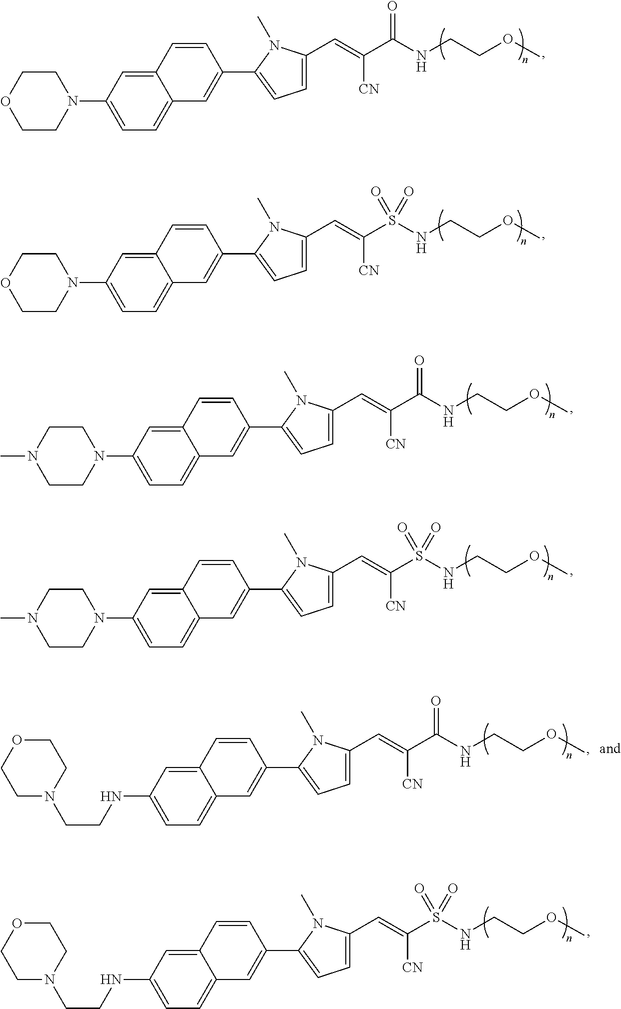

wherein n is an integer with value 1-50. In some embodiments of a compound of Formula I, n is an integer of value 1-10, e.g. n is 1, 2, 3, 4, 5, 6, 7, 8, 9, or 10.

In some embodiments, the compound of Formula I is selected from a group consisting of

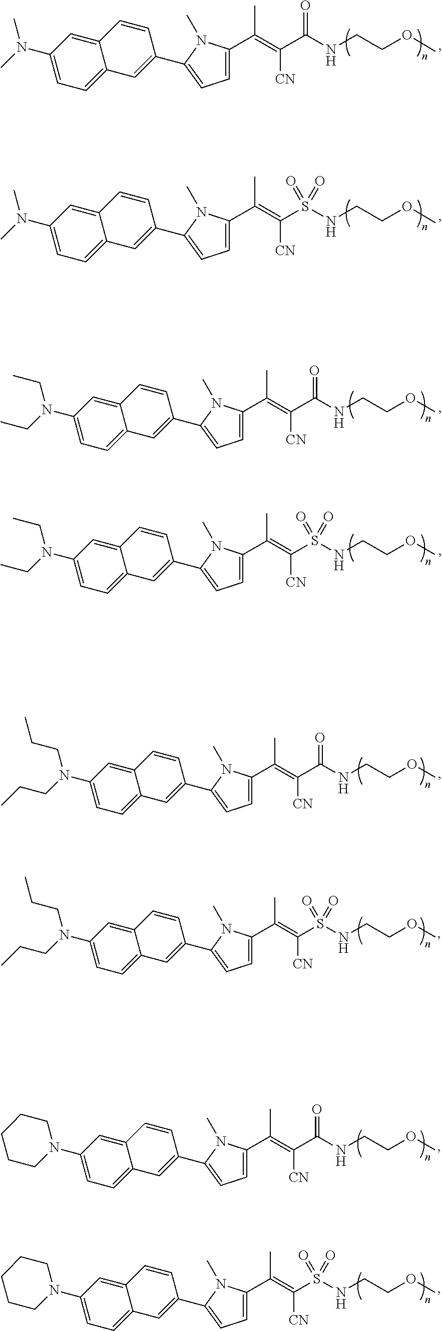

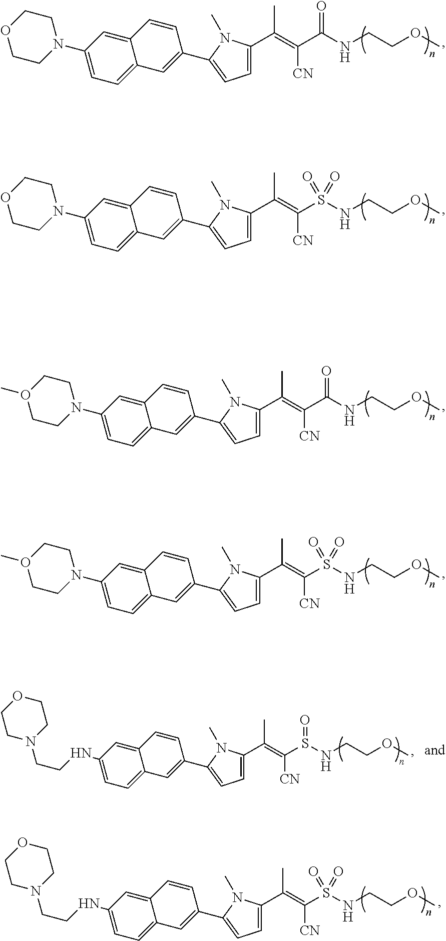

wherein n is an integer with value 1-50. In some embodiments of a compound of Formula I, n is an integer of value 1-10, e.g. n is 1, 2, 3, 4, 5, 6, 7, 8, 9, or 10.

In some embodiments, the compound of Formula I is selected from a group consisting of

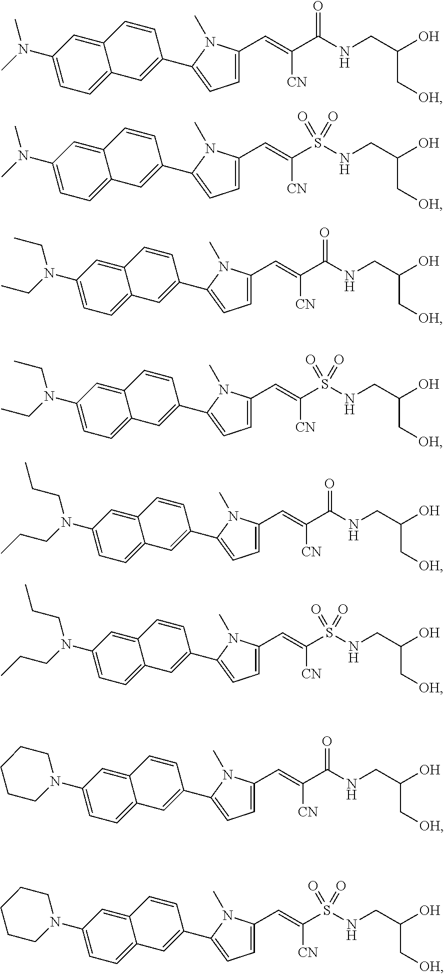

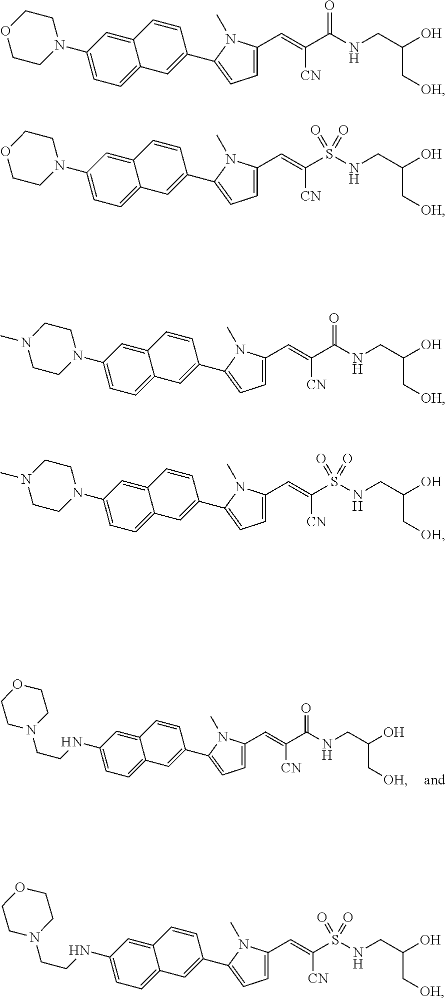

In some embodiments, the compound of Formula I is selected from a group consisting of

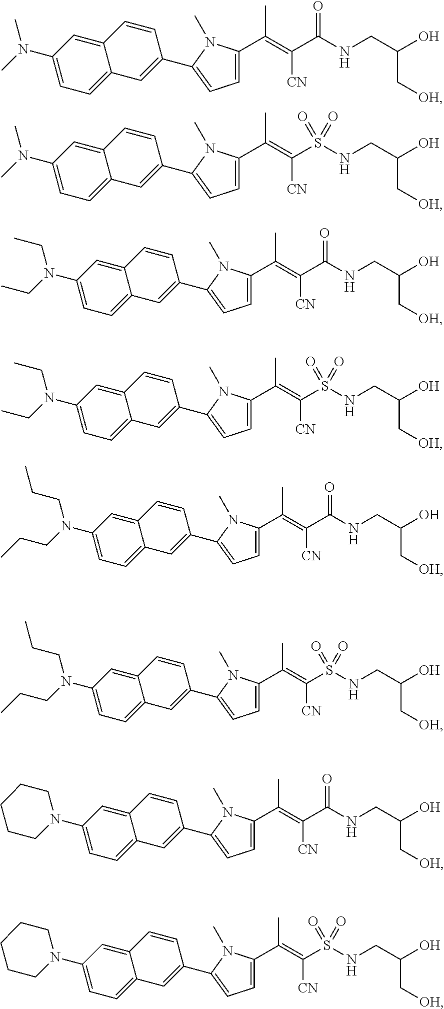

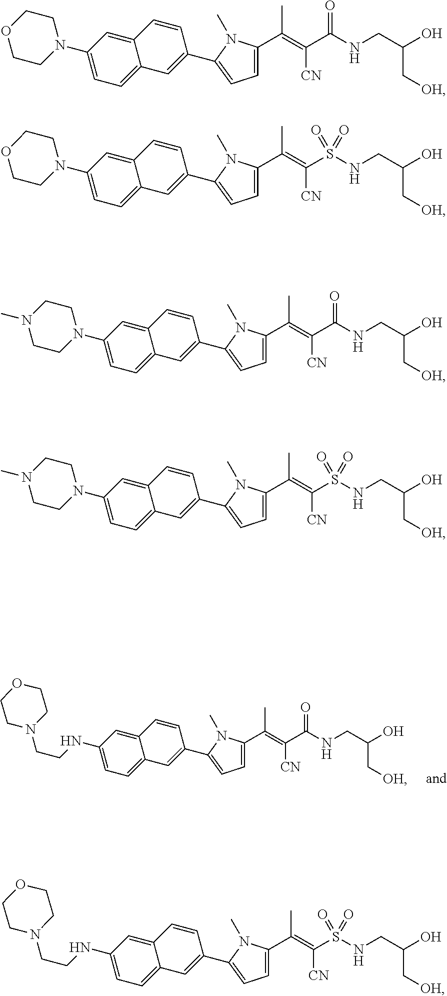

In some embodiments, the compound of Formula I is selected from a group consisting of

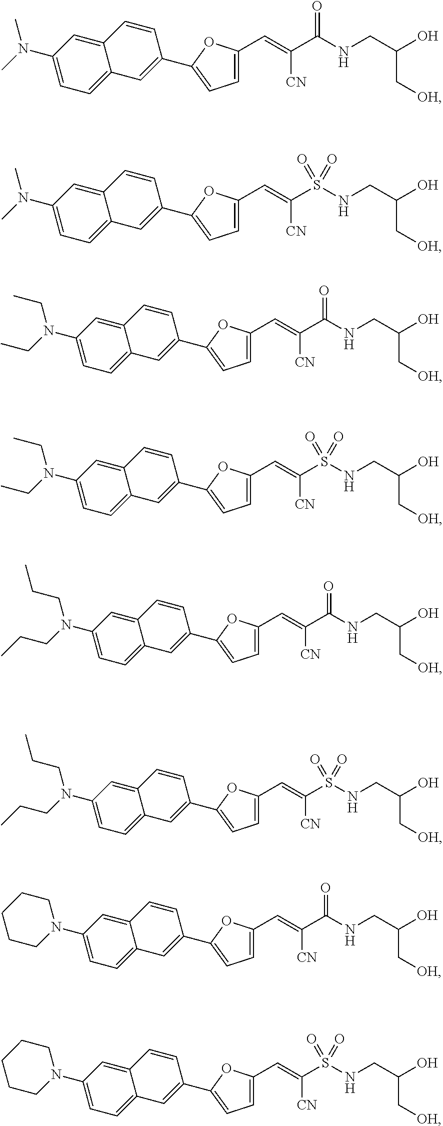

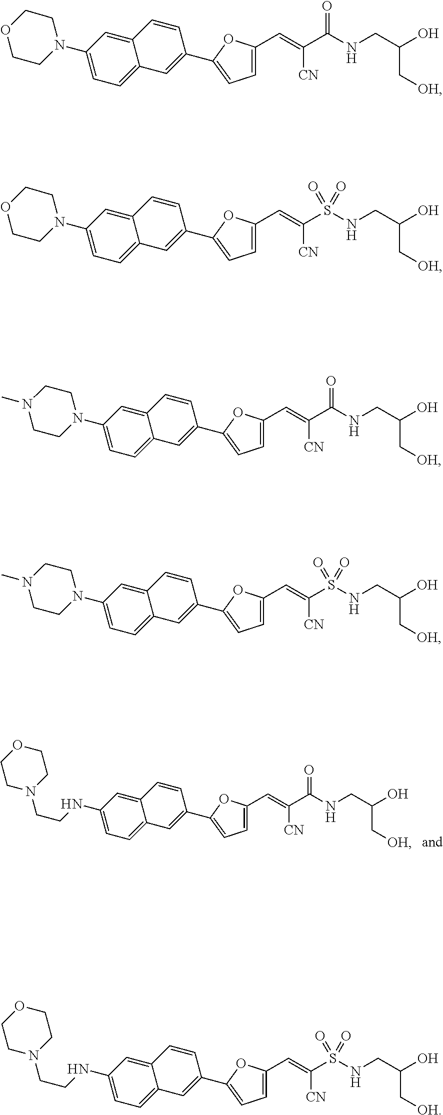 [SMILES: N#C/C(=C\c1ccc(-c2ccc3cc(N4CCOCC4)ccc3c2)o1)C(=O)NCC(O)CO ; N#C/C(=C\c1ccc(-c2ccc3cc(N4CCOCC4)ccc3c2)o1)S(=O)(=O)NCC(O)CO ; CN1CCN(c2ccc3cc(-c4ccc(/C=C(\C#N)C(=O)NCC(O)CO)o4)ccc3c2)CC1 ; CN1CCN(c2ccc3cc(-c4ccc(/C=C(\C#N)S(=O)(=O)NCC(O)CO)o4)ccc3c2)CC1 ; N#C/C(=C\c1ccc(-c2ccc3cc(NCCN4CCOCC4)ccc3c2)o1)C(=O)NCC(O)CO ; N#C/C(=C\c1ccc(-c2ccc3cc(NCCN4CCOCC4)ccc3c2)o1)S(=O)(=O)NCC(O)CO]

In some embodiments, the compound of Formula I is selected from a group consisting of

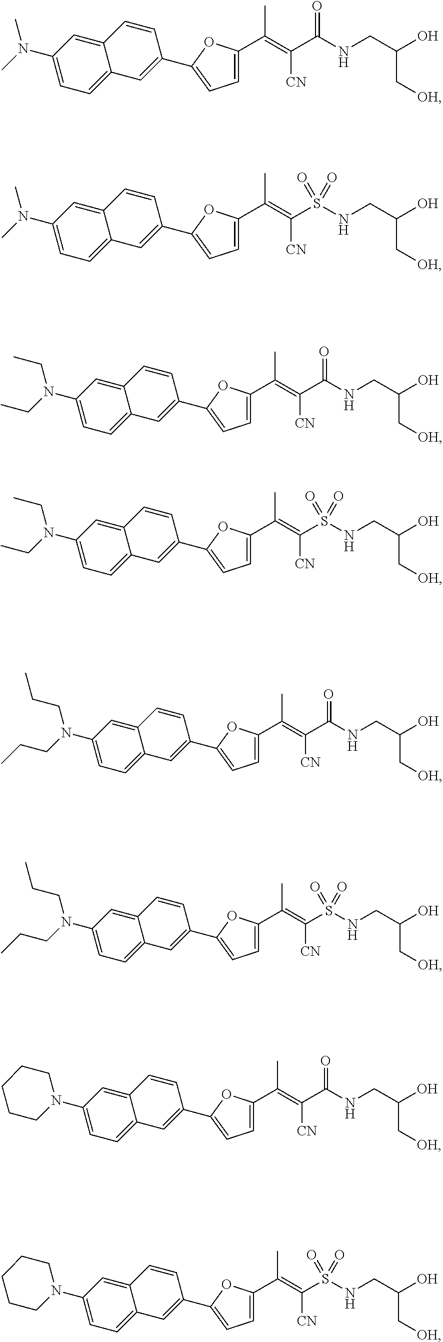

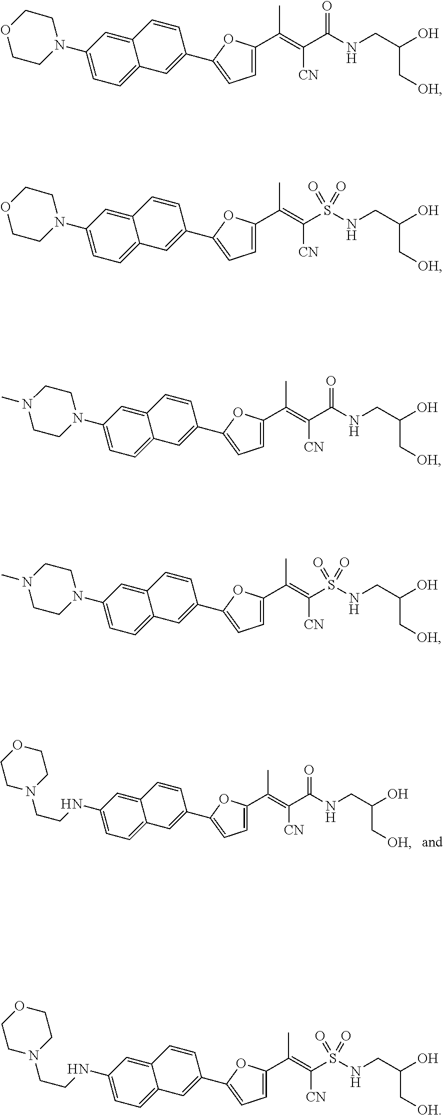 [SMILES: C/C(=C(/C#N)C(=O)NCC(O)CO)c1ccc(-c2ccc3cc(N4CCOCC4)ccc3c2)o1 ; C/C(=C(/C#N)S(=O)(=O)NCC(O)CO)c1ccc(-c2ccc3cc(N4CCOCC4)ccc3c2)o1 ; C/C(=C(/C#N)C(=O)NCC(O)CO)c1ccc(-c2ccc3cc(N4CCN(C)CC4)ccc3c2)o1 ; C/C(=C(/C#N)S(=O)(=O)NCC(O)CO)c1ccc(-c2ccc3cc(N4CCN(C)CC4)ccc3c2)o1 ; C/C(=C(/C#N)C(=O)NCC(O)CO)c1ccc(-c2ccc3cc(NCCN4CCOCC4)ccc3c2)o1 ; C/C(=C(/C#N)S(=O)(=O)NCC(O)CO)c1ccc(-c2ccc3cc(NCCN4CCOCC4)ccc3c2)o1]

In some embodiments, the compound of Formula I is selected from a group consisting of

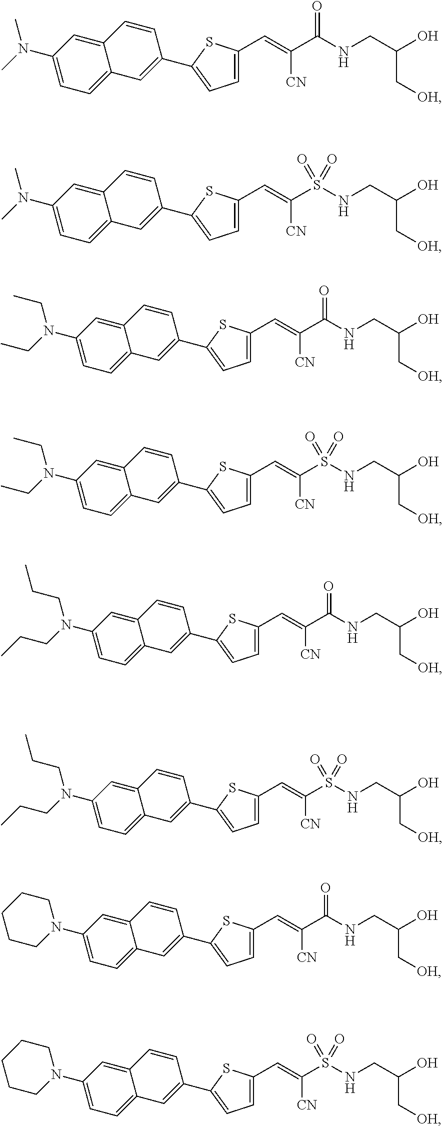

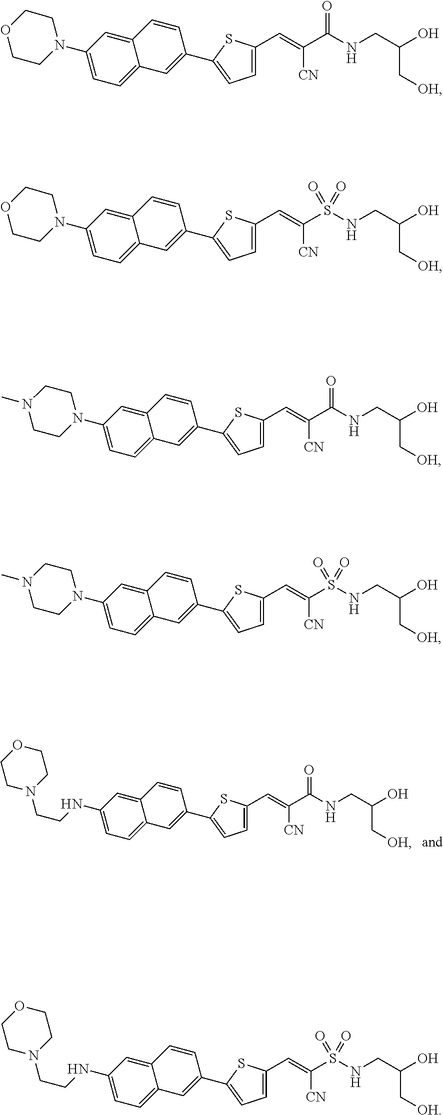 [SMILES: N#C/C(=C\c1ccc(-c2ccc3cc(N4CCOCC4)ccc3c2)s1)C(=O)NCC(O)CO ; N#C/C(=C\c1ccc(-c2ccc3cc(N4CCOCC4)ccc3c2)s1)S(=O)(=O)NCC(O)CO ; CN1CCN(c2ccc3cc(-c4ccc(/C=C(\C#N)C(=O)NCC(O)CO)s4)ccc3c2)CC1 ; CN1CCN(c2ccc3cc(-c4ccc(/C=C(\C#N)S(=O)(=O)NCC(O)CO)s4)ccc3c2)CC1 ; N#C/C(=C\c1ccc(-c2ccc3cc(NCCN4CCOCC4)ccc3c2)s1)C(=O)NCC(O)CO ; N#C/C(=C\c1ccc(-c2ccc3cc(NCCN4CCOCC4)ccc3c2)s1)S(=O)(=O)NCC(O)CO]

In some embodiments, the compound of Formula I is selected from a group consisting of

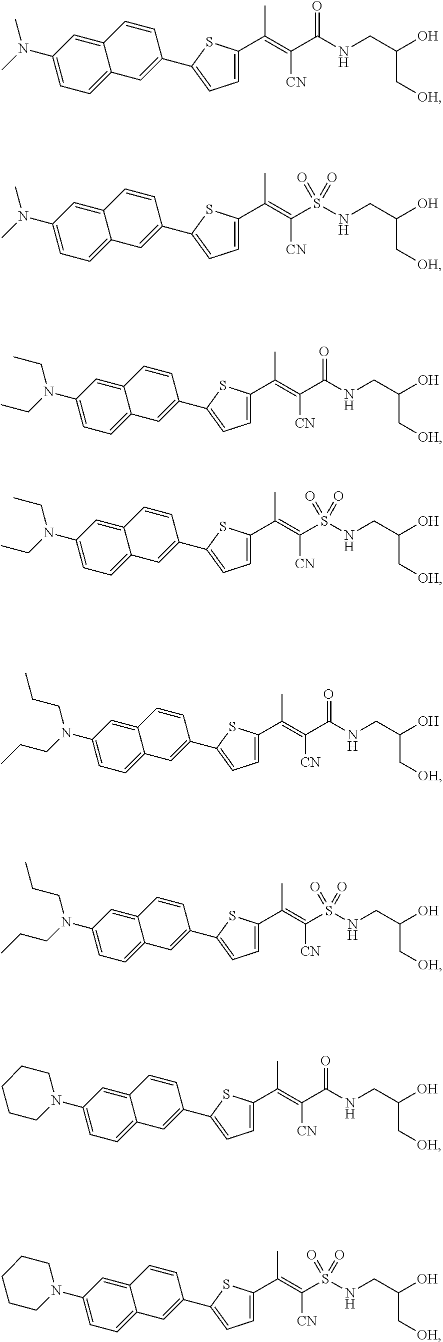

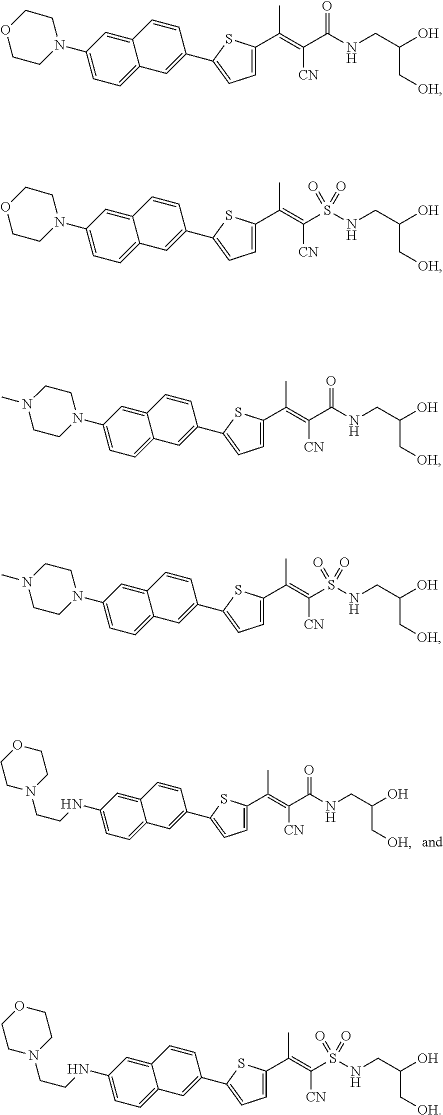 [SMILES: C/C(=C(/C#N)C(=O)NCC(O)CO)c1ccc(-c2ccc3cc(N4CCOCC4)ccc3c2)s1 ; C/C(=C(/C#N)S(=O)(=O)NCC(O)CO)c1ccc(-c2ccc3cc(N4CCOCC4)ccc3c2)s1 ; C/C(=C(/C#N)C(=O)NCC(O)CO)c1ccc(-c2ccc3cc(N4CCN(C)CC4)ccc3c2)s1 ; C/C(=C(/C#N)S(=O)(=O)NCC(O)CO)c1ccc(-c2ccc3cc(N4CCN(C)CC4)ccc3c2)s1 ; C/C(=C(/C#N)C(=O)NCC(O)CO)c1ccc(-c2ccc3cc(NCCN4CCOCC4)ccc3c2)s1 ; C/C(=C(/C#N)S(=O)(=O)NCC(O)CO)c1ccc(-c2ccc3cc(NCCN4CCOCC4)ccc3c2)s1]

In some embodiments, the compound of Formula I is selected from a group consisting of

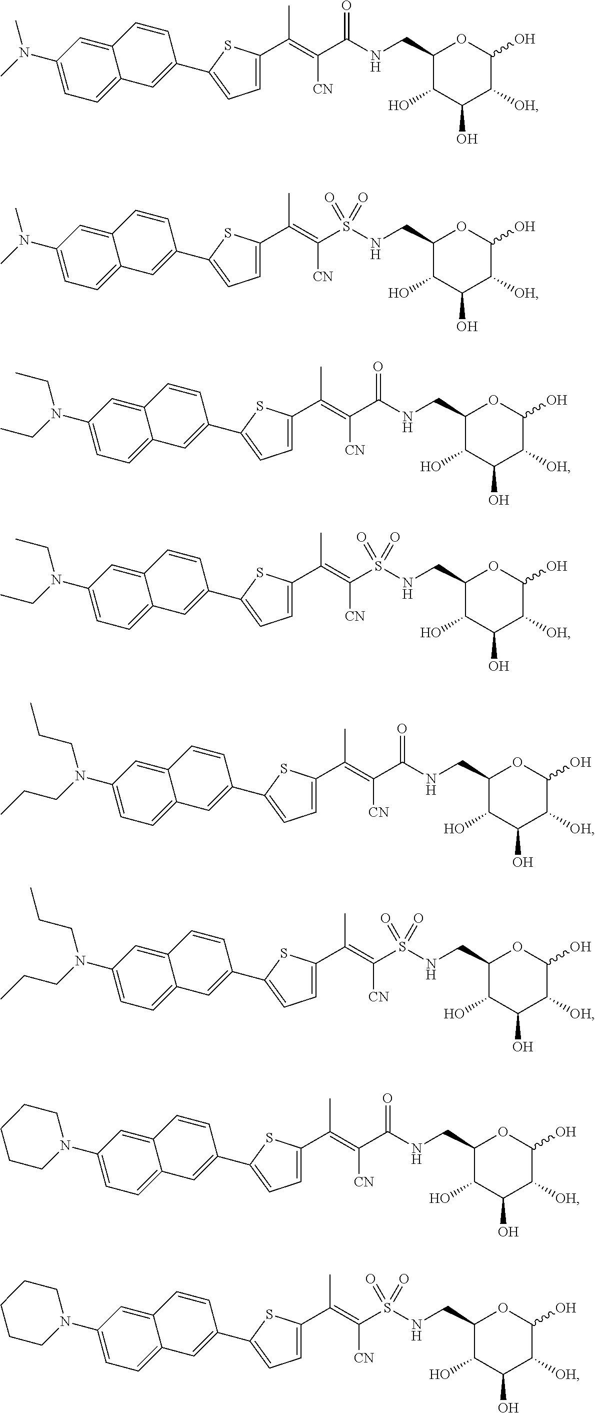

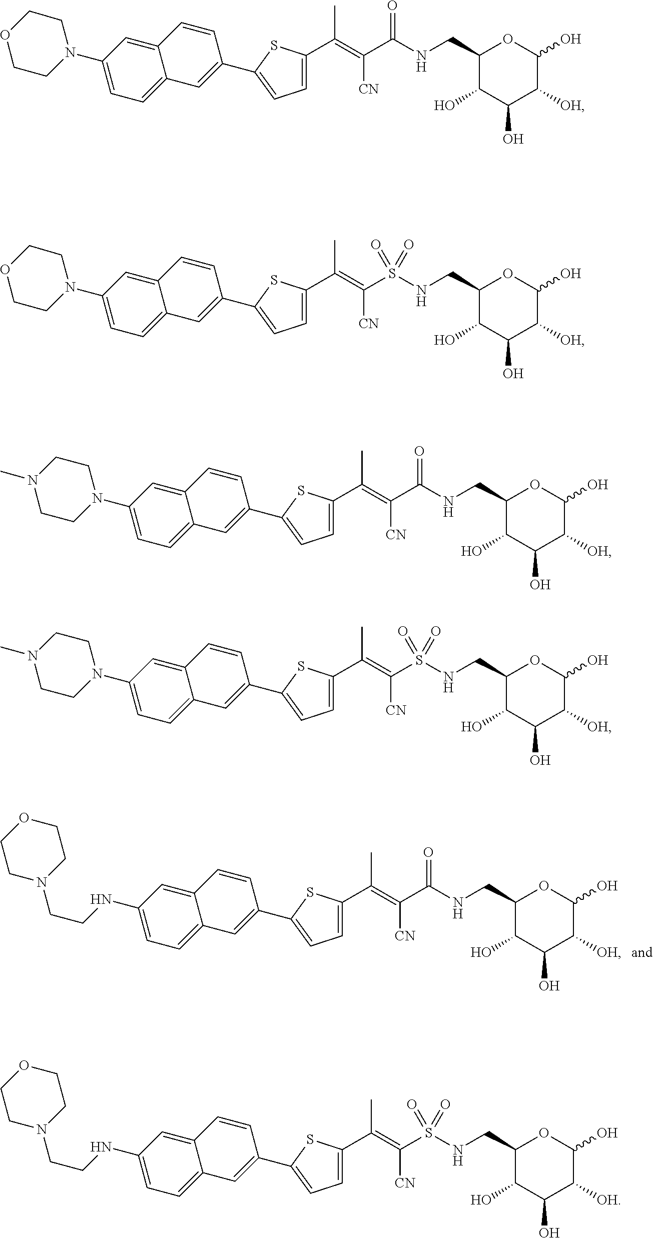

In some embodiments, the compound of Formula I is selected from a group consisting of

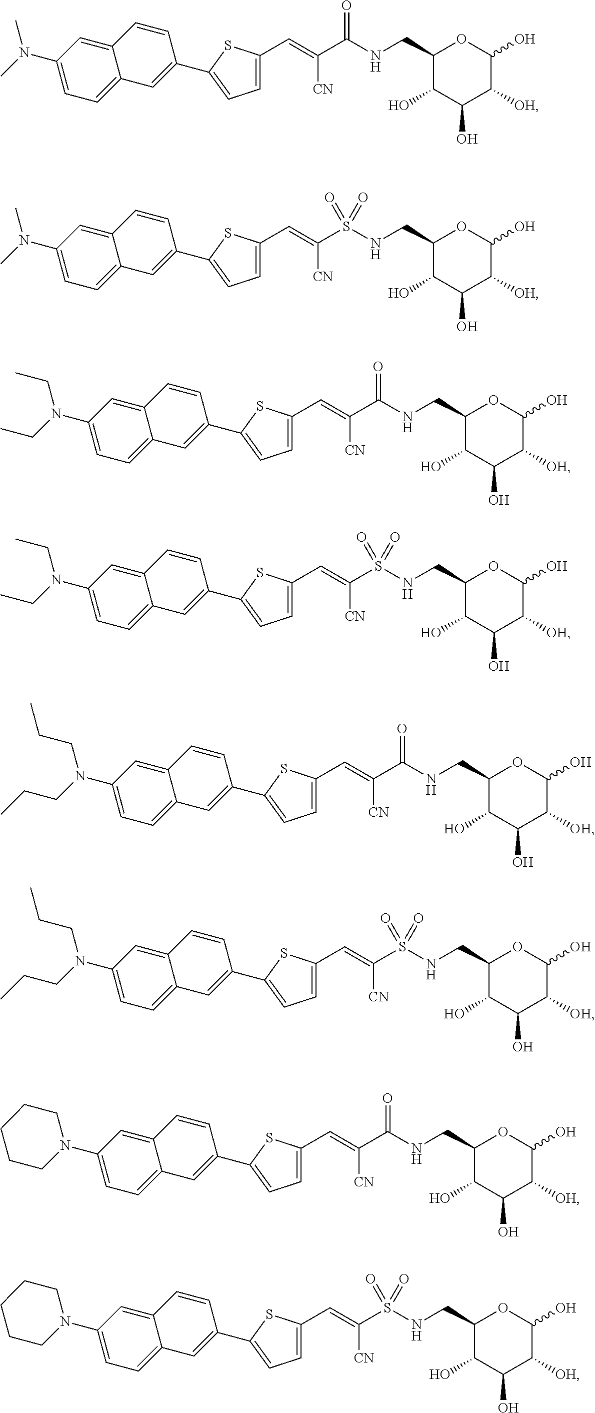

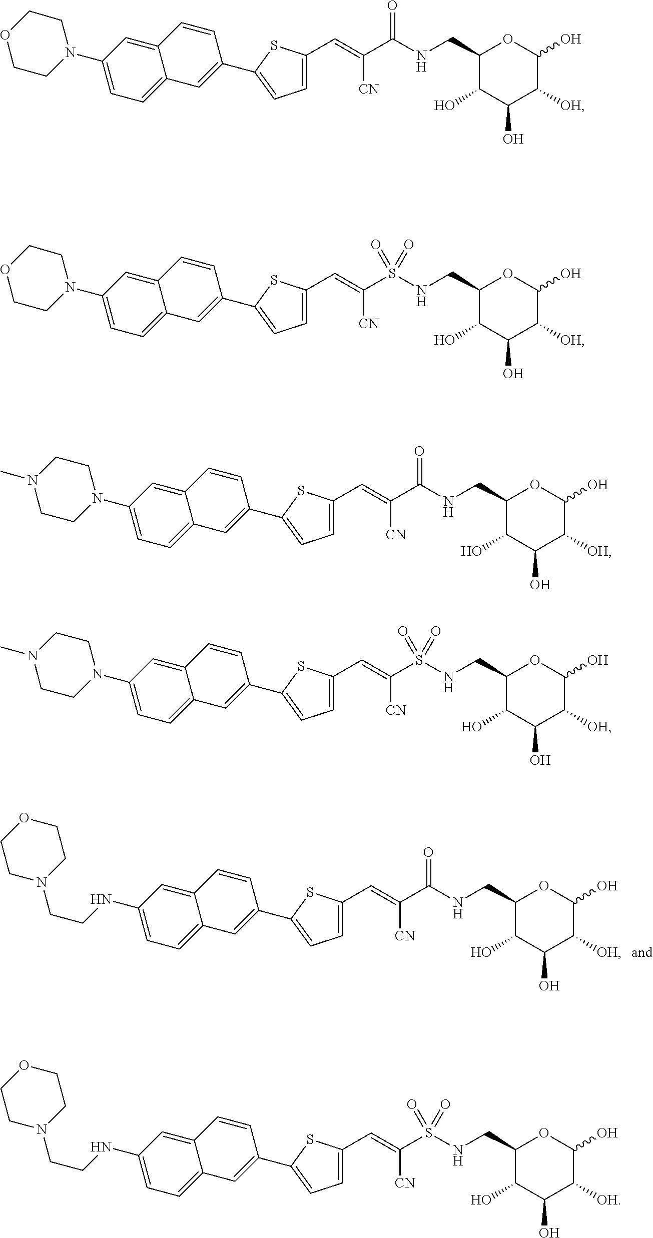

In some embodiments, the compound of Formula I is selected from a group consisting of

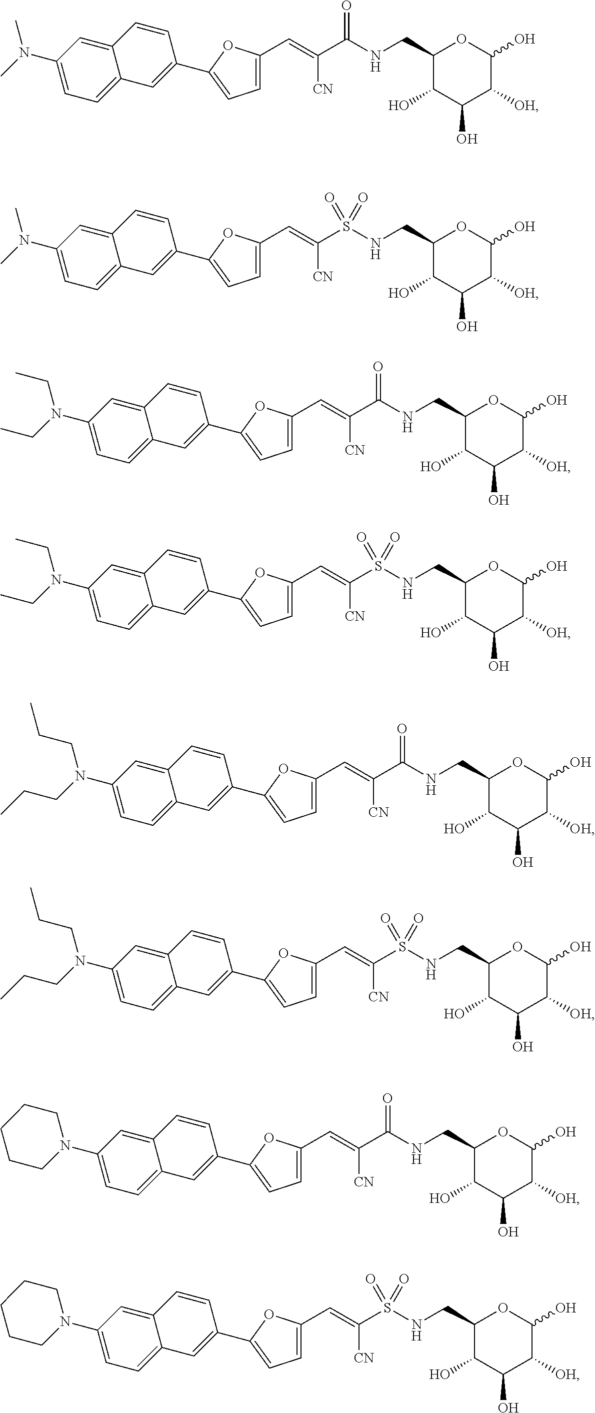

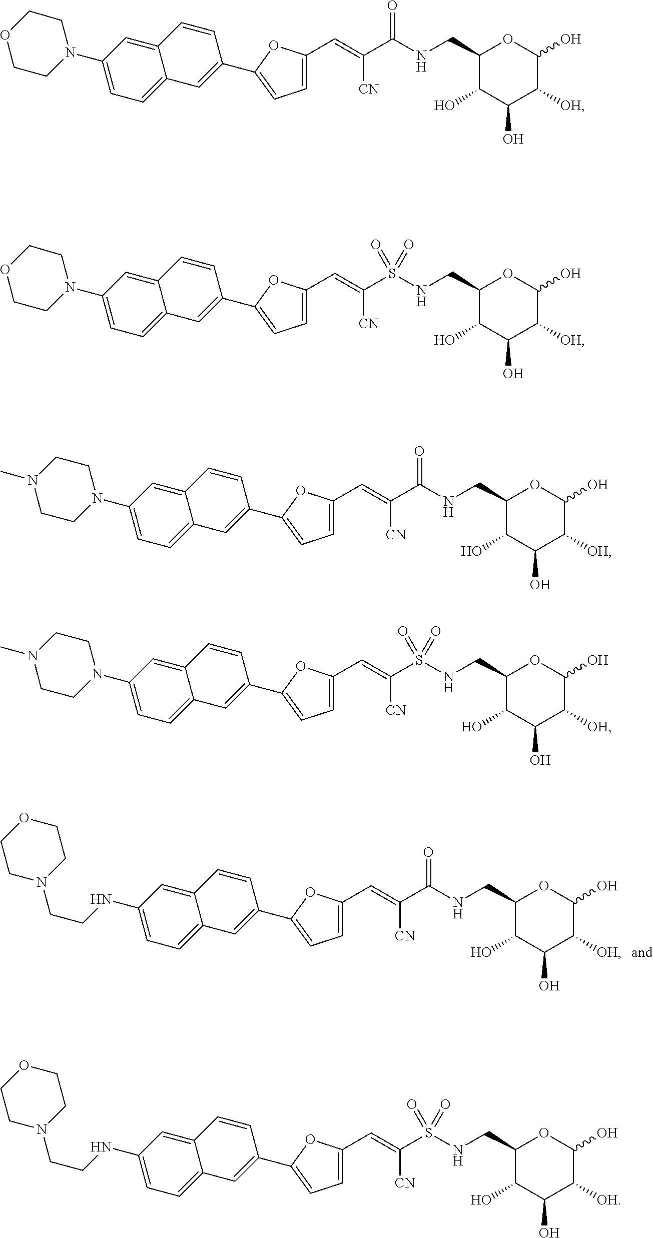

In some embodiments, the compound of Formula I is selected from a group consisting of

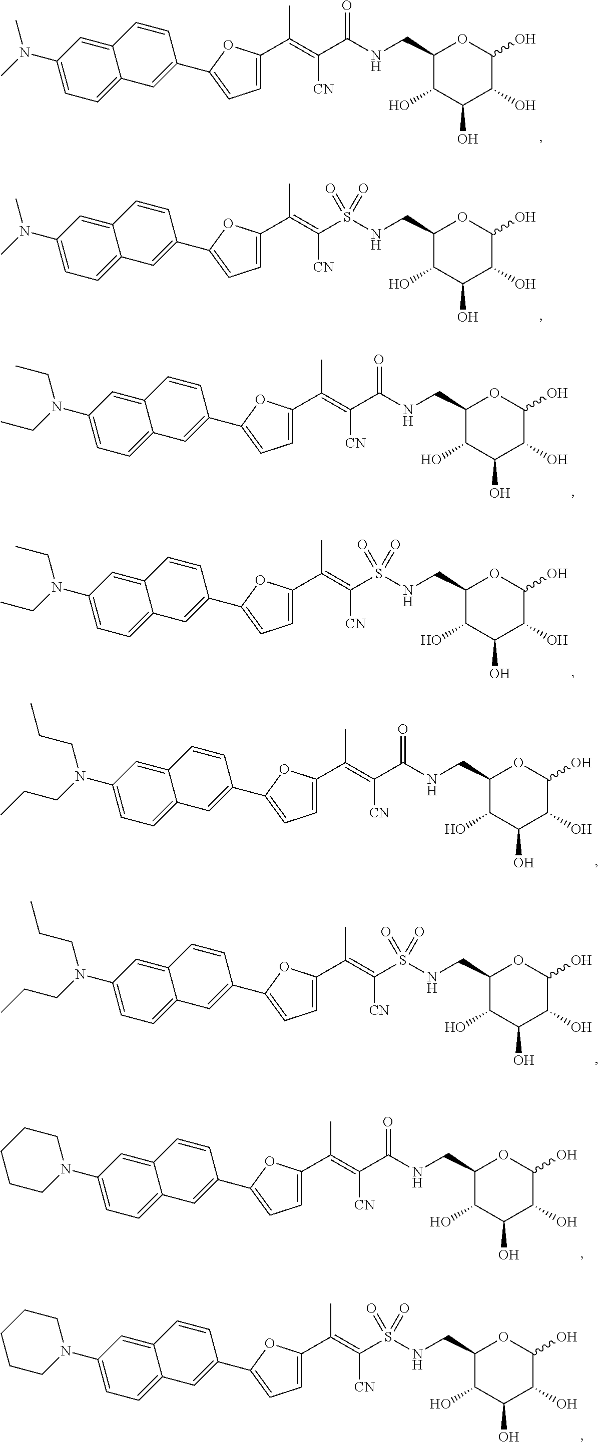

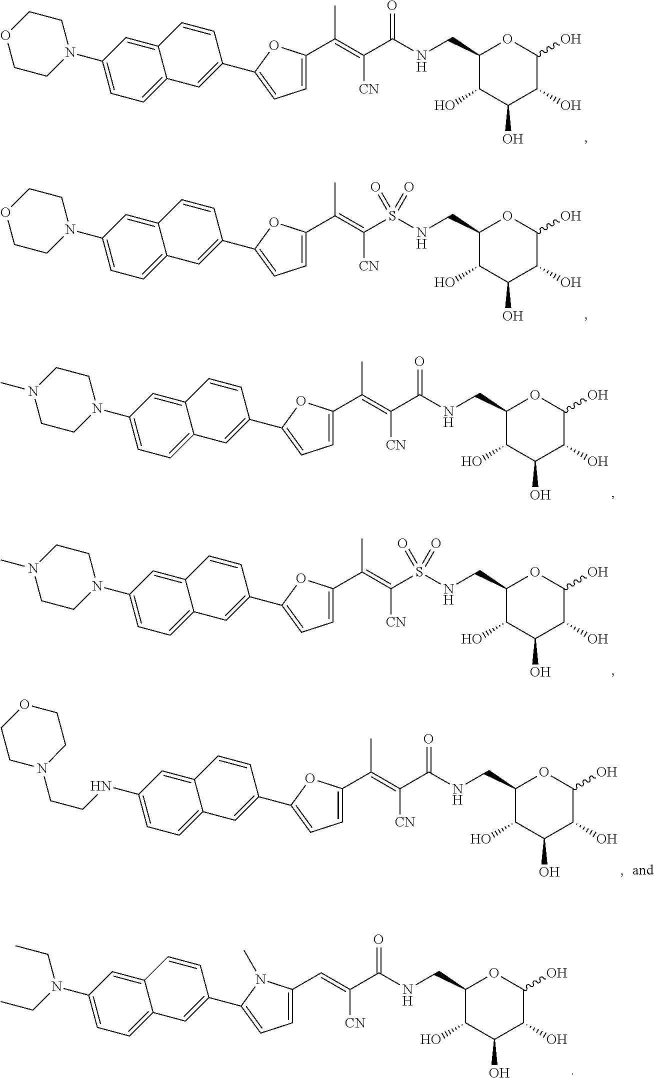

In some embodiments, the compound of Formula I is selected from a group consisting of

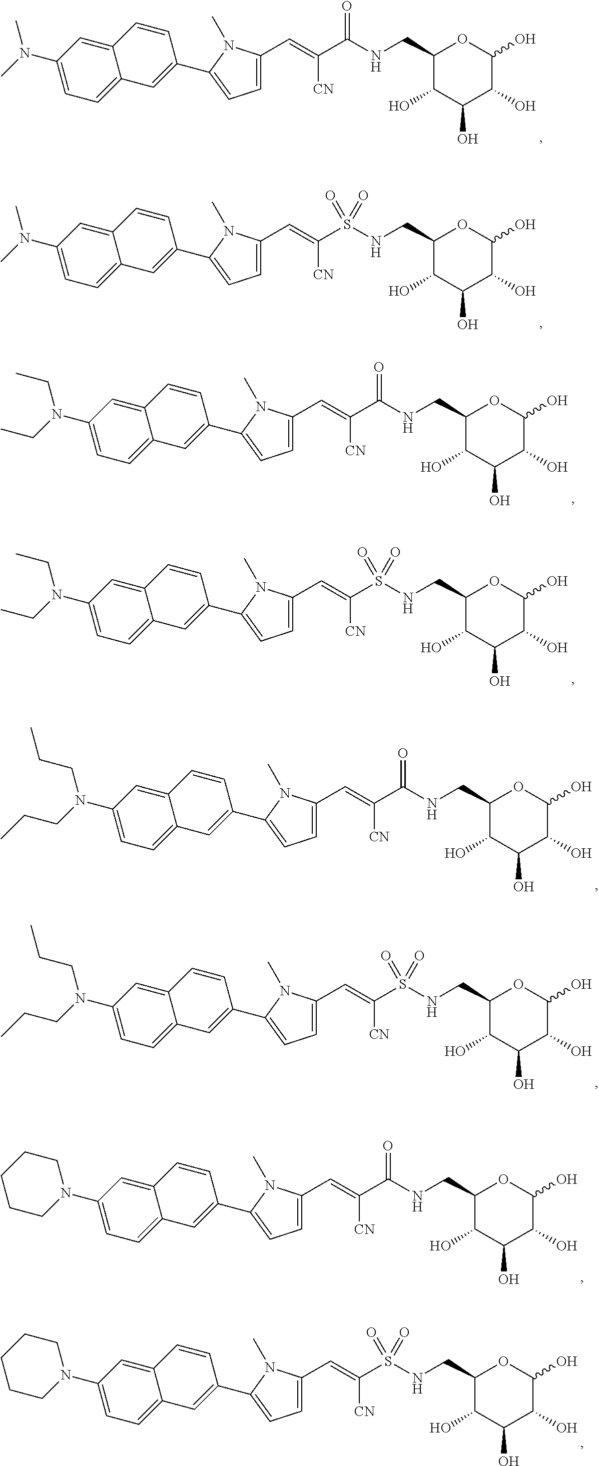

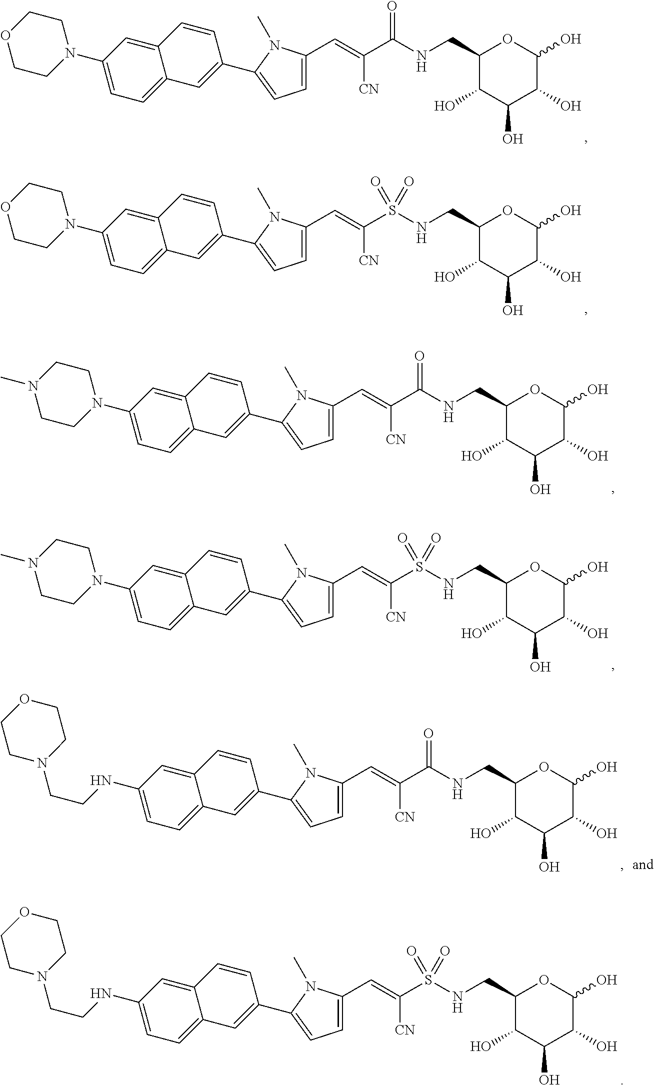

In some embodiments, the compound of Formula I is selected from a group consisting of

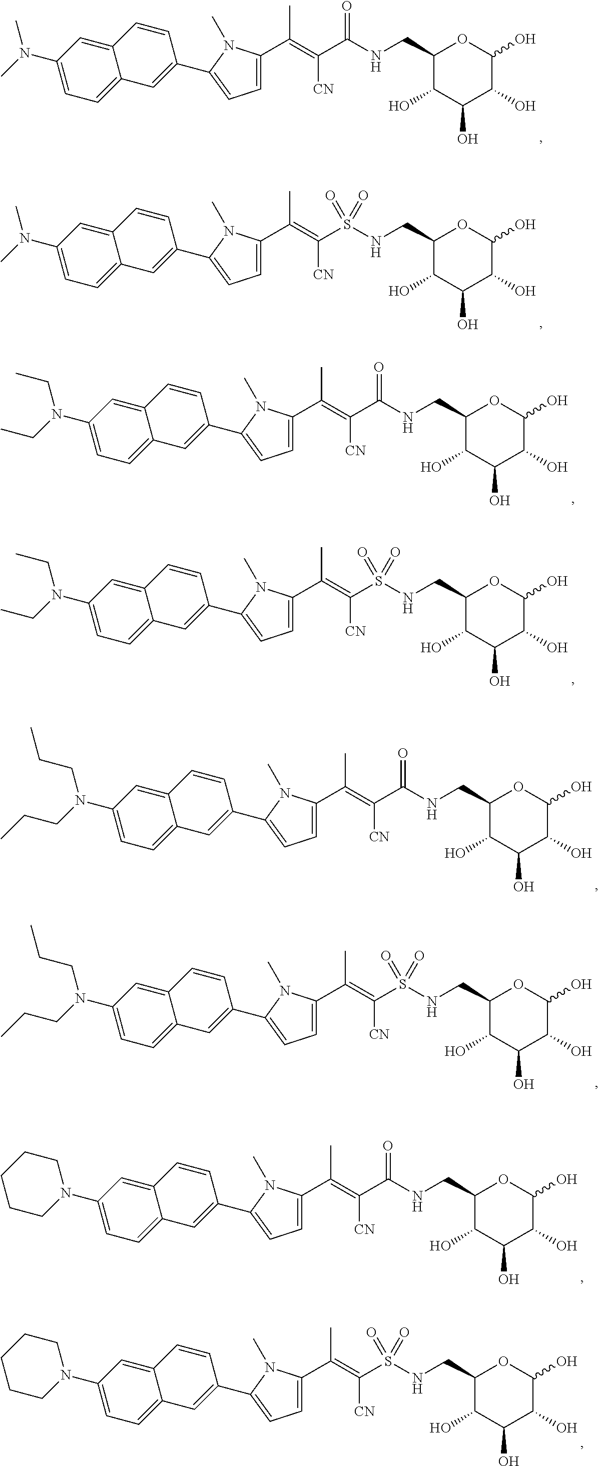

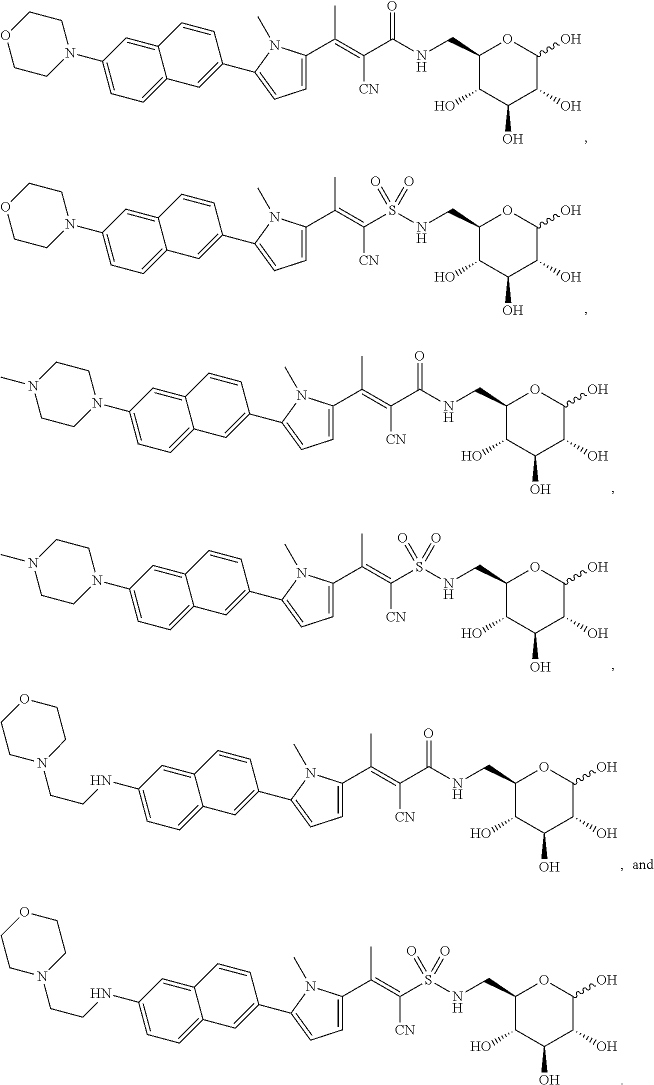

In some embodiments, the compound of Formula I is selected from a group consisting of

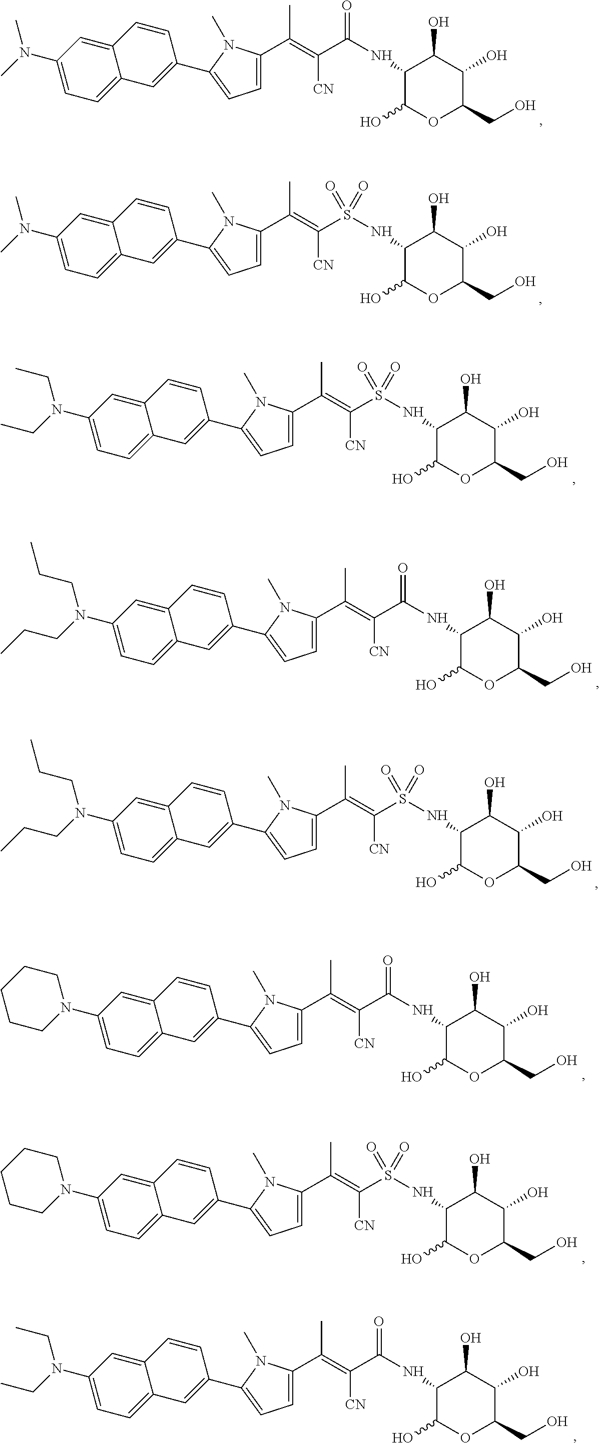

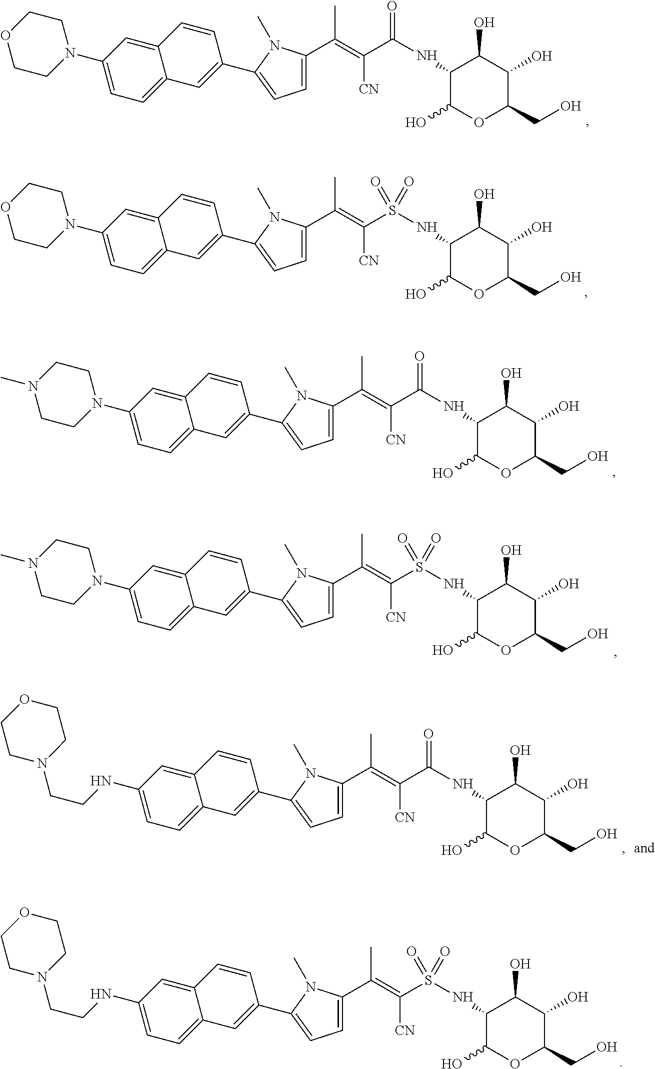

In some embodiments, the compound of Formula I is selected from a group consisting of

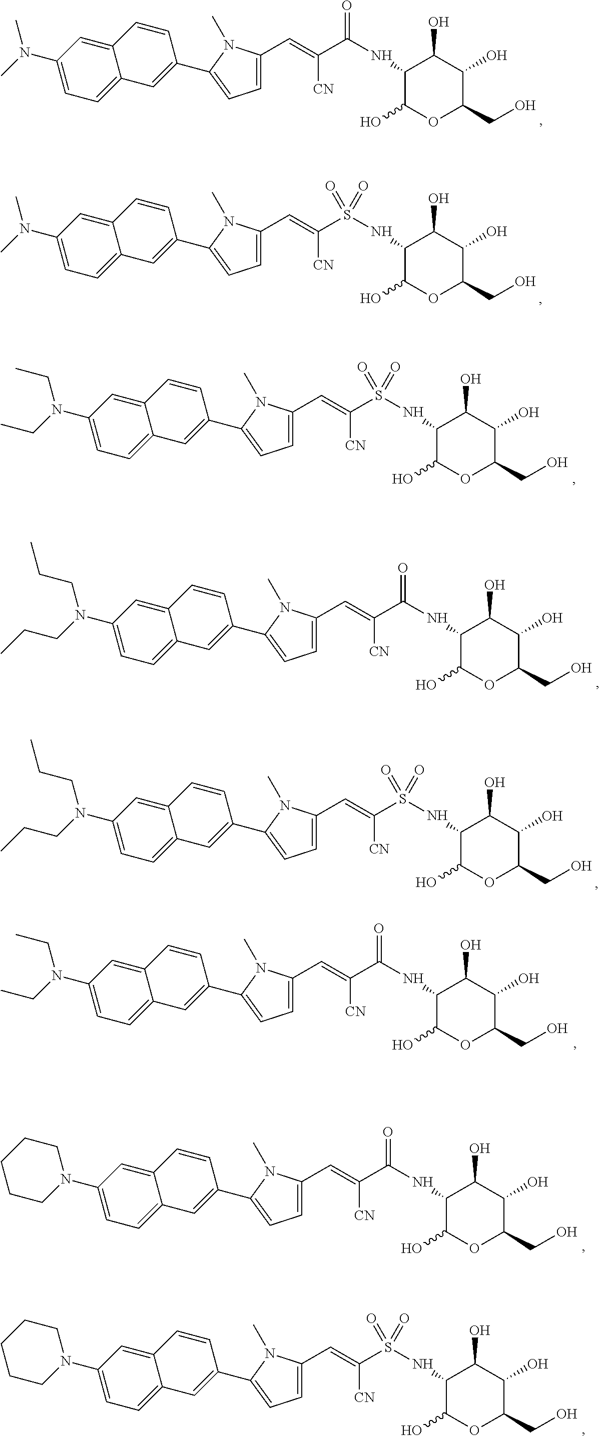

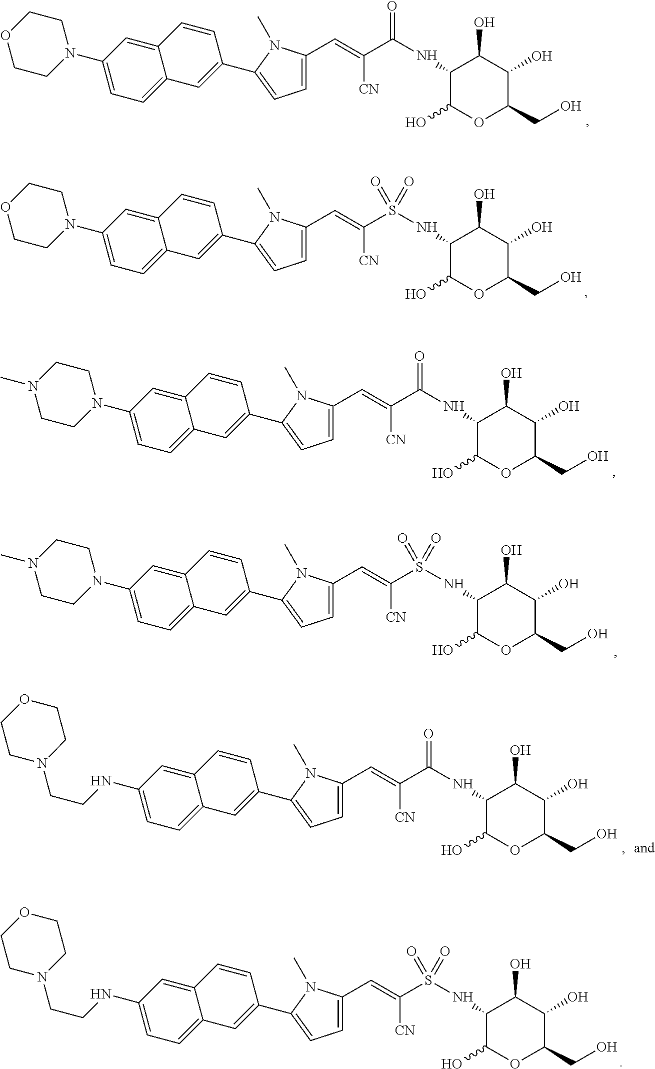

In some embodiments, the compound of Formula I is selected from a group consisting of

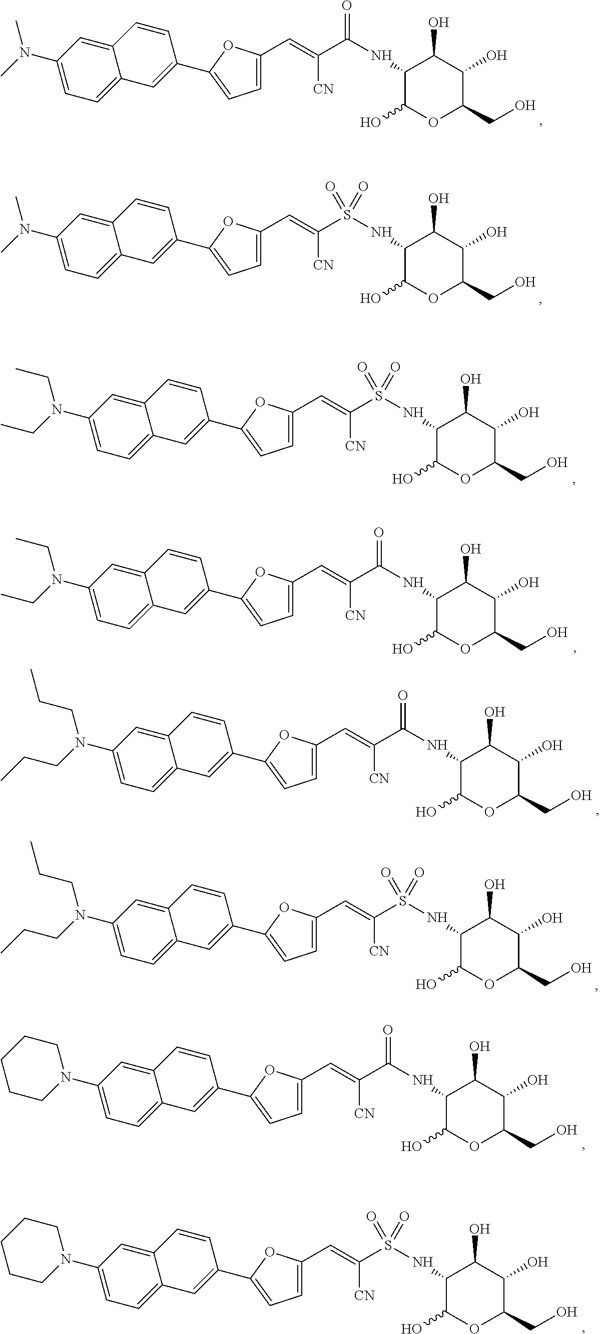

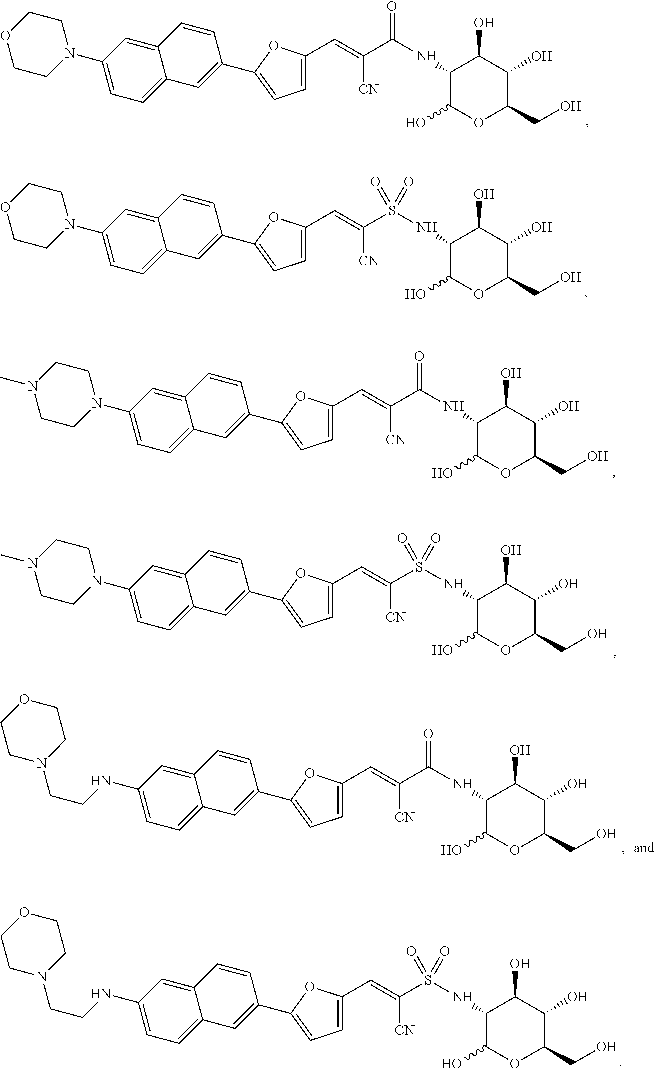

In some embodiments, the compound of Formula I is selected from a group consisting of

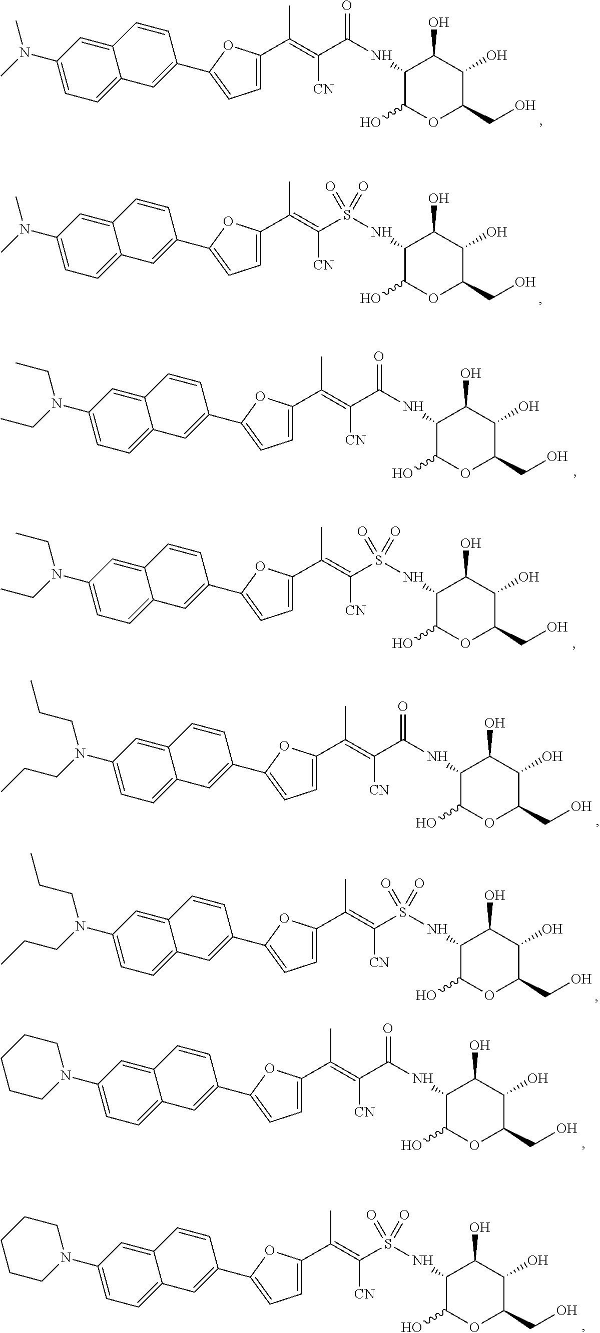

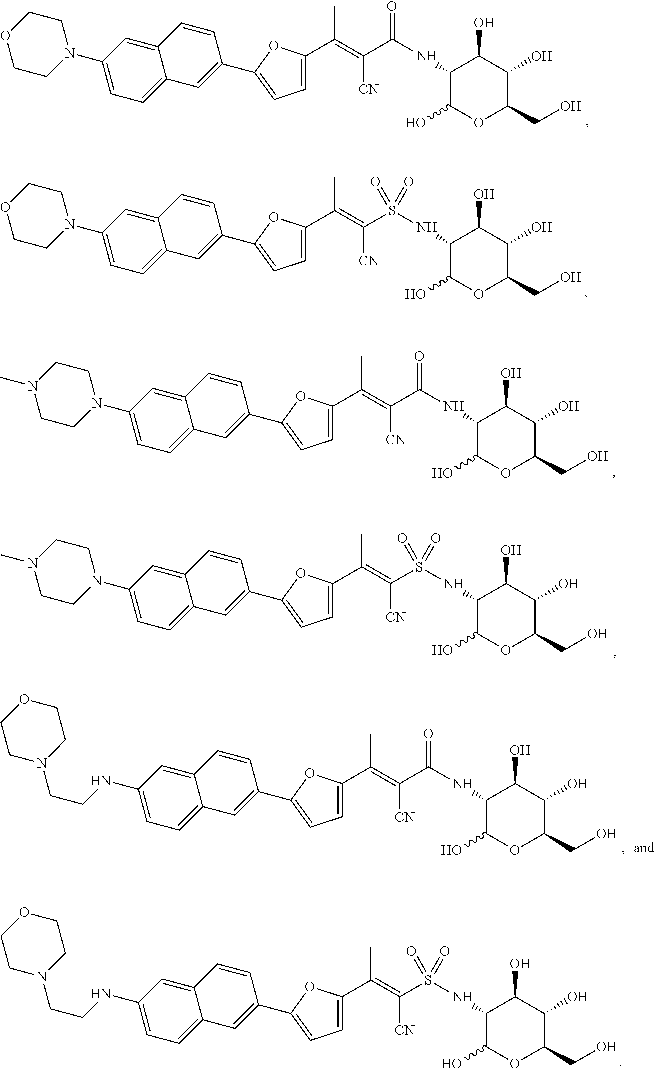

In some embodiments, the compound of Formula I is selected from a group consisting of

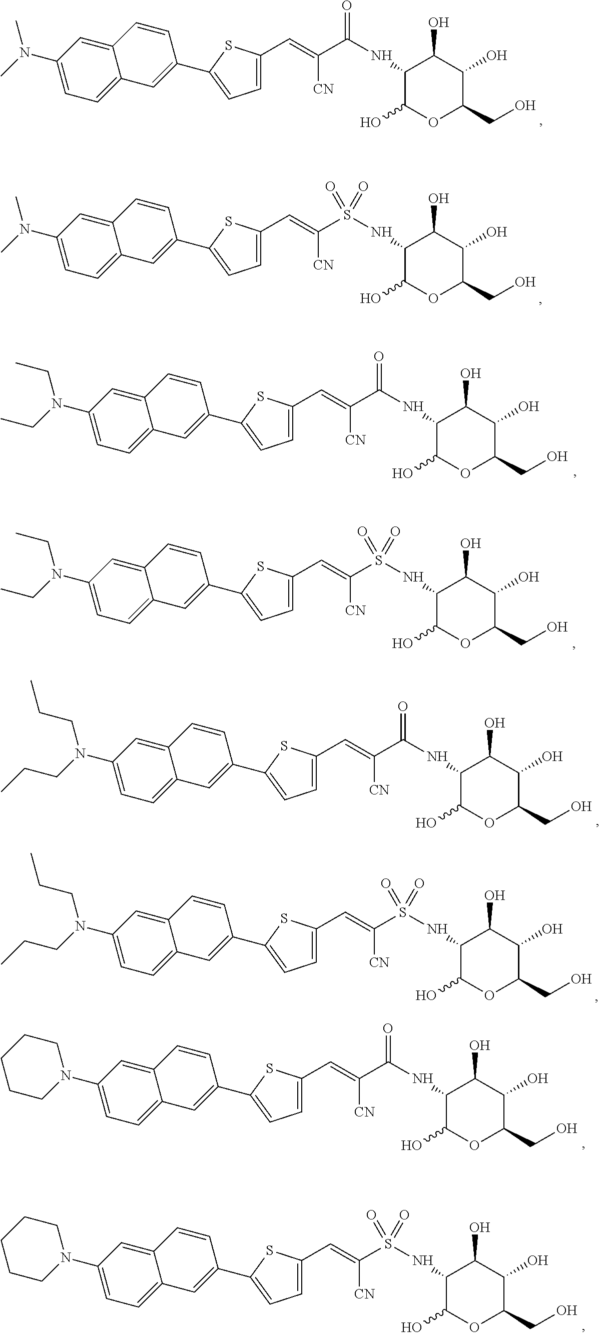

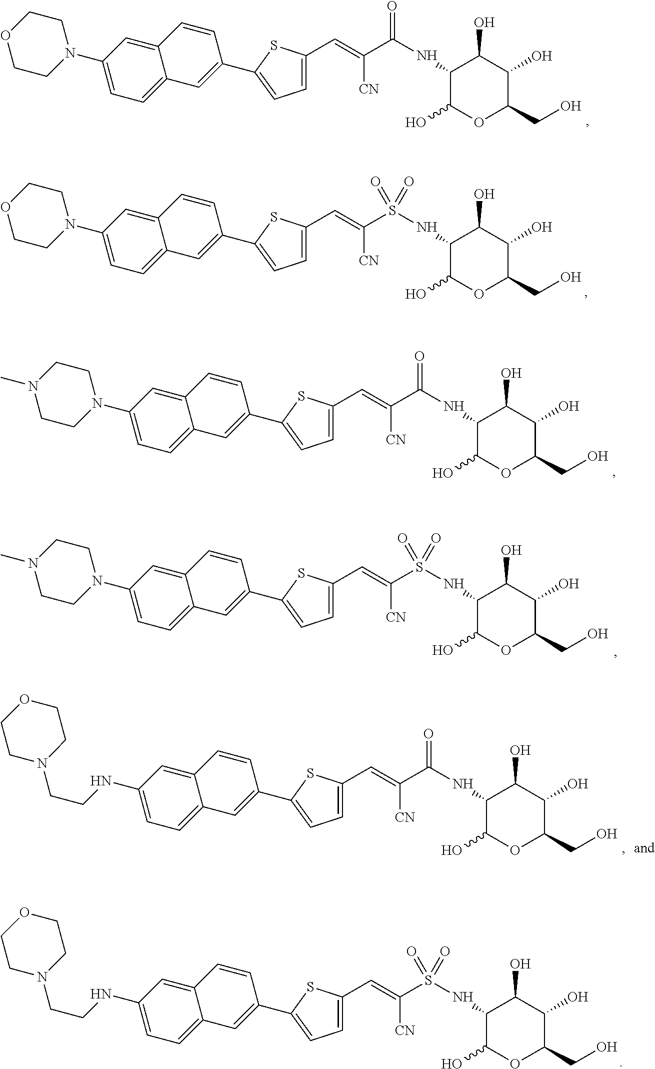

In some embodiments, the compound of Formula I is selected from a group consisting of

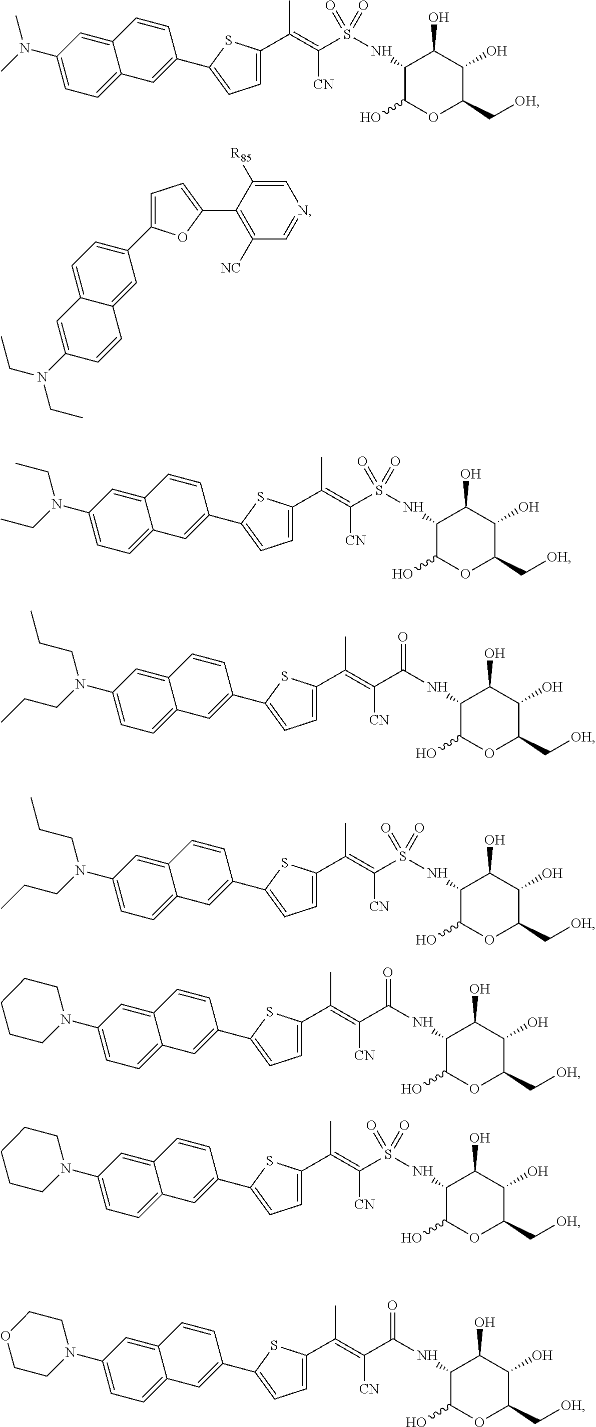

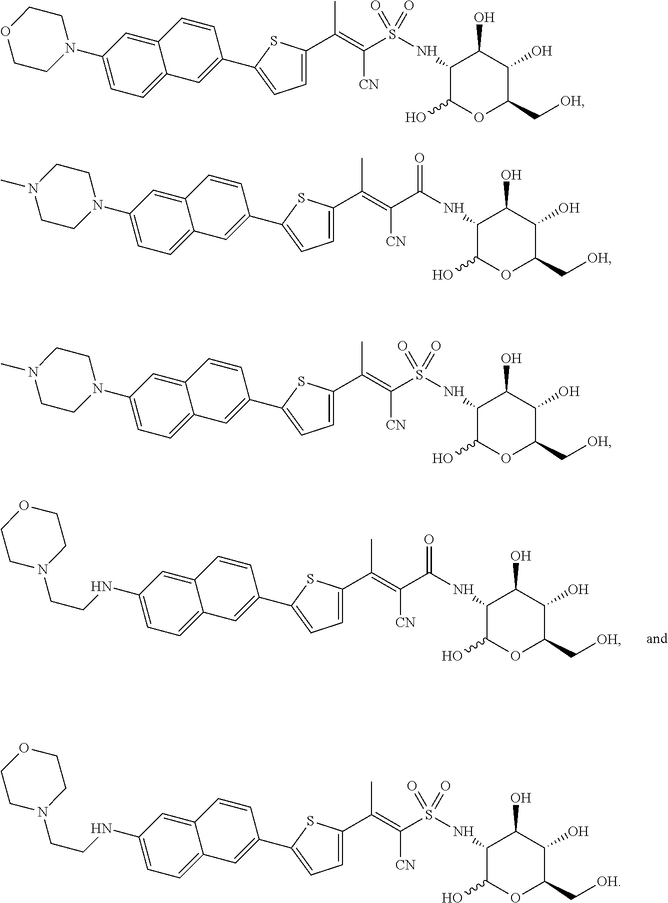

In some embodiments, the compound of Formula I is:

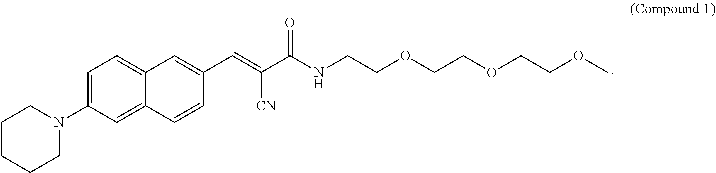 [SMILES: COCCOCCOCCNC(=O)/C(C#N)=C/c1ccc2cc(N3CCCCC3)ccc2c1]

In some cases, the compound of Formula I is 2-cyano-N-(2-(2-(2-methoxyethoxy)ethoxy)ethyl)-3-(6-(piperidin-1-yl)naphthalen-2-yl)acrylamide. In some cases, the compound of Formula I is (E)-2-cyano-N-(2-(2-(2-methoxyethoxy)ethoxy)ethyl)-3-(6-(piperidin-1-yl)naphthalen-2-yl)acrylamide. In some cases, the compound of Formula I is (Z)-2-cyano-N-(2-(2-(2-methoxyethoxy)ethoxy)ethyl)-3-(6-(piperidin-1-yl)naphthalen-2-yl)acrylamide.

In some embodiments, the compound of Formula I is:

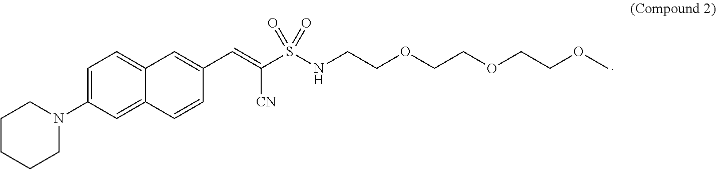 [SMILES: COCCOCCOCCNS(=O)(=O)/C(C#N)=C/c1ccc2cc(N3CCCCC3)ccc2c1]

In some cases, the compound of Formula I is 1-cyano-N-(2-(2-(2-methoxyethoxy)ethoxy)ethyl)-2-(6-(piperidin-1-yl)naphthalen-2-yl)ethenesulfonamide. In some cases, the compound of Formula I is (E)-1-cyano-N-(2-(2-(2-methoxyethoxy)ethoxy)ethyl)-2-(6-(piperidin-1-yl)naphthalen-2-yl)ethenesulfonamide. In some cases, the compound of Formula I is (Z)-1-cyano-N-(2-(2-(2-methoxyethoxy)ethoxy)ethyl)-2-(6-(piperidin-1-yl)naphthalen-2-yl)ethenesulfonamide.

In some embodiments, the compound of Formula I is:

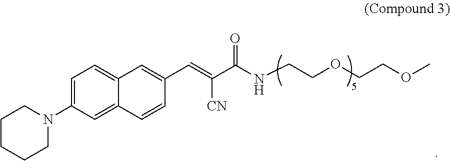

In some cases, the compound of Formula I is 2-cyano-N-(2,5,8,11,14,17-hexaoxanonadecan-19-yl)-3-(6-(piperidin-1-yl)naphthalen-2-yl)acrylamide. In some cases, the compound of Formula I is (E)-2-cyano-N-(2,5,8,11,14,17-hexaoxanonadecan-19-yl)-3-(6-(piperidin-1-yl)naphthalen-2-yl)acrylamide. In some cases, the compound of Formula I is (Z)-2-cyano-N-(2,5,8,11,14,17-hexaoxanonadecan-19-yl)-3-(6-(piperidin-1-yl)naphthalen-2-yl)acrylamide.

In some embodiments, the compound of Formula I is:

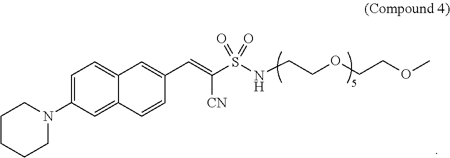

In some cases, the compound of Formula I is 1-cyano-N-(2,5,8,11,14,17-hexaoxanonadecan-19-yl)-2-(6-(piperidin-1-yl)naphthalen-2-yl)ethenesulfonamide. In some cases, the compound of Formula I is (E)-1-cyano-N-(2,5,8,11,14,17-hexaoxanonadecan-19-yl)-2-(6-(piperidin-1-yl)naphthalen-2-yl)ethenesulfonamide. In some cases, the compound of Formula I is (Z)-1-cyano-N-(2,5,8,11,14,17-hexaoxanonadecan-19-yl)-2-(6-(piperidin-1-yl)naphthalen-2-yl)ethenesulfonamide.

In some embodiments, the compound of Formula I is:

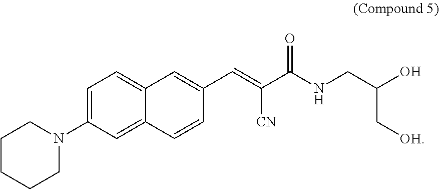 [SMILES: N#C/C(=C\c1ccc2cc(N3CCCCC3)ccc2c1)C(=O)NCC(O)CO]

In some cases, the compound of Formula I is 2-cyano-N-(2,3-dihydroxypropyl)-3-(6-(piperidin-1-yl)naphthalen-2-yl)acrylamide. In some cases, the compound of Formula I is (E)-2-cyano-N-(2,3-dihydroxypropyl)-3-(6-(piperidin-1-yl)naphthalen-2-yl)acrylamide. In some cases, the compound of Formula I is (Z)-2-cyano-N-(2,3-dihydroxypropyl)-3-(6-(piperidin-1-yl)naphthalen-2-yl)acrylamide.

In some embodiments, the compound of Formula I is:

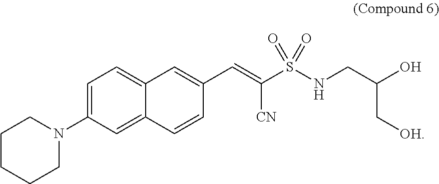 [SMILES: N#C/C(=C\c1ccc2cc(N3CCCCC3)ccc2c1)S(=O)(=O)NCC(O)CO]

In some cases, the compound of Formula I is 1-cyano-N-(2,3-dihydroxypropyl)-2-(6-(piperidin-1-yl)naphthalen-2-yl)ethenesulfonamide. In some cases, the compound of Formula I is (E)-1-cyano-N-(2,3-dihydroxypropyl)-2-(6-(piperidin-1-yl)naphthalen-2-yl)ethenesulfonamide. In some cases, the compound of Formula I is (Z)-1-cyano-N-(2,3-dihydroxypropyl)-2-(6-(piperidin-1-yl)naphthalen-2-yl)ethenesulfonamide.

In some embodiments, the compound of Formula I is:

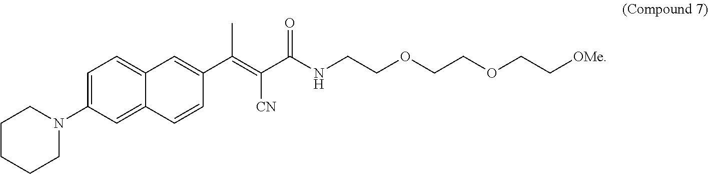 [SMILES: COCCOCCOCCNC(=O)/C(C#N)=C(\C)c1ccc2cc(N3CCCCC3)ccc2c1]

In some cases, the compound of Formula I is 2-cyano-N-(2-(2-(2-methoxyethoxy)ethoxy)ethyl)-3-(6-(piperidin-1-yl)naphthalen-2-yl)but-2-enamide. In some cases, the compound of Formula I is (E)-2-cyano-N-(2-(2-(2-methoxyethoxy)ethoxy)ethyl)-3-(6-(piperidin-1-yl)naphthalen-2-yl)but-2-enamide. In some cases, the compound of Formula I is (Z)-2-cyano-N-(2-(2-(2-methoxyethoxy)ethoxy)ethyl)-3-(6-(piperidin-1-yl)naphthalen-2-yl)but-2-enamide.

In some embodiments, the compound of Formula I is:

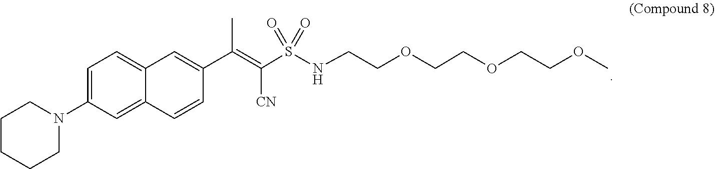 [SMILES: COCCOCCOCCNS(=O)(=O)/C(C#N)=C(\C)c1ccc2cc(N3CCCCC3)ccc2c1]

In some cases, the compound of Formula I is 1-cyano-N-(2-(2-(2-methoxyethoxy)ethoxy)ethyl)-2-(6-(piperidin-1-yl)naphthalen-2-yl)prop-1-ene-1-sulfonamide. In some cases, the compound of Formula I is (E)-1-cyano-N-(2-(2-(2-methoxyethoxy)ethoxy)ethyl)-2-(6-(piperidin-1-yl)naphthalen-2-yl)prop-1-ene-1-sulfonamide. In some cases, the compound of Formula I is (Z)-1-cyano-N-(2-(2-(2-methoxyethoxy)ethoxy)ethyl)-2-(6-(piperidin-1-yl)naphthalen-2-yl)prop-1-ene-1-sulfonamide.

In some embodiments, the compound of Formula I is:

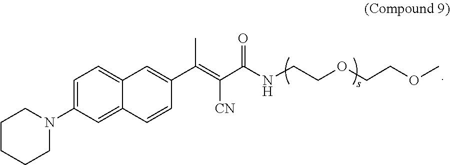

In some cases, the compound of Formula I is 2-cyano-N-(2,5,8,11,14,17-hexaoxanonadecan-19-yl)-3-(6-(piperidin-1-yl)naphthalen-2-yl)but-2-enamide. In some cases, the compound of Formula I is (E)-2-cyano-N-(2,5,8,11,14,17-hexaoxanonadecan-19-yl)-3-(6-(piperidin-1-yl)naphthalen-2-yl)but-2-enamide. In some cases, the compound of Formula I is (Z)-2-cyano-N-(2,5,8,11,14,17-hexaoxanonadecan-19-yl)-3-(6-(piperidin-1-yl)naphthalen-2-yl)but-2-enamide.

In some embodiments, the compound of Formula I is:

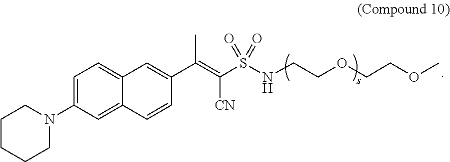

In some cases, the compound of Formula I is 1-cyano-N-(2,5,8,11,14,17-hexaoxanonadec an-19-yl)-2-(6-(piperidin-1-yl)naphthalen-2-yl)prop-1-ene-1-sulfonamide. In some cases, the compound of Formula I is (E)-1-cyano-N-(2,5,8,11,14,17-hexaoxanonadec an-19-yl)-2-(6-(piperidin-1-yl)naphthalen-2-yl)prop-1-ene-1-sulfonamide. In some cases, the compound of Formula I is (Z)-1-cyano-N-(2,5,8,11,14,17-hexaoxanonadec an-19-yl)-2-(6-(piperidin-1-yl)naphthalen-2-yl)prop-1-ene-1-sulfonamide.

In some embodiments, the compound of Formula I is:

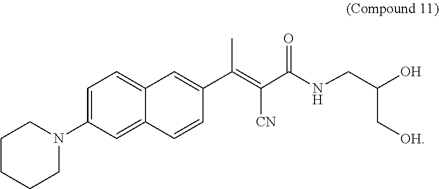 [SMILES: C/C(=C(/C#N)C(=O)NCC(O)CO)c1ccc2cc(N3CCCCC3)ccc2c1]

In some cases, the compound of Formula I is 2-cyano-N-(2,3-dihydroxypropyl)-3-(6-(piperidin-1-yl)naphthalen-2-yl)but-2-enamide. In some cases, the compound of Formula I is (E)-2-cyano-N-(2,3-dihydroxypropyl)-3-(6-(piperidin-1-yl)naphthalen-2-yl)but-2-enamide. In some cases, the compound of Formula I is (Z)-2-cyano-N-(2,3-dihydroxypropyl)-3-(6-(piperidin-1-yl)naphthalen-2-yl)but-2-enamide.

In some embodiments, the compound of Formula I is:

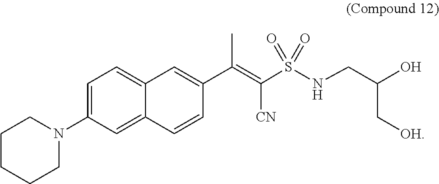 [SMILES: C/C(=C(/C#N)S(=O)(=O)NCC(O)CO)c1ccc2cc(N3CCCCC3)ccc2c1]

In some cases, the compound of Formula I is 1-cyano-N-(2,3-dihydroxypropyl)-2-(6-(piperidin-1-yl)naphthalen-2-yl)prop-1-ene-1-sulfonamide. In some cases, the compound of Formula I is (E)-1-cyano-N-(2,3-dihydroxypropyl)-2-(6-(piperidin-1-yl)naphthalen-2-yl)prop-1-ene-1-sulfonamide. In some cases, the compound of Formula I is (Z)-1-cyano-N-(2,3-dihydroxypropyl)-2-(6-(piperidin-1-yl)naphthalen-2-yl)prop-1-ene-1-sulfonamide.

In some embodiments, the compound of Formula I is:

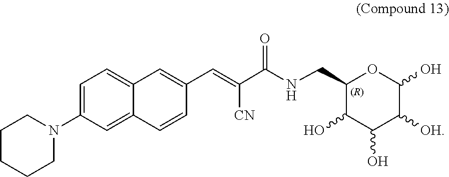

In some cases, the compound of Formula I is (R)-2-cyano-3-(6-(piperidin-1-yl)naphthalen-2-yl)-N-((3,4,5,6-tetrahydroxytetrahydro-2H-pyran-2-yl)methyl)acrylamide. In some cases, the compound of Formula I is (R,E)-2-cyano-3-(6-(piperidin-1-yl)naphthalen-2-yl)-N-((3,4,5,6-tetrahydroxytetrahydro-2H-pyran-2-yl)methyl)acrylamide. In some cases, the compound of Formula I is (R,Z)-2-cyano-3-(6-(piperidin-1-yl)naphthalen-2-yl)-N-((3,4,5,6-tetrahydroxytetrahydro-2H-pyran-2-yl)methyl)acrylamide.

In some embodiments, the compound of Formula I is:

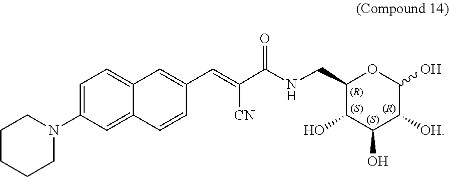

In some cases, the compound of Formula I is 2-cyano-3-(6-(piperidin-1-yl)naphthalen-2-yl)-N-(((2R,3S,4S,5R)-3,4,5,6-tetrahydroxytetrahydro-2H-pyran-2-yl)methyl)acrylamide. In some cases, the compound of Formula I is (E)-2-cyano-3-(6-(piperidin-1-yl)naphthalen-2-yl)-N-(((2R,3S,4S,5R)-3,4,5,6-tetrahydroxytetrahydro-2H-pyran-2-yl)methyl)acrylamide. In some cases, the compound of Formula I is (Z)-2-cyano-3-(6-(piperidin-1-yl)naphthalen-2-yl)-N-(((2R,3S,4S,5R)-3,4,5,6-tetrahydroxytetrahydro-2H-pyran-2-yl)methyl)acrylamide.

In some embodiments, the compound of Formula I is:

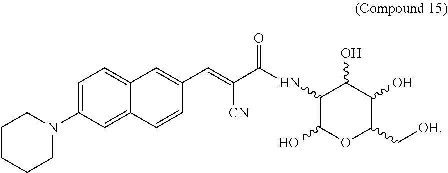 [SMILES: N#C/C(=C\c1ccc2cc(N3CCCCC3)ccc2c1)C(=O)NC1C(O)OC(CO)C(O)C1O]

In some cases, the compound of Formula I is 2-cyano-3-(6-(piperidin-1-yl)naphthalen-2-yl)-N-(2,4,5-trihydroxy-6-(hydroxymethyl)tetrahydro-2H-pyran-3-yl)acrylamide. In some cases, the compound of Formula I is (E)-2-cyano-3-(6-(piperidin-1-yl)naphthalen-2-yl)-N-(2,4,5-trihydroxy-6-(hydroxymethyl)tetrahydro-2H-pyran-3-yl)acrylamide. In some cases, the compound of Formula I is (Z)-2-cyano-3-(6-(piperidin-1-yl)naphthalen-2-yl)-N-(2,4,5-trihydroxy-6-(hydroxymethyl)tetrahydro-2H-pyran-3-yl)acrylamide.

In some embodiments, the compound of Formula I is:

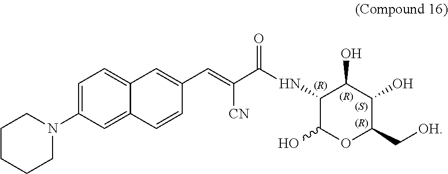

In some cases, the compound of Formula I is 2-cyano-3-(6-(piperidin-1-yl)naphthalen-2-yl)-N-((3R,4R,5S,6R)-2,4,5-trihydroxy-6-(hydroxymethyl)tetrahydro-2H-pyran-3-yl)acrylamide. In some cases, the compound of Formula I is (E)-2-cyano-3-(6-(piperidin-1-yl)naphthalen-2-yl)-N-((3R,4R,5S,6R)-2,4,5-trihydroxy-6-(hydroxymethyl)tetrahydro-2H-pyran-3-yl)acrylamide. In some cases, the compound of Formula I is (Z)-2-cyano-3-(6-(piperidin-1-yl)naphthalen-2-yl)-N-((3R,4R,5S,6R)-2,4,5-trihydroxy-6-(hydroxymethyl)tetrahydro-2H-pyran-3-yl)acrylamide.

In some embodiments, the compound of Formula I is:

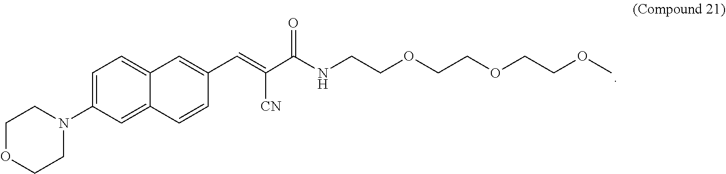 [SMILES: COCCOCCOCCNC(=O)/C(C#N)=C/c1ccc2cc(N3CCOCC3)ccc2c1]

In some cases, the compound of Formula I is 2-cyano-N-(2-(2-(2-methoxyethoxy)ethoxy)ethyl)-3-(6-morpholinonaphthalen-2-yl)acrylamide. In some cases, the compound of Formula I is (E)-2-cyano-N-(2-(2-(2-methoxyethoxy)ethoxy)ethyl)-3-(6-morpholinonaphthalen-2-yl)acrylamide. In some cases, the compound of Formula I is (Z)-2-cyano-N-(2-(2-(2-methoxyethoxy)ethoxy)ethyl)-3-(6-morpholinonaphthalen-2-yl)acrylamide.

In some embodiments, the compound of Formula I is:

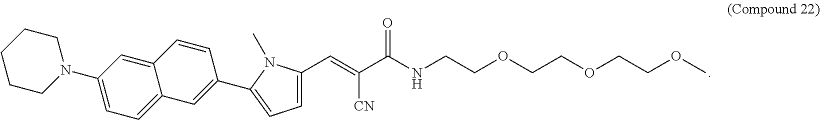 [SMILES: COCCOCCOCCNC(=O)/C(C#N)=C/c1ccc(-c2ccc3cc(N4CCCCC4)ccc3c2)n1C]

In some cases, the compound of Formula I is 2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide. In some cases, the compound of Formula I is (Z)-2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide. In some cases, the compound of Formula I is (E)-2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide.

The disclosure also provides compounds of Formula II, or a pharmaceutically acceptable salt thereof:

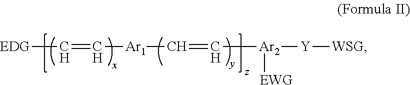

wherein
Ar₂ and each Ar₁ is independently C₁-C₁₄ arylene or C₁-C₁₄ heteroarylene, each optionally substituted with one more R₄₁;
each R₄₁ is independently halogen, —CN, —OR₄₂, —NR₄₃R₄₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R_{45;}
R₄₂, R₄₃ and R₄₄ are independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₄₅;
each R₄₅ is independently halogen, —OR₄₆, —NR₄₇R₄₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and
R₄₆, R₄₇ and R₄₈ are independently hydrogen or C₁-C₁₀ alkyl;
EDG is an electron donating group;
EWG is an electron withdrawing group;
WSG is a water soluble group;
X is C═O or SO₂;
Y is NH, or S;
each x is independently an integer from 0-10;
each w is independently an integer from 1-5;
each y is independently an integer from 0-10; and
z is an integer from 1-10

In some embodiments of a compound of Formula II, each of Ar₁ is independently a substituted or unsubstituted naphthylene or a substituted or unsubstituted phenylene.

In some embodiments of a compound of Formula II, Ar₂ is a substituted or unsubstituted naphthylene or a substituted or unsubstituted phenylene.

In some embodiments of a compound of Formula II, EDG is an electron donating group. In some embodiments of a compound of Formula II, EDG is any electron donor group known in the art. In some embodiments of a compound of Formula II, EDG is any atom or functional group that is capable of donating some of its electron density into a conjugated pi system via resonance or inductive electron withdrawal, thus making the pi system more nucleophilic. In some embodiments of a compound of Formula II, the EDG is —OR₄₉, —NR₅₀R₅₁, —SR₅₂, —PR₅₃R₅₄, —NR₅₅C(O)R₅₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₅₇; wherein
each R₅₇ is independently halogen, —OR₅₈, —NR₅₉R₆₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
each of R₄₉, R₅₀, R₅₁, R₅₂, R₅₃, R₅₄, R₅₅, R₅₆, R₅₈, R₅₉ and R₆₀ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₆₁ and wherein R₅₀ and R₅₁ are optionally joined together to form a heterocycloalkyl or heteroaryl optionally substituted with R₆₁;
each of R₆₁ is independently halogen, —OR₆₂, —NR₆₃R₆₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₆₅;
each of R₆₂, R₆₃ and R₆₄ is independently hydrogen or C₁-C₁₀ alkyl; and
each R₆₅ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

In some embodiments of a compound of Formula II, EDG is selected from a group consisting of

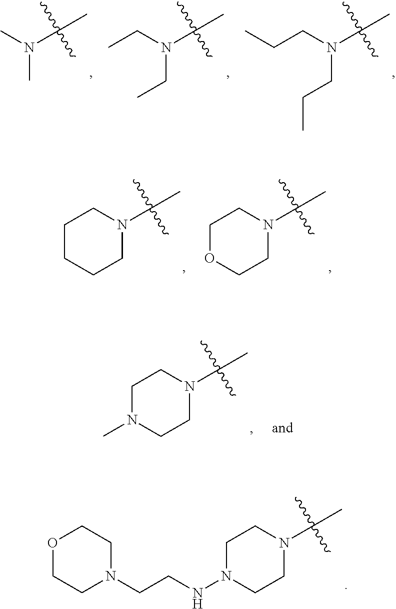

In some embodiments of a compound of Formula II, EDG is

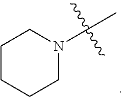 [SMILES: *N1CCCCC1]

In some embodiments of a compound of Formula II, EWG is an electron withdrawing group. In some embodiments of a compound of Formula II, EWG is any atom or group that is capable of drawing electron density from neighboring atoms towards itself, either by resonance or inductive effects. In some embodiments, EWG is halogen, —CN, —NO₂, —SO₃H, —CR₆₆R₆₇R₆₈, —COR₆₉, or —COOR₇₀; wherein
each R₆₆, R₆₇ and R₆₈ is independently hydrogen or halogen;
R₆₉ is halogen, hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₁;
R₇₀ is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₂;
each R₇₁ and R₇₂ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

In some embodiments of a compound of Formula II, EWG is selected from a group consisting of —F, —Cl, —Br, —C═O, NO₂, —CF₃, —CCl₃, —SO₃ and —CN. In some embodiments, the EWG is —F, —Cl, or —Br. In some embodiments of a compound of Formula II, the EWG is —CN.

In some embodiments of a compound of Formula II, WSG is a water soluble group. In some embodiments of a compound of Formula II, the WSG groups serve to alter the solubility of the compounds of Formula II in aqueous systems. In some embodiments of a compound of Formula II, WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₃; wherein
each R₇₃ is independently halogen, —OR₇₄, —NR₇₅R₇₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₇;
each R₇₄, R₇₅ and R₇₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₇;
each R₇₇ is independently halogen, —OR₇₈, —NR₇₉R₈₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and
each of R₇₈, R₇₉ and R₈₀ is independently hydrogen or C₁-C₁₀ alkyl.

In some embodiments of a compound of Formula II, the WSG is hydrogen.

In some embodiments of a compound of Formula II, the WSG is

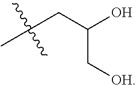 [SMILES: *CC(O)CO]

In other embodiments of a compound of Formula II, the WSG is polyethylene glycol, polypropylene glycol, co-polymer of polyethylene glycol and polypropylene glycol, or alkoxy derivatives thereof In some embodiments, WSG is

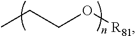

wherein n is an integer from 0-50 and R₈₁ is hydrogen, C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene. In some embodiments of a compound of Formula II, R₈₁ is hydrogen. In some embodiments of a compound of Formula II, R₈₁ is methyl. In some embodiments of a compound of Formula II, R₈₁ is ethyl. In some embodiments, R₈₁ is CH₂—C≡CH. In some embodiments of a compound of Formula II, n is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments of a compound of Formula II, n is an integer of value 1-10, 1-20, 1-30, 1-40, 1-50, 10-20, 10-30, 10-40, 10-50, 20-30, 20-40, 20-50, 30-40, 30-50, or 40-50. In some embodiments, n is 1, 2, 3, 4, 5, 6, 7, 8, 9, or 10. In some embodiments of a compound of Formula II, n is 0, 3 or 6.

In some embodiments of a compound of Formula II, the WSG is

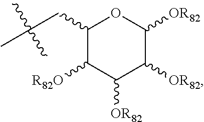

wherein each R₈₂ is hydrogen or C₁-C₁₀ alkyl. In some embodiments of a compound of Formula II, each R₈₂ is independently a hydrogen, methyl, ethyl, propyl, or butyl.

In some embodiments of a compound of Formula II, the WSG is

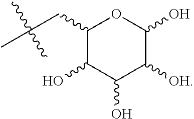 [SMILES: *CC1OC(O)C(O)C(O)C1O]

In some embodiments of a compound of Formula II, the WSG is

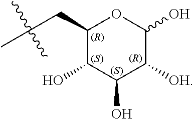

In some embodiments of a compound of Formula II, the WSG is

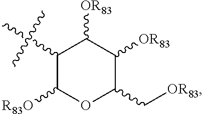

wherein each R₈₃ is hydrogen or C₁₋C₁₀ alkyl. In some embodiments of a compound of Formula II, each R₈₃ is independently a hydrogen, methyl, ethyl, propyl, or butyl.

In some embodiments of a compound of Formula II, WSG is

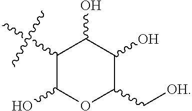

In some embodiment of a compound of Formula II, WSG is

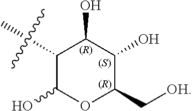

In some embodiments of a compound of Formula II, Y is absent, O, NH, or S. In some embodiments of a compound of Formula II, Y is absent (i.e. Y is a bond). In some embodiments of a compound of Formula II, Y is O. In some embodiments of a compound of Formula II, Y is NH. In some embodiments of a compound of Formula II, Y is S.

In some embodiments of a compound of Formula II, the variable x is an integer from 0-10. In some embodiments of a compound of Formula II, x is 0. In some embodiments of a compound of Formula II, x is 1. In some embodiments of a compound of Formula II, x is 2. In some embodiments of a compound of Formula II, x is 3. In some embodiments of a compound of Formula II, x is 4. In some embodiments of a compound of Formula II, x is 5. In some embodiments of a compound of Formula II, x is 6. In some embodiments of a compound of Formula II, x is 7. In some embodiments of a compound of Formula II, x is 8. In some embodiments of a compound of Formula II, x is 9. In some embodiments of a compound of Formula II, x is 10.

In some embodiments of a compound of Formula II, the variable y is an integer from 0-10. In some embodiments of a compound of Formula II, y is 0. In some embodiments of a compound of Formula II, y is 1. In some embodiments of a compound of Formula II, y is 2. In some embodiments of a compound of Formula II, y is 3. In some embodiments of a compound of Formula II, y is 4. In some embodiments of a compound of Formula II, y is 5. In some embodiments of a compound of Formula II, y is 6. In some embodiments of a compound of Formula II, y is 7. In some embodiments of a compound of Formula II, y is 8. In some embodiments of a compound of Formula II, y is 9. In some embodiments of a compound of Formula II, y is 10.

In some embodiments of a compound of Formula II, the variable z is an integer from 1-10. In some embodiments of a compound of Formula II, z is 1. In some embodiments of a compound of Formula II, z is 2. In some embodiments of a compound of Formula II, z is 3. In some embodiments of a compound of Formula II, z is 4. In some embodiments of a compound of Formula II, z is 5. In some embodiments of a compound of Formula II, z is 6. In some embodiments of a compound of Formula II, z is 7. In some embodiments of a compound of Formula II, z is 8. In some embodiments of a compound of Formula II, z is 9. In some embodiments of a compound of Formula II, z is 10.

In some embodiments, the compound according to Formula II is selected from a group consisting of:

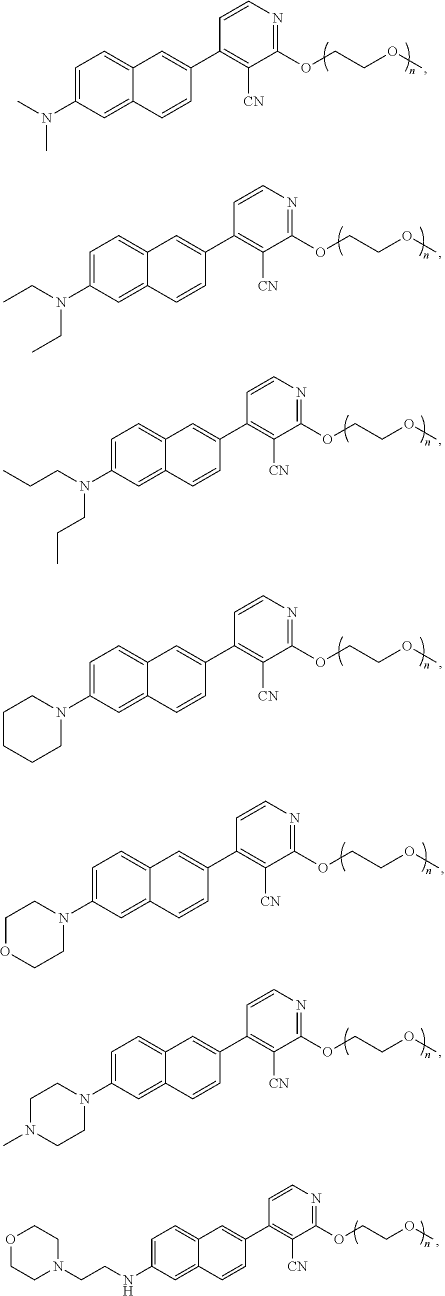

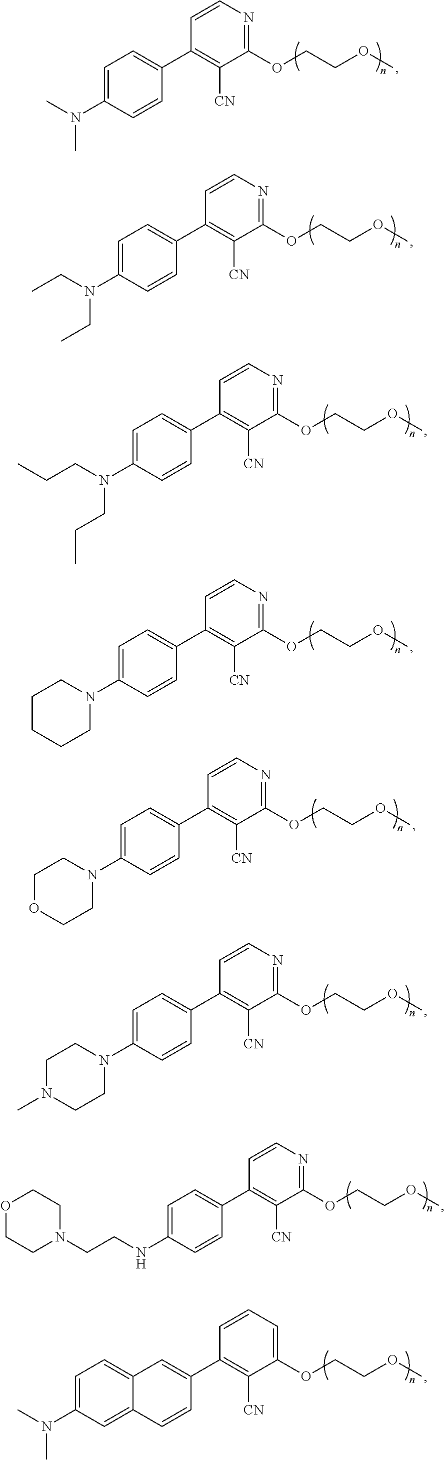

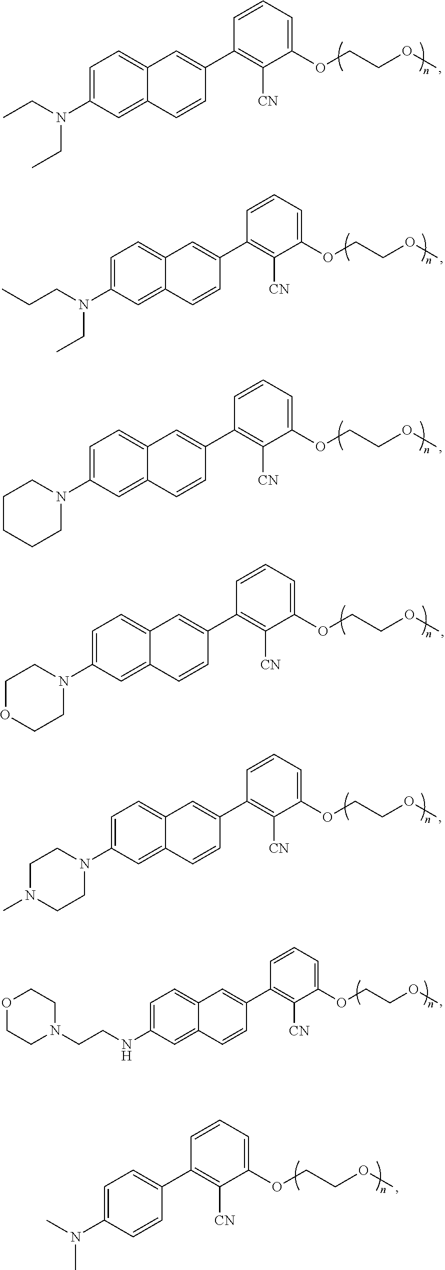

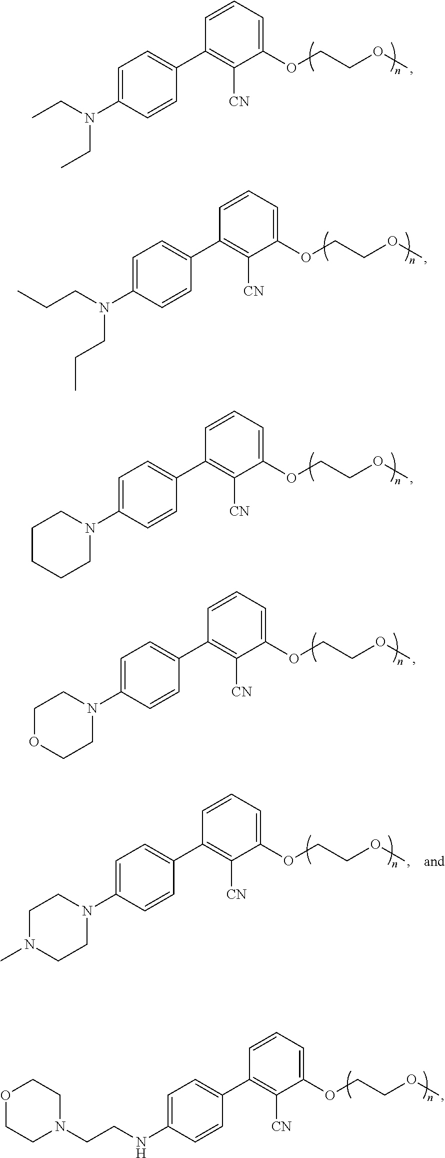

wherein n is an integer with value 0-50. In some embodiments of a compound of Formula II, n is a integer of value 0-10, e.g. n is 0, 1, 2, 3, 4, 5, 6, 7, 8, 9, or 10.

In some embodiments, the compound according to Formula II is selected from a group consisting of:

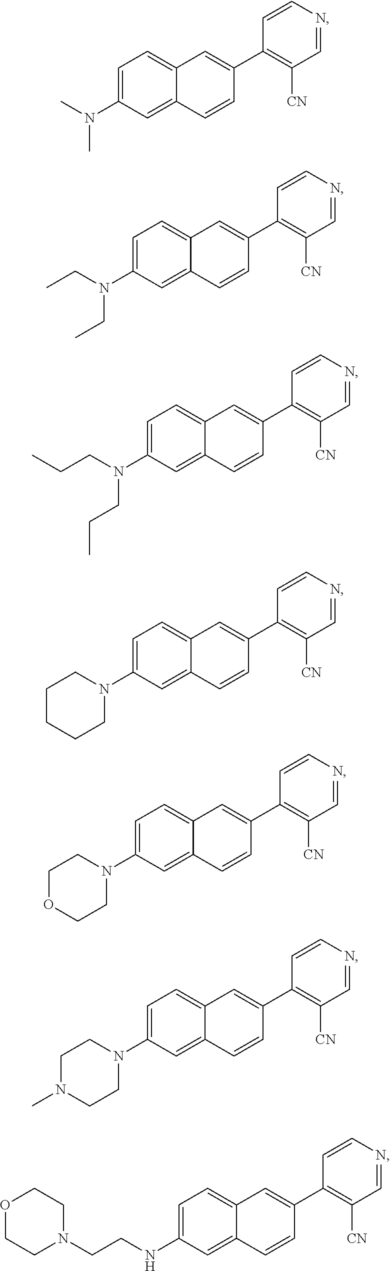

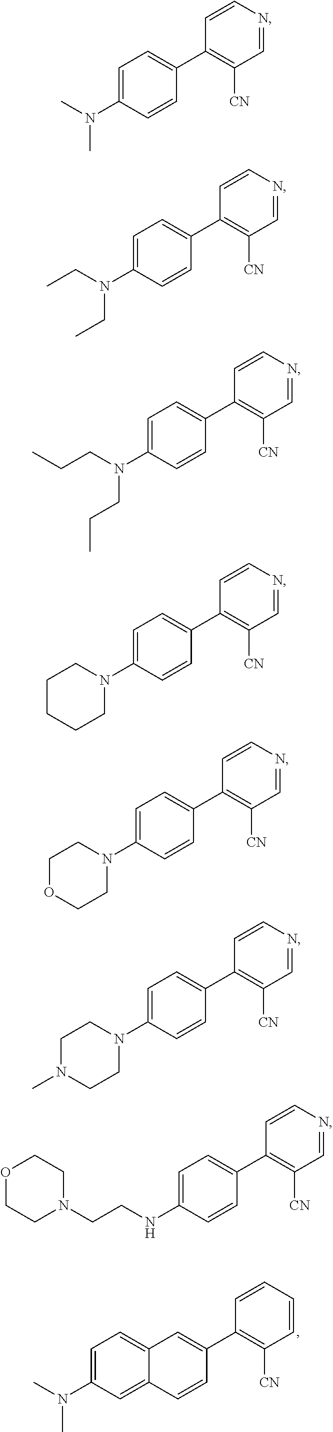

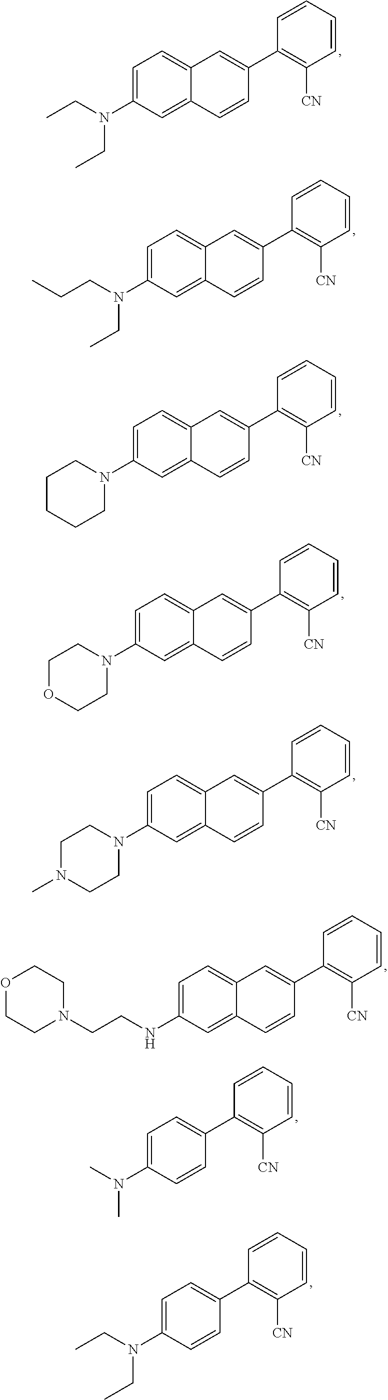

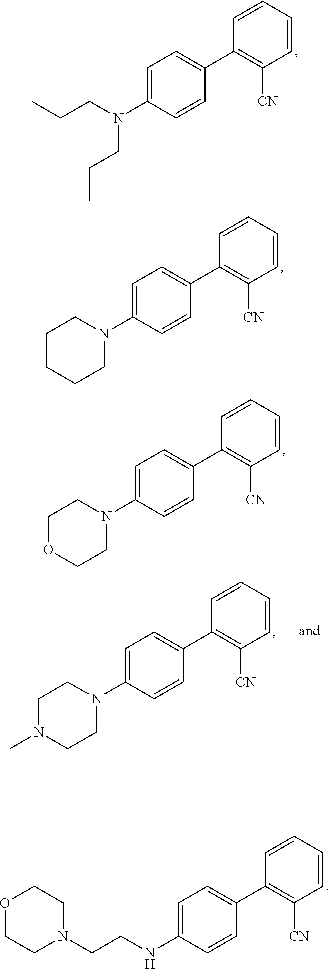 [SMILES: CCCN(CCC)c1ccc(-c2ccccc2C#N)cc1 ; N#Cc1ccccc1-c1ccc(N2CCCCC2)cc1 ; N#Cc1ccccc1-c1ccc(N2CCOCC2)cc1 ; CN1CCN(c2ccc(-c3ccccc3C#N)cc2)CC1 ; N#Cc1ccccc1-c1ccc(NCCN2CCOCC2)cc1]

In some embodiments, the compound of Formula II is selected from a group consisting of

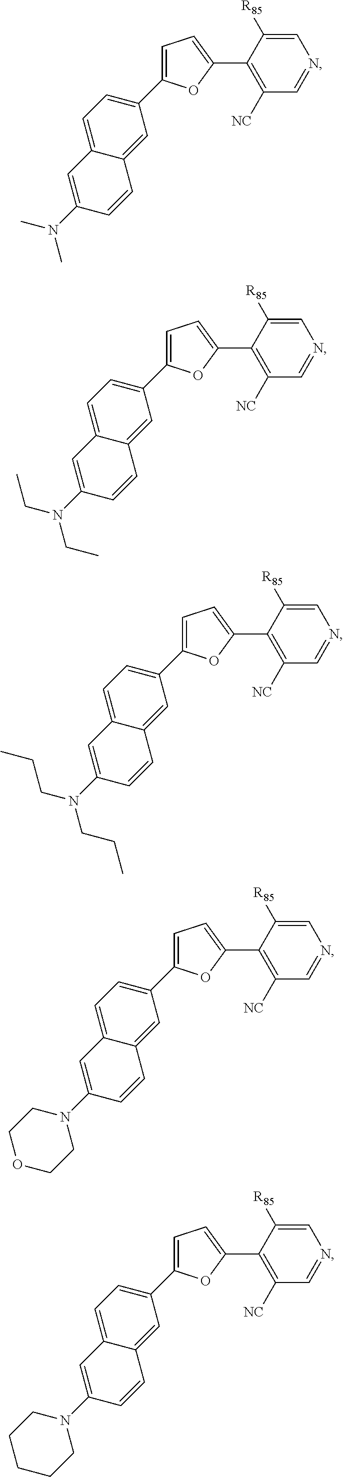

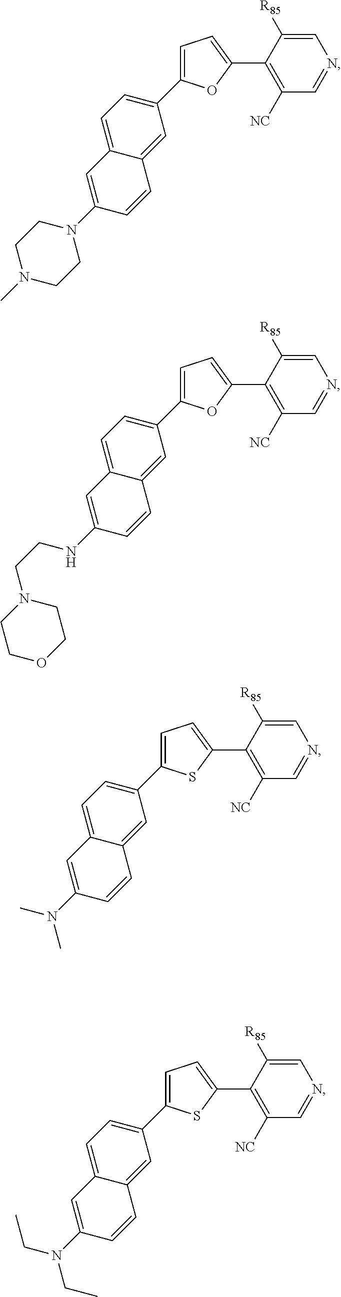

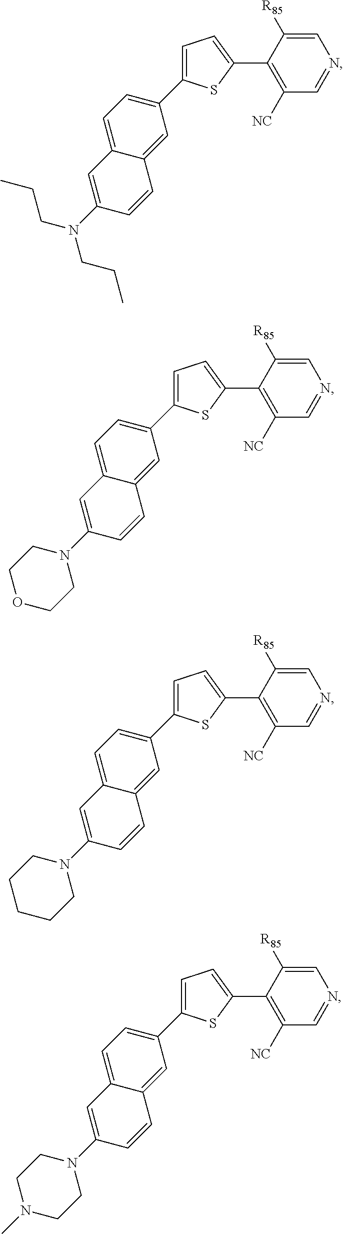

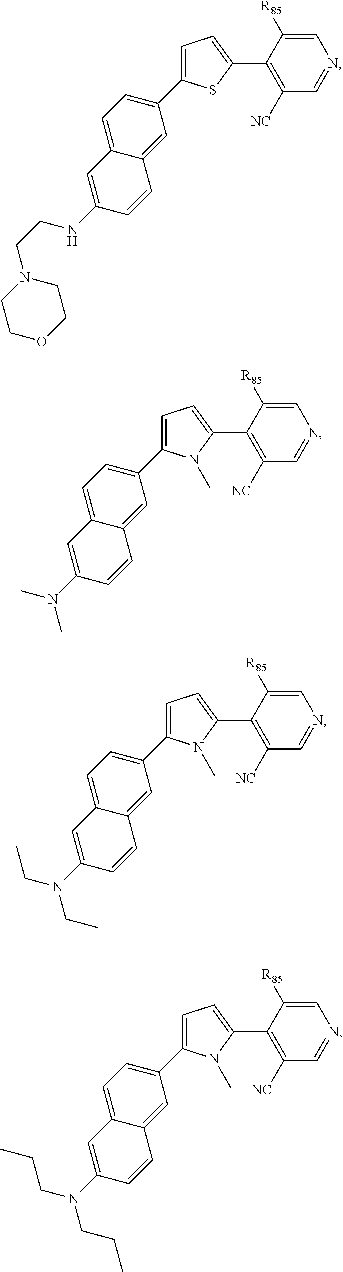

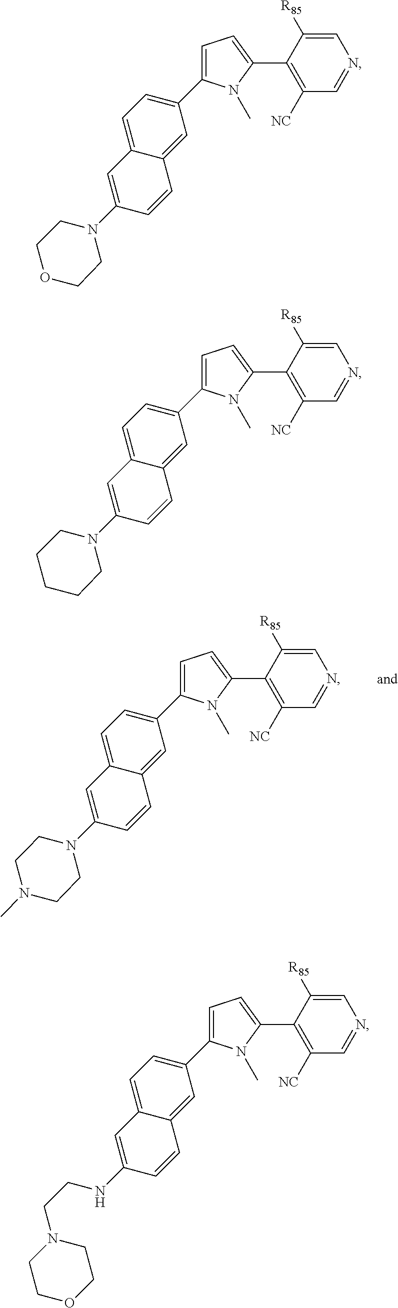

wherein R85 is H or CN.

In some embodiments, the compound of Formula II is selected from a group consisting of

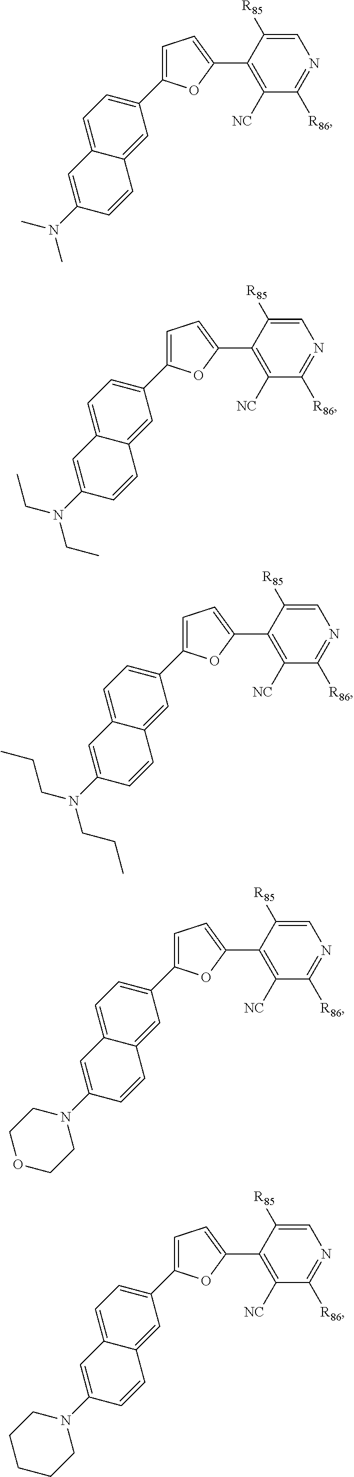

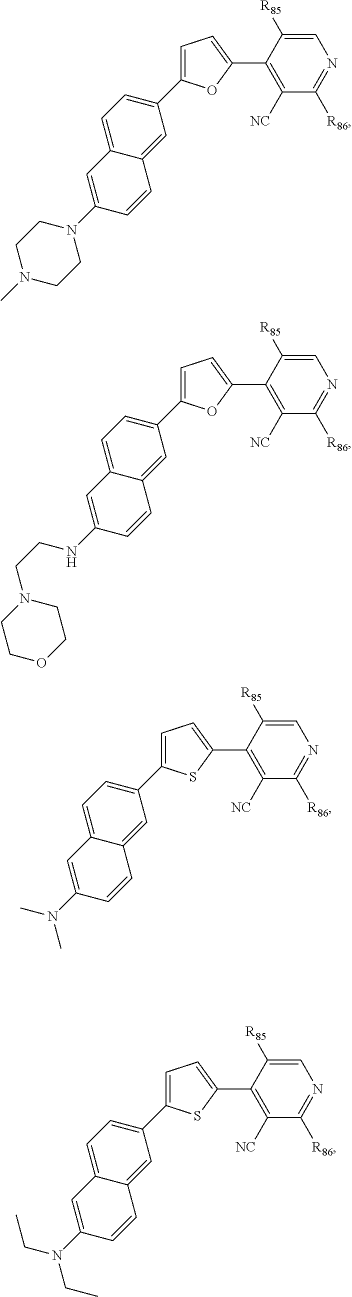

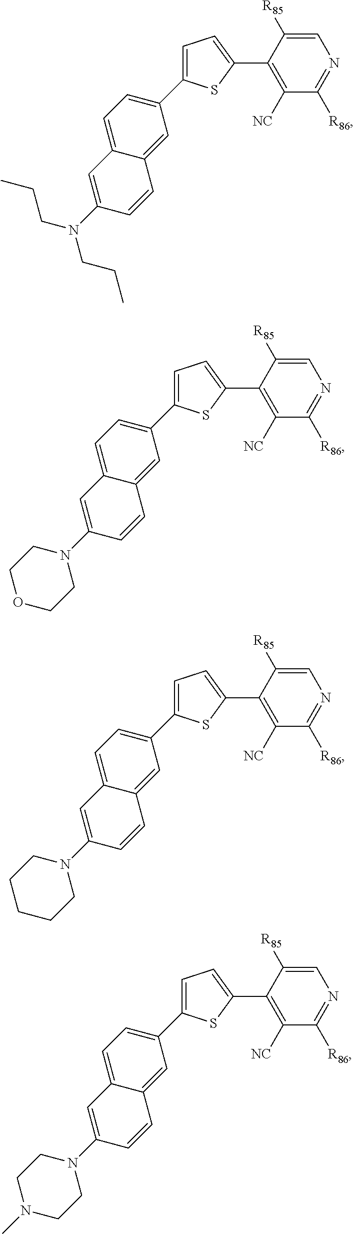

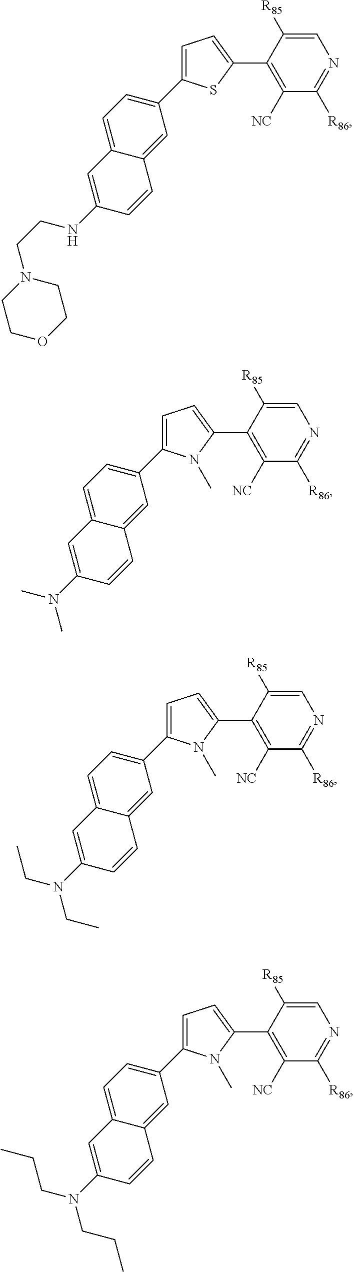

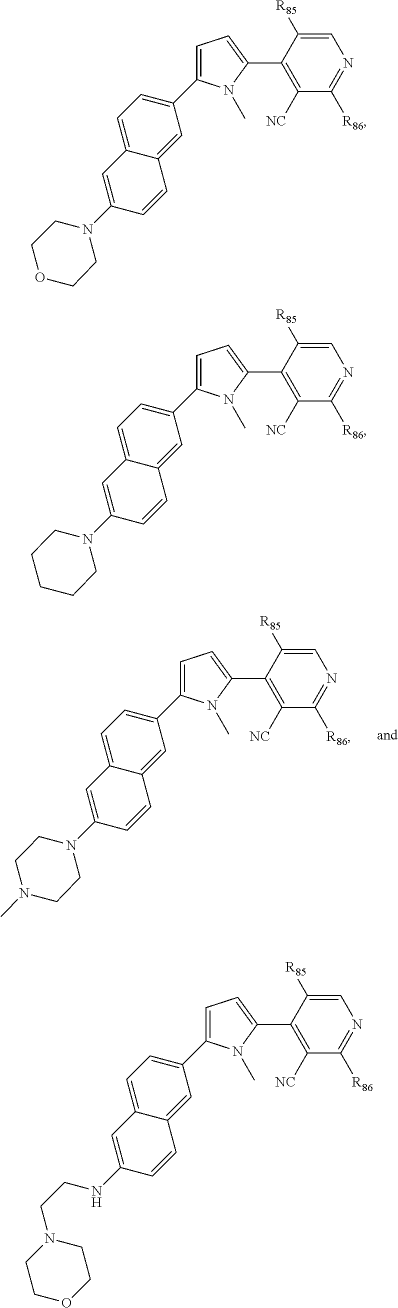

wherein R₈₅ is H or CN and R₈₆ is

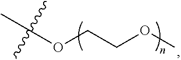

wherein n is an integer with value 0-50. In some embodiments of a compound of Formula II, n is a integer of value 0-10, e.g. n is 0, 1, 2, 3, 4, 5, 6, 7, 8, 9, or 10.

In some embodiments, the compound of Formula II is selected from a group consisting of

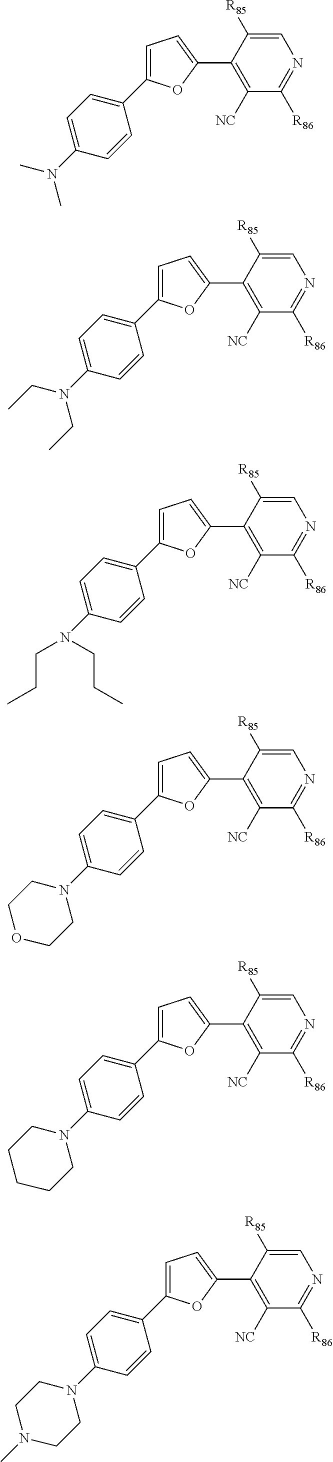

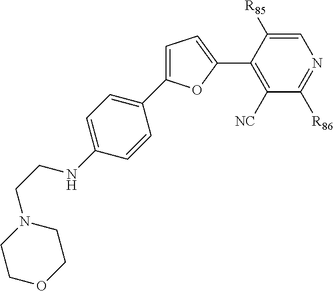

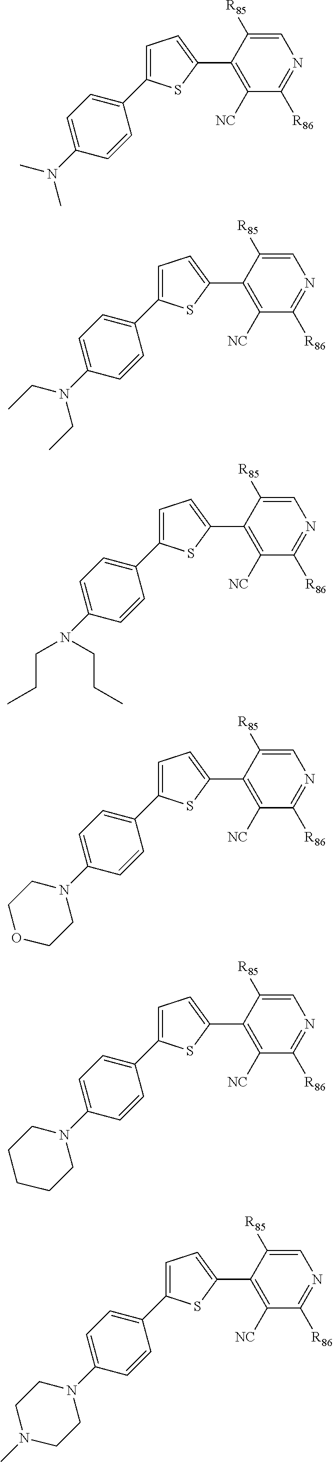

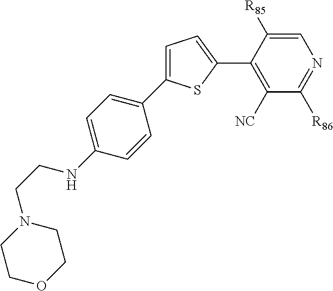

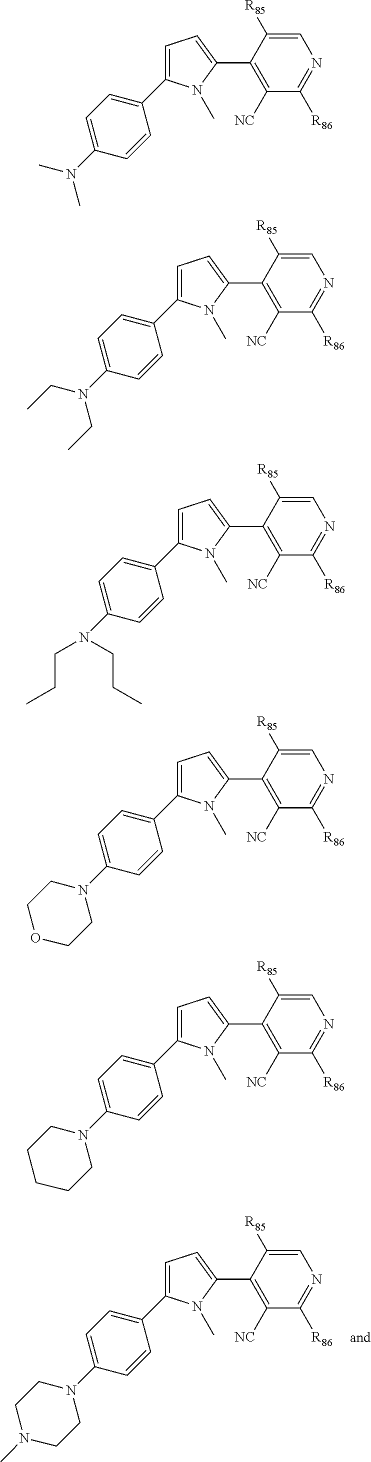

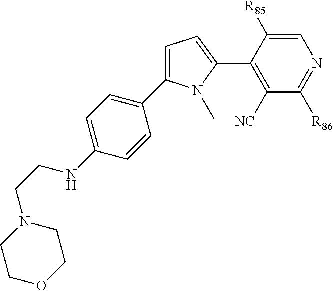

wherein R₈₅ is H or CN and R₈₆ is H.

In some embodiments, the compound of Formula II is selected from a group consisting of

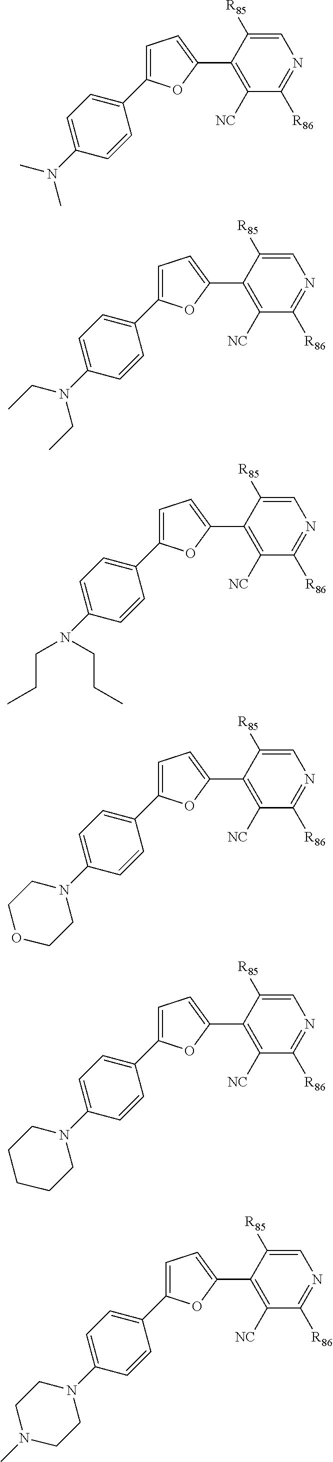

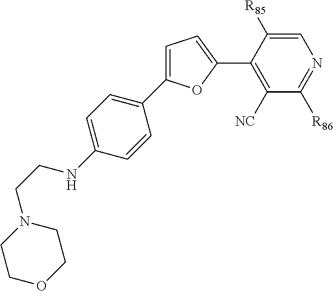

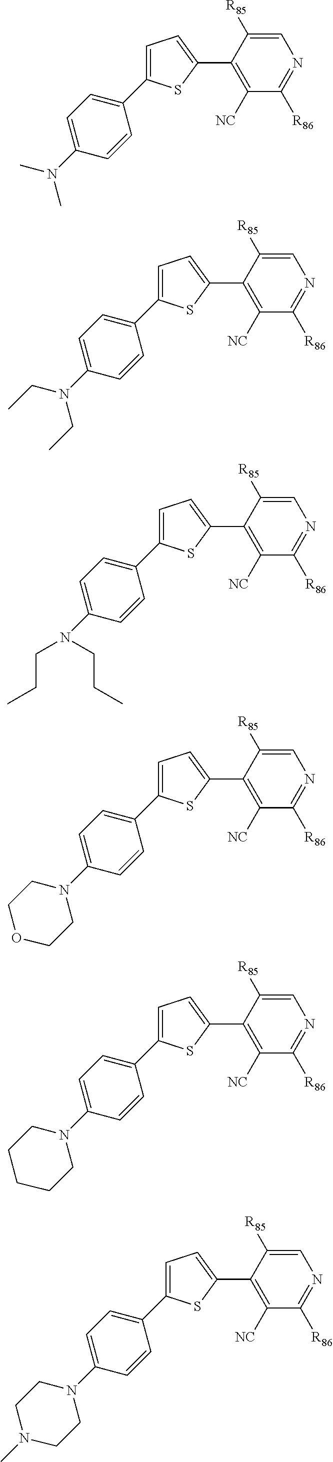

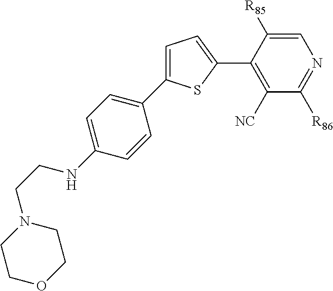

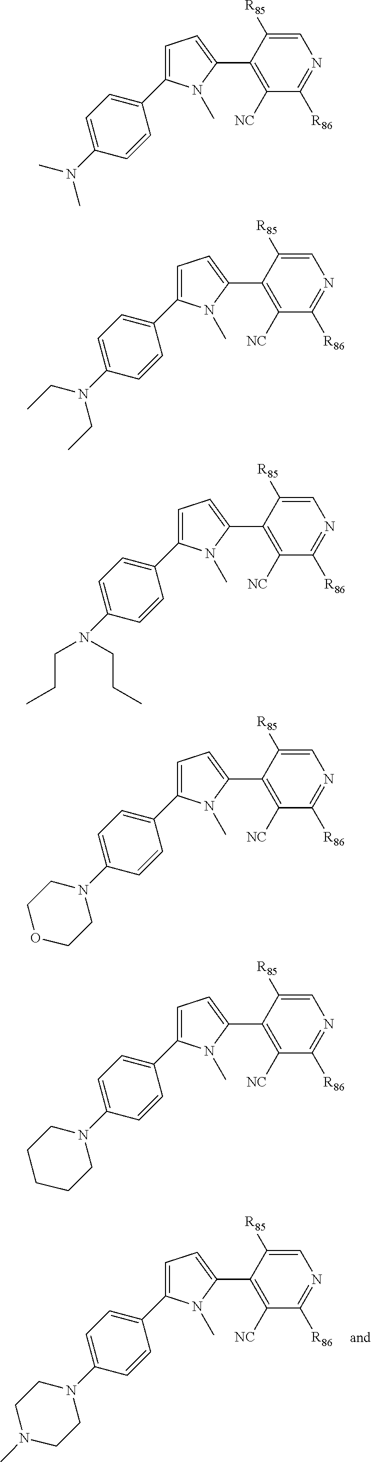

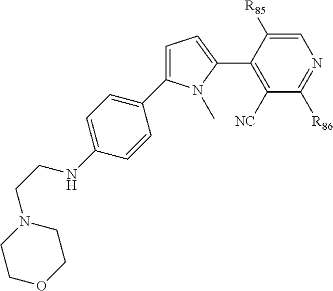

wherein R₈₅ is H or CN and R₈₆ is

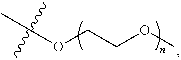

wherein n is an integer with value 0-50. In some embodiments of a compound of Formula II, n is a integer of value 0-10, e.g. n is 0, 1, 2, 3, 4, 5, 6, 7, 8, 9, or 10.

In some embodiments of a compound of Formula II, the compound of Formula II is

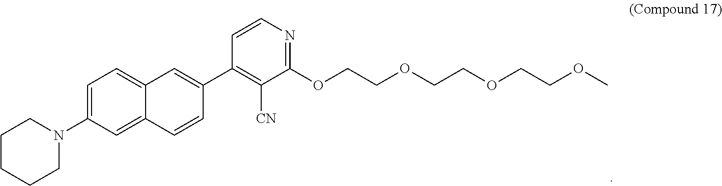 [SMILES: COCCOCCOCCOc1nccc(-c2ccc3cc(N4CCCCC4)ccc3c2)c1C#N]

In some embodiments of a compound of Formula II, the compound of Formula II is

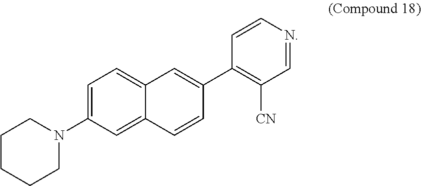 [SMILES: N#Cc1cnccc1-c1ccc2cc(N3CCCCC3)ccc2c1]

Some useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In some embodiments, the formulations of the disclosure are packaged in multidose form or in single dose units. In some cases, the formulations are packaged in multidose forms. In some embodiments, the formulations are packaged as single dose from. In some embodiments, of the disclosure single dose packaging of the formulations can offer several advantages over multi dose packaging including dosage control, increased patient compliance, improved product labeling, and reduced counterfeiting. In various examples single dosage packaging of the formulations of the disclosure can be in form of vials, ampoules, tubes, bottles, pouches, packettes, syringes or blister packs.

In certain examples, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds provided in the compositions of the present disclosure is less than 100%, 90%, 80%, 70%, 60%, 50%, 40%, 30%, 20%, 19%, 18%, 17%, 16%, 15%,14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more compounds of the disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds of the disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the disclosure is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more compounds of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more compounds of the disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

### Kits/Articles of Manufacture

The disclosure also provides a kit comprising a compound according to the disclosure. In some embodiments, the compounds of the disclosure are contained in a container as formulations. For example the kit comprises the compounds of the disclosure contained in a container as a sterile liquid formulation. In some embodiments, the compounds are placed in the containers as a sterile freeze-dried formulation. In some embodiments, the container is a vial, for example an amber vial. In some cases, the container is capable of protecting light sensitive compounds or formulation.

In some embodiments, such kits comprise a carrier, package, or container that is compartmentalized to receive one or more containers such as vials, tubes, and the like, wherein one or more of the container(s) comprise the compound of Formula I or Formula II. Suitable containers include, for example, bottles, vials, syringes, and test tubes. The containers are formed from a variety of materials such as glass or plastic. In some embodiments, the containers is chosen so as to protect, limit or minimize the exposure of the compounds of Formula I or Formula II to light. For example, the container is an amber vial.

The articles of manufacture provided herein contain packaging materials. Packaging materials for use in packaging include those found in, e.g., U.S. Pat. Nos. 5,323,907, 5,052,558 and 5,033,252. Examples of packaging materials include, but are not limited to, blister packs, bottles, tubes, inhalers, pumps, bags, vials, containers, syringes, bottles, and any packaging material suitable for a selected formulation and intended mode of administration and treatment. In some embodiments, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

In some embodiments, a kit includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. In some embodiments, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein.

### Methods of Use

In one aspect, the disclosure provides a method for detecting one or more amyloid or amyloid like proteins comprising contacting a compound according to according to any one of Formula I or II with a sample potentially comprising the amyloid or amyloid like protein to form a composition as disclosed herein, wherein in presence of an amyloid or amyloid like protein the compound forms a detectable complex, detecting the formation of the detectable complex such that the presence or absence of the detectable complex correlates with the presence or absence of the amyloid or amyloid like protein.

In some embodiments, the compositions of the instant disclosure are used for detecting one or more amyloid or amyloid like protein with high sensitivity. In some embodiments, the compounds predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% sensitivity. In some embodiments, the compounds are capable of detecting one or more amyloid or amyloid like protein with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity. In some cases the compounds are capable of detecting one or more amyloid or amyloid like protein with greater than 99.5%, 99.6%, 99.7%, 99.8% or 99.9% sensitivity.

In some embodiments, the compositions of the instant disclosure are used for detecting one or more amyloid or amyloid like protein with high specificity. In some embodiments, the compounds detect one or more amyloid or amyloid like protein with greater than 10%, 15%, 20%, 25%, 30%, 35%, 40%, 45%, 50%, 55%, 60%, 65%, 70%, 75%, 80%, 85%, 90%, 95% or 99% specificity. In some embodiments, the compounds are capable of detecting one or more amyloid or amyloid like protein with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity. In some cases the compounds are capable of detecting one or more amyloid or amyloid like protein with greater than 99.5%, 99.6%, 99.7%, 99.8% or 99.9% specificity.

In some embodiments, the compositions of the disclosure are used for detecting one or more amyloid or amyloid like protein with both high specificity and high specificity. In some embodiments, the compounds are capable of detecting one or more amyloid or amyloid like protein with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity and greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity.

Also provided herein is a method of determining the presence or absence of one or more disease or condition in a subject comprising administering to the subject an effective amount of a compound according to any one of Formula I or II, wherein in presence of the disease or condition the administered compound forms a detectable complex, and detecting the formation of the detectable complex such that presence or absence of detectable complex correlates with the presence or absence of the disease or condition. In some embodiments, the compounds of the disclosure are used for determining the presence or absence of one or more amyloid-based disease or condition, wherein in presence of the amyloid-based disease or condition the administered compound forms a detectable complex, and detecting the formation of the detectable complex such that presence or absence of detectable complex correlates with the presence or absence of the amyloid-based disease or condition. In some embodiments, the compounds of the disclosure are used for determining the presence or absence of one or more disease or condition characterized by protein aggregation or protein misfolding.

In some embodiments, the method includes comparing the amount of the detectable complex to a normal control value, wherein an increase in the amount of the complex compared to a normal control value indicates that said patient is suffering from or is at risk of developing the disease or condition.

In some embodiments, a single dose of the compounds of the disclosure are used to determining the presence or absence of multiple diseases disease or conditions in a subject. In some embodiments, a single dose is used to detect the presence or absence of 2, 3, 4, 5, 6, 7, 8, 9, 10, 11, 12, 13, 14, 15, 16, 17, 18, 19 or 20 diseases in a subject. In some cases, a single dose is used to determine the presence of 1, 2, 3, 4, or 5 disease or conditions.

In some embodiments, the compositions of the instant disclosure are used for diagnosis with high sensitivity. In some embodiments, the compounds predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% sensitivity. In some embodiments, the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity. In some cases the compounds are capable of diagnosis with greater than 99.5%, 99.6%, 99.7%, 99.8% or 99.9% sensitivity.

In some embodiments, the compositions of the instant disclosure are used for diagnosis with high specificity. In some embodiments, the compounds predict the presence and or absence of a disease with greater than 10%, 15%, 20%, 25%, 30%, 35%, 40%, 45%, 50%, 55%, 60%, 65%, 70%, 75%, 80%, 85%, 90%, 95% or 99% specificity. In some embodiments, the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity. In some cases the compounds are capable of diagnosis with greater than 99.5%, 99.6%, 99.7%, 99.8% or 99.9% specificity.

In some embodiments, the compounds of the disclosure are used for diagnosis with both high specificity and high specificity. In some embodiments, the compounds are capable of diagnosis with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity and greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% specificity.

In many cases, the compound is applied to a composition comprising a sample from a patient. The mixture is then assayed for a detectable change in the compound, such as a change in spectral properties of the compound, in response to contacting with a protein aggregate in the composition. In some cases the compound displayes changed emission spectra in response to contacting with a protein aggregate such as an amlyoid or a pre-eclampsia complex. The method includes bringing the sample suspected to contain the amyloid or amyloid like protein into contact with a compound of the disclosure, allowing the compound to bind to the amyloid or amyloid like protein to form a detectable complex, detecting the formation of the detectable complex and correlating the presence or absence of the detectable complex with the presence or absence of amyloid or amyloid like protein in the sample or specific body part or area. In some embodiments, the method includes comparing the amount of said detectable complex to a normal control value, wherein an increase in the amount of said detectable complex compared to a normal control value indicates that said patient is still suffering from a minimal residual disease.

Compounds as disclosed herein are used at a range of effective concentrations in detection compositions. Compounds are used, ofr example at μM concentrations of 0.01 μM., 0.05 μM, 0.1 μM, 0.2, 0.3, 0.4, 0.5, 0.6, 0.7, 0.8, 0.9, 1, 1.1, 1.2, 1.3, 1.4, 1.5, 1,=0.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2., 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 10, 15, 20, 25, 50, 75, 100 or greater than 100 μM.

Excitation wavelengths are selected for the specific compound and specific composition, and range in some cases from 300 nm to 500 nm, for example 300, 310, 320, 330, 340, 350, 360, 370, 380, 390, 400, 410, 420, 430, 440, 450, 460, 470, 480, 490, or 500 nm, or a value intermediate between any of these values. Emission spectra are selected for the specific compound and specific composition, and range in some cases from 450 nm to 650 nm, for example 450, 460, 470, 480, 490, 500, 510, 520, 530, 540, 550, 560, 570, 580, 590, 600, 610, 620, 630, 640, 650 nm, or a value intermediate between any of these values.

In various aspects throughout the disclosure, the detection of the detectable complex disclosure comprises illuminating the sample with light of an appropriate wavelength for a peak region of a fluorescent excitation spectrum for the detectable complex and detecting light received from the sample of an appropriate wavelength for a peak region of a fluorescent emission spectrum for the detectable complex. In some embodiments, the detectable complex is a complex of a compound of Formula I or II with an amyloid or amyloid-like protein. In some embodiments, the excitation spectrum has a peak at about 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 n, 490 nm, 500 nm, 510 nm, 520 nm, 530 nm, 540 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, or 900 nm. In some embodiments, the fluorescent excitation spectrum of the detectable complex has a peak at about 350-400, 350-450 nm, 350-500 nm, 350-550 nm, 350-600 nm, 400-450 nm, 400-500, 400-550 nm, 400-600 nm, 450-500 nm, 450-550 nm, 450-600 nm, 500-550, or 550-600 nm. In some embodiments, the fluorescent excitation spectrum of the detectable complex has a peak at about 350-400 nm, 400-500 nm or 450-500 nm. In some embodiments, the illuminating of the sample is at a wavelength within plus or minus about 100 nm, 90 nm, 80 nm, 70 nm, 60 nm, 50 nm, 40 nm, 30 nm, 20 nm, 10 nm, or 0 nm of the peak of the excitation spectrum. In some embodiments, the illuminating light has a wavelength of 300-500 nm, 350-450 nm, 400-500 nm. In some embodiments, the illuminating light has a wavelength of 400 nm.

In some embodiments, the emission spectrum has a peak of about 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 n, 490 nm, 500 nm, 510 nm, 520 nm, 530 nm, 540 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, or 900 nm. In some embodiments, the emission spectrum of the detectable complex has a peak at about 500-550 nm, for example at about 510-540 nm. In some embodiments, the emission spectrum of the detectable complex has a peak at about 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm or 540 nm. In some embodiments, the detecting of light received from the sample is at a wavelength within plus or minus about 100 nm, 90 nm, 80 nm, 70 nm, 60 nm, 50 nm, 40 nm, 30 nm, 20 nm, 10 nm, or 0 nm of the peak of the emission spectrum.

Any number of devices are suitable for the administration of excitation energy and detection of emission specta. In some cases, samples are delivered to cuvetes, such as quartz cuvetes, to facilitate analysis. In alternate cases, samples are measured in transparent plates such that multiple samples are measured simultaneously, or are measured one at a time. Sample containment structures, and excitation generation devices, and emission detection devices are well knowin to one of skill in the art.

Some preferred wavelenght/composition combinations are given in Table 1, below.

TABLE 1

| | | | Spectral | Slit | |
|---|---|---|---|---|---|
| AMDX | Concentration | Excitation | Sweep | Width^{a} | Emission |
| Probe | (μM) | (nm) | (nm) | (nm) | RFI (nm) |
| Compound 1 | 4 | 441 | 461-750 | 5 | 534 |
| Compound 2 | 1.3 | 440 | 460-750 | 5 | 535 |
| Compound 5 | 7.7 | 388 | 408-850 | 5 | 540 |
| Compound | 2.8 | 418 | 438-750 | 5 | 563 |
| 21 | | | | | |
| Compound | 1.9 | 446 | 466-750 | 5 | 554 |
| 22 | | | | | |

However, alternate values for each of these compounds are also suitable, and in some cases preferred excitation and emission spectra are selected in light of the spectral properties of the sample as well as the spectral properties of the detection molecule, or the detection device.

Detection of the aggregates is measured by a number of approaches. For example, in some cases the strength of signal at a given wavelength is proprtional to the amount of protein aggregate in a sample. Thus, by comparing the emission spectrum at select wavelength or wavelengths, one is able to assess the amount or proportion of protein agregate in a sample. One then is able to assess the relative risk of the patient from which the sample is obtained of having the disease theat corresponds to the protein aggregate being detected.

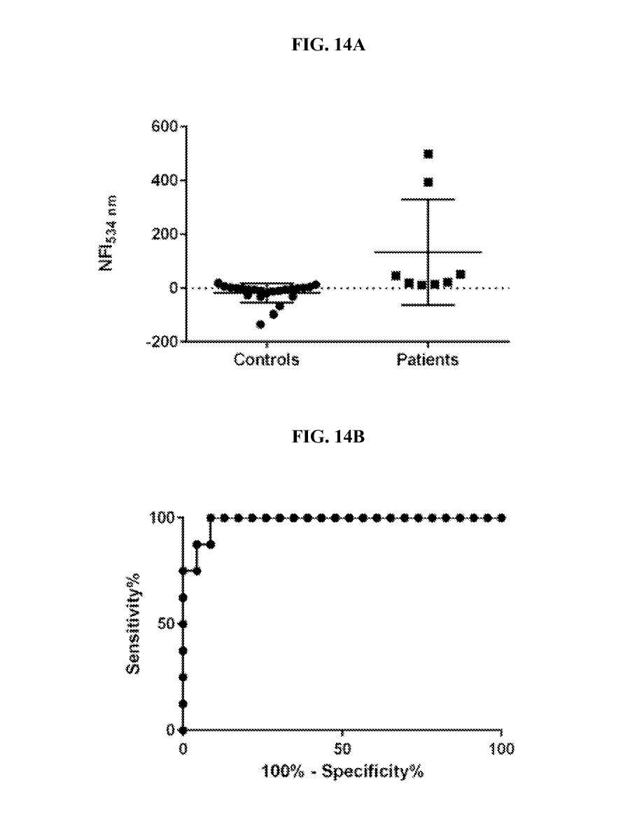

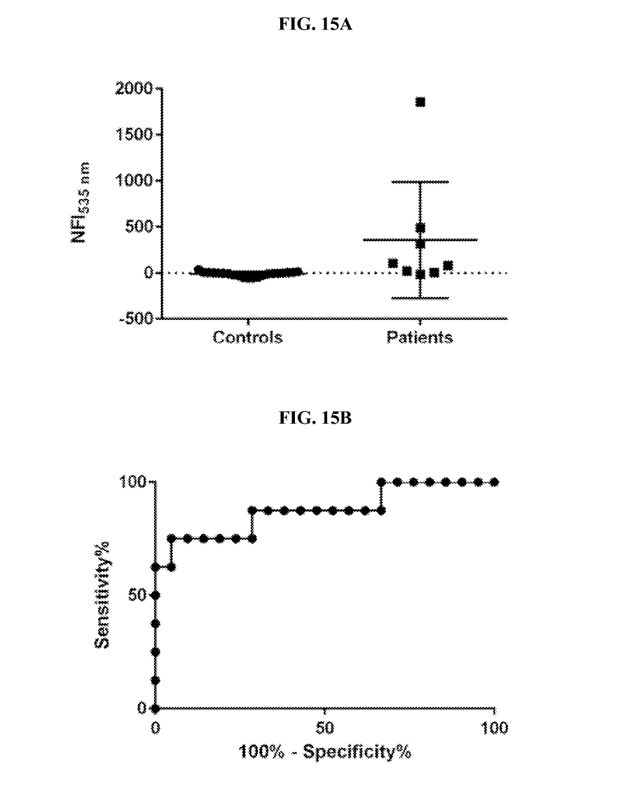

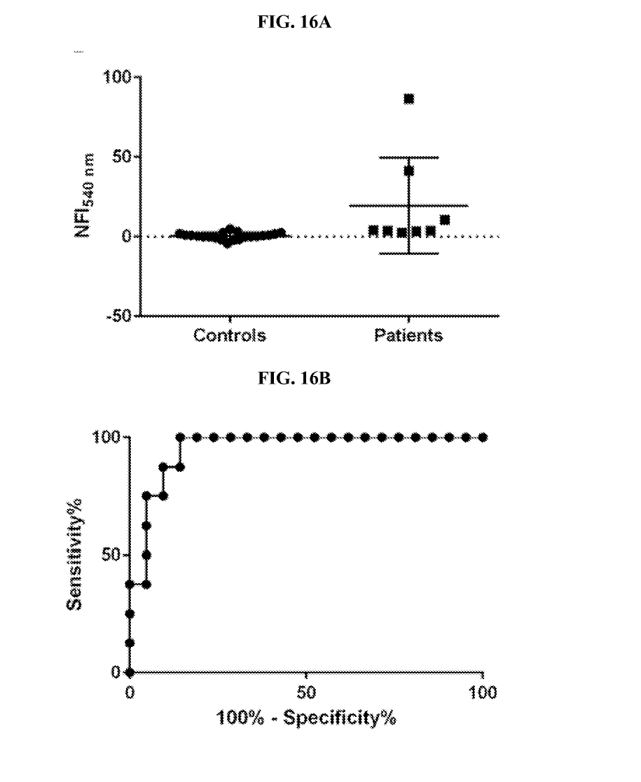

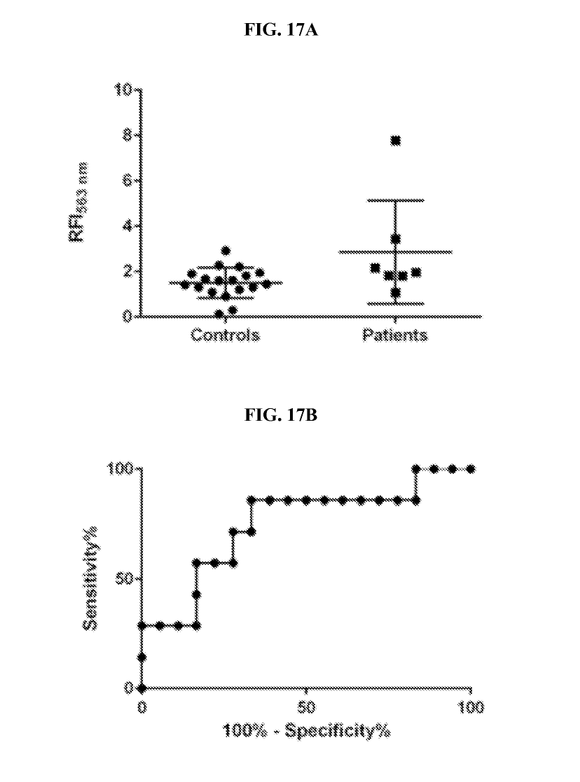

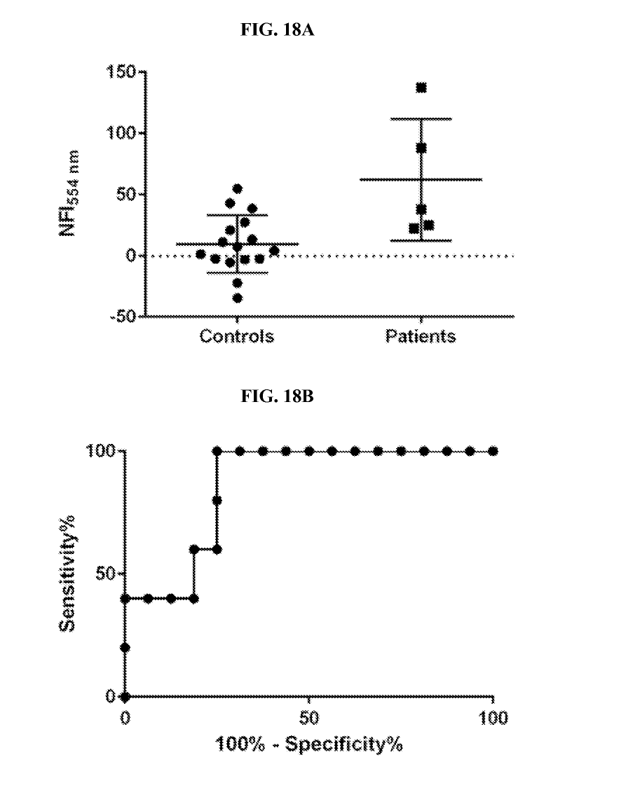

Reference is made to FIGS. 14A-19B, which depict molecules, test results and ROC results indicating sensitivity and specificity of a number of tests of specific compounds' performance at detecting protein aggregates in samples. It is seen, for example in FIG. 14B, that a compound disclosed herein is successfully used in a test disclosed herein having an ROC curve well off the diagnonal for the chart. The ROC indicates a test perfromance having a specificity of about 100% at a sensitivity above 75%, and a specificity of about 90% at a sensitivity of about 100%. Similarly one sees at FIG. 15B a compound that leads to a test performance having a specificity of about 100% at a sensitivity above 62%, and a specificity of about 60% at a sensitivity of about 90%. Similarly one sees at FIG. 16B a compound that leads to a test performance having a specificity of about 95% at a sensitivity above 75%, and a specificity of about 80% at a sensitivity of about 100%. Test having values intermediate on these ROC curves are also disclosed according to the ROC curves. One observes that the compounds disclosed herein and tested perfrom well both in terms of specificity and sensitivity of detection of the protein aggregates assayed for.

Thests using compounds disclosed herein are usefule for a number of methods related to protein aggregate diseases.

Also provided herein is a method of predicting responsiveness of a patient to a treatment, wherein the method includes bringing the sample suspected to contain the amyloid or amyloid like protein into contact with a compound of the disclosure, allowing the compound to bind to the amyloid or amyloid like protein to form a detectable complex, detecting the formation of the detectable complex and correlating the presence or absence of the detectable complex with the presence or absence of amyloid or amyloid like protein in the sample or specific body part or area. In some embodiments, the method includes comparing the amount of the detectable complex before and after onset of the treatment, wherein a decrease in the amount of the detectable complex indicate that the patient is being responsive to the treatment.

Provided herein is screening method, wherein the method comprises administering to a subject an effective amount of a compound of Formula I or II. In some embodiments, upon administration, the compound of Formula I or II form a detectable complex. In some embodiments, the method further comprise measuring a signal generated by the compound of Formula I or Formula II upon administration to the subject, or by the detectable complex formed by the compound of Formula I or II. In some embodiments, the method comprises making a clinical decision based on the measured signal.

The term ‘amyloid-based disease or condition’ refers to any disease or condition. The term also includes any disease or condition characterized by protein aggregation or protein misfolding. In some cases amyloid-based disease or condition is any disease or condition that is associated with the increased or decreased presence of amyloid or amyloid like proteins or proteins, such as the presence of amyloid plaques. In some embodiments, the amyloid based disease or condition is a neuronal disease or condition, for example, neurodegenerative diseases, in which amyloid-beta peptides, oligomers, fibrils, or plaques are implicated. Non limiting examples of amyloid-based neurodegenerative diseases include Alzheimer's disease, Parkinson's disease, Huntington's disease, Down's Syndrome, and spongiform encephalopathies such as, for example, bovine spongiform encephalopathy (mad cow disease) kuru, Creutzfeldt-Jakob disease, and fatal familial insomnia. In some embodiments, other amyloid based diseases that are detected, treated or prevented by the methods of the disclosure include reactive systemic amyloidosis, senile systemic amyloidosis (SSA), familial amyloid polyneuropathy (FAP), familial amyloid cardiomyopathy (FAC), prion disease, coronary heart disease, atherosclerosis, cerebral hemorrhage, AL amyloidosis, type 2 diabetes, diseases or conditions characterized by a loss of cognitive memory capacity such as, for example, mild cognitive impairment (MCI), Lewy body dementia (LBD), hereditary cerebral hemorrhage with amyloidosis (Dutch type) and the Guam Parkinson-Dementia complex. Other diseases which are based on or associated with amyloid-like proteins are progressive supranuclear palsy, multiple sclerosis, HIV-related dementia, ALS (amyotropic lateral sclerosis), inclusion-body myositis (IBM), Adult Onset Diabetes; endocrine tumors, and other diseases, including amyloid-associated ocular diseases that target different tissues of the eye, such as the visual cortex, including cortical visual deficits; the anterior chamber and the optic nerve, including glaucoma; the lens, including cataract due to beta-amyloid deposition; the vitreous, including ocular amyloidosis; the retina, including primary retinal degenerations and macular degeneration, in particular age-related macular degeneration; the optic nerve, including optic nerve drusen, optic neuropathy and optic neuritis; and the cornea, including lattice dystrophy.

In some embodiments, the compounds of the present disclosure can be employed for the treatment of Alzheimer's disease, Alzheimer's disease (AD), Parkinson's disease, Huntington's disease, amyotrophic, lateral sclerosis (ALS), Lewy body dementia (LBD), or Down's syndrome. In some embodiments, the compounds of the present disclosure can be employed for the detection, diagnosis, treatment and monitoring of Alzheimer's disease. Or the compounds of the present disclosure can be employed for the detection, diagnosis, treatment and monitoring of Creutzfeldt-Jakob disease (CJD).

Amyloid-based disease or condition also include ocular diseases associated with pathological abnormalities/changes in the tissues of the visual system, particularly associated with amyloid-beta-related pathological abnormalities/changes in the tissues of the visual system, such as, for example, neuronal degradation. Said pathological abnormalities occur, for example, in different tissues of the eye, such as the visual cortex leading to cortical visual deficits; the anterior chamber and the optic nerve leading to glaucoma; the lens leading to cataract due to beta-amyloid deposition; the vitreous leading to ocular amyloidosis; the retina leading to primary retinal degeneration and macular degeneration, for example age-related macular degeneration; the optic nerve leading to optic nerve drusen, optic neuropathy and optic neuritis; and the cornea leading to lattice dystrophy.

In some embodiments, the amyloid or amyloid like proteins and/or proteins that are detected using the methods of the disclosure include amyloid beta peptides (Aβ), prion peptide (PrP), alpha-synuclein, IAPP (amylin), huntingtin, calcitonin (ACal), atrial natriuretic factor (AANF), apolipoprotein A1 (ApoA1), serum amyloid A (SAA), medin (AMed), prolactin (APro), transthyretin (ATTR), lysozyme (ALys), beta 2 microglobulin (Aβ2M), gelsolin (AGel), keratoepithelin (Aker), cystatin (ACys), immunoglobulin light chain AL (AL), S-IBM or superoxide dismutase. In some embodiments, the amyloid peptide detected by the method of the disclosure is AP peptide, prion peptide, alpha-synuclein, or superoxide dismutase.

In some embodiments, the subjects for the methods of the instant disclosure are any mammal, for example, a primate (such as a human), canine, feline, ovine, bovine and the like. In some embodiments, biological samples that are used in the diagnosis of an amyloid-associated disease or condition for diagnosing a predisposition to an amyloid-associated disease or condition or for monitoring minimal residual disease in a patient or for predicting responsiveness of a patient to a treatment with a compound or a composition or a mixture according to the disclosure and as described herein before are, for example, fluids such as serum, plasma, saliva, gastric secretions, mucus, cerebrospinal fluid, lymphatic fluid, and the like, or tissue or cell samples obtained from an organism such as neural, brain, cardiac or vascular tissue. In some embodiments, any immunoassay known to those of ordinary skill in the art is used for determining the presence or absence of the amyloid or amyloid like protein in a sample such as, for example, assays which utilize indirect detection methods using secondary reagents for detection, ELISA's and immunoprecipitation and agglutination assays.

In some cases the compositions and methods disclosed herein are used in a test for pre-eclampsia or the potential to develop pre-eclampsia in a pregnant woman by assaying for the presence of amyloid in a urine sample from the pregnant woman, whereby presence of amyloid above a threshold is indicative of pre-eclampsia or the risk of developing pre-eclampsia.

In some cases the compositions and methods disclosed herein are used in a test for pre-eclampsia or the potential to develop pre-eclampsia in a pregnant woman by assaying for the presence of amyloid in a blood sample from the pregnant woman, whereby presence of amyloid above a threshold is indicative of pre-eclampsia or the risk of developing pre-eclampsia.

In some cases the compositions and methods disclosed herein are used in a test for pre-eclampsia or the potential to develop pre-eclampsia in a pregnant woman by assaying for the presence of amyloid in a sweat sample from the pregnant woman, whereby presence of amyloid above a threshold is indicative of pre-eclampsia or the risk of developing pre-eclampsia.

In some cases the compositions and methods disclosed herein are used in a test for pre-eclampsia or the potential to develop pre-eclampsia in a pregnant woman by assaying for the presence of amyloid in a mucous sample from the pregnant woman, whereby presence of amyloid above a threshold is indicative of pre-eclampsia or the risk of developing pre-eclampsia.

In some cases the compositions and methods disclosed herein are used in a test for Alzheimer's or the potential to develop Alzheimer's in a human by assaying for the presence of amyloid in a urine sample for the human, whereby presence of amyloid above a threshold is indicative of Alzheimer's or the risk of developing Alzheimer's.

In some cases the compositions and methods disclosed herein are used in a test for Alzheimer's or the potential to develop Alzheimer's in a human by assaying for the presence of amyloid in a blood sample for the human, whereby presence of amyloid above a threshold is indicative of Alzheimer's or the risk of developing Alzheimer's.

In some cases the compositions and methods disclosed herein are used in a test for Alzheimer's or the potential to develop Alzheimer's in a human by assaying for the presence of amyloid in a mucous sample for the human, whereby presence of amyloid above a threshold is indicative of Alzheimer's or the risk of developing Alzheimer's.

In some cases the compositions and methods disclosed herein are used in a test for Cerebral Amyloid Angiopathy or the potential to develop Cerebral Amyloid Angiopathy in a human by assaying for the presence of amyloid in a urine sample for the human, whereby presence of amyloid above a threshold is indicative of Cerebral Amyloid Angiopathy or the risk of developing Cerebral Amyloid Angiopathy.

In some cases the compositions and methods disclosed herein are used in a test for Cerebral Amyloid Angiopathy or the potential to develop Cerebral Amyloid Angiopathy in a human by assaying for the presence of amyloid in a blood sample for the human, whereby presence of amyloid above a threshold is indicative of Cerebral Amyloid Angiopathy or the risk of developing Cerebral Amyloid Angiopathy.

In some cases the compositions and methods disclosed herein are used in a test for Cerebral Amyloid Angiopathy or the potential to develop Cerebral Amyloid Angiopathy in a human by assaying for the presence of amyloid in a mucous sample for the human, whereby presence of amyloid above a threshold is indicative of Cerebral Amyloid Angiopathy or the risk of developing Cerebral Amyloid Angiopathy.

In some embodiments, thresholds for making a disease determination or disease risk determination are established by a number of approaches and vary among the diseases and amyloid proteins assayed. In some cases, amyloid/compound complex levels are compared to a negative control of a similar sample form an individual of known amyloid-free status or a pool of individuals of known amyloid free status. In some cases amyloid/compound levels of corresponding to known disease status for a given amyloid or a given disease are measured and recorded, such that a given individual's sample amyloid/compound level is compared not to a control sample value but to previously determined values of known disease status. Negative controls consistent with the disclosure herein include, for example, values determined for a sample of a healthy individual, values determined for a sample of a pool of healthy individuals; determination of values for a sample for which any amyloid has been removed, for example through selective degradation or nonspecific protease treatment, or through measurement of a sample comprising compound but no human sample, or levels known to correspond to the absence of disease or the absence of likelihood of developing the disease. Positive controls consistent with the disclosure herein include, for example, values determined for an individual known to suffer from the disease or disorder, values from an individual known to demonstrate no symptoms of the disease or disorder but known to later have developed symptoms of the disease or disorder, values determined for a sample of a pool of individuals known to suffer from the disease or disorder, values determined for a sample of a pool of individuals known to demonstrate no symptoms of the disease or disorder but known to later have developed symptoms of the disease or disorder, values for a sample for which an amyloid has been added, or levels known to correspond to disease or likelihood of developing the disease.

In some cases a risk evaluation is provided rather than a yes/no determination of disease status. In some embodiments, a disease risk status is evaluated as a proportion of amyloid/compound complex present above a background negative control level, or as a proportion of amyloid/compound complex present relative to a known threshold level corresponding to definitive or likely presence of the symptoms of the disorder.

In some cases multiple samples are taken over time, such as two samples, three samples, four samples, or more than four samples, such that variations in amyloid accumulation in a given sample source from an individual can be monitored. In some cases time points in which samples are acquired coincide with administration of a treatment measure, such as administration of a drug or therapy regime. In some cases time points in which samples are acquired are separated from one another by administration of a treatment measure, such as administration of a drug or therapy regimen. In some cases at least two sample collection time points are not separated by an instance of administration of a treatment regimen or drug. By, for example, comparing amyloid levels among or between time points, one may in some cases evaluate the efficacy of a treatment regimen or drug treatment, or assess the progression of an amyloid disorder or the accumulation of amyloid in an individual at risk of developing an amyloid disorder over time. In some cases a treatment regimen or drug treatment is adjusted in response to the information gained by such temporal monitoring of amyloid levels, by for example increasing dosage or frequency of administration, decreasing dosage or frequency of administration, or selecting an alternate or additional drug or treatment regimen.

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, represent a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Two or more heteroatoms may also be consecutive.

The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together (i.e. a fused ring aryl) or linked covalently. A fused ring aryl refers to multiple rings fused together wherein at least one of the fused rings is an aryl ring.

The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e. multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

An “arylene” and a “heteroarylene,” alone or as part of another substituent means a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

As used herein, the term “heteroatom” or “ring heteroatom” is meant to include oxygen (O), nitrogen (N), sulfur (S), phosphorus (P), and silicon (Si).

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present disclosure possess asymmetric carbon atoms (optical centers) or double bonds; the racemates, diastereomers, tautomers, geometric isomers and individual isomers are encompassed within the scope of the present disclosure. The compounds of the present disclosure do not include those which are known in the art to be too unstable to synthesize and/or isolate.

The compounds of the present disclosure may also contain unnatural proportions of atomic isotopes at one or more of the atoms that constitute such compounds. In some embodiments, the compounds may be radiolabeled with radioactive isotopes, such as for example tritium (³H), iodine-125 (¹²⁵O) or carbon-14 (¹⁴C). All isotopic variations of the compounds of the present disclosure, whether radioactive or not, are encompassed within the scope of the present disclosure.

The compounds of the present disclosure may also comprise a tag such as a biotin tag to facilitate purification of amyloid/compound complexes from the compositions disclosed herein.

The terms “treating” or “treatment” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. In some embodiments, the certain methods presented herein successfully treat cancer by decreasing the incidence of cancer, in inhibiting its growth and or causing remission of cancer.

An “effective amount” is an amount of a compound described herein sufficient to contribute to the treatment, prevention, or reduction of a symptom or symptoms of a disease, or to inhibit effects of an amyloid relative to the absence of the compound. Where recited in reference to a disease treatment, an “effective amount” may also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) a disease, or reducing the likelihood of the onset (or reoccurrence) of a disease or its symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an osteoclast or leukocyte relative to the absence of the antagonist.

Diseases or conditions that may be assayed for with the compounds of the present disclosure include diseases or conditions accompanied by protein that produces amyloid like morphology and disease or conditions associated with the formation of abnormal protein structures, protein aggregation, or protein misfolding. In the context of the present disclosure, an abnormal protein structure may be a protein structure that arises when a protein or peptide refolds from the three-dimensional structure, which it generally adopts in healthy individuals, into a different three-dimensional structure, which is associated with a pathological condition. In particular, in one aspect diseases or conditions that may be assayed for with the compounds of the present disclosure are diseases or conditions associated with amyloid or amyloid-like proteins. Such diseases may be referred to as amyloid based diseases or conditions. Amyloid based diseases or conditions, include any disease or condition that is associated with amyloid or amyloid-like protein and is characterized, in part, by the buildup of extracellular deposits of amyloid or amyloid-like material. In the context of this disclosure, amyloid based diseases or conditions also include disease or conditions accompanied by protein that produces amyloid like morphology. These diseases include, but are not limited to, neurological disorders such as Alzheimer's disease (AD), Parkinson's disease, Huntington's disease, diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex. Other diseases which are based on or associated with amyloid-like proteins are progressive supranuclear palsy, multiple sclerosis; Creutzfeldt Jacob disease, Parkinson's disease, HIV-related dementia, ALS (amyotropic lateral sclerosis), inclusion-body myositis (IBM), Adult Onset Diabetes; senile cardiac amyloidosis; endocrine tumors, and other diseases, including amyloid-associated ocular diseases that target different tissues of the eye, such as the visual cortex, including cortical visual deficits; the anterior chamber and the optic nerve, including glaucoma; the lens, including cataract due to beta-amyloid deposition; the vitreous, including ocular amyloidosis; the retina, including primary retinal degenerations and macular degeneration, in particular age-related macular degeneration; the optic nerve, including optic nerve drusen, optic neuropathy and optic neuritis; and the cornea, including lattice dystrophy.

The term “amyloid protein” is intended to denote a protein which is involved in the formation of fibrils, plaques and/or amyloid deposits, either by being part of the fibrils, plaques and/or deposits as such or by being part of the biosynthetic pathway leading to the formation of the fibrils, plaques and/or amyloid deposits. In the present context the term “protein” or is intended to mean both short peptides of from 2 to 10 amino acid residues, oligopeptides of from 11 to 100 amino acid residues, polypeptides of more than 100 amino acid residues, and full length proteins. The terms also encompass peptides having substantial similarity to amyloid proteins, such as, e.g., structural variants. The proteins may occur naturally or be synthetically constructed. The term amyloid protein or amyloid like protein also includes amyloidigenic proteins and proteins that produce amyloid like morphology.

The term “substantial similarity” means that two peptide sequences, when optimally aligned, share at least 50% sequence identity, or at least 60% sequence identity, or at least 70% sequence identity, or at least 80% sequence identity, or at least 90 percent sequence identity, or at least 95 percent sequence identity or more (e.g., 99% sequence identity). Preferably, residue positions, which are not identical, differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Residue positions, which are not identical may also be composed of peptide analogs, including unnatural amino acids or derivatives of such. Analogs typically differ from naturally occurring peptides at one, two or a few positions, often by virtue of conservative substitutions. Some analogs also include unnatural amino acids or modifications of N or C terminal amino acids at one, two or a few positions. Examples of unnatural amino acids are D-amino acids, alpha, alpha-disubstituted amino acids, N-alkyl amino acids, lactic acid, 4-hydroxyproline, y-carboxyglutamate, epsilon-N,N,N-trimethyllysi-ne, epsilon-N-acetyllysine, O-phosphoserine, N-acetylserine, N-formylmethionine, 3-methylhistidine, 5-hydroxylysine, omega.-N-methylarginine, and isoaspartic acid.

The term “a compound as disclosed herein” refers to a compound of Formula I or Formula II or both Formula I and Formula II.

The term “about” as disclosed herein in the context of a number refers to that number plus or minus 10% of that number, unless otherwise specified.

### EXAMPLES

### Example 1

### Synthesis of Compound 1

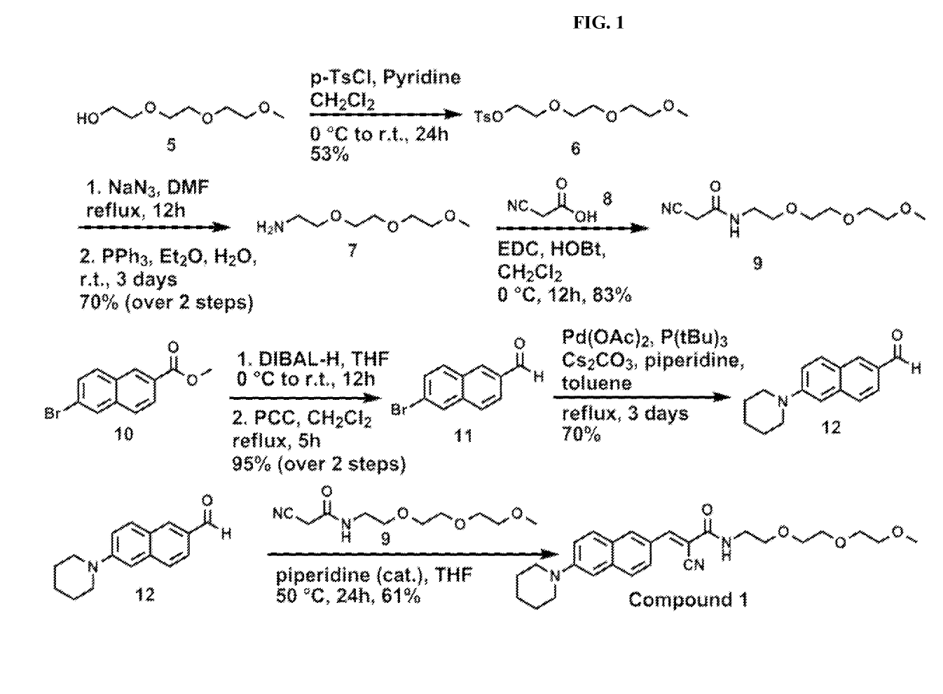

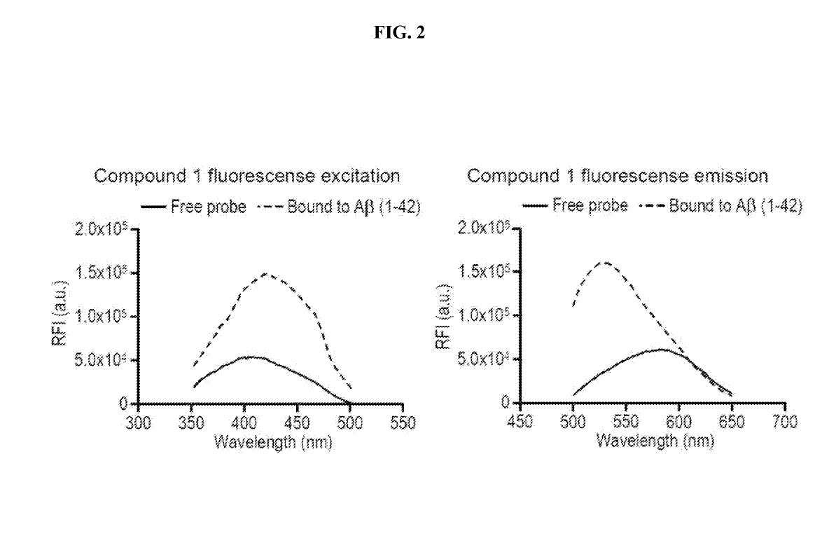

The synthesis of this compound is shown in FIG. 1. Commercially available triethyl glycol (TEG) monomethyl ether (5) was converted to the corresponding tosylate (6) in 53% yield by reaction with p-tosylchloride and pyridine in DCM at 0° C. for 24 h. The tosylate was treated with NaN₃ in DMF at reflux temperature for 12 h to obtain the corresponding azide in 60% yield. The resulting azide was then subjected to a Staudinger reduction to give the TEG-amine (7) in 70% yield over two steps. Amine (7) was coupled to cyanoacetic acid (8) using 1-Ethyl-3-(3-dimethylaminopropyl)carbodiimide (EDC) in 83% yield to give the cyano TEG-amide-acrylate (9). TEG-amide-acrylate (9) was then coupled with the piperidine-napthaldehyde (12), which was synthesized via a reduction and oxidation of commercially available bromo-napthlene methyl ester (10) to aldehyde (11) followed by a Buchwald-Hartwig amination with piperidine, to give compound 1 via Knoevenagel condensation in 61% yield.

Characterization Data:

R_{f}=0.25 (5% acetone/toluene);

¹H NMR (500 MHz, CDCl₃) δ 8.33 (s, 1H), 8.11 (s, 1H), 8.00-8.02 (dd, J=8.5 Hz, 1.5 Hz, 1H), 7.69-7.71 (d, J=9.5 Hz, 1H), 7.60-7.62 (d, J=8.5 Hz, 1H), 7.24-7.26 (m, 1H), 7.01 (bs, 1H), 6.84 (m, 1H), 3.64-3.66 (m, 6H), 3.62-3.63 (m, 4H), 3.54-3.55 (m, 2H), 3.35 (s, 3H), 3.12-3.34 (m, 4H), 1.69 (m, 4H), 1.61-1.62 (m, 2H);

¹³C (125 MHz, CDCl₃) δ 161.2, 152.9, 151.6, 137.2, 133.8, 130.3, 127.2, 126.6, 126.1, 125.7, 119.4, 117.8, 108.6, 100.5, 71.9, 70.6, 70.6, 70.5, 69.4, 59.0, 49.5, 40.2, 25.5, 24.3;

HRMS calcd for C₂₆H₃₂N₂O₅Na[M+Na]⁺ 474.2363, found 474.2363 by ESI.

### Example 2

### Synthesis of Compound 2

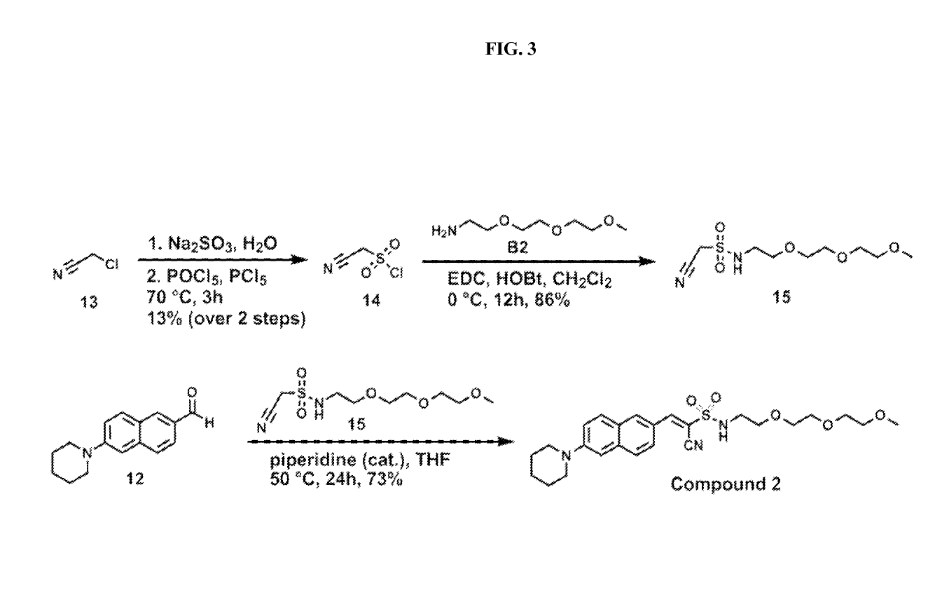

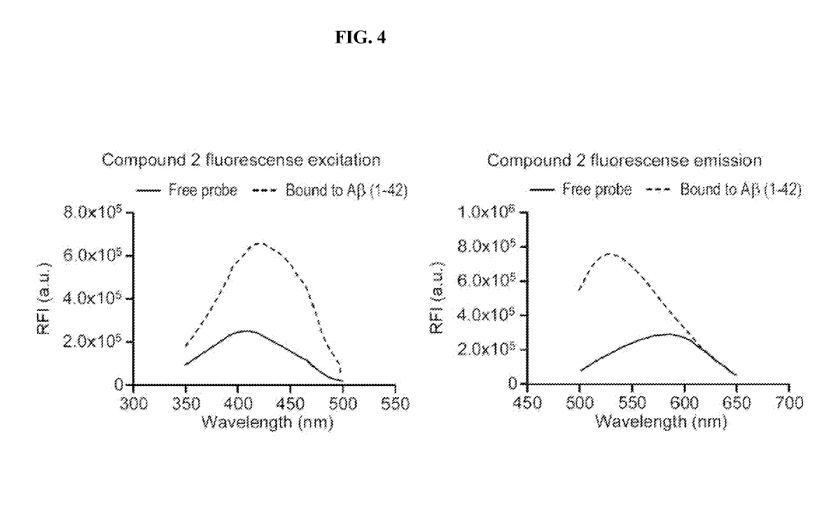

Synthesis of compound 2 is shown in FIG. 3. The synthesis began with converting chloroacetonitrile (13) to the sulfonic acid and then to the sulfonyl chloride (14) in 13% yield over two steps. Sulfonyl chloride (14) was found to be relatively unstable and therefore was quickly reacted with amine-TEG (7) to give sulfonamide-TEG (15) in 86% yield. Sulfonamide-TEG (15) was then condensed with aldehyde (12) to give compound 2 in 73% yield.

Characterization Data:

R_{f}=0.5 (100% diethyl ether);

¹H NMR (500 MHz, CDCl3) δ 8.12 (s, 1H), 8.05 (s, 1H), 7.98-8.00 (dd, J=9.0 Hz, 2.0 Hz, 1H), 7.74-7.76 (d, J=9.5 Hz, 1H), 7.64-7.66 (d, J=9.0 Hz, 1H), 7.29-7.31 (dd, J=9.0 Hz, 2.5 Hz, 1H), 7.05 (d, J=2.0 Hz, 1H), 5.48 (bs, 1H), 3.64-3.68 (m, 8H), 3.53-3.57 (m, 3H), 3.39-3.42 (m, 3H), 3.38 (s, 3H), 3.33-3.35 (m, 2H), 1.73 (m, 4H), 1.67 (m, 2H);

¹³C NMR (125 MHz, CDCl3) δ 161.2, 152.9, 151.6, 137.2, 133.8, 130.3, 127.2, 126.6, 126.1, 125.7, 119.4, 117.8, 108.6, 100.5, 71.9, 70.6, 70.6, 70.5, 69.4, 59.0, 49.5, 40.2, 25.5, 24.3;

HRMS calc. for C₂₅H₃₃N₃O₅SNa [M+Na]⁻ 510.2033, found 510.2035 by ESI.

### Example 3

### Synthesis of Compound 3

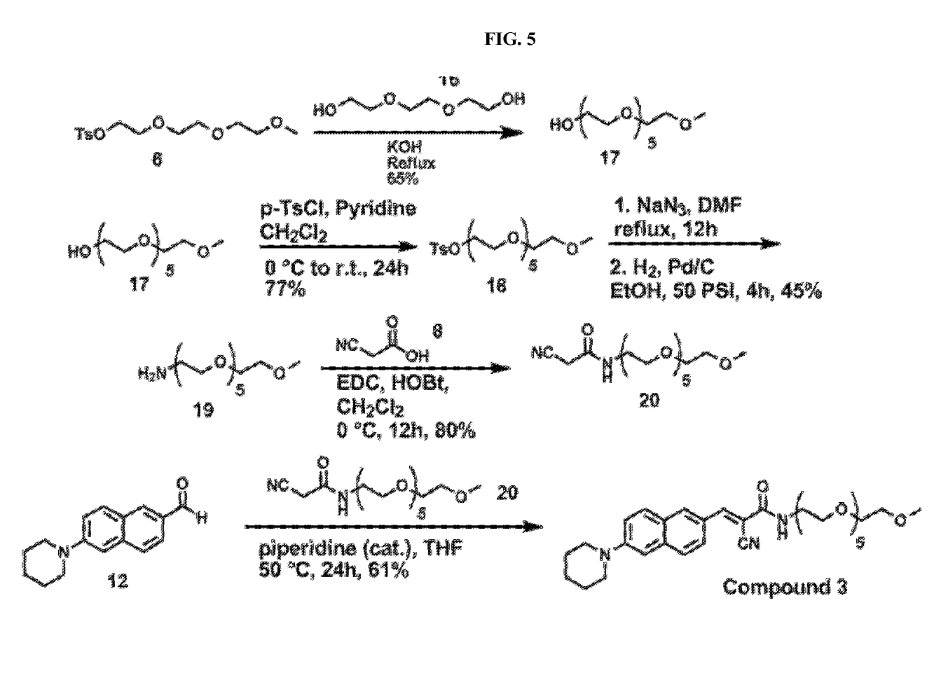

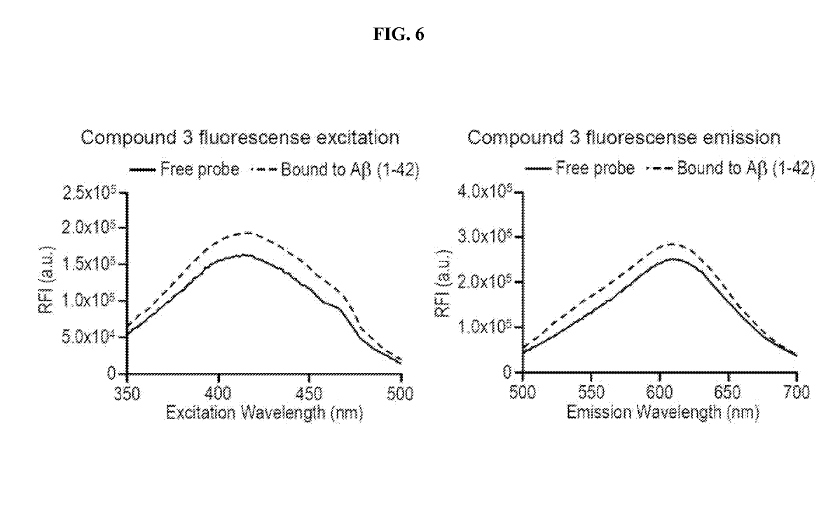

The synthesis of compound 3 is shown in FIG. 5. Synthesis of compound 3 began by coupling tosylate (6) to triethylene glycol (16) under basic conditions to yield hexaethylene glycol monomethyl ether (17) in 65% yield. Hexaethylene glycol monomethyl ether (17) was then converted to the corresponding tosylate (18) in 77% yield, followed by conversion to the azide. The azide was reduced to the corresponding amine (19) via a hydrogenation over activated palladium on carbon in 45% yield. The amine was coupled to cyanoacetic acid using 1-Ethyl-3-(3-dimethylaminopropyl)carbodiimide (EDC) in 80% yield to give the cyano-acetamide (20). The resulting amide (20) was coupled with previously synthesized piperidine-naphthalene (12) to give compound 3 via Knovenagel condensation in 61% yield.

Characterization Data:

R_{f}=0.35 (3% MeOH/EtOAc);

¹H NMR (500 MHz, δ, ppm, CDCl₃): 1.68 (m, 6H); 3.30 (s, 3H); 3.33 (m, 3H); 3.46 (t, 2H); 3.53-3.62 (m, 20H); 6.83 (bs, 1H); 6.99 (d, 2H); 7.22-7.24 (dd, 1H); 7.59 (d, 1H); 7.69 (d, 1H), 7.98 (dd, 1H), 8.10 (s, 1H);

¹³C NMR (125 MHz, δ, ppm, CDCl₃): 136.4, 132.9, 129.5, 126.4, 125.8, 125.2, 124.9, 118.6, 116.9, 107.8, 99.7, 71.0, 69.7, 69.6, 69.5, 68.6, 58.1, 51.9, 48.7, 39.3, 28.8, 24.7, 23.5, 7.2;

HRMS: calc. for C₃₂H₄₅N₃O₇: [M+Na]⁺ 606.3150, found 606.3151 by ESI.

### Example 4

### Synthesis of Compound 5

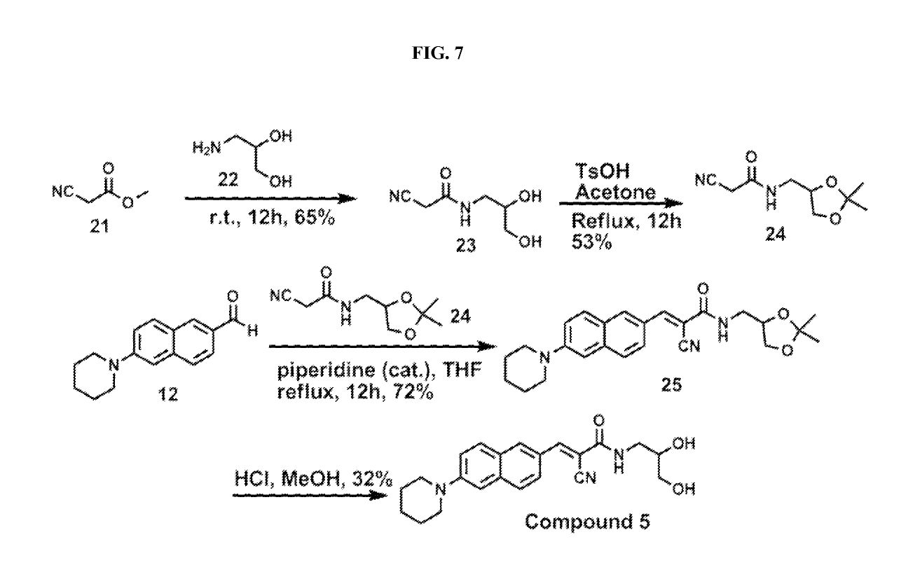

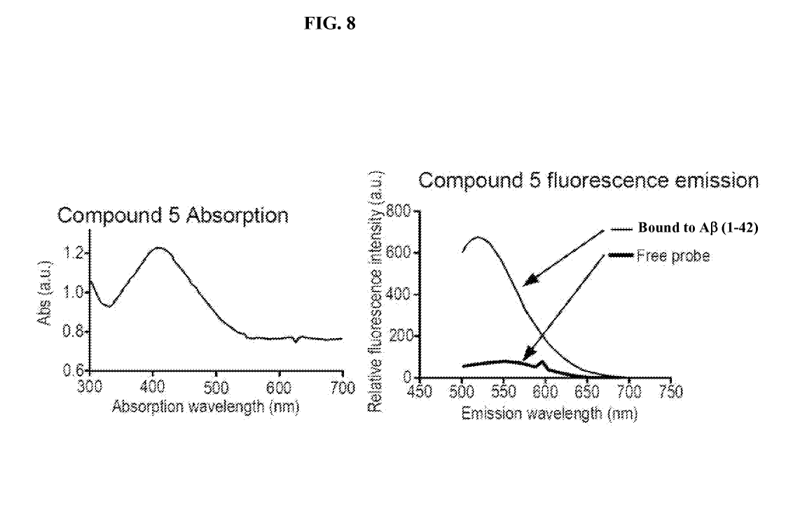

The synthesis of compound 5 is shown in FIG. 7. The synthesis began with commercially available methyl cyanoacetate (21), which was stirred with 3-amino-1,2-propanediol (22) at room temperature to yield the cyanoacetamide propanediol (23) in 65% yield. The propanediol was then reacted with acetone with p-toluenesulfonic acid to obtain the cyanoacetamide acetal (24) in 53% yield. Acetal (24) was then coupled with previously synthesized piperidine-naphthalene-aldehyde (12) via a Knovenagel condensation to yield the protected compound 25 in 72% yield. This protected conjugate was then deprotected under acid to yield the final compound 5 in 32% yield.

### Characterization Data:

¹H NMR (300 MHz, CD₃OD) δ 8.47 (s, 1H), 8.37 (s, 1H), 8.19 (dd, J=1.9, 1.8 Hz, 1H), 7.91 (d, J=9.1 Hz, 1H), 7.83 (d, J=8.6 Hz, 1H), 7.50 (dd, J=2.3, 2.2 Hz, 1H), 7.37 (d, J=2.0 Hz, 1H), 4.38 (d, J=5.3 Hz, 2H), 4.21-4.15 (m, 2H), 3.89-3.83 (m, 1H), 3.58-3.50 (m, 4H), 1.82-1.75 (m, 6H);

HRMS (ESI-TOF-MS): m/z calc. for C₂₂H₂₆N₃O₃⁺: 380.1969; found: 380.1988

### Example 5

### Synthesis of Compound 17

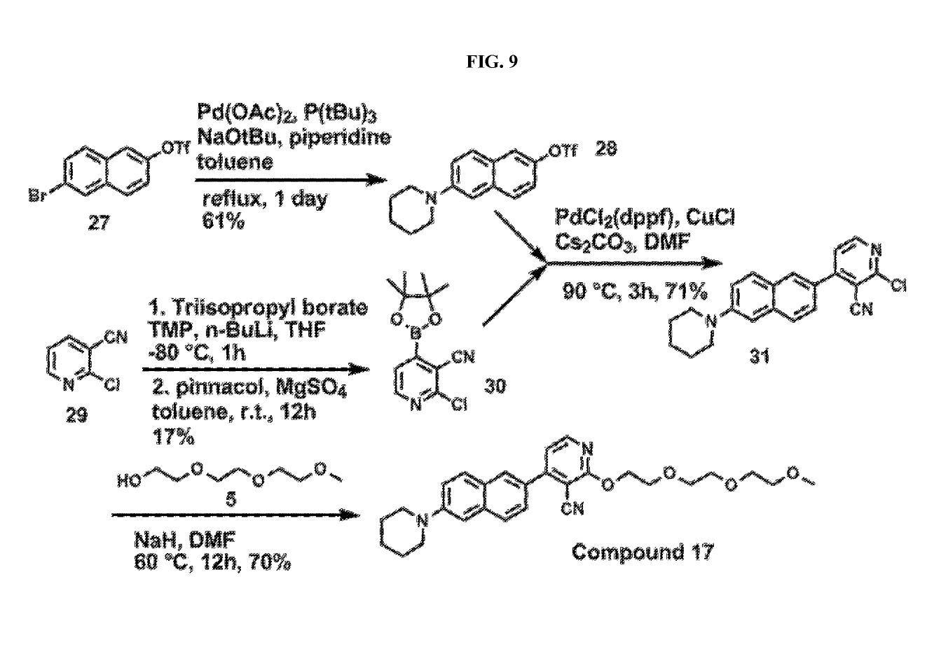

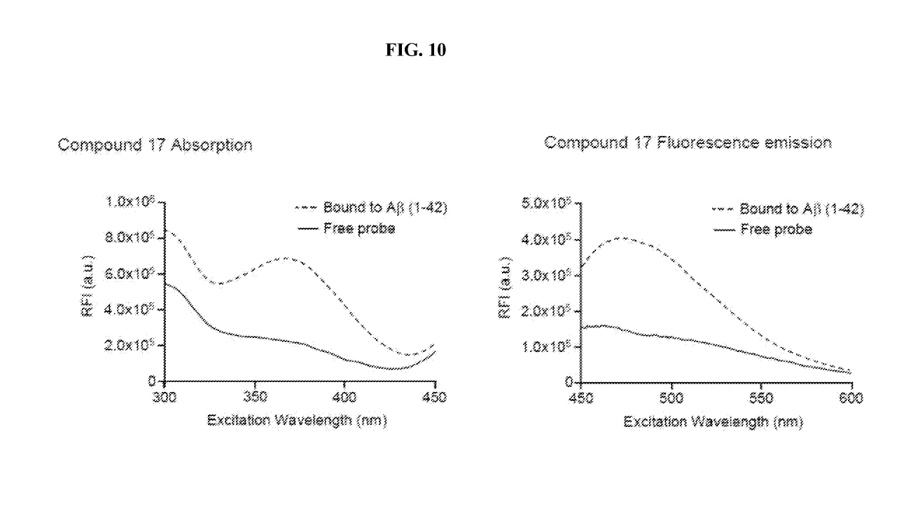

The synthesis of compound 17 is shown in FIG. 9. Synthesis of compound 17 began with commercially available 6-bromo-naphthalen-2-yl triflate (27) which was subjected to palladium-catalyzed Buchwald-Hartwig amination with piperidine to yield the piperidine-naphthalenetriflate (28) in 61% yield. Concomitantly, to 2-chloronicotinonitrile (29) was added the pinnacol boronic ester at the 4-position in a two-step reaction with triisopropyl borate installed first followed by reaction with pinnacol with an overall yield of 17%. The resulting nicotinyl boronate (30) was then coupled to the naphthalene-triflate (28) via a Suzuki cross-coupling to yield precursor (31). The precursor (31) was then reacted with commercially available triethylene glycol monomethyl ether (5) to yield compound 17 in 70% yield.

Characterization Data:

R_{f}=0.40 (5% MeOH/EtOAc);

¹H NMR (500 MHz, CDCl3) δ 8.29-8.30 (d, J=1H), 7.98 (s, 1H), 7.77 (s, 2H), 7.61 (s, 1H), 7.33-7.34 (d, J=Hz, 1H), 7.11-7.12 (d, 2H), 4.63-4.65 (t, 2H), 3.93-3.95 (m, 2H), 3.66-3.70 (m, 4H), 3.54-3.56 (m, 2H), 3.37 (s, 3H), 3.32 (bs, 4H), 1.76 (bs, 4H), 1.64 (bs, 2H);

¹³C (125 MHz, CDCl3) δ 165.16, 155.89, 150.38, 132.71, 129.03, 128.80, 128.51, 128.29, 126.52, 125.23, 123.67, 121.34, 117.61, 115.19, 109.43, 95.41, 72.03, 71.13, 7084, 70.70, 69.29, 67.14, 59.20, 53.07, 32.08, 29.83;

HRMS calc. for C₂₈H₃₃N₃O₄Na [M+Na]⁺ 498.2361, found 498.2363 by ESI.

### Example 9

### Synthesis of Compound 18

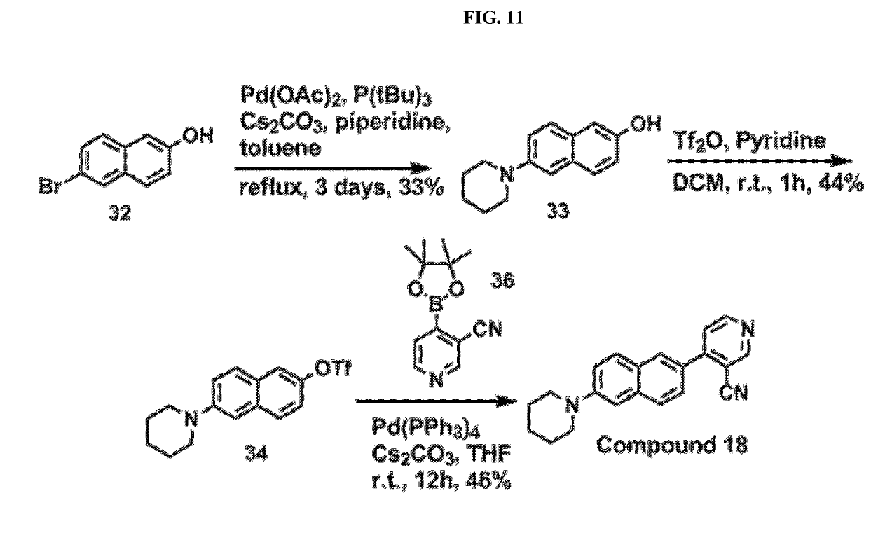

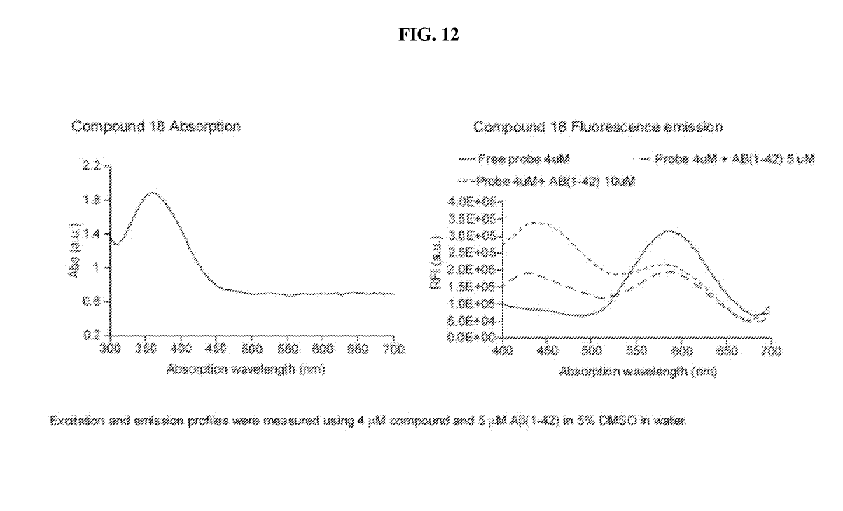

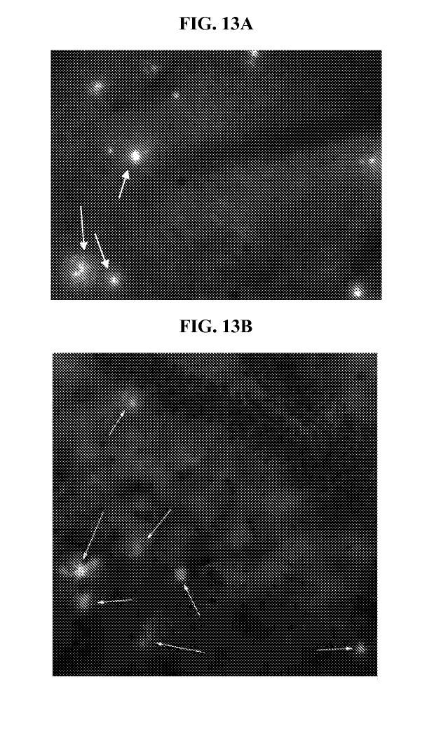

The synthesis of compound 18 is shown in FIG. 11. Synthesis of compound 18 began with commercially available 6-bromo-2-naphthanol 32, which underwent Buchwald-Hartwig amination with piperidine to yield 6-piperidinyl-2-naphthanol (33) in 35% yield. The naphthanol (33) was then protected as a triflate with triflic anhydride to obtain triflate (34) in 44% yield. The triflate was then subjected to palladium-catalyzed Suzuki coupling with boronic acid (36) to obtain compound 18 in 46% yield.

### Characterization Data:

¹H NMR (400 MHz, CDCl₃) δ 8.96 (s, 1H), 8.80 (s, 1H), 8.02 (s, 1H), 7.82 (s, 2H), 7.60 (dd, J=24.4, 6.6 Hz, 2H), 7.37 (s, 1H), 7.14 (s, 1H), 3.35 (s, 4H), 1.78 (s, 4H), 1.66 (s, 2H);

MS (ESI): m/z calc. for C₂₁H₁₉N₃: 313.16; found: 314.41. [M+H]⁺. HRMS (ESI-TOF-MS): m/z calc. for C₂₁H₂₀N₃⁺: 314.1652; found: 314.1664.

### Example 10

### Synthesis of Compound 21

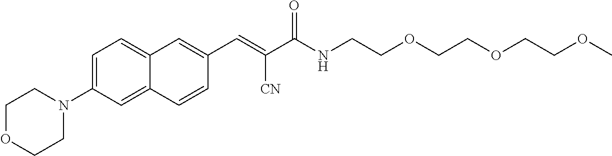 [SMILES: COCCOCCOCCNC(=O)/C(C#N)=C/c1ccc2cc(N3CCOCC3)ccc2c1]

### Step 1: Synthesis of 2-cyano-N-(2-(2-(2-methoxyethoxy)ethoxy)ethyl)acetamide

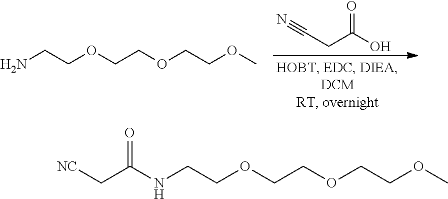 [SMILES: N#CCC(=O)O ; COCCOCCOCCN ; COCCOCCOCCNC(=O)CC#N]

Into a 100-mL 3-necked round-bottom flask, purged and maintained with an inert atmosphere of nitrogen, was placed 2-cyanoacetic acid (0.6 g, 1.00 equiv), 1-[2-(2-aminoethoxy)ethoxy]-2-methoxyethane (1.0 g, 6.13 mmol, 1.00 equiv), dichloromethane (20 mL), DIEA (1.82 g, 14.08 mmol, 2.00 equiv), HOBT (1.43 g, 10.58 mmol, 1.50 equiv), and EDC (2.03 g, 13.08 mmol, 1.50 equiv). The resulting solution was stirred overnight at room temperature. The reaction was then quenched by the addition of 100 mL of water. The resulting mixture was washed with 2×100 mL of brine. The solution was extracted with 2×100 mL of dichloromethane and the organic layers were combined. The mixture was dried over anhydrous sodium sulfate and concentrated under vacuum. The residue was applied onto a silica gel column with ethyl acetate/petroleum ether (1:1). This resulted in 1.2 g (85%) of 2-cyano-N-(2-(2-(2-methoxyethoxy)ethoxy)ethyl)acetamide as light yellow oil.

### Step 2: Synthesis of (6-bromonaphthalen-2-yl)methanol

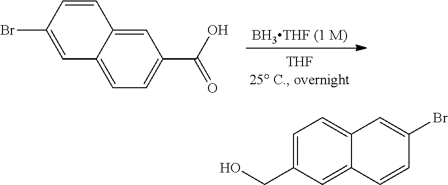 [SMILES: O=C(O)c1ccc2cc(Br)ccc2c1 ; OCc1ccc2cc(Br)ccc2c1]

Into a 250-mL 3-necked round-bottom flask was placed 6-bromo-2-naphthoic acid (10 g, 39.83 mmol, 1.00 equiv), tetrahydrofuran (40 mL). This was followed by the addition of BH₃THF (1 M) (80 mL, 2.00 equiv) dropwise with stirring at 0° C. The resulting solution was stirred overnight at 25° C. The reaction was then quenched by the addition of 100 mL of ice/water. The resulting solution was extracted with 2×200 mL of ethyl acetate and the organic layers were combined. The mixture was dried over anhydrous sodium sulfate and concentrated under vacuum. This resulted in 8 g (85%) of (6-bromonaphthalen-2-yl)methanol as a light yellow solid.

### Step 3: Synthesis of 6-bromo-2-naphthaldehyde

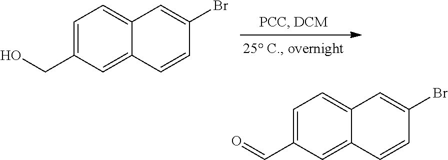 [SMILES: OCc1ccc2cc(Br)ccc2c1 ; O=Cc1ccc2cc(Br)ccc2c1]

Into a 250-mL 3-necked round-bottom flask was placed (6-bromonaphthalen-2-yl)methanol (8 g, 33.74 mmol, 1.00 equiv), dichloromethane (80 mL), and PCC (8.8 g, 40.78 mmol, 1.20 equiv). The resulting solution was stirred overnight at 25° C. The solids were filtered out and the filter cake was washed with EA. The filtrate was concentrated under vacuum. This resulted in 6.5 g (82%) of 6-bromo-2-naphthaldehyde as a light yellow solid.

### Step 4: Synthesis of 2-(6-bromonaphthalen-2-yl)-1,3-dioxolane

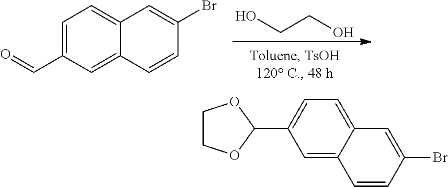

Into a 500-mL 3-necked round-bottom flask was placed 6-bromo-2-naphthaldehyde (6.5 g, 27.65 mmol, 1.00 equiv), toluene (130 mL), and ethane-1,2-diol (26 mL). This was followed by the addition of TsOH (478 mg, 2.79 mmol, 0.10 equiv) with stirring at room temperature. The resulting solution was stirred for 48 h at 120° C. in an oil bath. The mixture was cooled to room temperature. The reaction was then quenched by the addition of water. The resulting solution was extracted with 2×150 mL of ethyl acetate and the organic layers were combined. The mixture was washed with 2×150 mL of brine, dried over anhydrous sodium sulfate and concentrated under vacuum. The residue was applied onto a silica gel column with ethyl acetate/petroleum ether (1:20). This resulted in 4.0 g (52%) of 2-(6-bromonaphthalen-2-yl)-1,3-dioxolane as a white solid.

### Step 5: Synthesis of 4-(6-(1,3-dioxolan-2-yl)naphthalen-2-yl)morpholine

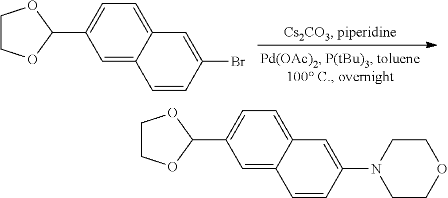

Into a 100-mL 3-necked round-bottom flask, purged and maintained with an inert atmosphere of nitrogen, was placed 2-(6-bromonaphthalen-2-yl)-1,3-dioxolane (900 mg, 3.22 mmol, 1.00 equiv), Cs₂CO₃ (2.1 g, 6.43 mmol, 2.00 equiv), Pd(OAc)₂ (87 mg, 0.39 mmol, 0.12 equiv), morpholine (563 g, 6.46 mol, 2.00 equiv), P(tBu)₃ (2.94 g, 14.55 mmol, 0.45 equiv), and toluene (9 mL). The resulting solution was stirred overnight at 100° C. in an oil bath. The mixture was cooled to room temperature. The resulting mixture was washed with 2×100 mL of water and 2×200 mL of brine. The solution was extracted with 2×80 mL of ethyl acetate and the organic layers were combined. The mixture was dried over anhydrous sodium sulfate and concentrated under vacuum. The residue was applied onto a silica gel column with ethyl acetate/petroleum ether (1:1). This resulted in 900 mg (98%) of 4-(6-(1,3-dioxolan-2-yl)naphthalen-2-yl)morpholine as a brown solid.

### Step 6: Synthesis of 6-morpholino-2-naphthaldehyde

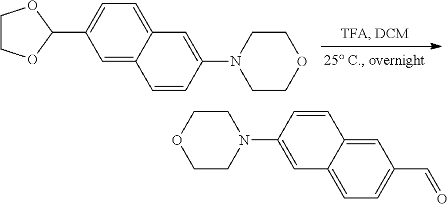

Into a 100-mL round-bottom flask was placed 4-(6-(1,3-dioxolan-2-yl)naphthalen-2-yl)morpholine (900 mg, 3.15 mmol, 1.00 equiv), dichloromethane (9 mL), and trifluoroacetic acid (5.4 g, 47.77 mmol, 15.00 equiv). The resulting solution was stirred overnight at 25° C. The mixture was concentrated under vacuum. The solution was extracted with 5×50 mL of ethyl acetate and the organic layers were combined. The mixture was washed with 2×50 mL of aqueous NaHCO₃, dried over anhydrous sodium sulfate and concentrated under vacuum. This resulted in 0.5 g (66%) of 6-morpholino-2-naphthaldehyde as a light yellow solid.

### Step 7: Synthesis of 2-cyano-N-(2-(2-(2-methoxyethoxy)ethoxy)ethyl)-3-(6-morpholinonaphthalen-2-yl)acrylamide

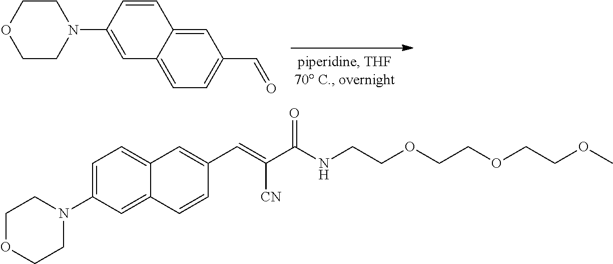

Into a 20-mL sealed tube was placed 6-morpholino-2-naphthaldehyde (300 mg, 1.24 mmol, 1.00 equiv), tetrahydrofuran (3 mL), 2-cyano-N-2-[2-(2-methoxyethoxy)ethoxy]ethylacetamide (247 mg, 1.07 mmol, 1.20 equiv), and piperidine (12 mg, 0.10 equiv). The resulting solution was stirred overnight at 70° C. in an oil bath. The mixture was cooled to room temperature. The resulting mixture was concentrated under vacuum and the crude product was purified by flash-prep-HPLC. The collected fractions were combined and concentrated under vacuum. This resulted in 118.4 mg (21%) of 2-cyano-N-(2-(2-(2-methoxyethoxy)ethoxy)ethyl)-3-(6-morpholinonaphthalen-2-yl)acrylamide as a yellow solid. LC-MS-PH-AMD-2015-005-1-0: (ES, m/z): [M+H]⁺:454. H-NMR-PH-AMD-2015-005-1-0: (300 MHz, DMSO-d₆, ppm): δ 8.37 (s, 1H), 8.35 (s, 1H), 8.33 (s, 1H), 8.06-8.03 (m, 1H), 7.89-7.67 (m, 2H), 7.50-7.46 (d, 1H), 7.25 (s, 1H), 3.81-3.77 (m, 4H), 3.54-3.51 (m, 8H), 3.44-3.39 (m, 4H), 3.39-3.34 (m, 4H), 3.22 (s, 3H).

### Example 11

### Synthesis of Compound 22

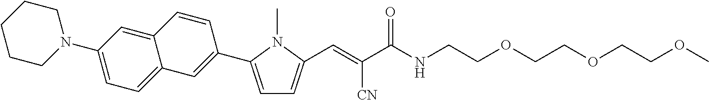 [SMILES: COCCOCCOCCNC(=O)/C(C#N)=C/c1ccc(-c2ccc3cc(N4CCCCC4)ccc3c2)n1C]

### Step 1: Synthesis of (2-((6-bromonaphthalen-2-yloxy)methoxy)ethyl)trimethylsilane

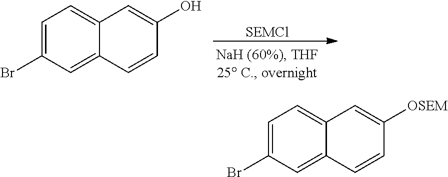

Into a 250-mL 3-necked round-bottom flask was placed 6-bromonaphthalen-2-ol (5.0 g, 22.41 mmol, 1.00 equiv) and tetrahydrofuran (50 mL). This was followed by the addition of sodium hydride (60%) (1.35 g, 33.6 mmol, 1.50 equiv) in several batches with stirring at 0° C. To this was added [2-(chloromethoxy)ethyl]trimethylsilane (5.6 g, 33.59 mmol, 1.50 equiv) with stirring. The resulting solution was stirred overnight at 25° C. The reaction was then quenched by the addition of 100 mL of water. The resulting solution was extracted with 2×100 mL of ethyl acetate and the organic layers were combined. The mixture was dried over anhydrous sodium sulfate and concentrated under vacuum. The residue was applied onto a silica gel column with ethyl acetate/petroleum ether (1:20). This resulted in 8.8 g (crude) of (2-((6-bromonaphthalen-2-yloxy)methoxy)ethyl)trimethylsilane as a white solid.

### Step 2: Synthesis of 1-(6-((2-(trimethylsilyl)ethoxy)methoxy)naphthalen-2-yl)piperidine

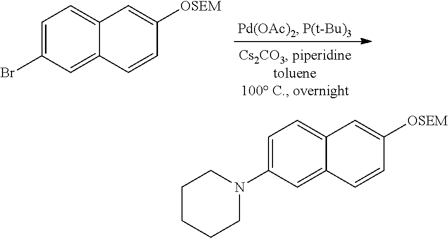

Into a 250-mL 3-necked round-bottom flask, purged and maintained with an inert atmosphere of nitrogen, was placed (2-((6-bromonaphthalen-2-yloxy)methoxy)ethyl)trimethylsilane (8.8 g, 24.91 mmol, 1.00 equiv), Pd(OAc)₂ (672 mg, 2.99 mmol, 0.12 equiv), P(t-Bu)₃ (22.7 g, 112.38 mmol, 0.45 equiv), Cs₂CO₃ (16.3 g, 50.03 mmol, 2.00 equiv), piperidine (4.3 g, 2.00 equiv), and toluene (44 mL). The resulting solution was stirred overnight at 100° C. The mixture was concentrated under vacuum and the residue was applied onto a silica gel column with ethyl acetate/petroleum ether (1:20). This resulted in 5.5 g (62%) of 1-(6-((2-(trimethylsilyl)ethoxy)methoxy)naphthalen-2-yl)piperidine as a light yellow solid. Chemical Formula: C₂₁H₃₁NO₂Si; LCMS: 358.2

### Step 3: Synthesis of 6-(piperidin-1-yl)naphthalen-2-ol

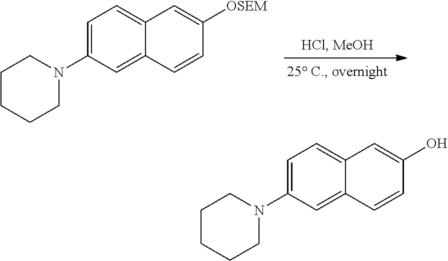

Into a 250-mL round-bottom flask was placed 1-(6-((2-(trimethylsilyl)ethoxy)methoxy)naphthalen-2-yl)piperidine (5.5 g, 15.38 mmol, 1.00 equiv), methanol (55 mL), and hydrogen chloride (20 mL). The resulting solution was stirred overnight at 25° C. The mixture was washed with 2×100 mL of water. The resulting solution was extracted with 2×100 mL of ethyl acetate and the organic layers were combined. The mixture was dried over anhydrous sodium sulfate and concentrated under vacuum. This resulted in 3.2 g (92%) of 6-(piperidin-1-yl)naphthalen-2-ol as a yellow solid. Chemical Formula: C₁₅H₁₇NO; LCMS: 228.3

### Step 4: Synthesis of 6-(piperidin-1-yl)naphthalen-2-yl trifluoromethanesulfonate

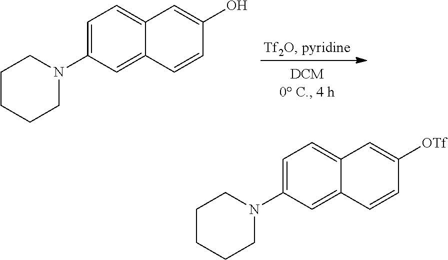 [SMILES: Oc1ccc2cc(N3CCCCC3)ccc2c1 ; O=S(=O)(Oc1ccc2cc(N3CCCCC3)ccc2c1)C(F)(F)F]

Into a 500-mL 3-necked round-bottom flask, purged and maintained with an inert atmosphere of nitrogen, was placed 6-(piperidin-1-yl)naphthalen-2-ol (2.6 g, 11.44 mmol, 1.00 equiv), pyridine (4.5 g, 56.89 mmol, 5.00 equiv), and dichloromethane (182 mL). This was followed by the addition of Tf₂O (38.2 g, 135.39 mmol, 12.00 equiv) dropwise with stirring at 0° C. The resulting solution was stirred for 4 h at 0° C. in a water/ice bath. The reaction was then quenched by the addition of 100 mL of water/ice. The resulting solution was extracted with 2×100 mL of dichloromethane and the organic layers were combined. The mixture was dried over anhydrous sodium sulfate and concentrated under vacuum. The residue was applied onto a silica gel column with ethyl acetate/petroleum ether (1:10). This resulted in 2.2 g (54%) of 6-(piperidin-1-yl)naphthalen-2-yl trifluoromethanesulfonate as a light yellow solid.

### Step 5: Synthesis of 1-(6-(4,4,5,5-tetramethyl-1,3,2-dioxaborolan-2-yl)naphthalen-2-yl)piperidine

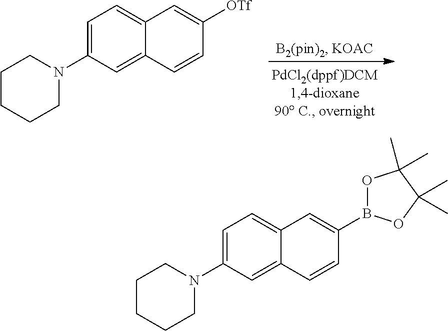 [SMILES: O=S(=O)(Oc1ccc2cc(N3CCCCC3)ccc2c1)C(F)(F)F ; CC1(C)OB(c2ccc3cc(N4CCCCC4)ccc3c2)OC1(C)C]

Into a 100-mL 3-necked round-bottom flask, purged and maintained with an inert atmosphere of nitrogen, was placed 6-(piperidin-1-yl)naphthalen-2-yl trifluoromethanesulfonate (500 mg, 1.39 mmol, 1.00 equiv), B₂(pin)₂ (420 mg, 1.65 mmol, 1.20 equiv), PdCl₂(dppf)DCM (120 mg, 0.15 mmol, 0.10 equiv), KOAc (410 mg, 4.18 mmol, 3.00 equiv), and 1,4-dioxane (5 mL). The resulting solution was stirred overnight at 90° C. in an oil bath. The mixture was concentrated under vacuum and the residue was applied onto a silica gel column with ethyl acetate/petroleum ether (1:10). This resulted in 0.6 g (crude) of 1-(6-(4,4,5,5-tetramethyl-1,3,2-dioxaborolan-2-yl)naphthalen-2-yl)piperidine as a light yellow solid. Chemical Formula: C₂₁H₂₈BNO₂; LCMS: 338.2.

### Step 6: Synthesis of 5-bromo-1-methyl-1H-pyrrole-2-carbaldehyde

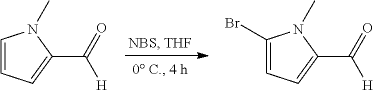

Into a 100-mL 3-necked round-bottom flask was placed 1-methyl-1H-pyrrole-2-carbaldehyde (1 g, 9.16 mmol, 1.00 equiv) in tetrahydrofuran (10 mL). This was followed by the addition of NBS (1.64 g, 9.21 mmol, 1.10 equiv) in several batches with stirring at 0° C. The resulting solution was stirred for 4 h at 0° C. The mixture was washed with 1×100 mL of water. The solution was extracted with 2×80 mL of ethyl acetate and the organic layers were combined. The mixture was dried over anhydrous sodium sulfate and concentrated under vacuum. The residue was applied onto a silica gel column with ethyl acetate/petroleum ether (1:10). This resulted in 0.3 g (17%) of 5-bromo-1-methyl-1H-pyrrole-2-carbaldehyde as a white solid. Chemical Formula: C₆H₆BrNO; LCMS: 188.

### Step 7: Synthesis of 1-methyl-5-(6-(piperidin-1-yl)naphthalen-2-yl)-1H-pyrrole-2-carbaldehyde

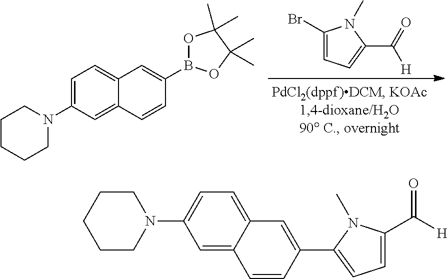

Into a 40-mL vial, purged and maintained with an inert atmosphere of nitrogen, was placed 1-(6-(4,4,5,5-tetramethyl-1,3,2-dioxaborolan-2-yl)naphthalen-2-yl)piperidine (150 mg, 0.44 mmol, 1.00 equiv), PdCl₂(dppf)DCM (36 mg, 0.10 equiv), potassium carbonate (123 mg, 0.89 mmol, 2.00 equiv), 1,4-dioxane/H₂O (5.5 mL), and 5-bromo-1-methyl-1H-pyrrole-2-carbaldehyde (100 mg, 0.53 mmol, 1.20 equiv). The resulting solution was stirred overnight at 90° C. in an oil bath. The mixture was washed with 1×50 mL of water. The solution was extracted with 2×50 mL of ethyl acetate and the organic layers were combined. The mixture was dried over anhydrous sodium sulfate and concentrated under vacuum. The residue was applied onto a silica gel column with ethyl acetate/petroleum ether (1:4). This resulted in 0.16 g (crude) of 1-methyl-5-(6-(piperidin-1-yl)naphthalen-2-yl)-1H-pyrrole-2-carbaldehyde as a yellow solid. Chemical Formula: C21H22N2O; LCMS: 319.3.

### Step 8: Synthesis of 2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide

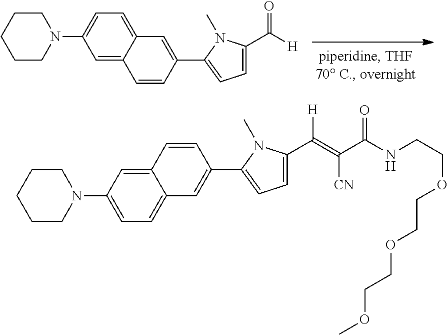 [SMILES: Cn1c(C=O)ccc1-c1ccc2cc(N3CCCCC3)ccc2c1 ; COCCOCCOCCNC(=O)/C(C#N)=C/c1ccc(-c2ccc3cc(N4CCCCC4)ccc3c2)n1C]

Into a 40-mL round-bottom flask, purged and maintained with an inert atmosphere of nitrogen, was placed 1-methyl-5-[6-(piperidin-1-yl)naphthalen-2-yl]-1H-pyrrole-2-carbaldehyde (160 mg, 0.50 mmol, 1.00 equiv), tetrahydrofuran (2 mL), piperidine (4.3 mg, 0.05 mmol, 0.10 equiv), and 2-cyano-N-2-[2-(2-methoxyethoxy)ethoxy]ethylacetamide (128 mg, 0.56 mmol, 1.10 equiv). The resulting solution was stirred overnight at 70° C. in an oil bath. The resulting mixture was concentrated under vacuum. The crude product was purified by flash-prep-HPLC. The collected fractions were combined and concentrated under vacuum. This resulted in 131.8 mg (49%) of 2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide as a yellow solid. LC-MS-PH-AMD-2015-005-3-0: (ES, m/z): [M+H]⁺: 531. H-NMR-PH-AMD-2015-005-3-0: (300 MHz, DMSO-d₆, ppm): δ 8.22-8.18 (m, 1H), 8.10 (s, 1H), 7.89 (s, 1H), 7.84-7.79 (m, 2H), 7.55-7.49 (m, 2H), 7.44-7.40 (m, 1H), 6.63-6.61 (d, J=4.5, 1H), 7.21 (s, 1H), 3.80 (s, 3H), 3.54-3.50 (m, 8H), 3.45-3.41 (m, 4H), 3.39-3.32 (m, 4H), 3.23 (s, 3H), 1.67-1.59 (m, 6H).

### Example 12

### Determination of Protein-Specific Emission Spectra for Known Amyloid Proteins

Amyloid complexes of each of a-1-antitrypsin, Alpha-synuclein, Amylin (IAAP), Apolipoprotein A1 and fragments, Atrial natriuretic factor, Beta microglobulin, beta-amyloid, Calcitonin, Curli (CsgA-R1), Curli (CsgA-R5), Cystatin, Gelsolin, Huntingtin, Huntingtin, Immunoglobulin light chain AL, insulin, Keratoepithelin, Lysozyme, Medin, p53, PAP85-120, prion proteins or fragments (PrPSc), Prolactin, Protegrin-1, SEM1 49-107, Semen derived enhancer of viral infection, serum amyloid A, S-IBM, superoxide dismutase 1, Transthyretin, and vasopressin receptor 2 are obtained and contacted with a compound as disclosed herein. An emission spectrum is determined for each amyloid complex.

### Example 13

### Amyloid Determination in a Sample

An unknown sample is obtained from an individual. A composition as disclosed herein is generated, and an emission spectrum is determined. The emission spectrum is compared with emission spectra as determined in Example 12, above. The amyloid complex in the unknown sample is hypothesized to comprise of the amyloid protein having a corresponding emission spectrum.

### Example 14

### Early Diagnosis of Pre-Eclampsia

A urine sample is obtained from a pregnant woman showing no signs or symptoms of pre-eclampsia. The sample is combined with a compound as described herein to form a composition as described herein. The composition is assayed for formation of amyloid/compound complexes using spectrophotometric methods.

Amyloid/compound complex levels are not detected above background. It is concluded that the individual is not expected to develop symptoms of pre-eclampsia.

### Example 15

### Side-by Side Comparison

A first aliquot of a urine sample is obtained from a pregnant woman showing no signs or symptoms of pre-eclampsia. The sample is combined with a compound as described herein to form a composition as described herein. The composition is assayed for formation of amyloid/compound complexes using spectrophotometric methods.

A second aliquot of the urine sample is obtained from the pregnant woman showing no signs or symptoms of pre-eclampsia. The sample is combined with Congo Red to form a detection composition. The composition is assayed for formation of amyloid/compound complexes using spectrophotometric methods

Amyloid/compound complex levels are not detected above background using Congo Red. Amyloid levels are detected above background using the compositions as disclosed herein, indicating an improvement of the compositions as disclosed herein over comparable compositions for which Congo Red is substituted.

It is concluded that the individual is expected to develop symptoms of pre-eclampsia.

### Example 16

### Tests of Selected Detection Moleculs on Patient Populations

Molecules were tested for their ability to detect misfolded protein aggregates in patient samples.

### Patient Population

Urine specimens were obtained from 29-31 patients. Included in the sample population were 16-17 healthy pregnant women, 1 male, 4-5 non-pregnant females, and 8 pregnant women diagnosed with pre-eclampsia (PE). The range in patient population is due to limited sample volume. Control samples are defined as individuals without PE. Patient samples are defined as pregnant women diagnosed with PE. The number of controls and patients were: compound 1: 23 controls and 8 patients, compound 2: 21 controls and 8 patients, compound 5: 21 controls and 8 patients, compound 21: 18 controls and 7 patients, compound 22: 16 controls and 5 patients. Urine specimens were aliquoted and stored at −80° C.

Evaluation of Urine Specimens with AMDX Probes

Binding of certain proteins in PE urine to AMDX probes results in an enhanced fluorescence signal. Urine (37.6 μL) was diluted into 1× PBS, pH 7.4 such that a 25% urine solution in PBS was obtained. Samples were analyzed on a Shimadzu 5301PC fluorimeter using a quartz cuvette. An emission scan was obtained for each urine specimen with and without AMDX probe using an excitation, spectral sweep and a slit width defined in Table 1. AMDX probe in DMSO was added to the 25% urine solution to obtain a final concentration specified in Table 1 and re-analyzed. A blank emission spectra of DMSO in 1× PBS, pH 7.4 was subtracted from the final urine/AMDX emission spectra. Using the parameters in Table 1, an emission scan of AMDX probe in 1× PBS, pH 7.4 was also obtained. Normalized fluorescence intensity (NFI) was calculated by taking the relative fluorescence intensity at a specific emission wavelength λ (RFI, Table 1) of the urine with AMDX probe minus the RFI_{λ} of urine without AMDX probe all divided by the RFI_{λ} of AMDX probe (see equation below).

$${NFI}_{λ} = \frac{( ( {RFI}_{λ} urine + AMDX probe ) - {RFI}_{λ} urine )}{{RFI}_{λ} AMDX probe}$$

Receiver operating characteristics (ROC) was applied using NFL_{λ}. Cutoffs were set as optimal when the sum of sensitivity and specificity was maximal. Excitaiton and emission spectra are as given in Table 1, above.

Analysis of AMDX probes with urine specimens obtained from non-PE individuals (controls) or pregnant women that were diagnosed with PE (patients) results are shown in Table 2.

TABLE 2
Sensitivity and specificity data.

| AMDX | | | Area under the |
|---|---|---|---|
| Probe | FIG. | Sensitivity/Specificity | curve |
| Compound 1 | 14A and | Cutoff of NFI₅₃₄ nm >5.631 | 0.9837 |
| | 14B | sensitivity of 100% | |
| | | specificity of 86.96% | |
| Compound 2 | 15A and | Cutoff of NFI_{535 nm} >18.41 | 0.8750 |
| | 15B | sensitivity of 75% | |
| | | specificity of 95.24% | |
| Compound 5 | 16A and | Cutoff of NFT_{540 nm} >2.46 | 0.9524 |
| | 16B | sensitivity of 100% | |
| | | specificity of 85.71% | |
| Compound | 17A and | A cutoff of NFI_{563 nm} >1.735 | 0.7460 |
| 21 | 17B | sensitivity of 85.71% | |
| | | specificity of 66.67% | |
| Compound | 18A and | A cutoff of NFI_{554 nm} >21.67 | 0.8625 |
| 22 | 18B | sensitivity of 100% | |
| | | specificity of 75% | |

One concludes that each of compounds 1, 2, 3, 4, and 5 is effective at detecting protein aggregates in urine samples with a high specificity and high sensitivity.

### Example 17

### Side-by Side Comparison

A first aliquot of a urine sample is obtained from a pregnant woman showing no signs or symptoms of pre-eclampsia. The sample is combined with a compound as described herein to form a composition as described herein. The composition is assayed for formation of amyloid/compound complexes using spectrophotometric methods, and a 30× increase in spectral signal is observed.

A second aliquot of the urine sample is obtained from the pregnant woman showing no signs or symptoms of pre-eclampsia. The sample is combined with ThT to form a detection composition. The composition is assayed for formation of amyloid/compound complexes using spectrophotometric methods, and a 3× increase in spectral signal is observed.

It is concluded that the individual is expected to develop symptoms of pre-eclampsia, and that the molecule tested out-performs ThT as a detection molecule.

What is claimed is:

1. A composition comprising a) a human sample selected from the list of human samples consisting of a tissue, a cell population, a cell, a fluid, and an extract and b) a compound of Formula I, or a pharmaceutically acceptable salt thereof: 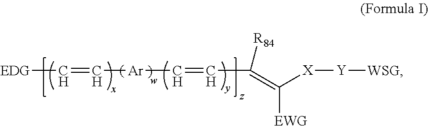
wherein
EDG is an electron donating group;
each Ar is independently C₁-C₁₄ arylene or C₁-C₁₄ heteroarylene, each optionally substituted with one more R₁;
each R₁ is independently halogen, —OR₂, —NR₃R₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R_{5;}
R₂, R₃ and R₄ are independently hydrogen, C₁-C₁₀ alkyl, C₁-C₁₀ heteroalkyl, C₁-C₁₀ cycloalkyl, C₁-C₁₀ heterocycloalkyl, arylene, or C₁-C₁₀ heteroarylene, each of which except for hydrogen is optionally substituted with one or more R₅;
each R₅ is independently halogen, —OR₆, —NR₇R₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
R₆, R₇, R₈ and R₈₄ are independently hydrogen or C₁-C₁₀ alkyl;
EWG is an electron withdrawing group;
WSG is a water soluble group;
X is C═O or SO₂;
Y is NH, or S;
each w is independently an integer from 1-5;
each x is independently an integer from 0-10; each y is independently an integer from 0-10; and
z is an integer from 1-10.

2. The composition of claim 1, wherein the compound of Formula I is 2-cyano-N-(2-(2-(2-methoxyethoxy)ethoxy)ethyl)-3-(6-(piperidin-1-yl)naphthalen-2-yl)acrylamide, 1-cyano-N-(2-(2-(2-methoxyethoxy)ethoxy)ethyl)-2-(6-(piperidin-1-yl)naphthalen-2-yl)ethenesulfonamide, 2-cyano-N-(2,3-dihydroxypropyl)-3-(6-(piperidin-1-yl)naphthalen-2-yl)acrylamide, 2-cyano-N-(2-(2-(2-methoxyethoxy)ethoxy)ethyl)-3-(6-morpholinonaphthalen-2-yl)acrylamide, 2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide, or a pharmaceutically acceptable salt thereof.

3. The composition of claim 1, wherein the compound of Formula I is 2-cyano-N-(2-(2-(2-methoxyethoxy)ethoxy)ethyl)-3-(6-(piperidin-1-yl)naphthalen-2-yl)acrylamide, or a pharmaceutically acceptable salt thereof.

4. The composition of claim 1, wherein the compound of Formula I is 1-cyano-N-(2-(2-(2-methoxyethoxy)ethoxy)ethyl)-2-(6-(piperidin-1-yl)naphthalen-2-yl)ethenesulfonamide, or a pharmaceutically acceptable salt thereof.

5. The composition of claim 1, wherein the compound of Formula I is 2-cyano-N-(2,3-dihydroxypropyl)-3-(6-(piperidin-1-yl)naphthalen-2-yl)acrylamide, or a pharmaceutically acceptable salt thereof.

6. The composition of claim 1, wherein the compound of Formula I is 2-cyano-N-(2-(2-(2-methoxyethoxy)ethoxy)ethyl)-3-(6-morpholinonaphthalen-2-yl)acrylamide, or a pharmaceutically acceptable salt thereof.

7. The composition of claim 1, wherein the compound of Formula I is 2-cyano-N-(2-(2-(2-methoxyethoxy)ethoxy)ethyl)-3-(1-methyl-5-(6-(piperidin-1-yl)naphthalen-2-yl)-1H-pyrrol-2-yl)acrylamide.

8. The composition of claim 1, wherein said sample comprises a cell.

9. The composition of claim 8, wherein said sample comprises a cell selected from the list consisting of a leukocyte, a monocyte, a peripheral blood leukocyte (PBL), a white blood cell, a red blood cell, a skin cell, cheek cell, a hair follicle cell, and a nerve cell.

10. The composition of claim 1, wherein said sample comprises a fluid.

11. The composition of claim 10, wherein said fluid is selected from the list of fluids consisting of as urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, and gastric acid.

12. The composition of claim 10, wherein said fluid is blood.

13. The composition of claim 10, wherein said fluid is saliva.

14. The composition of claim 10, wherein said fluid is urine.

15. The composition of claim 10, wherein said fluid is perspiration.

16. The composition of claim 10, wherein said fluid is tear fluid.

17. The composition of claim 10, wherein said fluid is lymph.

18. The composition of claim 10, wherein said fluid is vaginal fluid.

19. The composition of claim 10, wherein said fluid is semen.

20. The composition of claim 8, wherein said sample comprises a tumor sample.

21. The composition of claim 20, wherein said tumor sample is selected from the list consisting of a tumor tissue, a tumor cell, a tumor fluid, a partially homogenized tumor extract, and a fully homogenized tumor extract.

22. The composition of claim 20, wherein said tumor sample is a sample of a tumor associated with defective p53 protein activity.

23. The composition of any one of claims 1-22, wherein
EDG is —OR₉, —NR₁₀R₁₁, —SR₁₂, —PR₁₃R₁₄, —NR₁₅C(O)R₁₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₁₇;
each R₁₇ is independently halogen, —OR₁₈, —NR₁₉R₂₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
each of R₉, R₁₀, R₁₁, R₁₂, R₁₃, R₁₄, R₁₅, R₁₆, R₁₈, R₁₉ and R₂₀ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₂₁ and wherein R₁₀ and R₁₁ are optionally joined together to form a heterocycloalkyl or heteroaryl optionally substituted with R₂₁;
each of R₂₁ is independently halogen, —OR₂₂, —NR₂₃R₂₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₂₅;
each of R₂₂, R₂₃ and R₂₄ is independently hydrogen or C₁-C₁₀ alkyl; and
each R₂₅ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

24. The composition of any one of claims 1-22, wherein EDG is selected from a group consisting of

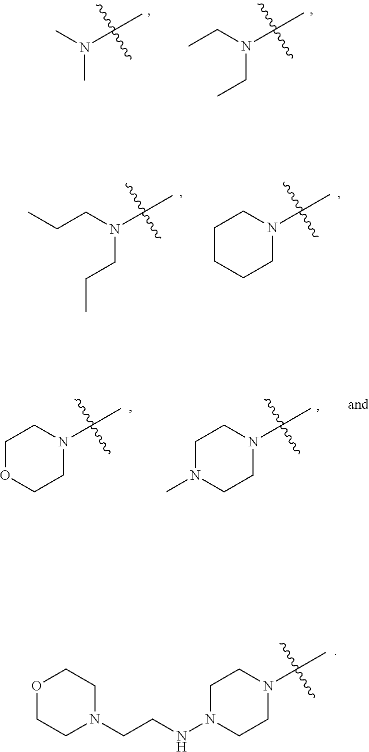

25. The composition of any one of claims 1-22, wherein EDG is

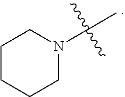 [SMILES: *N1CCCCC1]

26. The composition of any one of claims 1-22, wherein:
EWG is halogen, —CN, —NO₂, —SO₃H, —CR₂₆R₂₇R₂₈, —COR₂₉, or —COOR₃₀;
each R₂₆, R₂₇ and R₂₈ is independently hydrogen or halogen;
R₂₉ is halogen, hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₁;
R₃₀ is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₂; and
each R₃₁ and R₃₂ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

27. The composition of any one of claims 1-22, wherein EWG is selected from a group consisting of —F, —Cl, —Br, —C═O, NO₂, —CF₃, —CCl₃, —SO₃ and —CN.

28. The composition of any one of claims 1-22, wherein EWG is —CN.

29. The composition of any one of claims 1-22, wherein:
WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₃;
each R₃₃ is independently halogen, —OR₃₄, —NR₃₅R₃₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇;
each R₃₄, R₃₅ and R₃₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₃₇;
each R₃₇ is independently halogen, —OR₃₈, —NR₃₉R₄₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and
each of R₃₈, R₃₉ and R₄₀ is independently hydrogen or C₁-C₁₀ alkyl.

30. The composition of any one of claim 1, wherein WSG is

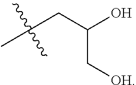 [SMILES: *CC(O)CO]

31. The composition of any one of claim 1, wherein WSG comprises polyethylene glycol, polypropylene glycol, co-polymer of polyethylene glycol and polypropylene glycol, or alkoxy derivatives thereof.

32. The composition of any one of claim 1, wherein WSG is 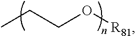
wherein n is an integer from 1-50 and R₈₁ is hydrogen, a C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

33. The composition of claim 32, wherein R₈₁ is methyl.

34. The composition of claim 32, wherein R₈₁ is CH₂—C≡CH.

35. The composition of claim 32, wherein n is 1, 2, 3, 4, 5, 6, 7, 8, 9, or 10.

36. The composition of claim 32, wherein n is 3 or 6.

37. The composition of claim 29, wherein the WSG is

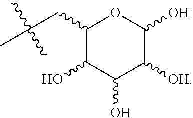 [SMILES: *CC1OC(O)C(O)C(O)C1O]

38. The composition of claim 37, wherein the WSG is

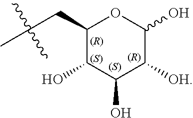

39. The composition of claim 29, wherein the WSG is

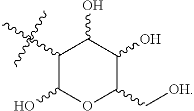

40. The composition of claim 39, wherein the WSG is

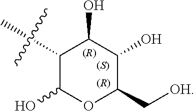

41. The composition of any one of claims 1-22, wherein R₈₄ is hydrogen.

42. The composition of any one of claims 1-22, wherein R₈₄ is methyl.

43. The composition of any one of claims 1-22, wherein the compound is selected from a group consisting of 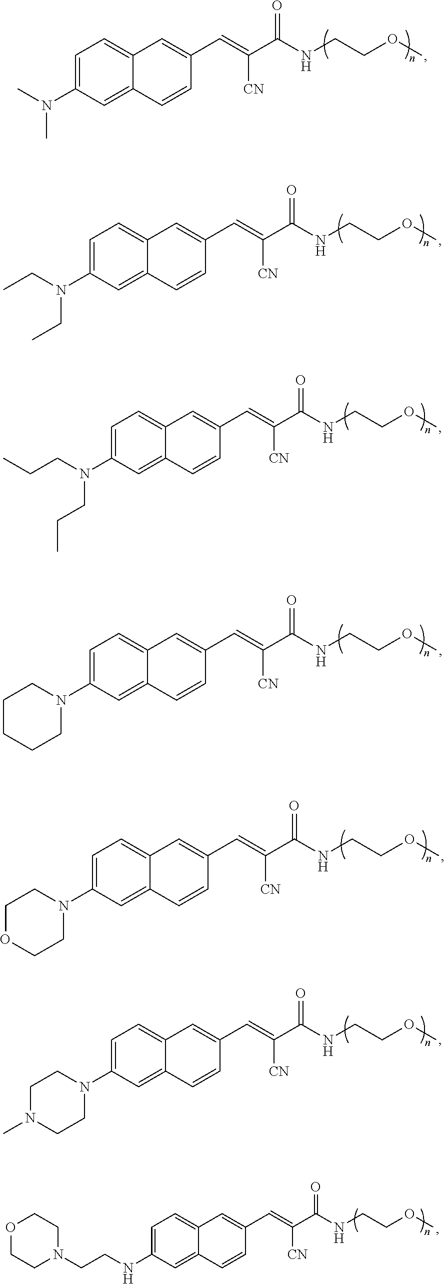 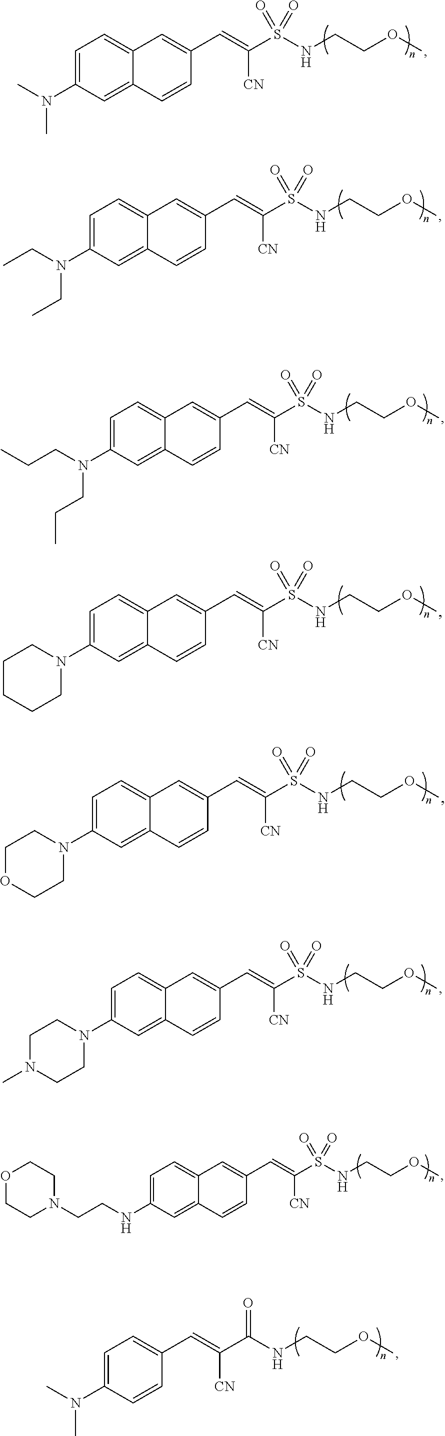 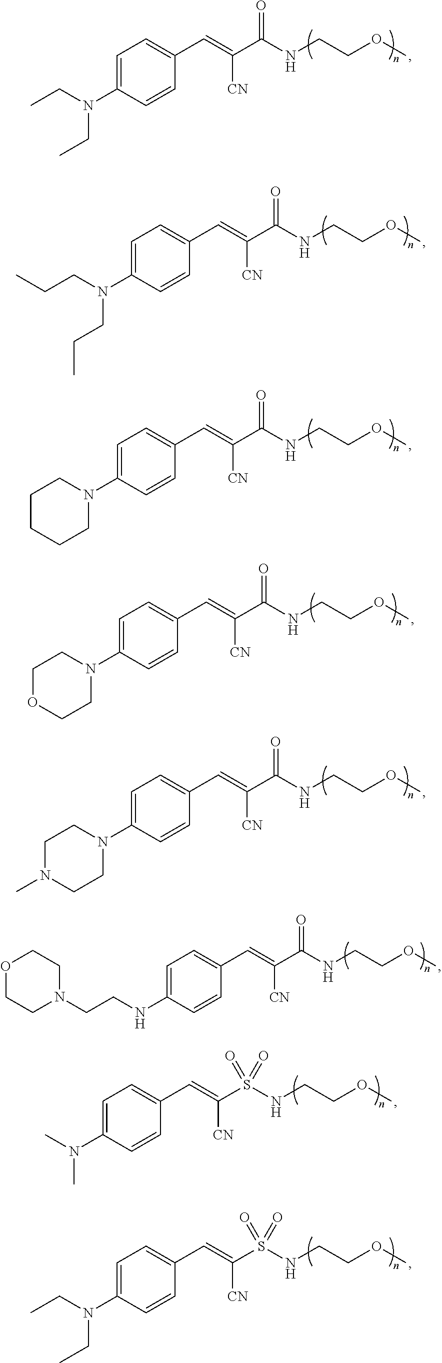 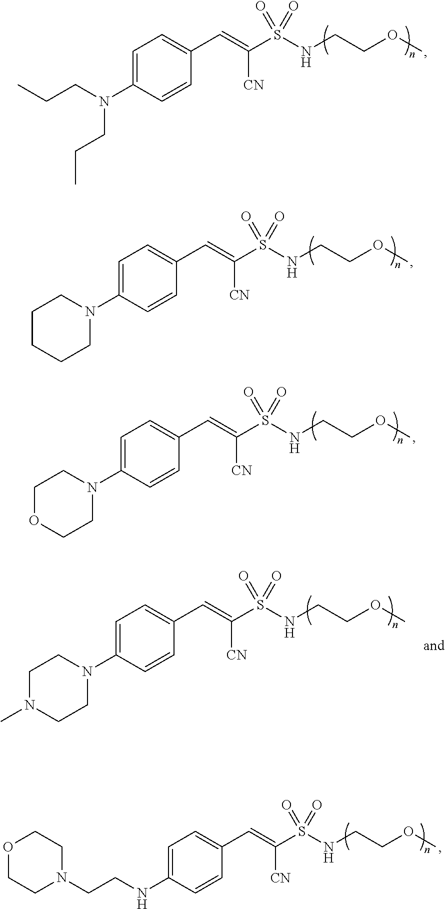
wherein n is an integer with value 1-10.

44. The composition of any one of claims 1-22, wherein the compound is selected from a group consisting of

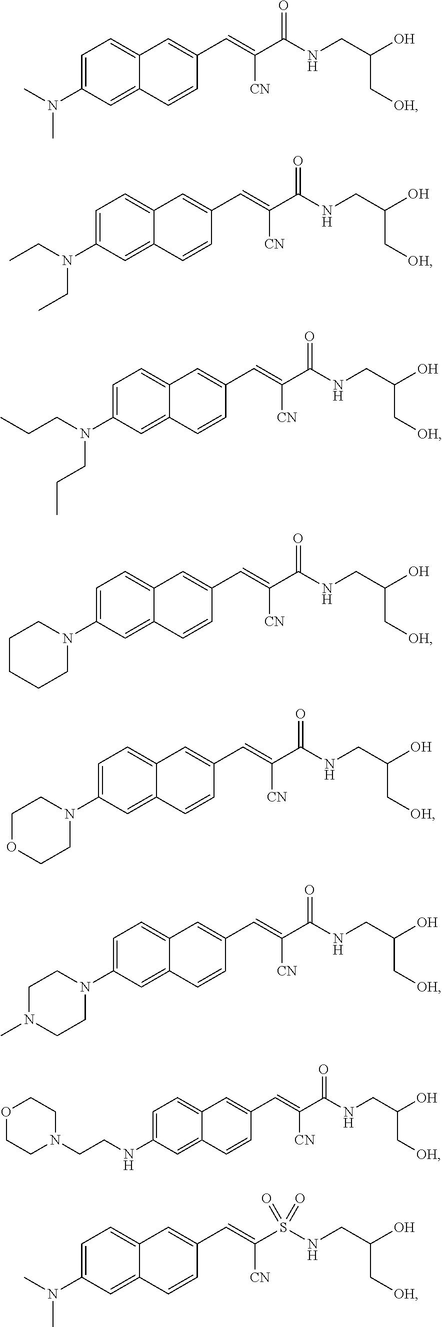

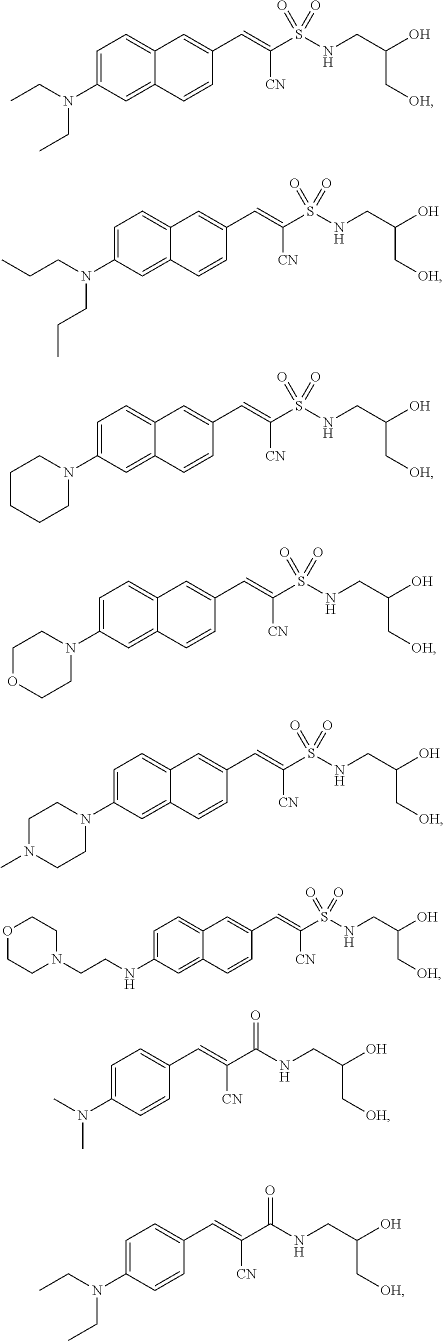

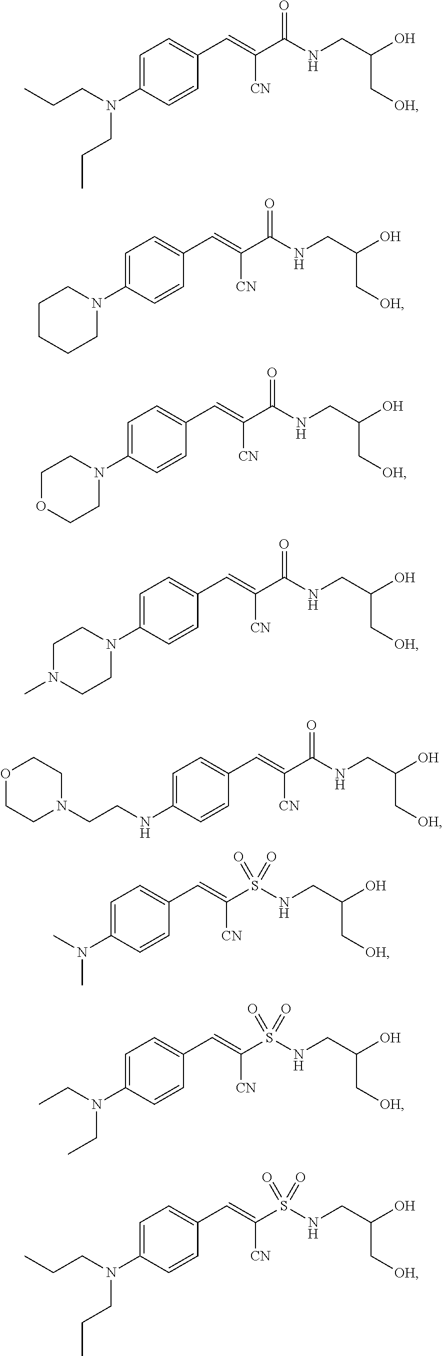

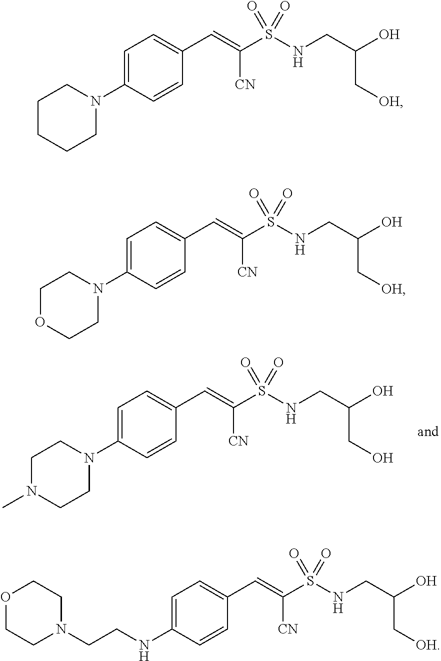 [SMILES: N#C/C(=C\c1ccc(N2CCCCC2)cc1)S(=O)(=O)NCC(O)CO ; N#C/C(=C\c1ccc(N2CCOCC2)cc1)S(=O)(=O)NCC(O)CO ; CN1CCN(c2ccc(/C=C(\C#N)S(=O)(=O)NCC(O)CO)cc2)CC1 ; N#C/C(=C\c1ccc(NCCN2CCOCC2)cc1)S(=O)(=O)NCC(O)CO]

45. The composition of any one of claims 1-22, wherein the compound is

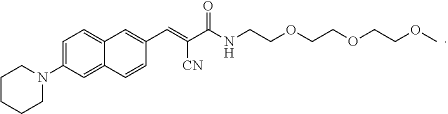 [SMILES: COCCOCCOCCNC(=O)/C(C#N)=C/c1ccc2cc(N3CCCCC3)ccc2c1]

46. The composition of any one of claims 1-22, wherein the compound is

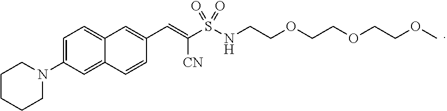 [SMILES: COCCOCCOCCNS(=O)(=O)/C(C#N)=C/c1ccc2cc(N3CCCCC3)ccc2c1]

47. The composition of any one of claims 1-22, wherein the compound is

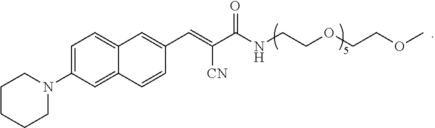

48. The composition of any one of claims 1-22, wherein the compound is

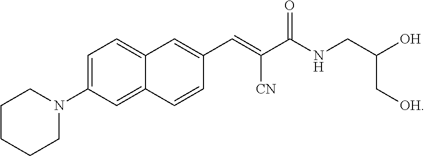 [SMILES: N#C/C(=C\c1ccc2cc(N3CCCCC3)ccc2c1)C(=O)NCC(O)CO]

49. The composition of any one of claims 1-22, wherein the compound is

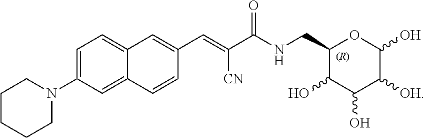

50. The composition of claim 49, wherein the compound is

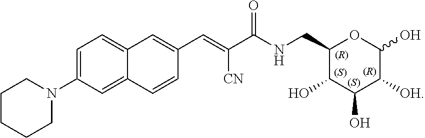

51. The composition of any one of claims 1-22, wherein the compound is

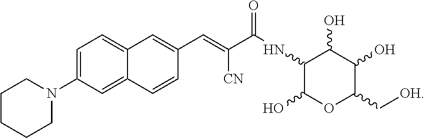 [SMILES: N#C/C(=C\c1ccc2cc(N3CCCCC3)ccc2c1)C(=O)NC1C(O)OC(CO)C(O)C1O]

52. The composition of claim 51, wherein the compound is

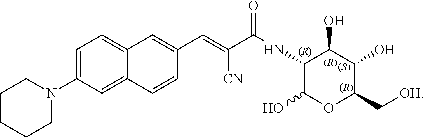

53. The composition of any one of claims 1-22, wherein the compound is

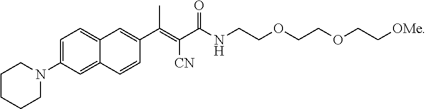 [SMILES: COCCOCCOCCNC(=O)/C(C#N)=C(\C)c1ccc2cc(N3CCCCC3)ccc2c1]

54. The composition of any one of claims 1-22, wherein the compound is

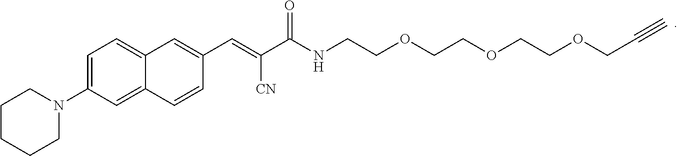 [SMILES: C#CCOCCOCCOCCNC(=O)/C(C#N)=C/c1ccc2cc(N3CCCCC3)ccc2c1]

55. The composition of any one of claims 1-22, wherein the compound is

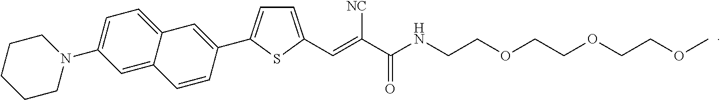 [SMILES: COCCOCCOCCNC(=O)/C(C#N)=C/c1ccc(-c2ccc3cc(N4CCCCC4)ccc3c2)s1]

56. The composition of any one of claims 1-22, wherein the compound is

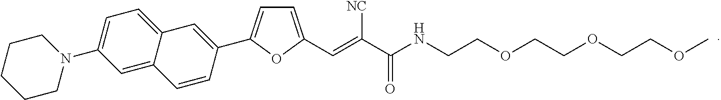 [SMILES: COCCOCCOCCNC(=O)/C(C#N)=C/c1ccc(-c2ccc3cc(N4CCCCC4)ccc3c2)o1]

57. The composition of any one of claims 1-22, wherein the compound is

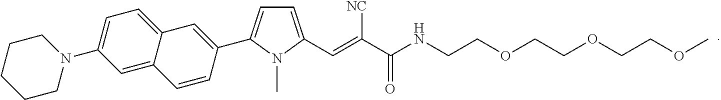 [SMILES: COCCOCCOCCNC(=O)/C(C#N)=C/c1ccc(-c2ccc3cc(N4CCCCC4)ccc3c2)n1C]

58. The composition of any one of claims 1-22, wherein the compound is

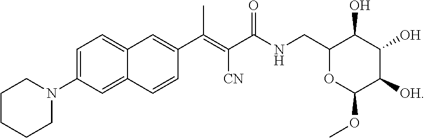 [SMILES: CO[C@H]1OC(CNC(=O)/C(C#N)=C(\C)c2ccc3cc(N4CCCCC4)ccc3c2)[C@@H](O)[C@H](O)[C@H]1O]

59. The composition of any one of claims 1-22, wherein the compound is

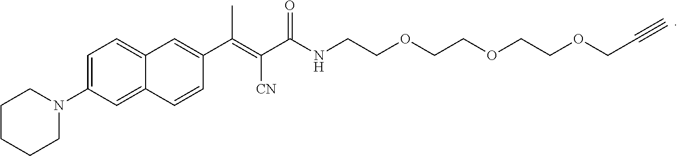 [SMILES: C#CCOCCOCCOCCNC(=O)/C(C#N)=C(\C)c1ccc2cc(N3CCCCC3)ccc2c1]

60. The composition of any one of claims 1-22, wherein the compound is

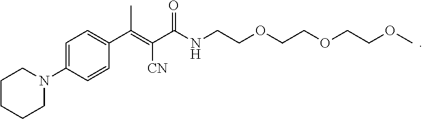 [SMILES: COCCOCCOCCNC(=O)/C(C#N)=C(\C)c1ccc(N2CCCCC2)cc1]

61. The composition of any one of claims 1-22, wherein the compound is

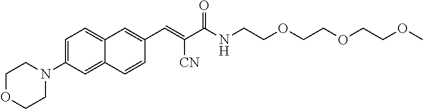 [SMILES: COCCOCCOCCNC(=O)/C(C#N)=C/c1ccc2cc(N3CCOCC3)ccc2c1]

62. A composition comprising a) a human sample selected from the list of human samples consisting of a tissue, a cell population, a cell, a fluid, and an extract and b) a compound of Formula II, or a pharmaceutically acceptable salt thereof: 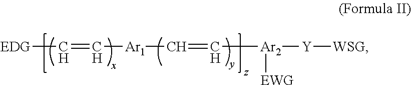
wherein
EDG is an electron donating group;
Ar₂ and each Ar₁ is independently C₁-C₁₄ arylene or C₁-C₁₄ heteroarylene, each optionally substituted with one more R₄₁;
each R₄₁ is independently halogen, —OR₄₂, —CN, —NR₄₃R₄₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene wherein the alkyl, heteroalkyl, cycloalkyl, heterocycloalkyl, arylene, or heteroarylene is optionally substituted with one or more R_{45;}
R₄₂, R₄₃ and R₄₄ are independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₄₅;
each R₄₅ is independently halogen, —OR₄₆, —NR₄₇R₄₈, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
R₄₆, R₄₇ and R₄₈ are independently hydrogen or C₁-C₁₀ alkyl;
EWG is an electron withdrawing group; Y is absent, O, NH, or S;
WSG is hydrogen or a water soluble group;
x is an integer from 0-10;
y is an integer from 0-10; and
z is an integer from 1-10.

63. The composition of claim 62, wherein said sample comprises a cell.

64. The composition of claim 63, wherein said sample comprises a cell selected from the list consisting of a leukocyte, a monocyte, a peripheral blood leukocyte (PBL), a white blood cell, a red blood cell, a skin cell, cheek cell, a hair follicle cell, and a nerve cell.

65. The composition of claim 62, wherein said sample comprises a fluid.

66. The composition of claim 63, wherein said fluid is selected from the list of fluids consisting of as urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, and gastric acid.

67. The composition of claim 63, wherein said fluid is blood.

68. The composition of claim 63, wherein said fluid is saliva.

69. The composition of claim 63, wherein said fluid is urine.

70. The composition of claim 63, wherein said fluid is perspiration.

71. The composition of claim 63, wherein said fluid is tears.

72. The composition of claim 63, wherein said fluid is lymph.

73. The composition of claim 63, wherein said fluid is vaginal fluid.

74. The composition of claim 63, wherein said fluid is semen.

75. The composition of claim 62, wherein said sample comprises a tumor sample.

76. The composition of claim 75, wherein said tumor sample is selected from the list consisting of a tumor tissue, a tumor cell, a tumor fluid, a partially homogenized tumor extract, and a fully homogenized tumor extract.

77. The composition of claim 75, wherein said tumor sample is a sample of a tumor associated with defective p53 protein activity.

78. The composition of any one of claims 62-75, wherein:
EDG is OR₄₉, NR₅₀R₅₁, —SR₅₂, —PR₅₃R₅₄, —NR₅₅C(O)R₅₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₅₇;
each R₅₇ is independently halogen, —OR₅₈, —NR₅₉R₆₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene;
each of R₄₉, R₅₀, R₅₁, R₅₂, R₅₃, R₅₄, R₅₅, R₅₆, R₅₈, R₅₉ and R₆₀ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, each of which except for hydrogen is optionally substituted with one or more R₆₁ and wherein R₅₀ and R₅₁ are optionally joined together to form a heterocycloalkyl or heteroaryl optionally substituted with R₆₁;
each of R₆₁ is independently halogen, —OR₆₂, —NR₆₃R₆₄, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₆₅;
each of R₆₂, R₆₃ and R₆₄ is independently hydrogen or C₁-C₁₀ alkyl; and
each R₆₅ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

79. The composition of any one of claims 62-75, wherein EDG is

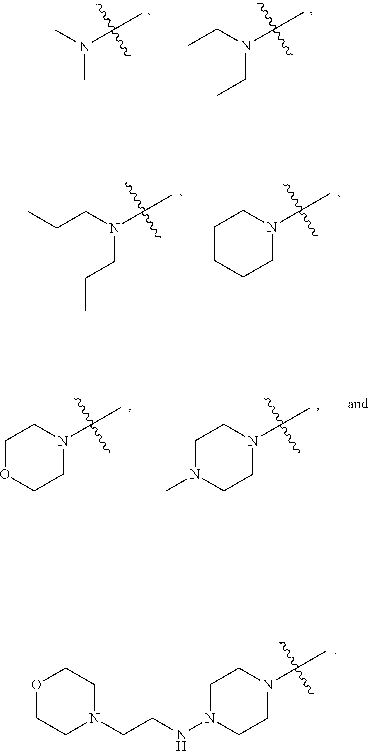

80. The composition of any one of claims 62-75, wherein EDG is

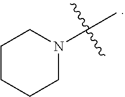 [SMILES: *N1CCCCC1]

81. The composition of any one of claims 62-75, wherein:
EWG is halogen, —CN, —NO₂, —SO₃H, —CR₆₆R₆₇R₆₈, COR₆₉, or COOR₇₀;
each R₆₆, R₆₇ and R₆₈ is independently hydrogen or halogen;
R₆₉ is halogen, hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₁;
R₇₀ is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₂; and
each R₇₁ and R₇₂ is independently C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

82. The composition of any one of claims 62-75, wherein EWG is selected from a group consisting of F, Cl, Br, —C═O, NO₂, —CF₃, —CCl₃, —SO₃ and —CN.

83. The composition of any one of claims 62-75, wherein EWG is —CN.

84. The composition of any one of claims 62-75, wherein Y is absent.

85. The composition of any one of claims 62-75, wherein:
WSG is hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₃;
each R₇₃ is independently halogen, —OR₇₄, —NR₇₅R₇₆, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₇;
each R₇₄, R₇₅ and R₇₆ is independently hydrogen, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene, wherein the alkyl, heteroalkyl, cycloalkyl, heterocycloalkyl, arylene, or heteroarylene is optionally substituted with one or more R₇₇;
each R₇₇ is independently halogen, —OR₇₈, —NR₇₉R₈₀, C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene; and
each of R₇₈, R₇₉ and R₈₀ is independently hydrogen or C₁-C₁₀ alkyl.

86. The composition of claim 85, wherein WSG is hydrogen.

87. The composition of claim 85, wherein WSG is

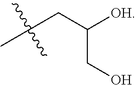 [SMILES: *CC(O)CO]

88. The composition of any one of claims 62-75, wherein WSG is polyethylene glycol, polypropylene glycol, co-polymer of polyethylene glycol and polypropylene glycol, or alkoxy derivatives thereof.

89. The composition of claim 88, wherein WSG is 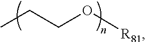
wherein n is an integer from 0-50 and R₈₁ is H, a C₁-C₁₀ alkyl, a C₁-C₁₀ alkenyl, or a C₁-C₁₀ alkynyl wherein each wherein the alkyl, alkenyl, or alkynyl is optionally substituted with one or more C₁-C₁₀ alkyl, C₁-C₁₀ heteroalkyl, C₁-C₁₀ cycloalkyl, C₁-C₁₀ heterocycloalkyl, C₁-C₁₀ arylene, or C₁-C₁₀ heteroarylene.

90. The composition of claim 89, wherein R₈₁ is methyl.

91. The composition of claim 89, wherein R₈₁ is CH₂—C≡CH.

92. The composition of claim 89, wherein n is 0, 1, 2, 3, 4, 5, 6, 7, 8, 9, or 10.

93. The composition of claim 89, wherein n is 3 or 6.

94. The composition of claim 85, wherein the WSG is

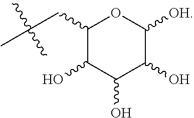 [SMILES: *CC1OC(O)C(O)C(O)C1O]

95. The composition of claim 94, wherein the WSG is

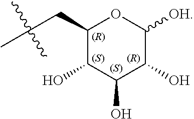

96. The composition of claim 85, wherein the WSG is

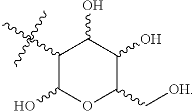

97. The composition of claim 96, wherein the WSG is

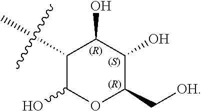

98. The composition of any one of claims 62-75, wherein each of Ar₁ is independently a naphthylene or a phenylene.

99. The composition of any one of claims 62-75, wherein Ar₂ is naphthylene or a phenylene.

100. The composition of any one of claims 62-75, wherein the compound is selected from a group consisting of 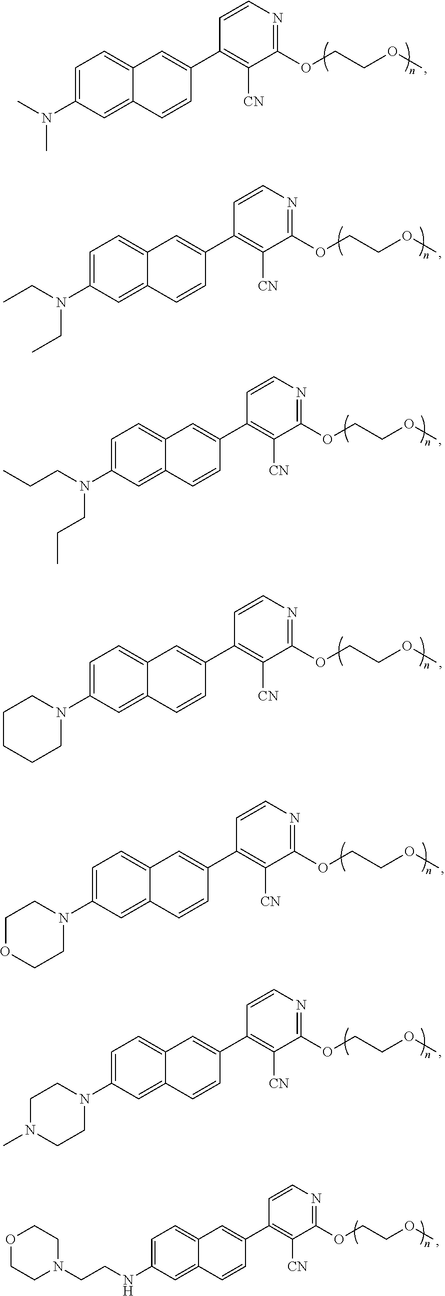 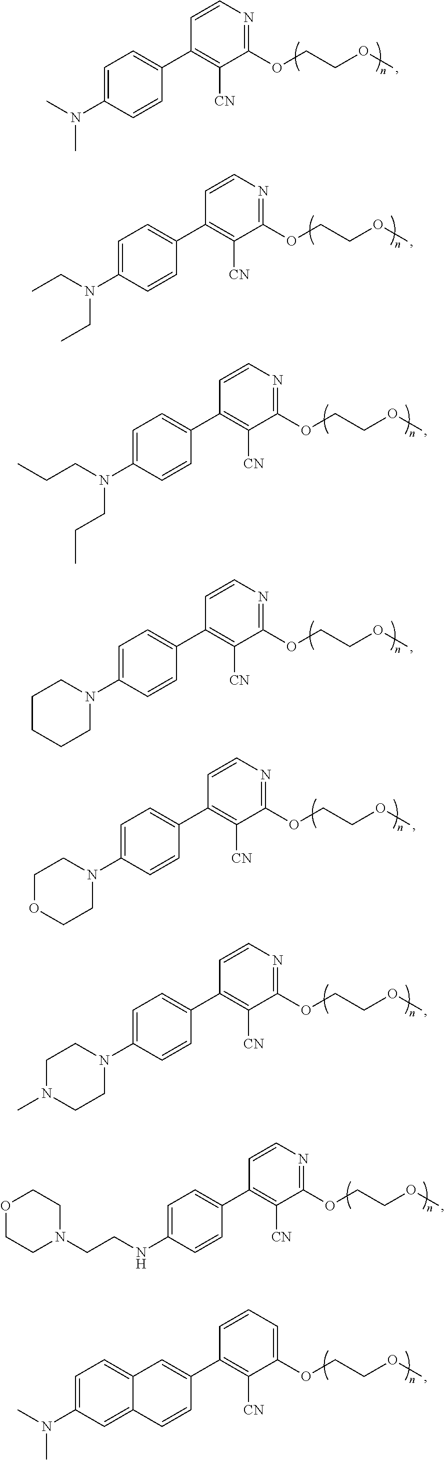 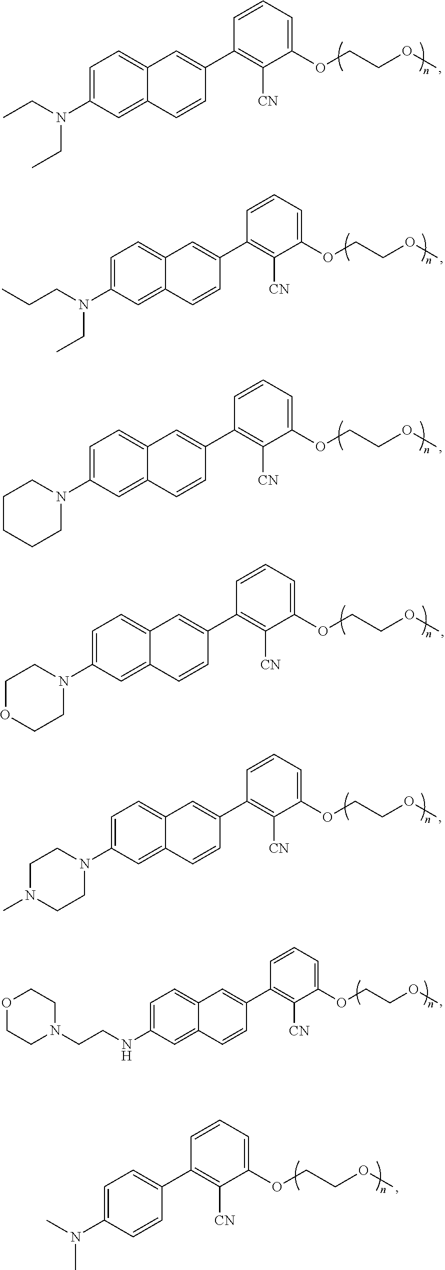 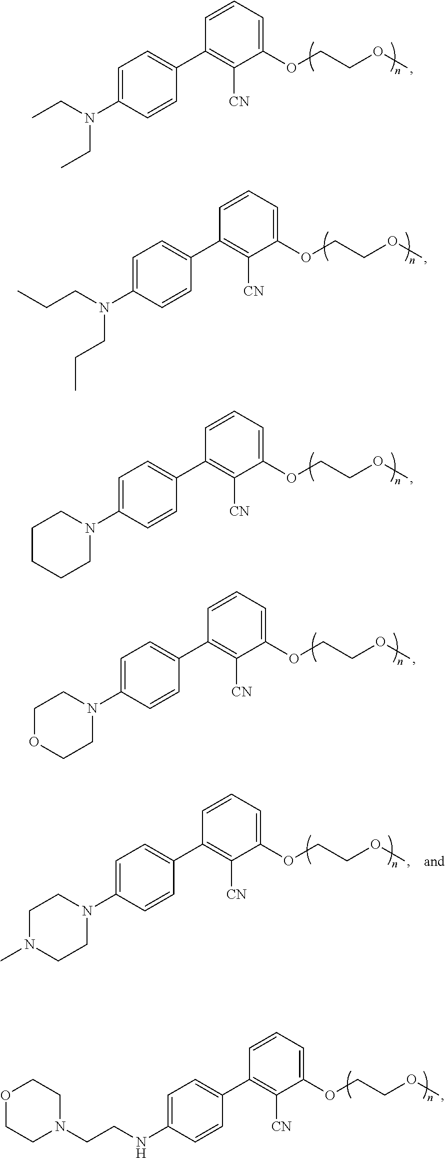
wherein n is an integer with value 0-10.

101. The composition of any one of claims 62-75, wherein the compound is selected from a group consisting of

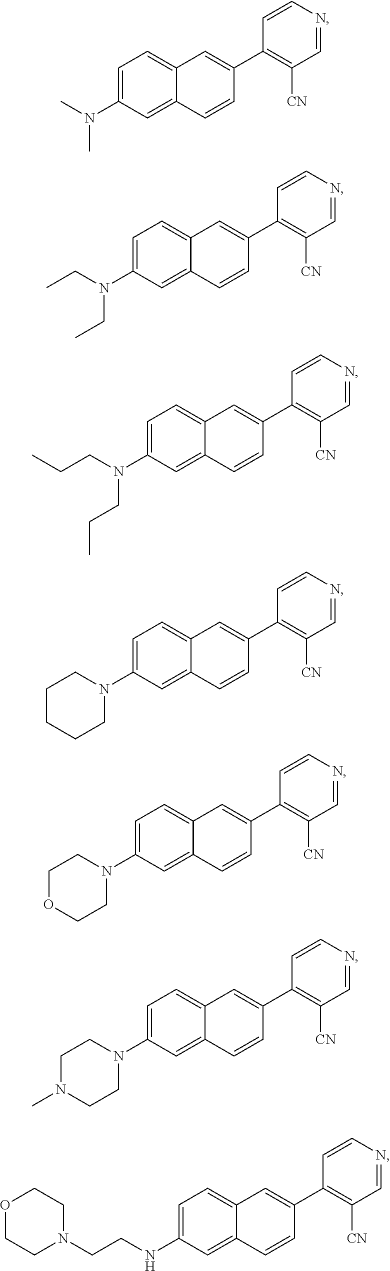

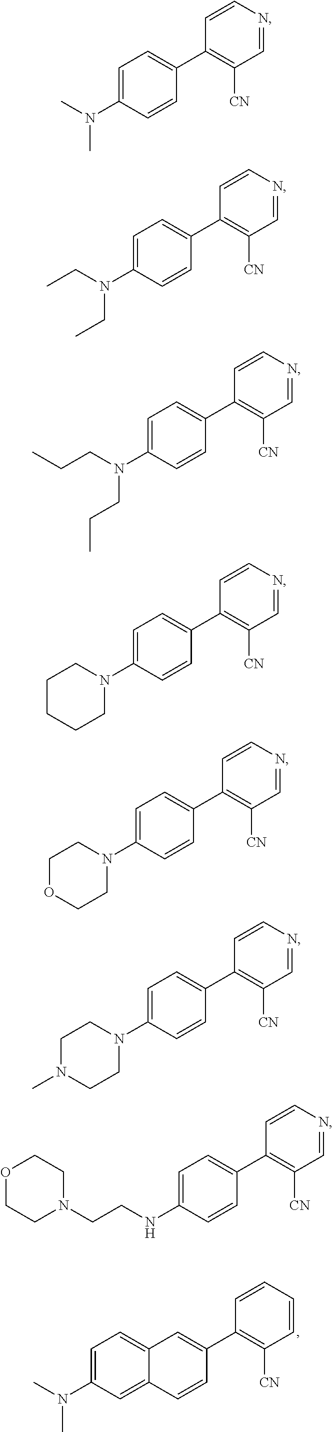

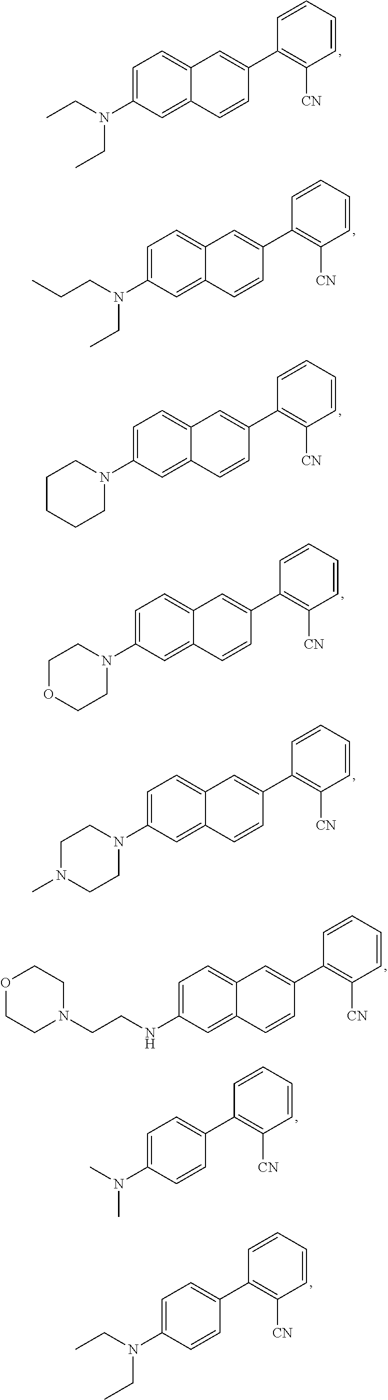

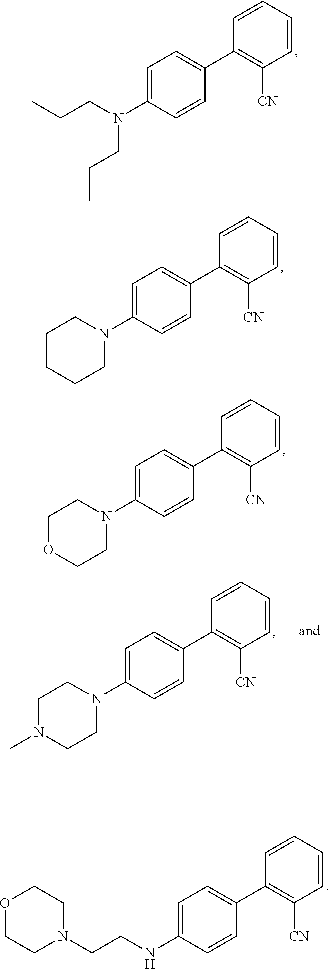 [SMILES: CCCN(CCC)c1ccc(-c2ccccc2C#N)cc1 ; N#Cc1ccccc1-c1ccc(N2CCCCC2)cc1 ; N#Cc1ccccc1-c1ccc(N2CCOCC2)cc1 ; CN1CCN(c2ccc(-c3ccccc3C#N)cc2)CC1 ; N#Cc1ccccc1-c1ccc(NCCN2CCOCC2)cc1]

102. The composition of any one of claims 62-75, wherein the compound is

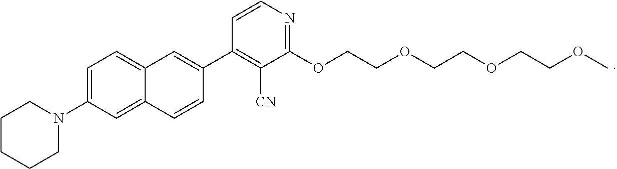 [SMILES: COCCOCCOCCOc1nccc(-c2ccc3cc(N4CCCCC4)ccc3c2)c1C#N]

103. The composition of any one of claims 62-75, wherein the compound is

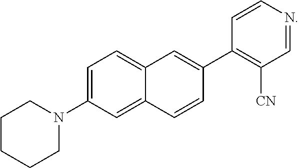 [SMILES: N#Cc1cnccc1-c1ccc2cc(N3CCCCC3)ccc2c1]

104. The composition of any one of claims 62-75, wherein the compound is

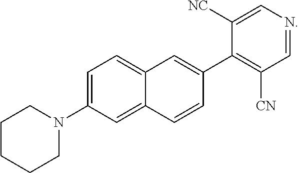 [SMILES: N#Cc1cncc(C#N)c1-c1ccc2cc(N3CCCCC3)ccc2c1]

105. The composition of any one of claims 62-75, wherein the compound is

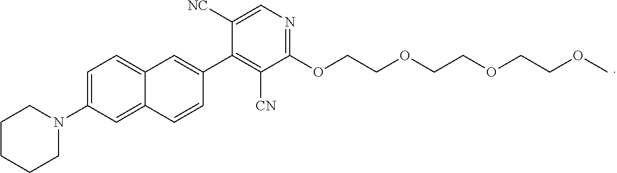 [SMILES: COCCOCCOCCOc1ncc(C#N)c(-c2ccc3cc(N4CCCCC4)ccc3c2)c1C#N]

106. A composition of any one of claims 1-105, comprising an amyloid protein complex.

107. The composition of claim 106, wherein the amyloid protein complex comprises a complex of beta-sheet bound protein monomers.

108. The composition of claim 106, wherein the amyloid protein complex comprises at least one of Aβ peptide, α-Synuclein, prion peptide, huntingtin, serum amyloid A, transthyretin, lysozyme, amylin, immunoglobulin light chain, semen derived enhancer of viral infection, PAB, SEM1, protegrin-1, CsgA-R5, and CsgA-R1, superoxide dismutase, insulin, and p53.

109. A composition of any one of claims 1-105, comprising an amyloid-like protein complex.

110. A method of identifying a person at risk of having an amyloid disease, comprising the steps of
assaying for an amyloid-compound complex in a composition of any one of claims 1-109, wherein the human sample is from said person.

111. The method of claim 110, comprising identifying said person as at risk of having an amyloid disease if the amyloid-compound complex is present above a threshold level.

112. The method of claim 110, comprising identifying said person as not at risk of having an amyloid disease if the amyloid-compound complex is not present above a threshold level.

113. The method of any one of claims 111-112, wherein the threshold level is an amyloid-compound complex level present in a standard sample.

114. The method of any one of claims 111-112, wherein the threshold level is an amyloid-compound complex level present in a negative control sample.

115. The method of any one of claims 111-112, wherein the threshold level is an amyloid-compound complex level present in a sample from an individual free of the amyloid disorder.

116. The method of any one of claims 111-112, wherein the threshold level is an amyloid-compound complex level present in a healthy individual.

117. The method of any one of claims 110-116, wherein the assaying comprises contacting the sample with fluorescence excitation energy.

118. The method of any one of claims 110-117, wherein the assaying comprises measuring fluorescence emission of the sample.

119. The method of any one of claims 110-118, wherein the disorder is selected from the list comprising Alzheimer's disease, Amyloid amyloidosis, Amyloid light chain amyloidosis, amyotrophic lateral sclerosis, apolipoprotein A1, myloidosis, bacterial homeostasis, breast tumors, Cerebral Amyloid Angiopathy, Creutzfeld-Jakob disease, Creutzfeldt-Jacob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, lymphoma, Lysozomal storage disorders, lysozyme amyloidosis, nephrogenic diabetes insipidus, p53-related cancers, Parkinson's disease, pre-eclampsia, Rheumatoid arthritis, senile systemic amyloidosis, skin tumors, Spongiform encephalitis, systemic AL amyloidosis, tumoral amyloidosis, and Type II diabetes.

120. The method of any one of claims 110-118, wherein the disorder is pre-eclampsia.

121. The method of claim 118, wherein the fluorescence emission spectrum corresponds to the amyloid of the amyloid-compound complex.

122. The method of claim 118, wherein the fluorescence emission spectrum indicates the identity of the amyloid of the amyloid-compound complex.

123. The method of claim 118, wherein the fluorescence emission spectrum of the amyloid-compound complex corresponds to the disorder.

124. A method of monitoring an amyloid disorder in a person, comprising the steps of comparing a first assay result taken from a first composition of any one of claims 1-109, wherein said first composition comprises a sample taken from said person at a first time, with a second assay result, taken from a second composition of any one of claims 1-109, wherein said second composition comprises a sample taken from said person at a second time.

125. The method of claim 124, wherein a treatment is administered to the person between the first time and the second time.

126. The method of claim 124, wherein a treatment is administered to the person prior to the first time.

127. The method of any one of claims 124-126, wherein said amyloid disorder is selected from the list consisting of Alzheimer's disease, Amyloid amyloidosis, Amyloid light chain amyloidosis, amyotrophic lateral sclerosis, apolipoprotein A1, myloidosis, bacterial homeostasis, breast tumors, Cerebral Amyloid Angiopathy, Creutzfeld-Jakob disease, Creutzfeldt-Jacob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, lymphoma, Lysozomal storage disorders, lysozyme amyloidosis, nephrogenic diabetes insipidus, p53-related cancers, Parkinson's disease, pre-eclampsia, Rheumatoid arthritis, senile systemic amyloidosis, skin tumors, Spongiform encephalitis, systemic AL amyloidosis, tumoral amyloidosis, and Type II diabetes.

128. The method of any one of claims 124-126, wherein said amyloid disorder is Alzheimer's.

129. The method of any one of claims 124-126, wherein said amyloid disorder is pre-eclampsia.

130. A method of detecting an amyloid disorder in a human subject comprising the steps of:
contacting a sample from the human subject to a molecule having a first spectal profile when unbound and a second spectal profile when bound to a protein aggregate;
determining a spectal profile for the sample contacted to the molecule;
wherein the second spectal profile indicates presence of the amyloid disorder; and
wherein the method has a sensitivity of at least 80% and a specificity of at least 80%.

131. The method of claim 130, wherein the sensitivity is at least 90%.

132. The method of claim 131, wherein the sensitivity is at least 95%.

133. The method of claim 132, wherein the sensitivity is at least 99%.

134. The method of claim 130, wherein the specificity is at least 85%.

135. The method of claim 130, wherein the first spectral profile indicates absence of the amyloid disorder.

136. The method of any one of claims 130-135, wherein the molecule is a molecule of Formula I, or a pharmaceutically acceptable salt thereof.

137. The method of any one of claims 130-135, wherein the molecule is a molecule of Formula II, or a pharmaceutically acceptable salt thereof.

138. The method of any one of claims 130-135, wherein the molecule is compound 1, or a pharmaceutically acceptable salt thereof:

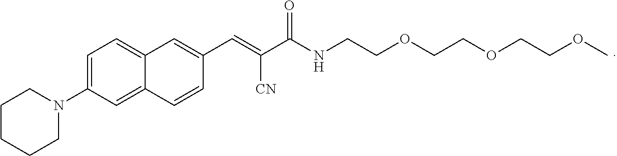 [SMILES: COCCOCCOCCNC(=O)/C(C#N)=C/c1ccc2cc(N3CCCCC3)ccc2c1]

139. The method of any one of claims 130-135, wherein the molecule is compound 2, or a pharmaceutically acceptable salt thereof:

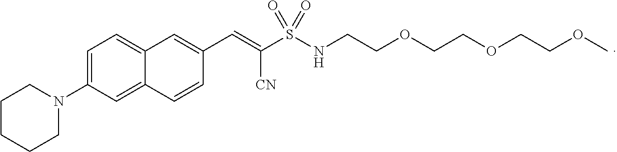 [SMILES: COCCOCCOCCNS(=O)(=O)/C(C#N)=C/c1ccc2cc(N3CCCCC3)ccc2c1]

140. The method of any one of claims 130-135, wherein the molecule is compound 5, or a pharmaceutically acceptable salt thereof:

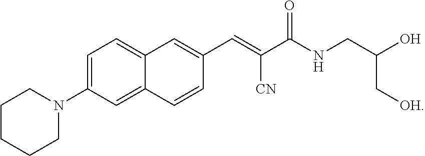 [SMILES: N#C/C(=C\c1ccc2cc(N3CCCCC3)ccc2c1)C(=O)NCC(O)CO]

141. The method of any one of claims 130-135, wherein the molecule is compound 21, or a pharmaceutically acceptable salt thereof:

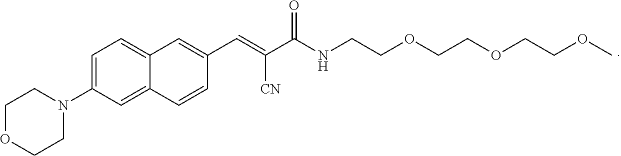 [SMILES: COCCOCCOCCNC(=O)/C(C#N)=C/c1ccc2cc(N3CCOCC3)ccc2c1]

142. The method of any one of claims 130-135, wherein the molecule is compound 22, or a pharmaceutically acceptable salt thereof:

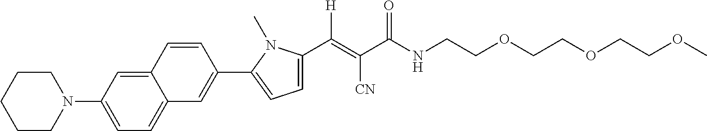 [SMILES: COCCOCCOCCNC(=O)/C(C#N)=C/c1ccc(-c2ccc3cc(N4CCCCC4)ccc3c2)n1C]

143. The method of any one of claims 130-135, wherein the amyloid disorder is selected from the list consisting of Alzheimer's disease, Amyloid amyloidosis, Amyloid light chain amyloidosis, amyotrophic lateral sclerosis, apolipoprotein A1, myloidosis, bacterial homeostasis, breast tumors, Cerebral Amyloid Angiopathy, Creutzfeld-Jakob disease, Creutzfeldt-Jacob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, lymphoma, Lysozomal storage disorders, lysozyme amyloidosis, nephrogenic diabetes insipidus, p53-related cancers, Parkinson's disease, pre-eclampsia, Rheumatoid arthritis, senile systemic amyloidosis, skin tumors, Spongiform encephalitis, systemic AL amyloidosis, tumoral amyloidosis, and Type II diabetes.

144. The method of any one of claims 130-135, wherein the amyloid disorder is pre-eclampsia.

145. The method of any one of claims 130-135, wherein the amyloid disorder comprises an aggregate of at least one of Aβ peptide, α-Synuclein, prion peptide, huntingtin, serum amyloid A, transthyretin, lysozyme, amylin, immunoglobulin light chain, semen derived enhancer of viral infection, PAB, SEMI, protegrin-1, CsgA-R5, and CsgA-R1, superoxide dismutase, insulin, and p53.

146. The method of any one of claims 130-135, wherein the sample comprises a cell selected from the list consisting of a leukocyte, a monocyte, a peripheral blood leukocyte (PBL), a white blood cell, a red blood cell, a skin cell, cheek cell, a hair follicle cell, and a nerve cell.

147. The method of any one of claims 130-135, wherein the sample comprises a fluid selected from the list of fluids consisting of as urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, cystic fluid, and gastric acid.

148. The method of any one of claims 130-135, wherein the sample comprises urine.

149. The method of any one of claims 130-135, wherein the sample comprises a tumor sample.

150. The composition of claim 149, wherein said tumor sample is selected from the list consisting of a tumor tissue, a tumor cell, a tumor fluid, a partially homogenized tumor extract, and a fully homogenized tumor extract.

151. The method of claim 130, wherein the amyloid disorder is pre-eclampsia, the sample comprises urine, the specificity is at least 99%, the sensitivity is at least 85%, and the molecule is compound 1 or a pharmaceutically acceptable salt thereof.

152. The method of claim 130, wherein the amyloid disorder is pre-eclampsia, the sample comprises urine, the specificity is at least 99%, the sensitivity is at least 85%, and the molecule is a molecule of Formula I or a pharmaceutically acceptable salt thereof.

153. The method of claim 130, wherein the amyloid disorder is pre-eclampsia, the sample comprises urine, the specificity is at least 99%, the sensitivity is at least 85%, and the molecule is a molecule of Formula II or a pharmaceutically acceptable salt thereof.
